SEQUENCE LISTING (28 sequences)

SEQ ID NO: 1 - protein, 420 aa, Homo sapiens
  REGION 1..420: note = MISC_FEATURE - Human Trophoblast glycoprotein having UniProt Accession Number:Q13641
  DOMAIN 32..355: note = Extracellular Domain
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 2 - protein, 420 aa, Callithrix jacchus
  REGION 1..420: note = MISC_FEATURE - Callithrix jacchus TPBG having UniProtKB Accession Number: F7I0T3
MPGGCSRGPAAGNGRLRLARLALVLLGWVSSSSPTSSASSSSSSAPFLASAVSAQPLLPGQCPALCECSEAARTVKCVNRNLTEVPTDLPPYVRNLFLTGNQLAVLPAGAFARVPPLAELAALNLSGSRLEEVQAGAFEHLPSLRQLDLSHNPLAVLSPFAFSGSNASVSAPSPLVELILNHIVPPEDERNNRSFEGMVVAALRAGGALHGLHRLELASNHFLYLPRDVLAQLPSLRHLDLSNNSLVSLTYVSFRNLTHLESLHLEDNALKVLHNGTLAELQGLPHVRVFLDNNPWVCDCHMADMVAWLKETEVVQGKYQLTCAFPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 3 - protein, 120 aa, synthetic construct
  REGION 1..120: note = Heavy chain variable region without a signal seqeunce
EVQLVQSGAEVKKPGASVKVSCKASGYSFTGYYMHWVRQAPGQGLEWMGRINPNNGVTLYNQKFKDRVTITRDTSTSTAYMELSSLRSEDTAVYYCARSTMITNYVMDYWGQGTLVTVSS

SEQ ID NO: 4 - protein, 107 aa, synthetic construct
  REGION 1..107: note = Light chain variable region without a signal seqeunce
EIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIK

SEQ ID NO: 5 - protein, 450 aa, synthetic construct
  REGION 1..450: note = Heavy chain without a signal sequence
EVQLVQSGAEVKKPGASVKVSCKASGYSFTGYYMHWVRQAPGQGLEWMGRINPNNGVTLYNQKFKDRVTITRDTSTSTAYMELSSLRSEDTAVYYCARSTMITNYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, synthetic construct
  REGION 1..214: note = Light chain without a signal seqeunce
EIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 5 aa, synthetic construct
  REGION 1..5: note = CDR-H1
GYYMH

SEQ ID NO: 8 - protein, 17 aa, synthetic construct
  REGION 1..17: note = CDR-H2
RINPNNGVTLYNQKFKD

SEQ ID NO: 9 - protein, 11 aa, synthetic construct
  REGION 1..11: note = CDR-H3
STMITNYVMDY

SEQ ID NO: 10 - protein, 11 aa, synthetic construct
  REGION 1..11: note = CDR-L1
KASQSVSNDVA

SEQ ID NO: 11 - protein, 7 aa, synthetic construct
  REGION 1..7: note = CDR-L2
YTSSRYA

SEQ ID NO: 12 - protein, 9 aa, synthetic construct
  REGION 1..9: note = CDR-L3
QQDYNSPPT

SEQ ID NO: 13 - protein, 19 aa, synthetic construct
  REGION 1..19: note = Signal sequence for the heavy chain
MDWTWRILFLVAAATGAHS

SEQ ID NO: 14 - protein, 19 aa, synthetic construct
  REGION 1..19: note = Signal sequence for light chain
MLPSQLIGFLLLWVPASRG

SEQ ID NO: 15 - protein, 233 aa, synthetic construct
MLPSQLIGFLLLWVPASRGDIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 468 aa, synthetic construct
MDWTWRILFLVAAATGAHSEVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 19 aa, synthetic construct
MDWTWRILFLVAAATGAHS

SEQ ID NO: 18 - protein, 19 aa, synthetic construct
MLPSQLIGFLLLWVPASRG

SEQ ID NO: 19 - protein, 236 aa, synthetic construct
MLPSQLIGFLLLWVPASRGDIQMTQSPSTLSASVGDRVTITCQASQSIGSELAWYQQKPGKAPKLLIYRASTLESGVPSRFSGSGSGTEFTLTISSLQPDDFATYYCQQGYTYSEIDNAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 471 aa, synthetic construct
MDWTWRILFLVAAATGAHSEVQLEESGGGLVKPGGSLRLSCAASGIDLSHYVVGWVRQAPGKGLEWVSIIYGSGRTYYANWAKGRFTISRDNSKNTLYLQMNSLRAEDTAVYYCARDASVSVYYWGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 19 aa, synthetic construct
MDWTWRILFLVAAATGAHS

SEQ ID NO: 22 - protein, 19 aa, synthetic construct
MLPSQLIGFLLLWVPASRG

SEQ ID NO: 23 - protein, 469 aa, synthetic construct
MDWTWRILFLVAAATGAHSEVQLVQSGAEVKKPGASVKVSCKASGYSFTGYYMHWVRQAPGQGLEWMGRINPNNGVTLYNQKFKDRVTITRDTSTSTAYMELSSLRSEDTAVYYCARSTMITNYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 469 aa, synthetic construct
MDWTWRILFLVAAATGAHSEVQLVQSGAEVKKPGASVKVSCKASGYSFTGYYMHWVRQAPGQGLEWMGRINPNNGVTLYNQKFKDRVTITRDTSTSTAYMELSSLRSEDTAVYYCARSTMITNYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 469 aa, synthetic construct; identical to SEQ ID NO: 23

SEQ ID NO: 26 - protein, 469 aa, synthetic construct; identical to SEQ ID NO: 24

SEQ ID NO: 27 - protein, 469 aa, synthetic construct; identical to SEQ ID NO: 24

SEQ ID NO: 28 - protein, 469 aa, synthetic construct; identical to SEQ ID NO: 23

(19) United States
(12) Patent Application Publication
(10) Pub. No.: US 2024/0245798 A1
(43) Pub. Date: Jul. 25, 2024

(54) NOVEL ANTI-TPBG ANTIBODY AND ANTIBODY-DRUG-CONJUGATES BASED THEREON, THERAPEUTIC METHODS AND USES THEREOF

(71) Applicant: Tubulis GmbH, Planegg/Martinsried (DE)

(72) Inventors: Jonas Helma-Smets, München (DE); Annette Vogl, München (DE); Saskia Schmitt, München (DE); Isabelle Mai, München (DE); Sarah Herterich, München (DE); Dominik Schumacher, München (DE); Marc-André Kasper, München (DE); Philipp Cyprys, Deisenhofen (DE); Marcus Gerlach, München (DE)

(21) Appl. No.: 18/489,627

(22) Filed: Oct. 18, 2023

(30) Foreign Application Priority Data
Oct. 18, 2022 (EP) 22202240.2
May 11, 2023 (EP) 23172971.6

(51) Int. Cl. A61K 47/68; A61P 35/00; C07K 16/30

(52) U.S. Cl. CPC A61K 47/6851; A61K 47/68037; A61K 47/6889; A61P 35/00; C07K 16/30; C07K 2317/77

(57) ABSTRACT

The present invention relates to novel anti-TPBG antibody comprising Fc silencing mutations such as leucine (L) to alanine (A) substitution at the position 234 and 235 (LALA mutations), antibody-drug-conjugates (ADCs) based thereon as well as to therapeutic methods and uses thereof, particularly in relation to cancer treatment.

### CROSS-REFERENCE TO RELATED APPLICATIONS

The present application claims the right of priority of European patent application EP22202240.2 filed with the European Patent Office on 18 Oct. 2022, the entire content of which is incorporated herein for all purposes.

The present application claims the right of priority of European patent application EP23172971.6 filed with the European Patent Office on 11 May 2023, the entire content of which is incorporated herein for all purposes.

### REFERENCE TO AN ELECTRONIC SEQUENCE LISTING

The contents of the electronic sequence listing (910271_405_SeqListing.xml; Size: 37,027 bytes; and Date of Creation: Oct. 18, 2023) is herein incorporated by reference in its entirety.

### TECHNICAL FIELD OF THE INVENTION

The present invention relates to novel anti-TPBG antibody comprising Fc silencing mutations such as leucine (L) to alanine (A) substitution at the position 234 and 235 (LALA mutations), novel antibody-drug-conjugates (ADCs) based thereon as well as to therapeutic methods and uses thereof, particularly in relation to cancer treatment.

### BACKGROUND OF THE INVENTION

The oncofetal antigen 5T4 (that can also be interchangeably referred to as trophoblast glycoprotein (TPBG), is a 72-kDa glycoprotein that is typically only expressed during embryonic development, whereas expression in normal adult tissues is very limited. Expression of 5T4, however, is reported to be significantly upregulated in many types of carcinomas, including, but not limited to, cancers of the lung, breast, stomach, prostate, colon, and ovaries, and its expression has been correlated with poor prognosis in multiple indications. Given these features, 5T4 has been regarded as a highly important cancer target and different therapeutic modalities are being tested to target 5T4, including antibody-drug conjugates, bispecific T-cell engagers, CAR-T approaches, and cancer vaccines. The humanized mAb H8 is known in the art (e.g., Shaw et al., 2000, Biochim Biophys Acta 2000 Dec. 15; 1524(2-3):238-46) and has been derived from the original murine 5T4 monoclonal antibody binding to TPBG (Hole and Stern, 1988 and Shaw et al., 2000). Several first-generation antibody-drug conjugates have also been developed in the past, which were discontinued in part due to linker-payload-related toxicities.

Accordingly, there is a need for novel antibodies with improved characteristics and ADCs based thereon that have improved toxicity profiles with greater functionality. The technical problem of the present invention is therefore to comply with this need.

The present invention complies with the needs inter alia by providing novel anti-TPBG antibody comprising Fc silencing mutations such as leucine (L) to alanine (A) substitution at the position 234 and 235 (LALA mutations) in Fc region capable of reducing effector function/s of immune cells and novel TPBG-targeting antibody-drug-conjugates (ADCs) based thereon, which were generated by applying the P5 conjugation technology (e.g., WO2018/041985), which is based on the modification of interchain-Cysteine residues with unsaturated phosphonamidate reagents, as well as to therapeutic methods and uses thereof, particularly in relation to cancer treatment.

### SUMMARY OF INVENTION

The technical problem is solved by the subject-matter as defined in the claims.

Accordingly, the present invention relates to an anti-TPBG antibody (e.g., an antibody against TPBG_HUMAN Trophoblast glycoprotein, e.g., having UniProt Accession Number: Q13641 or SEQ ID NO: 1), wherein said anti-TPBG antibody comprising Fc silencing mutations such as leucine (L) to alanine (A) substitution at the position 234 and 235 (LALA mutations) and is capable of the following: binding to human Trophoblast glycoprotein (TPBG) (e.g., having UniProt Accession Number: Q13641 or SEQ ID NO: 1); having cross-reactivity with white-tufted-ear marmoset (e.g., Callithrix jacchus) TPBG (e.g., having UniProtKB Accession Number: F710T3 or SEQ ID NO: 2); and internalization, preferably by the means of the antigen-mediated antibody internalization.

The present invention further relates to monoclonal human or humanized IgG1 anti-TPBG antibody, preferably comprising kappa (κ) light chain.

The present invention further relates to a hybridoma, wherein said hybridoma produces the antibody of the present invention.

The present invention further relates to a nucleic acid encoding the antibody of the present invention.

The present invention further relates to an expression vector comprising at least one of the nucleic acid molecules of the present invention.

The present invention further relates to an isolated host cell (e.g., an isolated recombinant host cell) comprising the vector and/or nucleic acid of the present invention.

The present invention further relates to an antibody drug conjugate (ADC) comprising the anti-TPBG antibody of the present invention.

The present invention further relates to an antibody drug conjugate (ADC) of the present invention comprising the anti-TPBG antibody of the present invention (e.g., humanized monoclonal TPBG-specific IgG1 antibody) conjugated to a cytotoxic payload/drug: (a) wherein the cytotoxic payload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or (b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or (c) wherein the cytotoxic payload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or (d) wherein the linker (L) comprises a valine-citrulline-PAB moiety or a valine-alanine-PAB moiety; and/or (e) wherein cytotoxic payload is Exatecan, conjugated via a chemical valine-citrulline-PAB or a valine-alanine-PAB release unit, wherein said release unit is cleavable by a protease. Preferably, a drug (e.g., cytotoxic moieties (e.g., cytotoxic payload/s, e.g. Tubulin disrupting agents, e.g., Topoisomerase-1 inhibitor/s, e.g., Auristatins or camptothecin, e.g., MMAE (monomethyl auristatin E) or MMAF (monomethyl auristatin F), e.g., Exatecan) to antibody ratio (DAR) is in the range between 0 and 20, preferably between 1 and 20, more preferably between 2 to 10; further preferably DAR is in the range from 4 to 8, most preferably DAR is 4 or 8.

The present invention further relates to a composition or kit comprising the anti-TPBG, antibody drug conjugate (ADC), hybridoma, nucleic acid, expression vector and/or host cell of the present invention.

The present invention further relates to method of synthesis of the antibody drug conjugates (ADCs) of the present invention.

The present invention further relates to methods of treatment and uses of the antibody, antibody drug conjugate (ADC), nucleic acid, expression vector, host cell, composition and/or kit of the present invention.

### Overview of the Sequence Listing

As described herein and unless otherwise stated references are made to UniProtKB Accession Numbers (https://www.uniprot.org/release-notes/2022-08-03-release, e.g., as available in UniProt release 2022_03, published Aug. 3, 2022).

This application contains a Sequence Listing in computer readable form, which is incorporated herein by reference.

### BRIEF DESCRIPTION OF THE DRAWINGS

The invention will be better understood with reference to the detailed description when considered in conjunction with the non-limiting examples and the accompanying drawings, respectively. The Figures show:

FIG. 1 shows the binding of the antibody of the present invention to human 5T4, evaluated by Flow Cytometry. Graph shows means of n=2±SEM.

FIGS. 2A and 2B show binding of the antibody of the present invention to human 5T4, evaluated by ELISA. Graph shows means of n=2±SEM.

FIG. 3 shows binding of the antibody of the present invention to white-tufted-ear marmoset 5T4, evaluated by Flow Cytometry. Graph shows means of n=2±SD.

FIG. 4 shows internalization of the antibody of the present invention evaluated via flow cytometry.

FIGS. 5A to 5C show in vitro cytotoxicity evaluated on targeted cell lines via Resazurin assay.

FIG. 6 shows bystander activity of the ADC.

FIG. 7 shows in vitro inhibition of Topoisomerase-1, by the delivery of Exatecan via the ADC.

FIG. 8 shows in vivo efficacy of the ADC in a mice xenograft model with respect to tumor volume reduction.

FIG. 9 shows tolerability of the ADC in a mice xenograft model with respect to the body weight.

FIG. 10 shows In vivo PK Evaluation of the ADC.

FIG. 11 shows the UV chromatogram of the LC/MS measurement of P5(PEG12)-COOH.

FIG. 12 shows the UV chromatogram of the LC/MS measurement of P5(PEG24)-OSu.

FIG. 13 the UV chromatogram of the LC/MS measurement of NH2-VC-PAB-Exatecan TFA salt.

FIG. 14 the UV chromatogram of the LC/MS measurement of NH2-VA-PAB-Exatecan TFA salt.

FIG. 15 the UV chromatogram of the LC/MS measurement of P5(PEG2)-VC-PAB-Exatecan.

FIG. 16 the UV chromatogram of the LC/MS measurement of P5(PEG12)-VC-PAB-Exatecan.

FIG. 17 the UV chromatogram of the LC/MS measurement of P5(PEG24)-VC-PAB-Exatecan.

FIG. 18 the UV chromatogram of the LC/MS measurement of P5(PEG12)-VA-PAB-Exatecan.

FIG. 19 the UV chromatogram of the LC/MS measurement of P5(PEG12)-Exatecan.

FIGS. 20A to 20C show analytical characterization of the ADC, synthesized and purified via CEX from the anti-TPBG antibody, conjugated to P5(PEG24)-VC-PAB-Exatecan. FIG. 20A) Analytical SEC, FIG. 20B) Analytical HIC, FIG. 20C) Analytical MS spectrum after conjugation. The Data clearly shows that the ADC is fully conjugated to DAR8 with only low amounts of aggregates being present after purification.

FIG. 21 shows exemplary sequences of the anti-TPBG antibody of the present invention.

FIG. 22 shows results of an ex vivo serum stability analysis of the anti-TPBG-P5(PEG24)-VC-PAB-Exatecan (DAR 8), which is a representative ADC of the present invention.

FIGS. 23A to 23V show results of an in vivo efficacy analysis of the anti-TPBG-P5(PEG24)-VC-PAB-Exatecan (DAR 8), which is a representative ADC of the present invention, in patient-derived xenograft models (PDX).

FIG. 24 shows results of an in vivo toxicity analysis of the anti-TPBG-P5(PEG24)-VC-PAB-Exatecan (DAR 8), which is a representative ADC of the present invention, in marmoset monkeys.

FIG. 25 shows a MS analysis of the anti-TPBG comparison ADC1, synthesized by the method described herein. Signals are annotated with the measured mass in Dalton and the absolute intensities. The Drug-to-Antibody ratio has been estimated from the MS signals to 4.2. LC: light chain of Anti-TPBG comparison antibody1, HC: heavy chain of Anti-TPBG comparison antibody 1.

FIG. 26 shows a MS analysis of the Anti-TPBG comparison ADC2, synthesized by the method described above. Signals are annotated with the measured mass in Dalton and the absolute intensities. The Drug-to-Antibody ratio has been estimated from the MS signals to 1.7. LC: light chain of Anti-TPBG comparison antibody2, HC: heavy chain of Anti-TPBG comparison antibody 2.

FIGS. 27A to 27D shows a cytotoxicity dose-response of anti-TPBG-P5(PEG24)-VC-PAB-Exatecan DAR 8 and a corresponding non-targeting isotype control conjugate compared to anti-TPBG comparison ADC1 on 4 different cell lines. Shown are mean and SD of two measurements, as well as a dose-response-fit. Fluorescence in % of medium control is equal to % of viable cells.

FIGS. 28A to 28C show a cytotoxicity dose-response of anti-TPBG-P5(PEG24)-VC-PAB-Exatecan DAR 8 and a corresponding non-targeting isotype control conjugate compared to Anti-TPBG comparison ADC2 on 3 different cell lines. Shown are mean and SD of two measurements, as well as a dose-response-fit. Fluorescence in % of medium control is equal to % of viable cells.

FIGS. 29A and 29B show a cytotoxicity dose-response of target negative cells (SW-620) after the transfer of cell culture supernatant of two different 5T4-positive cell lines (MDA-MB-468 and BXPC-3) that were pre-treated with serial dilutions of anti-TPBG-P5(PEG24)-VC-PAB-Exatecan DAR 8 or Anti-TPBG comparison ADC2 Shown are mean and SD of two measurements, as well as a dose-response-fit. Fluorescence in % of medium control is equal to % of viable cells.

FIG. 30 shows a cytotoxicity dose-response of serial dilutions of the anti-TPBG-P5(PEG24)-VC-PAB-Exatecan DAR 8 ADC in co-cultures of a target-positive (BX-PC-3) and target-negative cell line (SW-620). Cell killing curves are shown separately for each cell-line. Shown are mean and SD of two measurements, as well as a dose-response-fit.

FIGS. 31A to 31C show a dose-dependent induction of DNA-damage and apoptosis markers in response to treatment with increasing concentrations of anti-TPBG-P5(PEG24)-VC-PAB-Exatecan DAR8. A corresponding non-targeting isotype control conjugate was included as negative control. MDA-MB-468 cells (5T4^{high}) were treated with increasing concentrations (0.05-12 μg/ml) of anti-TPBG-P5(PEG24)-VC-PAB-Exatecan DAR8 or the Isotype ADC (Isotype-P5(PEG24)-VC-PAB-Exatecan DAR8) for 72 h, Cells were stained for cleaved PARP (left), Caspase 3 (middle) and pH2AX (right) and analyzed via flow cytometry. Graphs show means of n=2±SEM.

FIG. 32 shows binding of the Fc region of the anti-TPBG-HC-LALA antibody versus the anti-TPBG-HC-wt antibody to recombinant hexameric C1q complement protein which was measured in a HTRF (Homogenous Time-Resolved Fluorescence) based human C1q binding assay (HTRF Human C1q Binding Kit, Cisbio) according to manufacturer's instructions. In brief, serial dilutions 280 nM-11.6 nM of all tested antibodies (anti-TPBG mAb HC-wt, anti-TPBG mAb HC-LALA, α-MHC-I positive, IgG1 kit standard) were measured. HTRF ratio was calculated by dividing the acceptor emission signal at 665 nm by the donor emission signal at 620 nm and multiplying by 10000. Graph shows HTRF ratio at 70 nM concentration with the background (diluent only) HTRF ratio subtracted. Graph shows means of n=2±SD.

FIGS. 33A to 33D show dose-dependent binding of the Fc region of the anti-TPBG-HC-LALA antibody and the anti-TPBG-P5(PEG24)-VC-PAB-Exatecan DAR8 ADC versus the anti-TPBG-HC-wt antibody to recombinant human FcRn and FcγR which was measured using Lumit™ FcγR Binding Immunoassays (FcγRn, FcγRI, FcγRIIa/CD32 R131/H131 polymorphism, FcγRIIIa/CD16 V158/F158 polymorphism; Promega) according to the manufacturer's instructions. Serial dilutions of anti-TPBG HC-wt, anti-TPBG HC-LALA and anti-TPBG-P5(PEG24)-VC-PAB-Exatecan DAR8, a standard and trastuzumab as positive control were incubated with the Tracer-LgBiT and a FcγR-SmBiT. In the absence of an antibody analyte or if no interaction of the tested antibody with FcγRs takes place, Tracer-LgBiT binds to the FcγR-SmBiT target, resulting in maximum luminescence signal. In the case of successful interaction with FcγRs, the tested antibody/ADC will compete with Tracer-LgBiT for binding to the FcγR target, resulting in a concentration-dependent decrease in the luminescent signal. Luminescence was measured on a microplate reader Infinite M200 Pro (Tecan). Graphs show n=1.

FIGS. 34A to 34D show a calcein release-based antibody-dependent cellular cytotoxicity (ADCC) assay. Co-cultures of healthy donor (HD)-derived NK cells and calcein-stained target-positive tumor cells (MDA-MB-468 or DU-145), in a 4:1 ratio, were incubated with 15 μg/ml of indicated antibodies or ADCs (anti-MHC-I antibody served as positive control). The percentage of specific killing was calculated by dividing the calcein released by antibody-mediated cell killing from calcein released by Triton X-permeabilized cells (maximum killing). Graphs show means of n=2±SEM.

FIG. 35A shows the results of an in vivo efficacy analysis of the anti-TPBG-P5(PEG24)-VC-PAB-Exatecan (DAR 8), which is a representative ADC of the present invention, in a patient-derived head and neck cancer xenograft model (PDX, HN11218). Shown on the top is tumor volume over time after treatment once at day 0 with different dose levels of the anti-TPBG-P5(PEG24)-VC-PAB-Exatecan (DAR 8) (1, 3 and 5 mg ADC/kg bodyweight), an isotype control carrying the same amount of linker-payload (5 mg/kg) versus untreated (vehicle). Shown on the bottom are bodyweights of the animals treated with different dose levels of the anti-TPBG-P5(PEG24)-VC-PAB-Exatecan (DAR 8), an isotype control carrying the same amount of linker-payload versus untreated (vehicle). All results are shown as Mean and SEM of 8 animals per group. FIG. 35B shows the in vivo PK evaluation for total and intact ADC for the three dose levels of the dose-response efficacy study as Mean and SD from three measurements per time point from mice treated once at day 0 with different dose levels of the anti-TPBG-P5(PEG24)-VC-PAB-Exatecan (1, 3 and 5 mg ADC/kg bodyweight). In vivo PK evaluation has been performed as described in example 1.

FIGS. 36A and 36B show results of an in vivo efficacy analysis of the anti-TPBG-P5(PEG24)-VC-PAB-Exatecan (DAR 8), which is a representative ADC of the present invention, in a patient-derived NSCLC xenograft model (PDX, Lu9744). Shown on the left is tumor volume over time after treatment once at day 0 with different dose levels of the anti-TPBG-P5(PEG24)-VC-PAB-Exatecan (DAR 8) (1, 3 and 5 mg ADC/kg bodyweight) versus untreated (vehicle). Shown on the right are bodyweights of the animals treated with different dose levels of the anti-TPBG-P5(PEG24)-VC-PAB-Exatecan (DAR 8) versus untreated (vehicle). All results are shown as Mean and SEM of 4 animals per group.

### DETAILED DESCRIPTION OF THE INVENTION

The present invention is described in detail in the following and is also illustrated by the appended examples and figures.

The present inventors produced and characterized novel specific anti-TPBG antibody for specifically targeting the extracellular domain of TPBG. This is particularly advantageous as it relates to a new therapeutic method for treating cancer (e.g., preferably said cancer is selected from the group consisting of: Breast cancer, Head and neck cancer, Ovary cancer, Endometrium cancer, Uterine cervix cancer, Rectum cancer, Colon cancer, Esophagus cancer, Stomach cancer, Lung cancer, Kidney cancer, Adrenal gland cancer, Bladder cancer, Liver cancer, Sarcoma, Brain cancer, Nevi and Melanomas, Urogenital cancer, Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers, Retinoblastom, Thyroid cancer, Fallopian tube cancer; further preferably said cancer is a solid cancer, e.g., selected from the group consisting of: Breast cancer, Head and neck cancer, Ovarian cancer, Endometrial cancer, Uterus cancer (e.g., including cancers of the muscle sheets), Cervical cancer, Rectum cancer, Colon cancer, Anal cancer, Esophagus cancer, Stomach cancer, Lung cancer, Kidney cancer, Adrenal gland cancer, Bladder cancer, Liver cancer, Sarcoma (e.g., including osteosarcoma and Kaposi sarcoma), Brain cancer (e.g., including pituitary tumor/s), Nevi and Melanoma cancers, Skin cancers (e.g., including squamous cell carcinoma and melanoma), Urogenital cancer (e.g., ureter and bladder cancer, testicular cancer, prostate cancer, penile cancer), Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers (e.g., including lymphoma, leukemia, myeloma, Myelodysplastic syndromes, myelofibrosis), Eye cancers (e.g., including Retinoblastoma), Neuroendocrine tumors, Cancer of unknown primary (CUP)).

The novel antibody of the present invention is a humanized anti-5T4 monoclonal antibody (mAb) comprising Fc silencing mutations such as leucine (L) to alanine (A) substitution at the position 234 and 235 (LALA mutations).

The antibody drug conjugate (ADC) of the present invention comprising the anti-TPBG antibody of the present invention which is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties (e.g., cytotoxic payloads, e.g., Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan), preferably via one or more linkers, further preferably via one or more phosphonamidate linkers.

### Definitions

### Antibodies

An “antibody” when used herein may refer to a protein comprising one or more polypeptides (comprising one or more binding domains and/or antigen binding portion, preferably antigen binding domains) substantially or partially encoded by immunoglobulin genes or fragments of immunoglobulin genes. The term “immunoglobulin” (Ig) is used interchangeably with “antibody” herein. The recognized immunoglobulin genes include the kappa, lambda, alpha, gamma, delta, epsilon and mu constant region genes, as well as myriad immunoglobulin variable region genes. In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2, with IgG being preferred in the context of the present invention. An antibody of the present invention is also envisaged which has an IgE constant domain or portion thereof that is bound by the Fc epsilon receptor I. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each light chain includes an N-terminal variable (V) domain (VL) and a constant (C) domain (CL). Each heavy chain includes an N-terminal V domain (VH), three or four C domains (CHs), and a hinge region. The constant domains are not involved directly in binding an antibody to an antigen, but can exhibit various effector functions, such as participation of the antibody dependent cellular cytotoxicity (ADCC). If an antibody should exert ADCC, it is preferably of the IgG1 subtype, while the IgG4 subtype would not have the capability to exert ADCC.

The term “antibody” also includes, but is not limited to, but encompasses monoclonal, monospecific, poly- or multi-specific antibodies such as bispecific antibodies, humanized, camelized, human, single-chain, chimeric, synthetic, recombinant, hybrid, mutated, grafted, and in vitro generated antibodies, with chimeric or humanized antibodies being preferred. The term “humanized antibody” is commonly defined for an antibody in which the specificity encoding CDRs of HC and LC have been transferred to an appropriate human variable frameworks (“CDR grafting”). The term “antibody” also includes scFvs, single chain antibodies, diabodies or tetrabodies, domain antibodies (dAbs) and nanobodies. In terms of the present invention, the term “antibody” shall also comprise bi-, tri- or multimeric or bi-, tri- or multifunctional antibodies having several antigen binding sites.

Furthermore, the term “antibody” as employed in the invention also relates to derivatives of the antibodies (including fragments) described herein. A “derivative” of an antibody comprises an amino acid sequence which has been altered by the introduction of amino acid residue substitutions, deletions or additions. Additionally, a derivative encompasses antibodies which have been modified by a covalent attachment of a molecule of any type to the antibody or protein. Examples of such molecules include sugars, PEG, hydroxyl-, ethoxy-, carboxy- or amine-groups but are not limited to these. In effect the covalent modifications of the antibodies lead to the glycosylation, pegylation, acetylation, phosphorylation, amidation, without being limited to these.

The antibody of the present invention is preferably an “isolated” antibody. “Isolated” when used to describe antibodies disclosed herein, means an antibody that has been identified, separated and/or recovered from a component of its production environment. Preferably, the isolated antibody is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antibody will be prepared by at least one purification step.

Antibodies described herein can be used for diagnostic purposes, including sample testing and in vivo imaging, and for this purpose the antibody (or binding fragment thereof) can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing. The detectable labels can be any of the various types used currently in the field of in vitro diagnostics, including particulate labels including metal sols such as colloidal gold, isotopes, chromophores including fluorescent markers, biotin, luminescent markers, phosphorescent markers and the like, as well as enzyme labels that convert a given substrate to a detectable marker, and polynucleotide tags that are revealed following amplification such as by polymerase chain reaction. A biotinylated antibody would then be detectable by avidin or streptavidin binding. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{l,2-dioxetane-3,2′-(5′-chloro)tricyclo{3.3.1.1 3,7}decan}-4-yl) phenyl phosphate (CSPD), as well as CDP and CDP-star® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptors); and B cell activation. In order to exert effector functions an antibody, so to say, recruits effector cells.

As used herein the term “antigen binding portion” refers to a fragment of immunoglobulin (or intact antibody), and encompasses any polypeptide comprising an antigen-binding fragment or an antigen-binding domain. Preferably, the fragment such as Fab, F(ab′), F(ab′)₂, Fv, scFv, Fd, disulfide-linked Fvs (sdFv), and other antibody fragments that retain antigen-binding function as described herein. Typically, such fragments would comprise an antigen-binding domain and have the same properties as the antibodies described herein. Accordingly, said fragment is preferably also capable of binding to an extracellular domain of the TPBG.

As used herein, the term “specifically binds” refers to antibodies or fragments or derivatives thereof that specifically bind to TPBG protein and do not specifically bind to another protein. The antibodies or fragments or derivatives thereof according to the invention bind to a TPBG protein through the variable domain of the antibody.

The pairing of a VH and VL together forms a single antigen-binding site. The CH domain most proximal to VH is designated as CH1. Each L chain is linked to an H chain by one covalent disulfide bond, while the two H chains are linked to each other by one or more disulfide bonds depending on the H chain isotype. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1 or H-CDR1 (or CRD-H1), H2 or H-CDR2 (or CDR-H2) and H3 or H-CDR3 (or CDR-H3), while CDR constituents on the light chain are referred to as L1 or L-CDR1 (or CRD-L1), L2 or L-CDR2 (or CDR-L2), and L3 or L-CDR3 (or CDR-L3).

The term “variable” refers to the portions of the immunoglobulin domains that exhibit variability in their sequence and that are involved in determining the specificity and binding affinity of a particular antibody (i.e., the “variable domain(s)”). Variability is not evenly distributed throughout the variable domains of antibodies; it is concentrated in sub-domains of each of the heavy and light chain variable regions. These sub-domains are called “complementarity determining regions” (CDRs).

The terms “CDR”, and its plural “CDRs”, refer to a complementarity determining region (CDR) of which three make up the binding character of a light chain variable region (L1-CDR, L2-CDR and L3-CDR) and three make up the binding character of a heavy chain variable region (H1-CDR, H2-CDR and H3-CDR). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. However, the numbering in accordance with the so-called Kabat system is preferred.

The more conserved (i.e., non-hypervariable) portions of the variable domains are called the “framework” regions (FRM). The variable domains of naturally occurring heavy and light chains each comprise four FRM regions, largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.). The constant domains are not directly involved in antigen binding, but exhibit various effector functions, such as, for example, antibody-dependent, cell-mediated cytotoxicity and complement activation.

The term “binding domain” characterizes in connection with the present invention a domain of a polypeptide which specifically binds/interacts with a given target epitope. An “epitope” is antigenic and thus the term epitope is sometimes also referred to herein as “antigenic structure” or “antigenic determinant”. Thus, the binding domain is an “antigen-interaction-site”. The term “antigen-interaction-site” defines, in accordance with the present invention, a motif of a polypeptide, which is able to specifically interact with a specific antigen or a specific group of antigens, e.g. the identical antigen in different species. Said binding/interaction is also understood to define a “specific recognition”.

The terms “antigen-binding domain”, “antigen binding portion”, “antigen-binding fragment” and “antibody binding region” when used herein refer to a part of an antibody molecule that comprises amino acids responsible for the specific binding between antibody and antigen. The part of the antigen that is specifically recognized and bound by the antibody is referred to as the “epitope” as described herein above. As mentioned above, an antigen-binding domain may typically comprise an antibody light chain variable region (VL) and an antibody heavy chain variable region (VH).

The term “epitope” also refers to a site on an antigen (in the context of the present invention, the antigen is TPBG protein) to which the antibody molecule binds. Preferably, an epitope is a site on a molecule (in the context of the present invention, the antigen is a TPBG protein) against which a antibody or antigen binding portion thereof, preferably an antibody will be produced and/or to which an antibody will bind. For example, an epitope can be recognized by a antibody or antigen binding portion thereof. A “linear epitope” is an epitope where an amino acid primary sequence comprises the epitope recognized. A linear epitope typically includes at least 3, and more usually, at least 5, for example, about 8 to about 10 amino acids in a unique sequence.

The term “cross reactivity” may refer to the ability of an antibody to react with similar antigenic sites on different proteins.

The term “specifically” in this context may mean that the antibody or antigen binding portion thereof binds to target TPBG, but does not binds to another protein. The term “another protein” includes any protein including proteins closely related to or being homologous to TPBG protein against which the antibody or antigen binding portion thereof is directed to. However, the term “another protein” does not include that the antibody or antigen binding portion thereof cross-reacts with TPBG protein from another species different from that against which the antibody or antigen binding portion thereof was generated.

Thus, cross-species specific antibody or antigen binding portion thereof directed against TPBG protein are preferably contemplated by the present invention.

The term “K_{D}” may refer to the equilibrium dissociation constant, a ratio of k_{off}/kₒₙ, between the antibody and its antigen or between the variable regions of one heavy and one light chain of an antibody or fragment or derivative thereof and their antigen (e.g., TPBG, e.g., full-length TPBG and/or one or more fragments thereof, preferably said one or more fragments comprising at least an extracellular domain (ECD) of said TPBG (e.g., said ECD comprising at least amino acids 32-355 of the human TPBG having UniProt Accession Number: Q13641 or SEQ ID NO: 1) and/or one or more fragments of said ECD (e.g., having length from about 15 to about 30 amino acids) and is measured in vitro. K_{D} and affinity are inversely related.

As used herein, the term “affinity” may refer to the binding strength between the variable regions of one heavy and one light chain of an antibody or fragment or derivative thereof and their antigen (e.g., TPBG, e.g., full-length TPBG and/or one or more fragments thereof, preferably said one or more fragments comprising at least an extracellular domain (ECD) of said TPBG (e.g., said ECD comprising at least amino acids 32-355 of the human TPBG having UniProt Accession Number: Q13641 or SEQ ID NO: 1) and/or one or more fragments of said ECD (e.g., having length from about 15 to about 30 amino acids) and is measured in vitro. Affinity determines the strength of the interaction between an epitope and an antibody's antigen binding site. Affinity can be calculated using the following formula:

$$KA = [ AB - AG ] / [ AB ] * [ AG ] = {k}_{on} / {k}_{off}$$

wherein:
KA=affinity constant
[AB]=molar concentration of unoccupied binding sites on the antibody
[AG]=molar concentration of unoccupied binding sites on the antigen
[AB-AG]=molar concentration of the antibody-antigen complex

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is effected e.g. by post-translational modifications like glycosylation, acetylation, phosphorylation and the like. Such modifications are well known in the art.

The term “immune cells” refers to cells which are capable of producing antibodies. The immune cells of particular interest herein are lymphoid cells derived, e.g. from spleen, peripheral blood lymphoctes (PBLs), lymph node, inguinal node, Peyers patch, tonsil, bone marrow, cord blood, pleural effusions and tumor-infiltrating lymphocytes (TIL).

A type of antibody variant encompassed by the present invention is an amino acid substitution variant. These variants have at least one, two, three, four, five, six, seven, eight, nine or ten amino acid residues in the TPBG antibody molecule replaced by a different residue. The sites of greatest interest for substitutional mutagenesis include the CDRs of the heavy and/or light chain, in particular the hypervariable regions, but FR alterations in the heavy and/or light chain are also contemplated.

For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

Generally, if amino acids are substituted in one or more or all of the CDRs of the heavy and/or light chain, it is preferred that the then-obtained “substituted” sequence is at least 60% (e.g., at least 65%, at least 70%, at least 75%, at least 80%, at least 85%, at least 90%, at least 95%, at least 96%, at least 97%, at least 98%, at least 99% or 100%), more preferably 65%, even more preferably 70%, particularly preferable 75%, more particularly preferable 80% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the TPBG antibody may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions listed in Table I, herein) is envisaged as long as the antibody retains its capability to specifically bind to TPBG protein and/or its CDRs have an identity to the then substituted sequence (at least 60% ((e.g., at least 65%, at least 70%, at least 75%, at least 80%, at least 85%, at least 90%, at least 95%, at least 96%, at least 97%, at least 98%, at least 99% or 100%), more preferably 65%, even more preferably 70%, particularly preferable 75%, more particularly preferable 80% identical to the “original” CDR sequence).

Conservative substitutions are shown in Table I under the heading of “preferred substitutions”. If such substitutions result in a change in biological activity, then more substantial changes, denominated “exemplary substitutions” in Table I, or as further described below in reference to amino acid classes, may be introduced and the products screened for a desired characteristic.

### Chemical Modifications

The antibodies or antigen-binding variants or fragments thereof used in accordance with of the invention may be modified. Typical modifications conceivable in the context of the invention include, e.g., chemical modifications as described in the following.

Possible chemical modifications of the antibody or antigen-binding variants or fragments thereof include acylation or acetylation of the amino-terminal end or amidation or esterification of the carboxy-terminal end or, alternatively, on both. The modifications may also affect the amino group in the side chain of lysine or the hydroxyl group of threonine. Other suitable modifications include, e.g., extension of an amino group with polypeptide chains of varying length (e.g., XTEN technology or PASylation®), N-glycosylation, O-glycosylation, and chemical conjugation of carbohydrates, such as hydroxyethyl starch (e.g., HESylation®) or polysialic acid (e.g., PolyXen® technology). Chemical modifications such as alkylation (e. g., methylation, propylation, butylation), arylation, and etherification may be possible and are also envisaged.

### Antibody Drug Conjugate (ADC)

The therm antibody drug conjugate (or ADC) as used herein may refer to any antibody according to present invention conjugated to one or more drug moieties (e.g., cytotoxic payload/s). Preferably, the antibody drug conjugate (ADC) of the present invention comprising the anti-TPBG antibody of the present invention (e.g., humanized monoclonal TPBG-specific IgG1 antibody) conjugated to one or more cytotoxic payloads: (a) wherein the cytotoxic payload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or (b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or (c) wherein the cytotoxic payload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or (d) wherein the linker (L) comprises a valine-citrulline-PAB moiety or a valine-alanine-PAB moiety; and/or (e) wherein cytotoxic payload is Exatecan, conjugated via a chemical valine-citrulline-PAB or a valine-alanine-PAB release unit, wherein said release unit is cleavable by a protease. As used herein, a “linker” (L) may refer to any chemical moiety that is capable of linking the antibody of the present invention with one or more drug moieties (e.g., cytotoxic moieties (e.g., cytotoxic payloads, e.g., Tubulin disrupting agents, e.g., Topoisomerase-1 inhibitor/s, e.g., Auristatins or camptothecin, e.g., MMAE (monomethyl auristatin E) or MMAF (monomethyl auristatin F), e.g., Exatecan); Preferably L is a phosphonamidate linker; further preferably linker L comprising a valine-citrulline-PAB moiety or a valine-alanine-PAB moiety; most preferably the linker L is cleavable (e.g., susceptible to enzymatic cleavage).

### Sequence Identity

The term “% identity” or “% sequence identity” as used herein may refer to the percentage of pair-wise identical residues—following (homologous) alignment of a sequence of a polypeptide of the invention with a sequence in question—with respect to the number of residues in the longer of these two sequences. Percent identity is determined by dividing the number of identical residues by the total number of residues and multiplying the product by 100.

The percentage of sequence homology or sequence identity can, for example, be determined herein using the BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1) including the propeptide sequences, preferably using the wild type protein scaffold as reference in a pairwise comparison. It is calculated as the percentage of numbers of “positives” (homologous amino acids) indicated as result in the BLASTP program output divided by the total number of amino acids selected by the program for the alignment.

The term “TPBG” refers to oncofetal antigen 5T4 (also known as trophoblast glycoprotein (TPBG), both terms can be used interchangeably herein) and generally comprises all known isoforms. Preferably said TPBG is a human TPBG having SEQ ID NO: 1 or UniProtKB Accession Number: Q13641 or SEQ ID NO: 1.

### Vector

The nucleic acid of the invention may also be in the form of, may be present in and/or may be part of a vector.

The term “vector” refers a nucleic acid molecule used as a vehicle to transfer (foreign) genetic material into a host cell and encompasses—without limitation—plasmids, viruses, cosmids and artificial chromosomes such as bacterial artificial chromosomes (BACs) and yeast artificial chromosomes (YACs). In general, engineered vectors comprise an origin of replication, a multicloning site and a selectable marker. The vector itself is generally a nucleotide sequence, commonly a DNA sequence that comprises an insert (transgene) and a larger sequence that serves as the “backbone” of the vector. Vectors may encompass additional elements besides the transgene insert and a backbone including gene regulation elements, genetic markers, antibiotic resistances, reporter genes, targeting sequences, or protein purification tags. Particularly envisaged within the context of the invention are expression vectors (expression constructs) for expression of the transgene in the host cell, which generally comprise—in addition to the transgene—gene regulation sequences.

An expression vector is, in general, a vector that can provide for expression of the antibodies of the present invention in vitro and/or in vivo (i.e. in a suitable host cell, host organism and/or expression system). The person skilled in the art will readily understand that choice of a particular vector include depends, e.g., on the host cell, the intended number of copies of the vector, whether transient or stable expression of the antibody of the present invention is envisaged, and so on.

“Transient expression” results from the introduction of a nucleic acid (e.g. a linear or non-linear DNA or RNA molecule) or vector that is incapable of autonomous replication into a recipient host cell. Expression of the transgene occurs through the transient expression of the introduced sequence.

However, “stable expression” of the nucleic acid sequence as described herein will often be preferred and may be accomplished by either stably integrating the nucleic acid sequence into the host cell's genome or by introducing a vector comprising the nucleic acid sequence of the invention and being capable of autonomously replicating into the host cell.

The vector provided herein is in particular envisaged to comprise a gene regulation element operably linked to the DNA sequence encoding antibody of the present invention.

The term “gene regulation element” refers to DNA sequences necessary for the expression of an operably linked coding sequence in a particular host organism. The term “gene regulation element” includes controllable transcriptional promoters, operators, enhancers, silencers, transcriptional terminators, 5′ and 3′ untranslated regions which interact with host cellular proteins to carry out transcription and translation and other elements that may control gene expression including initiation and termination codons. The precise nature of the regulatory regions needed for gene expression may vary from organism to organism. Prokaryotic gene regulation elements, for example, include a promoter, optionally an operator sequence, and a ribosome binding site (RBS), whereas gene regulation elements for eukaryotic cells comprise promoters, polyadenylation (poly-A) signals, and enhancers.

The gene regulation element is envisaged to be “operably linked” to the gene to be expressed, i.e. placed in functional relationship with the same. For instance, a promoter or enhancer is “operably linked” to a coding nucleic acid sequence if it affects the transcription of the sequence. The DNA sequences being “operably linked” may or may not be contiguous. Linking is typically accomplished by ligation at convenient restriction sites or synthetic oligonucleotide adaptors or linkers.

### Host Cell

Further provided herein is a host cell (e.g., recombinant and/or isolated host cell) comprising the vector as described herein.

A variety of host cells can be employed for expressing the nucleic acid sequence encoding antibodies as described herein. Host cells can be prepared using genetic engineering methods known in the art. The process of introducing the vector into a recipient host cell is also termed “transformation” or “transfection” hereinafter. The terms are used interchangeably herein.

Host cell transformation typically involves opening transient pores or “holes” in the cell wall and/or cell membrane to allow the uptake of material. Illustrative examples of transformation protocols involve the use of calcium phosphate, electroporation, cell squeezing, dendrimers, liposomes, cationic polymers such as DEAE-dextran or polyethylenimine, sonoporation, optical transfection, impalefection, nanoparticles (gene gun), magnetofection, particle bombardement, alkali cations (cesium, lithium), enzymatic digestion, agitation with glass beads, viral vectors, or others. The choice of method is generally dependent on the type of cell being transformed, the vector to be introduced into the cell and the conditions under which the transformation is taking place.

As used herein, the term “host cell” may refer to any cell or cell culture acting as recipients for the vector or isolated nucleic acid sequence encoding the Abs as described herein. Suitable host cells include prokaryotic or eukaryotic cells, and also include but are not limited to bacteria, yeast cells, fungi cells, plant cells, and animal cells such as insect cells and mammalian cells, e.g., murine, rat, macaque or human.

E.g., the Abs can be produced in bacteria. In addition to prokaryotes, eukaryotic microbes such as filamentous fungi or yeast are suitable cloning or expression hosts for the TPBG-antibodies of the invention. Illustrative examples include Saccharomyces cerevisiae, Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of glycosylated antibody construct of the invention may also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art.

Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO), mouse sertoli cells (TM4); monkey kidney cells (CVI ATCC CCL 70); African green monkey kidney cells (VERO-76, ATCC CRL1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2,1413 8065); mouse mammary tumor (MMT 060562, ATCC CCL5 1); TRI cells; MRC 5 cells; FS4 cells; and human hepatoma cells (Hep G2).

### Patients

The term “patient” or “subject” as used herein refers to a human or non-human animal, generally a mammal. Particularly envisaged is a mammal, such as a rabbit, a mouse, a rat, a Guinea pig, a hamster, a dog, a cat, a pig, a cow, a goat, a sheep, a horse, a monkey, an ape or preferably a human. Thus, the methods, uses and compounds described in this document are in general applicable to both human and veterinary disease.

### Treatment

The term “treatment” in all its grammatical forms includes therapeutic or prophylactic treatment. A “therapeutic or prophylactic treatment” comprises prophylactic treatments aimed at the complete prevention of clinical and/or pathological manifestations or therapeutic treatment aimed at amelioration or remission of clinical and/or pathological manifestations of the diseases. The term “treatment” thus also includes the amelioration or prevention of cancer.

In the context with the present invention the term “therapeutic effect” in general refers to the desirable or beneficial impact of a treatment, e.g. amelioration or remission of the disease manifestations. The term “manifestation” of a disease is used herein to describe its perceptible expression, and includes both clinical manifestations, hereinafter defined as indications of the disease that may be detected during a physical examination and/or that are perceptible by the patient (i.e., symptoms), and pathological manifestations, meaning expressions of the disease on the cellular and molecular level. The therapeutic effect of treatment with the TPBG-ADC of the present invention can be assessed using routine methods in the art, e.g. measuring leukemia burden by blood/bone marrow analysis (cytomorphology, flow cytometry, genetics), clinical chemistry or radiologic procedures (e.g. CT) Additionally or alternatively it is also possible to evaluate the general appearance of the respective patient (e.g., fitness, well-being) which will also aid the skilled practitioner to evaluate whether a therapeutic effect has been elicited. The skilled person is aware of numerous other ways which are suitable to observe a therapeutic effect of the compounds of the present invention.

### Dose

Preferably, a therapeutically effective amount of the compound as described herein is administered. By “therapeutically effective amount” is meant an amount of the compound as described herein that elicits a therapeutic effect. The exact dose of Ab-TPBG-ADC of the present invention will depend on the purpose of the treatment (e.g. remission induction, maintenance), and will be ascertainable by one skilled in the art using known techniques. Adjustments for route of administration, age, body weight, general health, sex, diet, time of administration, drug interaction and the severity of the condition may be necessary, and will be ascertainable with routine experimentation by those skilled in the art.

### Administration

A variety of routes are applicable for administration of the compound according to the present invention, including, but not limited to, orally, topically, transdermally, subcutaneously, intravenously, intraperitoneally, intramuscularly or intraocularly, preferably subcutaneously and/or intravenously. However, any other route may readily be chosen by the person skilled in the art if desired.

### Composition

It is envisaged to administer the TPBG antibodies and/or ADCs of the present invention in the form of a pharmaceutical composition.

The term “pharmaceutical composition” particularly refers to a composition suitable for administering to a human, i.e., a composition that is preferably sterile and/or contains components which are pharmaceutically acceptable. However, compositions suitable for administration to non-human animals are also envisaged herein. Preferably, a pharmaceutical composition comprises an Ab-TPBG-ADC of the present invention together with one or more pharmaceutical excipients. The term “excipient” includes fillers, binders, disintegrants, coatings, sorbents, antiadherents, glidants, preservatives, antioxidants, flavoring, coloring, sweeting agents, solvents, co-solvents, buffering agents, chelating agents, viscosity imparting agents, surface active agents, diluents, humectants, carriers, diluents, preservatives, emulsifiers, stabilizers or tonicity modifiers. Pharmaceutical compositions of the invention can be formulated in various forms, e.g. in solid, liquid, gaseous or lyophilized form and may be, inter alia, in the form of an ointment, a cream, transdermal patches, a gel, powder, a tablet, solution, an aerosol, granules, pills, suspensions, emulsions, capsules, syrups, liquids, elixirs, extracts, tincture or fluid extracts or in a form which is particularly suitable for the desired method of administration.

The pharmaceutical composition of the present invention may further comprise one or more additional agents. Preferably, said agents are therapeutically effective for treatment the diseases described herein and present in the composition in a therapeutically effective amount.

In view of the above, the present invention hence also provides a pharmaceutical composition comprising one or more TPBG antibodies and/or ADCs of the present invention. Said pharmaceutical composition is particularly intended for use in a method of therapeutic and/or prophylactic treatment of cancer.

### Kit

A kit is also provided herein. The kit may be a kit of two or more parts, and comprises the TPBG antibodies and/or ADCs of the present invention, preferably in a therapeutically effective amount and in a pharmaceutically acceptable form. The components of the kit may be contained in a container or vials. The kit is envisaged to comprise additional agents useful in treating cancer, as described elsewhere herein.

### Chemical Groups

Unless otherwise indicated, the term “alkyl” by itself or as part of another term in general refers to a substituted or unsubstituted straight chain or branched, saturated hydrocarbon having the indicated number of carbon atoms; e.g., “—(C₁-C₈)alkyl” or “—(C₁-C₁₀)alkyl” refer to an alkyl group having from 1 to 8 or 1 to 10 carbon atoms, respectively). When the number of carbon atoms is not indicated, the alkyl group may have from 1 to 8 carbon atoms. Representative straight chain —(C₁-C₈)alkyl groups include, but are not limited to, -methyl, -ethyl, -n-propyl, -n-butyl, -n-pentyl, -n-hexyl, -n-heptyl and -n-octyl; branched —(C₁-C₈)alkyl groups include, but are not limited to, -isopropyl, -sec-butyl, -isobutyl, -tert-butyl, -isopentyl, and -2-methylbutyl. In some aspects, an alkyl group may be unsubstituted. Optionally, an alkyl group may be substituted, such as e.g. with one or more groups.

Unless otherwise indicated, the term “alkylene” by itself or as part of another term, in general refers to a substituted or unsubstituted branched or straight chain, saturated hydrocarbon radical of the stated number of carbon atoms, preferably 1-10 carbon atoms (—(C₁-C₁₀)alkylene-) or preferably 1 to 8 carbon atoms (—(C₁-C₈)alkylene-), and having two monovalent radical centers derived by the removal of two hydrogen atoms from the same or two different carbon atoms of a parent alkane. When the number of carbon atoms is not indicated, the alkylene group may have from 1 to 8 carbon atoms. Typical alkylene radicals include, but are not limited to: methylene (—CH₂—), 1,2-ethylene (—CH₂CH₂—), 1,3-n-propylene (—CH₂CH₂CH₂—), and 1,4-n-butylene (—CH₂CH₂CH₂CH₂—). In some aspects, an alkylene group may be unsubstituted. Optionally, an alkylene group may be substituted, such as e.g. with one or more groups.

Unless otherwise indicated, the term “alkenyl” by itself or as part of another term in general refers to a substituted or unsubstituted straight chain or branched, unsaturated hydrocarbon having a double bond and the indicated number of carbon atoms; e.g., “—(C₂-C₈)alkenyl” or “—(C₂-C₁₀)alkenyl” refer to an alkenyl group having from 2 to 8 or 2 to 10 carbon atoms, respectively). When the number of carbon atoms is not indicated, the alkenyl group may have from 2 to 8 carbon atoms. Representative —(C₂-C₈)alkenyl groups include, but are not limited to, -ethenyl, -1-propenyl, -2-propenyl, -1-butenyl, -2-butenyl, -isobutenyl, -1-pentenyl, -2-pentenyl, -3-methyl-1-butenyl, -2-methyl-2-butenyl, and -2,3-dimethyl-2-butenyl. In some aspects, an alkenyl group may be unsubstituted. Optionally, an alkenyl group may be substituted, such as e.g. with one or more groups.

Unless otherwise indicated, the term “alkenylene” by itself of as part of another term, in general refers to a substituted or unsubstituted unsaturated branched or straight chain hydrocarbon radical of the stated number of carbon atoms, preferably 2-10 carbon atoms (—(C₂-C₁₀)alkenylene-) or preferably 2 to 8 carbon atoms (—(C₂-C₈)alkenylene-), and having a double bond, and having two monovalent radical centers derived by the removal of two hydrogen atoms from the same or two different carbon atoms of a parent alkene. When the number of carbon atoms is not indicated, the alkenylene group may have from 2 to 8 carbon atoms. Typical alkenylene radicals include, but are not limited to: -ethenylene-, -1-propenylene-, 2-propenylene-, -1-butenylene-, -2-butenylene-, -isobutenylene-, -1-pentenylene-, -2-pentenylene-, -3-methyl-1-butenylene-, -2-methyl-2-butenylene-, and -2,3-dimethyl-2-butenylene-. In some aspects, an alkenylene group may be unsubstituted. Optionally, an alkenylene group may be substituted, such as e.g. with one or more groups.

Unless otherwise indicated, the term “alkynyl” by itself or as part of another term in general refers to a substituted or unsubstituted straight chain or branched, unsaturated hydrocarbon having a triple bond and the indicated number of carbon atoms; e.g., “—(C₂-C₈)alkynyl” or “—(C₂-C₁₀)alkynyl” refer to an alkynyl group having from 2 to 8 or 2 to 10 carbon atoms, respectively). When the number of carbon atoms is not indicated, the alkynyl group may have from 2 to 8 carbon atoms. Representative —(C₂-C₈)alkynyl groups include, but are not limited to, -acetylenyl, -1-propynyl, -2-propynyl, -1-butynyl, -2-butynyl, -1-pentynyl, -2-pentynyl and -3-methyl-1-butynyl. In some aspects, an alkynyl group may be unsubstituted. Optionally, an alkynyl group may be substituted, such as e.g. with one or more groups.

Unless otherwise indicated, the term “alkynylene” by itself of as part of another term, in general refers to a substituted or unsubstituted, branched or straight chain, unsaturated hydrocarbon radical of the stated number of carbon atoms, preferably 2-10 carbon atoms (—(C₂-C₁₀)alkynylene-) or preferably 2 to 8 carbon atoms (—(C₂-C₈)alkynylene-), and having a triple bond, and having two monovalent radical centers derived by the removal of two hydrogen atoms from the same or two different carbon atoms of a parent alkyne. When the number of carbon atoms is not indicated, the alkynylene group may have from 2 to 8 carbon atoms. Typical alkynylene radicals include, but are not limited to: -ethynylene-, -1-propynylene-, -2-propynylene-, -1-butynylene-, -2-butynylene-, -1-pentynylene-, -2-pentynylene- and -3-methyl-1-butynylene-. In some aspects, an alkynylene group may be unsubstituted. Optionally, an alkynylene group may be substituted, such as e.g. with one or more groups.

Unless otherwise indicated, the term “aryl,” by itself or as part of another term, in general means a substituted or unsubstituted monovalent carbocyclic aromatic hydrocarbon radical of 6 to 20 carbon atoms (preferably 6 to 14 carbon atoms, more preferably 6 to 10 carbon atoms, in very preferred embodiments 6 carbon atoms) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, and biphenyl. An exemplary aryl group is a phenyl group. In some aspects, an aryl group may be unsubstituted. Optionally, an aryl group may be substituted, such as e.g. with one or more groups.

Unless otherwise indicated, the term “arylene”, by itself or as part of another term, in general is an aryl group as defined above wherein one of the hydrogen atoms of the aryl group is replaced with a bond (i.e., it is divalent) and can be in the para, meta, or ortho orientations as shown in the following structures, with phenyl as the exemplary group:

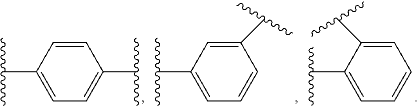

In selected embodiments, the arylene is an aryl group as defined above wherein two or more of the hydrogen atoms of the aryl group are replaced with a bond (i.e., the arylene can be trivalent). In some aspects, an arylene group may be unsubstituted. Optionally, an alkynylene group may be substituted, such as e.g. with one or more groups.

Unless otherwise indicated, the term “heterocycle” or “heterocyclic ring”, by itself or as part of another term, in general refers to a monovalent substituted or unsubstituted aromatic or non-aromatic monocyclic or bicyclic ring system having the indicated number of carbon atoms (e.g., “(C₃-C₈)heterocycle” or “(C₃-C₁₀)heterocycle” refer to a heterocycle having from 3 to 8 or from 3 to 10 carbon atoms, respectively) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a (C₃-C₈)heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. In some aspects, a heterocycle group may be unsubstituted. Optionally, a heterocycle group may be substituted, such as e.g. with one or more groups.

Unless otherwise indicated, the term “heterocyclo” or “heterocyclic ring”, by itself or as part of another term, in general refers to a heterocycle group as defined above and having the indicated number of carbon atoms (e.g., (C₃-C₈)heterocycle or (C₃-C₁₀)heterocycle) wherein one of the hydrogen atoms of the heterocycle group is replaced with a bond (i.e., it is divalent). In selected embodiments, the heterocyclo is a heterocycle group as defined above wherein two or more of the hydrogen atoms of the heterocycle group are replaced with a bond (i.e., the heterocyclo can be trivalent). In some aspects, a heterocyclo or heterocyclic ring may be unsubstituted. Optionally, a heterocyclo or heterocyclic ring may be substituted, such as e.g. with one or more groups.

Unless otherwise indicated, the term “carbocycle” or “carbocyclic ring” by itself or as part of another term, in general refers to a monovalent, substituted or unsubstituted aromatic or non-aromatic monocyclic or bicyclic carbocyclic ring system having the indicated number of carbon atoms (e.g., “(C₃-C₈)carbocycle” or “(C₃-C₁₀)carbocycle” refer to a carbocycle having from 3 to 8 or from 3 to 10 carbon atoms, respectively) derived by the removal of one hydrogen atom from a ring atom of a parent ring system. As illustrative but non-limiting examples the carbocycle may be a 3-, 4-, 5-, 6-, 7- or 8-membered carbocycle. Representative (C₃-C₃)carbocycles include, but are not limited to, phenyl, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. In some aspects, a carbocycle may be unsubstituted. Optionally, a carbocycle may be substituted, such as e.g. with one or more groups.

Unless otherwise indicated, the term “carbocyclo” or “carbocyclic ring”, by itself or as part of another term, in general refers to a carbocycle group as defined above having the indicated number of carbon atoms (e.g., “(C₃-C₈)carbocyclo” or “(C₃-C₁₀)carbocyclo” refer to a carbocyclo or carbocyclic ring having from 3 to 8 or from 3 to 10 carbon atoms, respectively), wherein another of the hydrogen atoms of the carbocycle groups is replaced with a bond (i.e., it is divalent). In selected embodiments, the carbocyclo or carbocyclic ring is a carbocycle group as defined above, wherein two or more of the hydrogen atoms of the carbocycle group are replaced with a bond (i.e., the carbocyclo or carbocyclic ring can be trivalent). In some aspects, a carbocyclo or carbocyclic ring may be unsubstituted. Optionally, a heterocyclo or heterocyclic ring may be substituted, such as e.g. with one or more groups.

Unless otherwise indicated, the term “heteroalkyl”, by itself or in combination with another term, may mean, unless otherwise stated, a stable straight or branched chain hydrocarbon, or combinations thereof, fully saturated or containing from 1 to 3 degrees of unsaturation, consisting of the stated number of carbon atoms (e.g., (C₁-C₈)heteroalkyl or (C₁-C₁₀)heteroalkyl) and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CH—O—CH₃, —Si(CH₃)₃, —CH₂—CH═N—O—CH₃, and —CH═CH—N(CH₃)—CH₃. Up to two heteroatoms may be consecutive, such as, for example, —CH₂—NH—OCH₃ and —CH₂—O—Si(CH₃)₃. In preferred embodiments, a (C₁-C₄)heteroalkyl or heteroalkylene has 1 to 4 carbon atoms and 1 or 2 heteroatoms and a (C₁-C₃)heteroalkyl or heteroalkylene has 1 to 3 carbon atoms and 1 or 2 heteroatoms. In some aspects, a heteroalkyl or heteroalkylene is saturated. In some aspects, a heteroalkyl or heteroalkylene may be unsubstituted. Optionally, a heteroalkyl or heteroalkylene may be substituted, such as e.g. with one or more groups.

Unless otherwise indicated, the term “heteroalkylene” by itself or as part of another substituent means a divalent group derived from heteroalkyl (as described above) having the indicated number of carbon atoms (e.g., (C₁-C₈)heteroalkylene or (C₁-C₁₀)heteroalkylene), as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied. In selected embodiments, the heteroalkylene is a heteroalkyl group defined above wherein two or more of the hydrogen atoms of the heteroalkyl group are replaced with a bond (i.e., the heteroalkylene can be trivalent). In some aspects, a heteroalkyl or heteroalkylene may be saturated. In some aspects, a heteroalkylene is unsubstituted. Optionally, a heteroalkylene may be substituted, such as e.g. with one or more groups.

The term “halogen”, unless defined otherwise, in general refers to elements of the 7^{th} main group; preferably fluorine, chlorine, bromine and iodine; more preferably fluorine, chlorine and bromine; even more preferably, fluorine and chlorine.

The term “substituted”, “optionally substituted”, “optionally may be substituted” or the like, unless otherwise indicated, in general means that one or more hydrogen atoms can be each independently replaced with a substituent. Typical substituents include, but are not limited to, —X, —R, —O—, —OR, —SR, —S—, —NR₂, —NR₃, ═NR, —CX₃, —CN, —OCN, —SCN, —N═C═O, —NCS, —NO, —NO₂, ═N₂, —N₃, —NRC(═O)R, —C(═O)R, —C(═O)NR₂, —SO₃—, —SO₃H, —S(═O)₂R, —OS(═O)₂OR, —S(═O)₂NR, —S(═O)R, —OP(═O)(OR)₂, —P(═O)(OR)₂, —PO₄₃—, —PO₃H₂, —C(═O)R, —C(═O)X, —C(═S)R, —CO₂R, —CO₂, —C(═S)OR, —C(═O)SR, —C(═S)SR, —C(═O)NR₂, —C(═S)NR₂, or —C(═NR)NR₂, where each X is independently a halogen: —F, —C, —Br, or —I; and each R is independently —H, —(C₁-C₂₀)alkyl (such as e.g. —(C₁-C₁₀)alkyl or —(C₁-C₈)alkyl), —(C₆-C₂₀)aryl, (such as e.g. —(C₆-C₁₀)aryl or, preferably, —C₆-aryl), —(C₃-C₁₄)heterocycle (such as e.g. —(C₃-C₁₀)heterocycle or —(C₃-C₈)heterocycle), a protecting group, or a prodrug moiety. Typical substituents also include (═O).

The term “aliphatic or aromatic residue”, as used herein, in general refers to an aliphatic substituent, such as e.g. but not limited to an alkyl residue, which, however, can be optionally substituted by further aliphatic and/or aromatic substituents. As non-limiting examples an aliphatic residue can be a nucleic acid, an enzyme, a co-enzyme, a nucleotide, an oligonucleotide, a monosaccharide, a polysaccharide, a polymer, a fluorophore, optionally substituted benzene, etc., as long as the direct link of such a molecule to the core structure (in case of R¹, e.g., the link to the oxygen atom bound to the phosphorus) is aliphatic. An aromatic residue is a substituent, wherein the direct link to the core structure is part of an aromatic system, e.g., an optionally substituted phenyl or triazolyl or pyridyl or nucleotide; as non-limiting example if the direct link of the nucleotide to the core structure is for example via a phenyl-residue. The term “aromatic residue”, as used herein, also includes a heteroaromatic residue.

The term “peptide”, unless otherwise indicated, in general refers to an organic compound comprising two or more amino acids covalently joined by peptide bonds (amide bond). Peptides may be referred to with respect to the number of constituent amino acids, i.e., a dipeptide contains two amino acid residues, a tripeptide contains three, etc. Peptides containing 30 or fewer amino acids may be referred to as oligopeptides, while those, for example, with more than 30 amino acid residues may be referred to as polypeptides. Amino acids and peptides according to the disclosure can also be modified at functional groups. Non limiting examples are saccharides, e.g., N-Acetylgalactosamine (GalNAc), or protecting groups, e.g., Fluorenylmethoxycarbonyl (Fmoc)-modifications or esters.

### Pharmaceutically Acceptable Salt

The present disclosure also relates to a “pharmaceutically acceptable salt”. Any pharmaceutically acceptable salt can be used. In particular, the term “pharmaceutically acceptable salt” refers to a salt of a conjugate or compound of the invention that is pharmaceutically acceptable and that possesses the desired pharmacological activity of the parent compound. In particular, such salts have low toxicity and may be inorganic or organic acid addition salts and base addition salts. Specifically, such salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, purely by way of example, sodium, potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like. A counterion or anionic counterion can be used in a quaternary amine to maintain electronic neutrality. Exemplary counterions include halide ions (e.g., F⁻, Cl⁻, Br⁻, I⁻), NO₃⁻, ClO₄⁻, OH—, H₂PO₄⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, and the like), and carboxylate ions (e.g., acetate, ethanoate, propanoate, benzoate, glycerate, lactate, tartrate, glycolate, and the like).

### Solvate

As used herein, the term “solvate” may refer to an aggregate that comprises one or more molecules of a conjugate or compound described herein with one or more molecules of solvent. The solvent may be water, in which case the solvate may be a hydrate. Alternatively, the solvent may be an organic solvent. Thus, the conjugates or compounds of the present disclosure may exist as a hydrate, including a monohydrate, dihydrate, hemihydrate, sesquihydrate, trihydrate, tetrahydrate and the like, as well as the corresponding solvated forms. The compounds of the invention may be true solvates, while in other cases, the compounds of the invention may merely retain adventitious water or be a mixture of water plus some adventitious solvent.

In some aspects/embodiments the present invention relates to an anti-TPBG antibody (e.g., antibody binding portion thereof), wherein said anti-TPBG antibody comprising Fc silencing mutations such as leucine (L) to alanine (A) substitution at the position 234 and 235 (LALA mutations) and is capable of the following: binding to human Trophoblast glycoprotein (TPBG) (e.g., having UniProt Accession Number: Q13641 or SEQ ID NO: 1); having cross-reactivity with white-tufted-ear marmoset (e.g., Callithrix jacchus) TPBG (e.g., having UniProtKB Accession Number: F710T3 or SEQ ID NO: 2); and internalization, preferably by the means of the antigen-mediated antibody internalization.

In some aspects/embodiments the present invention relates to a monoclonal human or humanized IgG1 anti-TPBG antibody, preferably comprising kappa (κ) light chain.

In some aspects/embodiments the present invention relates to a hybridoma, wherein said hybridoma produces the antibody of the present invention.

In some aspects/embodiments the present invention relates to a nucleic acid encoding the antibody of the present invention.

In some aspects/embodiments the present invention relates to an expression vector comprising at least one of the nucleic acid molecules of the present invention.

The present invention further relates to In some aspects/embodiments the present invention relates to an isolated host cell (e.g., an isolated recombinant host cell) comprising the vector and/or nucleic acid of the present invention.

In some aspects/embodiments the present invention relates to an antibody drug conjugate (ADC) comprising the anti-TPBG antibody of the present invention.

In some aspects/embodiments the present invention relates to an antibody drug conjugate (ADC) of the present invention comprising the anti-TPBG antibody of the present invention (e.g., humanized monoclonal TPBG-specific IgG1 antibody) conjugated to a cytotoxic payload: (a) wherein the cytotoxic payload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or (b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or (c) wherein the cytotoxic payload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or (d) wherein the linker (L) comprises a valine-citrulline-PAB moiety or a valine-alanine-PAB moiety; and/or (e) wherein cytotoxic payload is Exatecan, conjugated via a chemical valine-citrulline-PAB or a valine-alanine-PAB release unit, wherein said release unit is cleavable by a protease. Preferably, a drug (e.g., cytotoxic moieties (e.g., cytotoxic payload/s, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan) to antibody ratio (DAR) is in the range between 0 and 20; further preferably DAR is in the range from 4 to 8, most preferably DAR is 4 or 8.

In some aspects/embodiments the present invention relates to an antibody drug conjugate (ADC) having the formula (I):

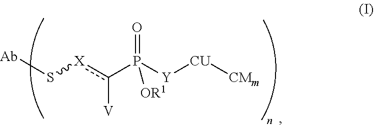

or a pharmaceutically acceptable salt or solvate thereof;

wherein:
Ab is an anti-TPBG antibody as described herein;
[symbol] is a double bond; or
[symbol] is a bond;
V is absent when [symbol] is a double bond; or
V is H or (C₁-C₈)alkyl when [symbol] is a bond;
X is R₃—C when [symbol] is a double bond; or
X is

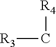

when [symbol] is a bond;
Y is NR⁵, S, O, or CR⁶R⁷;
R¹ is an optionally substituted aliphatic residue or an optionally substituted aromatic residue;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
CU is a connector unit;
CM is a cytotoxic moiety;
m is an integer ranging from 1 to 10; and
n is an integer ranging from 1 to 20.

Preferably R³ is H or (C₁-C₈)alkyl; more preferably R³ is H. Preferably R⁴, when present is H or (C₁-C₈)alkyl; more preferably R⁴, when present, is H. Preferably R⁵, when present is H or (C₁-C₈)alkyl; more preferably R⁵, when present, is H. Preferably R⁶, when present is H or (C₁-C₈)alkyl; more preferably R⁶, when present, is H. Preferably R⁷, when present is H or (C₁-C₈)alkyl; more preferably R⁷, when present, is H.

Preferably, [symbol] is a double bond; V is absent; X is R₃—C; and R³ is H or an optionally substituted aliphatic residue or an optionally substituted aromatic residue; preferably R³ is H or (C₁-C₈)alkyl; more preferably R³ is H.

More preferably, [symbol] represents a double bond; V is absent; X represents R₃—C, and R₃ represents H or (C₁-C₈)alkyl. Preferably, R³ represents H or (C₁-C₆)alkyl, more preferably H or (C₁-C₄)alkyl, still more preferably H or (C₁-C₂)alkyl. In preferred embodiments, R₃ is H.

In some embodiments, [symbol] may be a bond; V is H or (C₁-C₈)alkyl, preferably V is H; X is

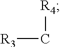

R₃ is H or an optionally substituted aliphatic residue or an optionally substituted aromatic residue; more preferably R³ is H or (C₁-C₈)alkyl, more preferably R³ is H; R⁴ is H or an optionally substituted aliphatic residue or an optionally substituted aromatic residue;
preferably, R⁴ is H or (C₁-C₈)alkyl, preferably R⁴ is H.

In some embodiments, [symbol] may represent a bond; V may be H or (C₁-C₈)alkyl; X may represent

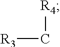

and R₃ and R₄ may independently represent H or (C₁-C₈)alkyl. Preferably, R₃ and R₄ independently represent H or (C₁-C₆)alkyl, more preferably H or (C₁-C₄)alkyl, still more preferably H or (C₁-C₂)alkyl. Preferably, R₃ and R₄ are the same; even more preferably, R₃, R₄ and V are the same. More preferably, R₃ and R₄ are both H. Preferably, V is H or (C₁-C₆)alkyl, more preferably H or (C₁-C₄)alkyl, still more preferably H or (C₁-C₂)alkyl. Even more preferably, V is H. In preferred embodiments, R₃, R₄ and V are each H.

The integer m ranges from 1 to 10. Accordingly, the integer m may be 1, 2, 3, 4, 5, 6, 7, 8, 9 or 10. Preferably, the integer m ranges from 1 to 4. More preferably, the integer m is 1 or 2. Even more preferably, the integer m is 1.

The integer n ranges from 1 to 20. Accordingly, the integer n may be 1, 2, 3, 4, 5, 6, 7, 8, 9, 10, 11, 12, 13, 14, 15, 16, 17, 18, 19 or 20. Preferably, the integer n ranges from 1 to 10. More preferably, the integer n ranges from 2 to 10. Still more preferably, the integer n ranges from 4 to 10. Still more preferably, the integer n ranges from 6 to 10. Still more preferably, the integer n is 6, 7, 8, 9 or 10. Still more preferably, the integer n ranges from 7 to 10. Still more preferably, the integer n is 7, 8 or 9. Still more preferably, the integer n is 7 or 8. Even more preferably, the integer n is 8.

The integer n ranges from 1 to 20. Preferably, the integer n ranges from 1 to 10. More preferably, the integer n ranges from 2 to 8. Still more preferably, the integer n is 2, 3, 4, 5 or 6. Still more preferably, the integer n ranges from 3 to 6. Still more preferably, the integer n is 3, 4 or 5. Still more preferably, the integer n is 4 or 5. Even more preferably, the integer n is 4.

Preferably, m is an integer ranging from 1 to 4, more preferably 1 or 2, still more preferably 1; and preferably n is an integer ranging from 1 to 20, more preferably from 1 to 10, still more preferably from 2 to 10, still more preferably from 4 to 10, still more preferably from 6 to 10, still more preferably n is 6, 7, 8, 9 or 10, still more preferably n ranges from 7 to 10, still more preferably n is 7, 8 or 9, still more preferably n is 7 or 8, even more preferably n is 8.

Preferably, m is an integer ranging from 1 to 4, preferably 1 or 2, more preferably 1; and preferably n is an integer ranging from 1 to 20, more preferably from 1 to 10, still more preferably from 2 to 8; still more preferably n is 2, 3, 4, 5 or 6; still more preferably n ranges from 3 to 6; still more preferably n is 3, 4 or 5; still more preferably n is 4 or 5, even more preferably n is 4.

Preferably, m is 1; and preferably n is an integer ranging from 1 to 20, more preferably from 1 to 10, still more preferably from 2 to 10, still more preferably from 4 to 10, still more preferably from 6 to 10, still more preferably n is 6, 7, 8, 9 or 10, still more preferably n ranges from 7 to 10, still more preferably n is 7, 8 or 9, still more preferably n is 7 or 8, even more preferably n is 8. Accordingly, preferably, m is 1 and n is an integer ranging from 1 to 20. More preferably, m is 1 and n is an integer ranging from 1 to 10. Still more preferably, m is 1 and n is an integer ranging from 2 to 10. Still more preferably, m is 1 and n is an integer ranging from 4 to 10. Still more preferably, m is 1 and n is an integer ranging from 6 to 10. Still more preferably, m is 1 and n is 6, 7, 8, 9 or 10. Still more preferably, m is 1 and n is an integer ranging from 7 to 10. Still more preferably, m is 1 and n is 7, 8 or 9. Still more preferably, m is 1 and n is 7 or 8. Even more preferably, m is 1 and n is 8.

Preferably, m is 1; and preferably n is an integer ranging from 1 to 20, more preferably from 1 to 10, still more preferably from 2 to 8; still more preferably n is 2, 3, 4, 5 or 6, still more preferably n ranges from 3 to 6; still more preferably n is 3, 4 or 5; still more preferably n is 4 or 5, even more preferably n is 4. Accordingly, preferably, m is 1 and n is an integer ranging from 1 to 20. More preferably, m is 1 and n is an integer ranging from 1 to 10. Still more preferably, m is 1 and n is an integer ranging from 2 to 8. Still more preferably, m is 1 and n is 2, 3, 4, 5 or 6. Still more preferably, m is 1 and n ranges from 3 to 6. Still more preferably, m is 1 and n is 3, 4 or 5. Still more preferably, m is 1 and n is 4 or 5. Even more preferably, m is 1 and n is 4.

In some embodiments, the number of cytotoxic moieties CM per antibody Ab may be from 1 to 20. Preferably, the number of cytotoxic moieties CM per antibody Ab is from 1 to 14. More preferably, the number of cytotoxic moieties CM per antibody Ab is from 2 to 14. More preferably, the number of cytotoxic moieties CM per antibody Ab is from 4 to 14. Still more preferably, the number of cytotoxic moieties CM per antibody Ab is from 5 to 12. Still more preferably, the number of cytotoxic moieties CM per antibody Ab is from 6 to 12. Still more preferably, the number of cytotoxic moieties CM per antibody Ab is from 7 to 10. Even more preferably, the number of cytotoxic moieties CM per antibody Ab is 8.

In some embodiments, the number of cytotoxic moieties CM per antibody Ab may be from 1 to 20. Preferably, the number of cytotoxic moieties CM per antibody Ab is from 1 to 14. More preferably, the number of cytotoxic moieties CM per antibody Ab is from 1 to 12. More preferably, the number of cytotoxic moieties CM per antibody Ab is from 2 to 10. Still more preferably, the number of cytotoxic moieties CM per antibody Ab is from 2 to 8. Still more preferably, the number of cytotoxic moieties CM per antibody Ab is from 2 to 6. Still more preferably, the number of cytotoxic moieties CM per antibody Ab is from 3 to 5. Even more preferably, the number of cytotoxic moieties CM per antibody Ab is 4.

Ab is an anti-TPBG antibody as described herein. Any anti-TPBG antibody as described herein may be used.

### Group Y

The group Y is selected from the group consisting of NR⁵, S, O, and CR⁶R⁷. R⁵ is H; or an optionally substituted aliphatic residue or an optionally substituted aromatic residue; preferably R⁵ is H or (C₁-C₈)alkyl; more preferably R⁵ is H. R⁶ is H; or an optionally substituted aliphatic residue or an optionally substituted aromatic residue; preferably R⁶ is H or (C₁-C₈)alkyl; more preferably R⁶ is H. R⁷ is H; or an optionally substituted aliphatic residue or an optionally substituted aromatic residue; preferably R⁷ is H or (C₁-C₈)alkyl, more preferably R⁷ is H.

Preferably, Y is selected from the group consisting of NH, S, O and CH₂. More preferably, Y is NH, S or O. In some embodiments, Y is CH₂. In some embodiments, Y is 0. In some embodiments, Y is S.

In very preferred embodiments, Y is NH.

### Group R¹

R¹ is an optionally substituted aliphatic residue or optionally substituted aromatic residue.

R¹ may represent optionally substituted (C₁-C₈)alkyl.

R¹ may represent (C₁-C₈)alkyl optionally substituted with at least one of F, Cl, Br, I, —NO₂, —N((C₁-C₈)alkyl)H, —NH₂, —N₃, —N((C₁-C₈)alkyl)₂, ═O, (C₃-C₈)cycloalkyl, —S—S—((C₁-C₈)alkyl), (C₂-C₈)alkenyl or (C₂-C₈)alkynyl.

R¹ may represent optionally substituted phenyl.

R¹ may represent phenyl optionally independently substituted with at least one of (C₁-C₈)alkyl, F, Cl, I, Br, —NO₂, —N((C₁-C₈)alkyl)H, —NH₂ or —N((C₁-C₈)alkyl)₂.

R¹ may represent an optionally substituted 5- or 6-membered heteroaromatic ring such as e.g. pyridyl.

R¹ may represent (C₁-C₈)alkyl, (C₁-C₈)alkyl substituted with —S—S—(C₁-C₈)alkyl, (C₁-C₈)alkyl substituted with optionally substituted phenyl; or phenyl; or phenyl substituted with —NO₂.

R¹ may represent methyl, ethyl, propyl or butyl, preferably methyl or ethyl, more preferably ethyl.

### Polyethylene Glycol Unit

Preferably, R¹ is a polyethylene glycol unit. In some embodiments, R¹ is a polyethylene glycol unit comprising of from 1 to 100, preferably of from 2 to 50, more preferably of from 3 to 45, still more preferably of from 4 to 40, still more preferably of from 6 to 35, even more preferably of from 8 to 30 ethylene glycol subunits each having the structure:

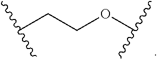 [SMILES: *CCO*]

Throughout the present specification, the structure

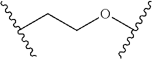 [SMILES: *CCO*]

is denoted as an “ethylene glycol subunit”.

Preferably, R¹ is a polyethylene glycol unit comprising of from 16 to 30, more preferably of from 20 to 28, still more preferably 22, 23, 24, 25 or 26, even more preferably 23, 24 or 25 ethylene glycol subunits each having the structure:

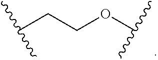 [SMILES: *CCO*]

In preferred embodiments, R¹ is a polyethylene glycol unit comprising 24 or about 24 ethylene glycol subunits each having the structure:

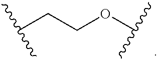 [SMILES: *CCO*]

More preferably, R¹ is a polyethylene glycol unit having the structure:

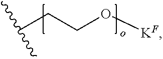

wherein:
[symbol] indicates the position of the 0 attached to the phosphorus;
K^{F} is H (hydrogen) or a capping group as described herein; preferably K^{F} is selected from the group consisting of —H (hydrogen), —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; more preferably K^{F} is H; and
o is an integer ranging from 1 to 100.

The integer o denotes the number of repeating units in

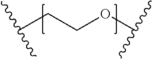

the polyethylene glycol unit. The integer o may range from 1 to 100. Preferably, o ranges from 2 to 50. More preferably, o ranges from 3 to 45. Still more preferably, o ranges from 4 to 40. Still more preferably, o ranges from 6 to 35. Even more preferably, o ranges from 8 to 30. In some embodiments, o is 12 or about 12. Even more preferably, o ranges from 16 to 30. Even more preferably, o ranges from 20 to 28. Even more preferably, o is 22, 23, 24, 25 or 26. Even more preferably, o is 23, 24 or 25. In preferred embodiments, o is 24 or about 24.

In general, in the polyethylene glycol unit, polydisperse polyethylene glycols, monodisperse polyethylene glycols, and discrete polyethylene glycols can be used. Polydisperse polyethylene glycols are a heterogenous mixture of sizes and molecular weights, whereas monodisperse polyethylene glycols are typically purified from heterogenous mixtures and therefore provide a single chain length and molecular weight. Preferred polyethylene glycol units are discrete polyethylene glycols, i.e. compounds that are synthesized in step-wise fashion and not via a polymerization process. Discrete polyethylene glycols provide a single molecule with defined and specified chain length.

The polyethylene glycol unit provided herein comprises one or multiple polyethylene glycol chains. The polyethylene glycol chains can be linked together, for example, in a linear, branched or star shaped configuration. Optionally, at least one of the polyethylene glycol chains may be derivatized at one end for covalent attachment to the oxygen atom bound to the phosphorus.

The polyethylene glycol unit will be attached to the antibody drug conjugate (or intermediate thereof) at the oxygen atom which is bound to the phosphorus. The other terminus (or termini) of the polyethylene glycol unit will be free and untethered and may take the form of a hydrogen, methoxy, carboxylic acid, alcohol or other suitable functional group, such as e.g. any capping group as described herein. The methoxy, carboxylic acid, alcohol or other suitable functional group acts as a cap for the terminal polyethylene glycol subunit of the polyethylene glycol unit. By untethered, it is meant that the polyethylene glycol unit will not be attached at that untethered site to a cytotoxic moiety (CM), to a receptor binding molecule, or to a component of the connector unit (CU) connecting a cytotoxic moiety and/or an antibody (Ab). For those embodiments wherein the polyethylene glycol unit comprises more than one polyethylene glycol chain, the multiple polyethylene glycol chains may be the same or different chemical moieties (e.g., polyethylene glycols of different molecular weight or number of subunits). The multiple polyethylene glycol chains are attached to the oxygen atom bound to the phosphorus at a single attachment site. The skilled artisan will understand that the polyethylene glycol unit in addition to comprising repeating polyethylene glycol subunits may also contain non-polyethylene glycol material (e.g., to facilitate coupling of multiple polyethylene glycol chains to each other or to facilitate coupling to the oxygen atom bound to the phosphorus). Non-polyethylene glycol material refers to the atoms in the polyethylene glycol unit that are not part of the repeating —CH₂CH₂O— subunits. In embodiments provided herein, the polyethylene glycol unit can comprise two monomeric polyethylene glycol chains linked to each other via non-polyethylene glycol elements. In other embodiments provided herein, the polyethylene glycol unit can comprise two linear polyethylene glycol chains attached to a central core that is attached to the oxygen atom bound to the phosphorus (i.e., the polyethylene glycol unit is branched).

There are a number of polyethylene glycol attachment methods available to those skilled in the art, [see, e.g., EP 0 401 384 (coupling PEG to G-CSF); U.S. Pat. No. 5,757,078 (PEGylation of EPO peptides); U.S. Pat. No. 5,672,662 (polyethylene glycol and related polymers mono substituted with propionic or butanoic acids and functional derivatives thereof for biotechnical applications); U.S. Pat. No. 6,077,939 (PEGylation of an N-terminal .alpha.-carbon of a peptide); and Veronese (2001) Biomaterials 22:405-417 (review article on peptide and protein PEGylation)].

In preferred embodiments, the polyethylene glycol unit is directly attached to the oxygen atom bound to the phosphorus. In these embodiments, the polyethylene glycol unit does not comprise a functional group for attachment to the oxygen atom bound to the phosphorous, i.e. the oxygen atom is directly bound to a carbon atom of the polyethylene glycol unit, preferably to a CH₂ of the polyethylene glycol unit.

In one group of embodiments, the polyethylene glycol unit comprises at least 1 ethylene glycol subunit, preferably at least 2 ethylene glycol subunits, more preferably at least 3 ethylene glycol subunits, still more preferably at least 4 ethylene glycol subunits, still more preferably at least 6 ethylene glycol subunits, even more preferably at least 8 ethylene glycol subunits. In some such embodiments, the polyethylene glycol unit comprises no more than about 100 ethylene glycol subunits, preferably no more than about 50 ethylene glycol units, more preferably no more than about 45 ethylene glycol subunits, more preferably no more than about 40 ethylene glycol subunits, more preferably no more than about 35 ethylene glycol subunits, even more preferably no more than about 30 ethylene glycol subunits.

In one group of embodiments, the polyethylene glycol unit comprises one or more linear polyethylene glycol chains each having at least 1 ethyleneglycol subunit, preferably at least 2 ethylene glycol subunits, more preferably at least 3 ethylene glycol subunits, still more preferably at least 4 ethylene glycol subunits, still more preferably at least 6 ethylene glycol subunits, even more preferably at least 8 ethylene glycol subunits. In preferred embodiments, the polyethylene glycol unit comprises a combined total of at least 1 ethylene glycol subunit, preferably at least 2 ethylene glycol subunits, more preferably at least 3, still more preferably at least 4, still more preferably at least 6, or even more preferably at least 8 ethylene glycol subunits. In some such embodiments, the polyethylene glycol unit comprises no more than a combined total of about 100 ethylene glycol subunits, preferably no more than a combined total of about 50 ethylene glycol subunits, more preferably no more than a combined total of about 45 ethylene glycol subunits, still more preferably no more than a combined total of about 40 ethylene glycol subunits, still more preferably no more than a combined total of about 35 ethylene glycol subunits, even more preferably no more than a combined total of about 30 ethylene glycol subunits.

In another group of embodiments, the polyethylene glycol unit comprises a combined total of from 1 to 100, preferably of from 2 to 50, more preferably of from 3 to 45, still more preferably of from 4 to 40, still more preferably of from 6 to 35, even more preferably of from 8 to 30 ethylene glycol subunits. In any one of these embodiments, the ethylene glycol subunit may be any ethylene glycol subunit as described herein.

In another group of embodiments, the polyethylene glycol unit comprises one or more linear polyethylene glycol chains having a combined total of from 1 to 100, preferably 2 to 50, more preferably 3 to 45, still more preferably 4 to 40, still more preferably 6 to 35, even more preferably 8 to 30 ethylene glycol subunits.

In another group of embodiments, the polyethylene glycol unit is a linear single polyethylene glycol chain having at least 1 ethylene glycol subunit, preferably at least 2 ethylene glycol subunits, more preferably at least 3 ethylene glycol subunits, still more preferably at least 6 ethylene glycol subunits, even more preferably at least 8 ethylene glycol subunits. Optionally, in any one of these embodiments the linear single polyalkylene glycol chain may be derivatized.

In another group of embodiments, the polyethylene glycol unit is a linear single polyethylene glycol chain having from 1 to 100, preferably 2 to 50, more preferably 3 to 45, more preferably 4 to 40, more preferably 6 to 35, more preferably 8 to 30 ethylene glycol subunits. Optionally, in any one of these embodiments the linear single polyethylene glycol chain may be derivatized.

Exemplary linear polyethylene glycol units that can be used as R¹, in any one of the embodiments provided herein, are as follows:

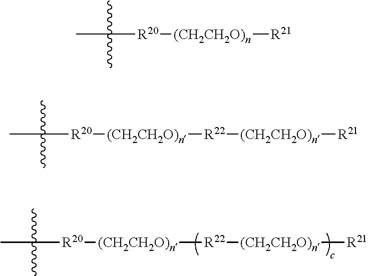

wherein the wavy line indicates the site of attachment to the oxygen atom bound to the phosphorus;
R²⁰ is a PEG attachment unit; preferably, R²⁰ is absent;
R²¹ is a PEG capping unit (herein, R²¹ is also denoted as “K^{F}”);
R²² is a PEG coupling unit (i.e. for coupling multiple PEG subunit chains together;
n is independently selected from 1 to 100, preferably from 2 to 50, more preferably from 3 to 45, more preferably from 4 to 40, still more preferably from 6 to 35, even more preferably from 8 to 30;
e is 2 to 5;
each n′ is independently selected from 1 to 100, preferably from 2 to 50, more preferably from 3 to 45, more preferably from 4 to 40, still more preferably from 6 to 35, even more preferably from 8 to 30. In preferred embodiments, there are at least 1, preferably at least 2, more preferably at least 3, more preferably at least 4, more preferably at least 6, even more preferably at least 8 ethylene glycol subunits in the polyethylene glycol unit. In some embodiments, there are no more than 100, preferably no more than 50, more preferably no more than 45, more preferably no more than 40, more preferably no more then 35, even more preferably no more than 30 ethylene glycol subunits in the polyethylene glycol unit. When R²⁰ is absent, a (CH₂CH₂O) subunit is directly bound to the oxygen atom, which is attached to the phosphorus.

Preferably, the linear polyethylene glycol unit is

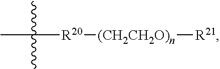

wherein the wavy line indicates the site of attachment to the oxygen atom bound to the phosphorus; R²⁰, R²¹ (also denoted herein as “K^{F}”) and n are as defined herein; more preferably R²⁰ is absent. In preferred embodiments, n is 12 or about 12. In preferred embodiments, n is 24 or about 24. Preferably, R²¹ is H.

The polyethylene glycol attachment unit R²⁰, when present, is part of the polyethylene glycol unit and acts to link the polyethylene glycol unit to the oxygen atom bound to the phosphorus. In this regard, the oxygen atom bound to the phosphorus forms a bond with the polyethylene glycol unit. In exemplary embodiments, the PEG attachment unit R²⁰, when present, is selected from the group consisting of *—(C₁-C₁₀)alkyl-^{#}, *-arylene-^{#}, *—(C₁-C₁₀)alkyl-O—#, *—(C₁-C₁₀)alkyl-C(O)—#, *—(C₁-C₁₀)alkyl-C(O)O—#, *—(C₁-C₁₀)alkyl-NH—#, *—(C₁-C₁₀)alkyl-S—#, *—(C₁-C₁₀)alkyl-C(O)—NH—#, *—(C₁-C₁₀)alkyl-NH—C(O)—#, and *—CH₂—CH₂SO₂—(C₁-C₁₀)alkyl-^{#}; wherein * denotes the attachment point to the oxygen bound to the phosphorus, and # denotes the attachment point to the ethylene glycol unit.

The PEG coupling unit R²², when present, is part of the polyethylene glycol unit and is non-PEG material that acts to connect two or more chains of repeating —CH₂CH₂O— subunits. In exemplary embodiments, the PEG coupling unit R²², when present, is independently selected from the group consisting of *—(C₁-C₁₀)alkyl-C(O)—NH—#, *—(C₁-C₁₀)alkyl-NH—C(O)—#, *—(C₂-C₁₀)alkyl-NH—#, *—(C₂-C₁₀)alkyl-O—#, *—(C₁-C₁₀)alkyl-S—#, or *—(C₂-C₁₀)alkyl-NH-^{#}; wherein * denotes the attachment point to an oxygen atom of an ethylene glycol subunit, and # denotes the attachment point to a carbon atom of another ethylene glycol subunit.

The group R²¹, also denoted herein as “K^{F}”, in exemplary embodiments is H (hydrogen), or may be a capping group, as described herein; preferably, R²¹ is independently selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂. In some embodiments R²¹ may be —(C₁-C₁₀)alkyl, in particular methyl. More preferably, R²¹ is H.

Illustrative linear polyethylene glycol units, which can be used as R¹ in any one of the embodiments provided herein, are as follows.

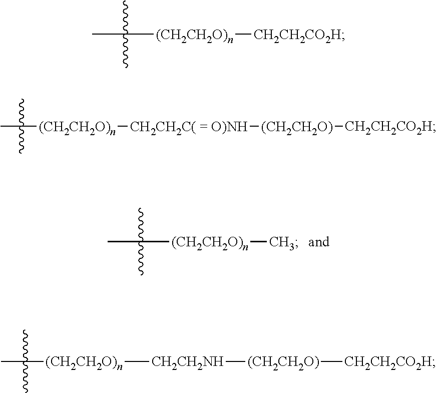

wherein the wavy line indicates the site of attachment to the oxygen atom which is bound to the phosphorus; and each n is from 1 to 100, preferably from 2 to 50, more preferably from 3 to 45, still more preferably from 4 to 40, still more preferably from 6 to 35, even more preferably from 8 to 30. In some embodiments, n is about 12. In some embodiments, n is about 24.

In some embodiments, the polyethylene glycol unit is from about 300 daltons to about 5 kilodaltons; from about 300 daltons, to about 4 kilodaltons; from about 300 daltons, to about 3 kilodaltons; from about 300 daltons, to about 2 kilodaltons; or from about 300 daltons, to about 1 kilodalton. In some such aspects, the polyethylene glycol unit may have at least 6 ethylene glycol subunits or at least 8 ethylene glycol subunits. In some such aspects, the polyethylene glycol unit may have at least 6 ethylene glycol subunits or at least 8 ethylene glycol subunits but no more than 100 ethylene glycol subunits, preferably no more than 50 ethylene glycol subunits. In some embodiments, the polyethylene glycol unit is a polyethylene glycol unit being from about 300 daltons to about 5 kilodaltons; from about 300 daltons to about 4 kilodaltons; from about 300 daltons to about 3 kilodaltons; from about 300 daltons to about 2 kilodaltons; or from about 300 daltons to about 1 kilodalton. In some such aspects, the polyethylene glycol unit may have at least 6 ethylene glycol subunits or at least 8 ethylene glycol subunits. In some aspects, the polyethylene glycol unit has at least 6 ethylene glycol subunits or at least 8 ethylene glycol subunits but no more than 100 ethylene glycol subunits, preferably no more than 50 ethylene glycol subunits.

In some embodiments, when R¹ is a polyethylene glycol unit, there are no other ethylene glycol subunits and/or alkylene glycol subunits present in the antibody drug conjugate of formula (I) (i.e., no ethylene glycol subunits and/or alkylene glycol subunits are present in any of the other components of the antibody drug conjugate, such as e.g. in the connector unit (CU) as provided herein). In other aspects, when R¹ is a polyethylene glycol unit, there are no more than 8, no more than 7, no more than 6, no more than 5, no more than 4, no more than 3, no more than 2 or no more than 1 other ethylene glycol subunits and/or alkylene glycol subunits present in the conjugate of formula (I) (i.e., no more than 8, 7, 6, 5, 4, 3, 2, or 1 other ethylene glycol and/or alkylene glycol subunits are present in other components of the conjugate, such as e.g. in the connector unit (CU) as provided herein).

It will be appreciated that when referring to ethylene glycol subunits, and/or alkylene glycol subunits, and depending on context, the number of subunits can represent an average number, e.g., when referring to a population of conjugates or intermediate compounds, and using polydisperse polyethylene glycols.

### “CU”: Connector Unit

The present disclosure provides antibody drug conjugates (ADCs), where an anti-TPBG antibody (Ab), as described herein, is linked to a cytotoxic moiety (CM). In accordance with the present disclosure, the antibody (Ab) may be linked, via the group Y and covalent attachment by a connector unit CU, to the cytotoxic moiety. As used herein, a “connector unit” CU is any chemical moiety that is capable of linking a group Y, such as e.g. NH, to another moiety, such as a cytotoxic moiety. In particular, a connector unit CU forms part of a linker (L) which is capable of linking the antibody of the present invention with one or more cytotoxic moieties CM (or drug moieties or cytotoxic payloads), as described herein. In this regard, it is again referred to the formula (I) described herein:

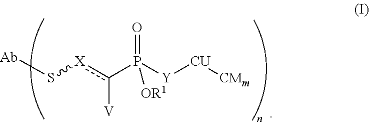

Accordingly, the cytotoxic moiety CM can be linked to Y through a connector unit CU. In formula (I) Ab, [symbol] , V, X, Y, R¹, CU, CM, m and n are as defined herein. The connector unit CU serves to connect the Y with the cytotoxic moiety (CM). The connector unit CU is any chemical moiety that is capable of linking Y to the cytotoxic moiety CM. In particular, the connector unit CU attaches Y to the cytotoxic moiety CM through covalent bond(s). The connector unit is a bifunctional or multifunctional moiety which can be used to link a cytotoxic moiety CM and Y to form antibody drug conjugates of formula (I). The terms “connector unit”, “connector reagent”, “linker reagent”, “cross-linking reagent”, “linker derived from a cross-linking reagent” and “linker” may be used interchangeably throughout the present disclosure.

Connector units CU can be susceptible to cleavage (cleavable connector unit) such as enzymatic cleavage, acid-induced cleavage, photo-induced cleavage and disulfide bond cleavage. Enzymatic cleavage includes, but is not limited to, protease-induced cleavage, peptidase-induced cleavage, esterase-induced cleavage, glycosidase-induced cleavage, phosphatase-induced cleavage, and sulfatase-induced cleavage, preferably at conditions under which the cytotoxic moiety and/or the antibody (Ab) remains active. Alternatively, connector units can be substantially resistant to cleavage (e.g., stable connector unit or non-cleavable connector unit). In some aspects, the connector unit CU may be a procharged connector unit, a hydrophilic connector unit, a PEG-based connector unit, or a dicarboxylic acid based connector unit. Accordingly, in some embodiments of any one of the antibody drug conjugates disclosed herein the connector unit (CU) is selected from the group consisting of a cleavable connector unit, a non-cleavable connector unit, a hydrophilic connector unit, a PEG-based connector unit, a procharged connector unit, a peptidic connector unit and a dicarboxylic acid based connector unit. Preferably, the connector unit CU is a cleavable connector unit. In some embodiments, the connector unit CU is a non-cleavable connector unit.

Preferably, as described herein, the connector unit CU is cleavable. In some embodiments, CU is a connector unit susceptible to enzymatic cleavage. In some embodiments, CU is an acid-labile connector unit, a photo-labile connector unit, a peptidase cleavable connector unit, a protease cleavable connector unit, an esterase cleavable connector unit, a glycosidase cleavable connector unit, a phosphatase cleavable connector unit, a sulfatase cleavable connector unit, a disulfide bond reducible connector unit, a hydrophilic connector unit, a procharged connector unit, a PEG-based connector unit, or a dicarboxylic acid based connector unit. Preferably, the connector unit CU is cleavable by a protease, a glucuronidase, a sulfatase, a phosphatase, an esterase, or by disulfide reduction. Preferably, the connector unit is a peptidase cleavable connector unit. Other preferred connector units are cleavable by a protease.

A non-cleavable connector unit is any chemical moiety capable of linking (or connecting) a cytotoxic moiety to Y in a stable, covalent manner and does not fall off under the categories listed herein for cleavable connector units. Thus, non-cleavable connector units are substantially resistant to acid-induced cleavage, photo-induced cleavage, peptidase-induced cleavage, protease-induced cleavage, glycosidase-induced cleavage, phosphatase-induced cleavage, esterase-induced cleavage and disulfide bond cleavage. Furthermore, non-cleavable refers to the ability of the chemical bond in the connector unit or adjoining to the connector unit to withstand cleavage induced by an acid, photo labile-cleaving agent, a peptidase, a protease, a glycosidase, a phosphatase, an esterase, or a chemical or physiological compound that cleaves a disulfide bond, at conditions under which the cytotoxic moiety or the antibody (Ab) does not lose its activity.

Acid-labile connector units are connector units cleavable at acidic pH. For example, certain intracellular compartments, such as endosomes and lysosomes, have an acidic pH (pH 4-5), and provide conditions suitable to cleave acid-labile connector units.

Some connector units can be cleaved by peptidases, i.e. peptidase cleavable connector units. In this regard, certain peptides are readily cleaved inside or outside cells, see e.g. Trout et al., 79 Proc. Natl. Acad. Sci. USA, 626-629 (1982) and Umemoto et al. 43 Int. J. Cancer, 677-684 (1989). Peptides are composed of α-amino acids and peptidic bonds, which chemically are amide bonds between the carboxylate of one amino acid and the amino group of a second amino acid.

Some connector units CU can be cleaved by esterases, i.e. esterase cleavable connector units. In this regard, certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

Procharged connector units are derived from charged cross-linking reagents that retain their charge after incorporation into an antibody drug conjugate. Examples of procharged connector units (or linkers) can be found in US 2009/0274713.

Preferably, as described herein, the connector unit CU is cleavable. As illustrative examples, the connector unit may be cleavable by a protease, a glucuronidase, a sulfatase, a phosphatase, an esterase, or by disulfide reduction. Preferably, the connector unit CU is cleavable by a protease. More preferably, the connector unit is cleavable by a cathepsin, such as, in particular, cathepsin B. The connector unit may comprise a dipeptide moiety, such as e.g. a valine-citrulline moiety or a valine-alanine moiety, which can be cleaved by a cathepsin such as cathepsin B. Accordingly, in some embodiments the connector unit comprises a valine-citrulline moiety. In some embodiments the connector unit comprises a valine-alanine moiety. The connector unit may comprise a cleavage site. The term “cleavage site” may refer to a chemical moiety which is recognized by an enzyme, followed by cleavage, e.g. by way of hydrolysis. As an illustrative example, a cleavage site is a sequence of amino acids, which is recognized by a protease or a peptidase, and hydrolyzed by said protease or peptidase. In some embodiments, the cleavage site is a dipeptide. In some embodiments, the cleavage site is a valine-citrulline moiety. In some embodiments, the cleavage site is a valine-alanine moiety.

### Second Spacer Unit

In preferred embodiments, the connector unit (CU) comprises a second spacer unit -A- which is bound to the —Y—. The second spacer unit serves to connect a —Y— to another part of the connector unit, when present, or to a cytotoxic moiety (—CM). As readily appreciated by a person skilled in the art, this depends on whether another part of the connector unit is present or not. The second spacer unit (-A-) may be any chemical group or moiety which is capable to connect a —Y— to another part of the connector unit, when present, or to a cytotoxic moiety (—CM), depending on whether another part of the connector unit is present or not. In this regard, the —Y—, as described herein, is bonded to the second spacer unit (-A-). The second spacer unit (-A-) may comprise or may be a functional group that is capable to form a bond to another part of the connector unit, when present, or to the cytotoxic moiety (—CM). Again, this depends on whether another part of the connector is present or not. Preferably, the functional group, which is capable to form a bond to another part of the connector unit, or to a cytotoxic moiety (—CM), is a carbonyl group which is depicted as, e.g.,

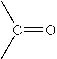

### or —C(O)—.

The second spacer unit may be any spacer known to a person skilled in the art, for example, a straight or branched hydrocarbon-based moiety. The second spacer unit can also comprise cyclic moieties, such as e.g., but not limited to, aromatic moieties. If the second spacer unit is a hydrocarbon-based moiety, the main chain of the second spacer moiety may comprise only carbon atoms but can also contain heteroatoms such as oxygen (O), nitrogen (N) or sulfur (S) atoms, and/or can contain carbonyl groups (C═O). The second spacer unit may comprise or may be, for example, a (C₁-C₂₀) carbon atom chain. In typical embodiments of hydrocarbon-based second spacer units, the spacing moiety comprises between 1 to about 150, 1 to about 100, 1 to about 75, 1 to about 50, or 1 to about 40, or 1 to about 30, or 1 to about 20, including 2, 3, 4, 5, 6, 7, 8, 9, 10, 11, 12, 13, 14, 15, 16, 17, 18 and 19 main chain atoms. A person skilled in the art knows to select suitable second spacer units.

In some embodiments, the second spacer unit (-A-), when present, is selected from the group consisting of *—(C₁-C₁₀)alkylene-C(O)—#, *—(C₃-C₈)carbocyclo-C(O)—#, *-arylene-C(O)—#, *—(C₁-C₁₀)alkylene-arylene-C(O)—#, *-arylene-(C₁-C₁₀)alkylene-C(O)—#, *—(C₁-C₁₀)alkylene-(C₃-C₈)carbocyclo-C(O)—#, *—(C₃-C₈)carbocyclo-(C₁-C₁₀)alkylene-C(O)—#, *—(C₃-C₈)heterocyclo-C(O)—#, *—(C₁-C₁₀)alkylene-(C₃-C₈)heterocyclo-C(O)—#, and *—(C₃-C₈)heterocyclo-(C₁-C₁₀)alkylene-C(O)—#; * denotes the attachment point to the —Y—; and # denotes the attachment point to another part of the connector unit, when present, or to a cytotoxic moiety (—CM), depending on whether another part of the connector unit is present or not. Preferably, the second spacer unit (-A-), when present, is selected from the group consisting of *—(C₃-C₈)carbocyclo-C(O)—#, *-arylene-C(O)—#, and *—(C₃-C₈)heterocyclo-C(O)—#; * denotes the attachment point to the —Y—; and # denotes the attachment point to another part of the connector unit, when present, or to a cytotoxic moiety (—CM), depending on whether another part of the connector unit is present or not.

In other embodiments, the second spacer unit (-A-), when present, may be selected from the group consisting of *—(C₁-C₁₀)alkylene-^{#}, *—(C₃-C₈)carbocyclo-^{#}, *-arylene-^{#}, *—(C₁-C₁₀)alkylene-arylene-^{#}, *-arylene-(C₁-C₁₀)alkylene-^{#}, *—(C₁-C₁₀)alkylene-(C₃-C₈)carbocyclo-^{#}, *—(C₃-C₈)carbocyclo-(C₁-C₁₀)alkylene-^{#}, *—(C₃-C₈)heterocyclo-^{#}, *—(C₁-C₁₀)alkylene-(C₃-C₈)heterocyclo-^{#}, and *—(C₃-C₈)heterocyclo-(C₁-C₁₀)alkylene-^{#}; * denotes the attachment point to the —Y—; and # denotes the attachment point to another part of the connector unit, when present, or to a cytotoxic moiety (—CM), depending on whether another part of the connector unit is present or not. Preferably, the second spacer unit (-A-), when present, may be selected from the group consisting of *—(C₃-C₈)carbocyclo-^{#}, *-arylene-^{#}, and *—(C₃-C₈)heterocyclo-^{#}; * denotes the attachment point to the —Y—; and # denotes the attachment point to another part of the connector unit, when present, or to a cytotoxic moiety (—C), depending on whether another part of the connector unit is present or not.

Preferably, the second spacer unit -A- is

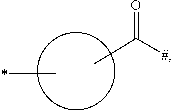

wherein

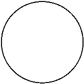

is a five- or six-membered carbocyclic ring; * denotes the attachment point to the μ-Y—; and # denotes the attachment point to another part of the connector unit, when present, or to a cytotoxic moiety (—CM), depending on whether another part of the connector unit is present or not. The carbocyclic ring may be aromatic or non-aromatic. Preferably, the second spacer unit -A- is

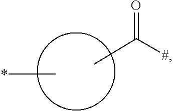

wherein

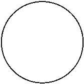

is a five- or six-membered heterocyclic ring comprising 1, 2, or 3 heteroatoms independently selected from the group consisting of N, O and S; * denotes the attachment point to the —Y—; and # denotes the attachment point to another part of the connector unit, when present, or to a cytotoxic moiety (—CM), depending on whether another part of the connector unit is present or not. The heterocyclic ring may be aromatic or non-aromatic.

More preferably,

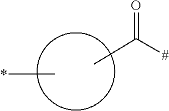

is selected from the group consisting of

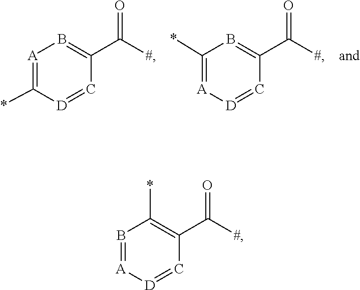

wherein each of A, B, C and D is independently selected from N (nitrogen) and C—H; preferably, at least one of A, B, C and D is C—H; more preferably, at least two of A, B, C and D are C—H; still more preferably, at least three of A, B, C and D are C—H, even more preferably, each of A, B, C and D are C—H; * denotes the attachment point to the —Y—; and # denotes the attachment point to another part of the connector unit, when present, or to a cytotoxic moiety (—CM), depending on whether another part of the connector unit is present or not. Still more preferably,

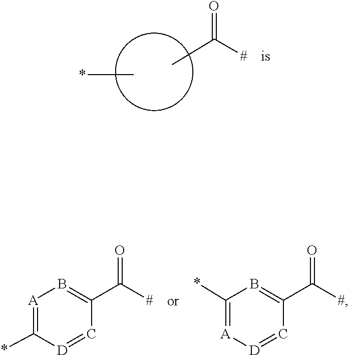

wherein each of A, B, C and D is independently selected from N (nitrogen) and C—H; preferably, at least one of A, B, C and D is C—H; more preferably, at least two of A, B, C and D are C—H; still more preferably, at least three of A, B, C and D are C—H, even more preferably, each of A, B, C and D are C—H; wherein * denotes the attachment point to the —Y—; and # denotes the attachment point to another part of the connector unit, when present, or to a cytotoxic moiety (—CM), depending on whether another part of the connector unit is present or not. Even more preferably,

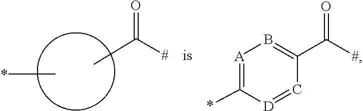

wherein each of A, B, C and D is independently selected from N (nitrogen) and C—H; preferably, at least one of A, B, C and D is C—H; more preferably, at least two of A, B, C and D are C—H; still more preferably, at least three of A, B, C and D are C—H, even more preferably, each of A, B, C and D are C—H; wherein * denotes the attachment point to the —Y—; and # denotes the attachment point to another part of the connector unit, when present, or to a cytotoxic moiety (—CM), depending on whether another part of the connector unit is present or not. In very preferred embodiments, the second spacer unit A is

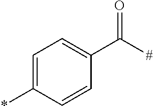

wherein * denotes the attachment point to the —Y—; and # denotes the attachment point to another part of the connector unit, when present, or to a cytotoxic moiety (—CM), depending on whether another part of the connector unit is present or not.

In other embodiments, the second spacer unit (-A-) may be

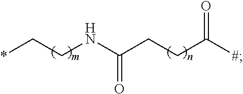

and m and n are each, independently, an integer of e.g. from 0 to 20, 0 to 15, 1 to 10, 1 to 8, 1 to 6, 1 to 4, 1 to 3, 1 to 2, or 1, preferably m is 1 and n is 1; * indicates the position of the —Y—, and # denotes the attachment point to another part of the connector unit, when present, or to a cytotoxic moiety (—CM), depending on whether another part of the connector unit is present or not. Such second spacer unit may be optionally substituted, e.g. with one or two (C₁-C₈)alkyl, in particular at the carbon adjacent to the asterisk (*).
Connector Unit *-Aₐ-W_{w}—B_{b}-#

In some embodiments, the connector unit CU has the formula: *-Aₐ-W_{w}-B_{b}##, wherein: -A- is a second spacer unit, as described herein; a is 0 or 1; each —W— is independently an amino acid; w is independently an integer ranging from 0 to 12; —B— is a first spacer unit; and b is 0 or 1; * denotes the attachment point to the —Y—; and ## denotes the attachment point to the cytotoxic moiety CM. Herein, the notation “W_{w}”, or —“W_{w}—”, or the like, i.e. the combination of W and the associated integer w, is also denoted as “amino acid unit”. Examples for suitable second spacer units, amino acid units and first spacer units are described, e.g., in WO 2004/010957 A2.

In the connector unit having the structure *-Aₐ-W_{w}—B_{b}-^{#}#, the second spacer unit serves to connect a —Y— to the amino acid unit —W_{w}—. The second spacer unit (-A-) may be any second spacer unit as described herein. When present, the second spacer unit (-A-) may be any chemical group or moiety which is capable to link a —Y— to the amino acid unit. Alternatively, the second spacer unit may link the —Y— to the first spacer unit, in case no amino acid unit is present. Alternatively, the second spacer unit may link the —Y— to the cytotoxic moiety (—C), in case no first spacer unit and no amino acid unit are present. In this regard, the —Y—, as described herein, is bonded to the second spacer unit (-A-). The second spacer unit (-A-) may comprise or may be a functional group that is capable to form a bond to an amino acid unit (—W_{w}—), or to a first spacer unit (—B—), or to a cytotoxic moiety (—CM), depending on whether an amino acid unit (—W_{w}—) and/or a first spacer unit (—B—) is present or not. Preferably, the functional group, which is capable to form a bond to an amino acid unit (—W_{w}—), in particular to the N terminus of the amino acid unit, or to a first spacer unit (—B—), or to a cytotoxic moiety (—CM), is a carbonyl group which is depicted as, e.g.

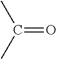

or —C(O)—. The integer a associated with the second spacer unit may be 0 or 1. Preferably, the integer a is 1. Alternatively, in other embodiments the second spacer unit is absent (a=0).

The amino acid unit (—W_{w}—), when present, may link the second spacer unit A to the first spacer unit B in case the first spacer unit is present. Alternatively, the amino acid unit may link the second spacer unit to the cytotoxic moiety (CM) in case the first spacer unit is absent. Alternatively, the amino acid unit may link the Y to the first spacer unit in case the second spacer unit is absent. Alternatively, the amino acid unit may link the Y to the cytotoxic moiety in case the first spacer unit and the second spacer unit are absent.

The amino acid unit —W_{w}— may be a dipeptide (w=2), a tripeptide (w=3), a tetrapeptide (w=4), a pentapeptide (w=5), a hexapeptide (w=6), a heptapeptide (w=7), an octapeptide (w=8), a nonapeptide (w=9), a decapeptide (w=10), an undecapeptide (w=11) or a dodecapeptide (w=12).

In some embodiments, the amino acid unit can comprise natural amino acids. In some embodiments, the amino acid unit can comprise non-natural amino acids.

In any one of the embodiments described herein, each amino acid of the amino acid unit, except for amino acids which are not chiral such as e.g. glycine, may be independently in the L configuration or in the D configuration. Preferably, in any one of the embodiments described herein each amino acid of the amino acid unit, except for amino acids which are not chiral such as e.g. glycine, is in the L configuration (i.e., in the naturally occurring configuration).

Preferably, when a second spacer unit (-A-) is present, in any one of the embodiments described herein the N terminus of the amino acid unit —W_{w}— is bound to the second spacer unit (A), more preferably via a carbonyl group of the second spacer unit. Preferably, in any one of the embodiments described herein, the C terminus of the amino acid unit —W_{w}— is bound to a first spacer unit (B) in case a first spacer unit is present. Alternatively, in any one of the embodiments described herein, the C terminus of the amino acid unit —W_{w}— may be bound to the cytotoxic moiety (—CM) in case a first spacer unit is absent. In other embodiments, the N-terminus of the amino acid unit —W_{w}— may be bound to the first spacer unit (B), when present, and the C-terminus may be bound to the second spacer unit A, when present.

In some embodiments, w may be 1 or 2. Preferably, the amino acid unit W_{w} is a dipeptide (w=2). In the dipeptide each amino acid independently may have the formula denoted below in the square brackets:

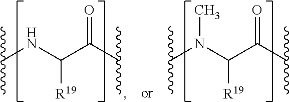

wherein R¹⁹ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

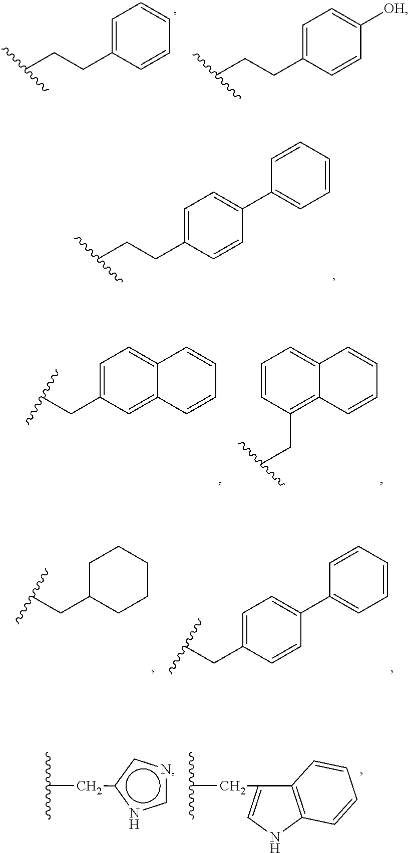

The amino acid unit can be enzymatically cleaved by one or more enzymes, including but not limited to a tumor-associated protease, preferably a cathepsin, more preferably cathepsin B, to liberate the cytotoxic moiety (—CM), which in one embodiment is protonated in vivo upon release to provide a free cytotoxic moiety (CM). Illustrative —W_{w}— units are represented by formula (VII).

Accordingly, the —W_{w}— unit may be a dipeptide of formula (VII):

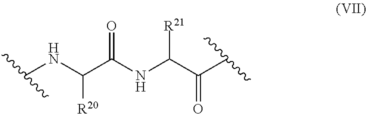

wherein R²⁰ and R²¹ are as follows:

| R²⁰ | R²¹ |
|---|---|
| benzyl | (CH₂)₄NH₂; |
| methyl | (CH₂)₄NH₂; |
| isopropyl | (CH₂)₄NH₂: |
| Isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 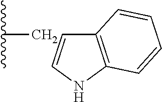 | (CH₂)₃NHCONH₂; |
| benzyl | methyl; and |
| benzyl | (CH₂)₃NHC(═NH)NH₂; |

Exemplary amino acid units include, but are not limited to, units of formula (VII) where: R²⁰ is benzyl and R²¹ is —(CH₂)₄NH₂ (Phe-Lys); R²⁰ is isopropyl and R²¹ is —(CH₂)₄NH₂ (Val-Lys); R²⁰ is isopropyl and R²¹ is —(CH₂)₃NHCONH₂ (Val-Cit).

Useful —W_{w}— units can be designed and optimized in their selectivity for enzymatic cleavage by a particular enzyme, for example, a tumor-associated protease. In one embodiment, a —W_{w}— unit is that whose cleavage is catalyzed by cathepsin B, C and/or D, or a plasmin protease (“tumor-associated proteases”). Preferably, the —W_{w}— unit is cleaved by cathepsin B. Suitable linkers, which can be cleaved by a protease, are described, e.g., in G. M. Dubowchik et al., “Cathepsin B-Labile Dipeptide Linkers for Lysosomal Release of Doxorubicin from Internalizing Immunoconjugates; Model Studies of Enzymatic Drug Release and Antigen-Specific In Vitro Anticancer Activity”, Bioconjugate Chem., Vol. 13, No. 4, 2002, 855-869; S. C. Jeffrey et al., “Dipeptide-based highly potent doxorubicin antibody conjugate”, Bioorg. Med. Chem. Lett. 16 (2006), 358-362; and M. S. Kung Sutherland et al., “SGN-CD33A: a novel CD33-targeting antibody-drug conjugate using a pyrrolobenzodiazepine dimer is active in models of drug-resistant AML”, Blood, 22 Aug. 2013, volume 122, number 8, 1455-1463.

When R¹⁹, R²⁰ or R²¹ is other than hydrogen, the carbon atom to which R¹⁹, R²⁰ or R²¹ is attached is chiral. Each carbon atom to which R¹⁹, R²⁰ or R²¹ is attached may be independently in the (S) or (R) configuration. Preferably, each carbon atom to which R¹⁹, R²⁰ or R²¹ is attached, when chiral, is in the (S) configuration.

In one preferred embodiment, the amino acid unit is valine-citrulline (i.e. Val-Cit or VC). In another preferred embodiment, the amino acid unit is valine-alanine (i.e. Val-Ala or VA). In another preferred embodiment, the amino acid unit is alanine-alanine (i.e. Ala-Ala or AA). In another preferred embodiment, the amino acid unit is phenylalanine-lysine (i.e. Phe-Lys or FK). Such connector units are illustrative examples for a connector unit which can be cleaved by a protease, such as e.g. cathepsin B.

The notation of peptides used herein throughout this specification follows the conventional nomenclature. Accordingly, the N-terminus of a peptide is written on the left, and the C-terminus of the peptide is written on the right. As an illustrative but non-limiting example, in the dipeptide valine-citrulline (i.e. Val-Cit or VC), the valine has the N-terminus, and the citrulline has the C-terminus. Preferably, in any one of the embodiments described herein, when a second spacer unit (-A-) is present, the N-terminus of a peptide, such as e.g. of a dipeptide (as illustrative non-limiting example: Val-Cit), is bound to the second spacer unit (-A-), more preferably via a carbonyl group of the second spacer unit, and the C-terminus of the peptide is bound to a first spacer unit (—B—), in case a first spacer unit (—B—) is present, or to the camptothecin moiety (—C) in case a first spacer unit (—B—) is absent.

In yet another embodiment, the amino acid unit is N-methylvaline-citrulline. In yet another embodiment, the amino acid unit is selected from the group consisting of 5-aminovaleric acid, homophenylalanine-lysine, tetraisoquinolinecarboxylate-lysine, cyclohexylalanine-lysine, isonepecotic acid-lysine, betaalanine-lysine, and isonepecotic acid.

Preferably, the amino acid unit is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, the amino acid unit is valine-citrulline (i.e. Val-Cit or VC) or valine-alanine (i.e. Val-Ala or VA). Even more preferably, the amino acid unit is valine-citrulline (i.e. Val-Cit or VC).

In some embodiments, the amino acid unit is selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). Preferably, the amino acid unit is selected from the group consisting of valine-glutamine (i.e. Val-Gln or VQ), leucine-glutamine (i.e. Leu-Gln or LQ), and phenylalanine-glutamin (i.e. Phe-Gln or FQ). More preferably, the amino acid unit is valine-glutamine (i.e. Val-Gln or VQ) or leucine-glutamine (i.e. Leu-Gln or LQ). Connector units which comprise amino acid units according to these embodiments can be illustrative examples for a connector unit which is cleavable, in particular by a protease, such as e.g. a cathepsin (e.g., cathepsin B). The amino acid units of these embodiments and further suitable amino acid units are disclosed, e.g., in Salomon et al., “Optimizing Lysosomal Activation of Antibody-Drug Conjugates (ADCs) by Incorporation of Novel Cleavable Dipeptide Linkers”, Mol. Pharmaceutics 2019, 16, 12, 4817-4825.

The first spacer unit (B), when present, may link an amino acid unit (W_{w}) to the cytotoxic moiety when an amino acid unit is present. Alternatively, the first spacer unit (B) may link the second spacer unit (A) to the cytotoxic moiety (CM) when the amino acid unit is absent. The first spacer unit may link the cytotoxic moiety to the Y when both the amino acid unit and second spacer unit are absent.

The integer b may be 0 or 1. In preferred embodiments, the integer b is 1. Alternatively, in other embodiments, the integer b is 0, and the first spacer unit is absent.

The first spacer unit (—B—) may be of two general types: self-immolative and non-self-immolative. A non-self-immolative first spacer unit is one in which part or all of the first spacer unit remains bound to the cytotoxic moiety (CM) after cleavage, particularly enzymatic, of an amino acid unit (—W_{w}—) of the linker (L). Alternatively, an exemplary compound containing a self-immolative first spacer unit can release a cytotoxic moiety —CM without the need for a separate hydrolysis step. In an exemplary embodiment, a self-immolative first spacer unit is a PAB group that is linked to —W_{w}— via the amino nitrogen atom of the PAB group, and connected directly to —CM via a carbonate, carbamate or ether group. Without being bound by any particular theory or mechanism, Scheme 2 depicts a possible mechanism of drug release of a PAB group which is attached directly to a drug moiety -D, via a carbamate or carbonate group espoused by Toki et al. (2002) J Org. Chem. 67:1866-1872. Herein, the drug moiety D is also denoted as cytotoxic moiety CM.

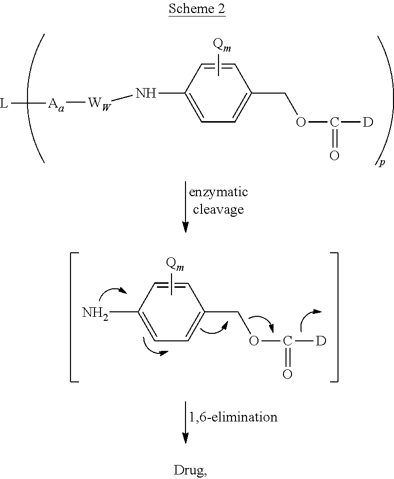

wherein Q is —(C₁-C₈)alkyl, —O—(C₁-C₈)alkyl, -halogen, -nitro or -cyano; m is an integer ranging from 0 to 4, preferably m is 0, 1 or 2, more preferably m is 0 or 1, still more preferably m is 0; and p ranges from 1 to 20.

Without being bound by any particular theory or mechanism, Scheme 3 depicts a possible mechanism of drug release of a PAB group which is attached directly to a drug moiety -D via an ether or amine linkage. Herein, the drug moiety D is also denoted as cytotoxic moiety CM.

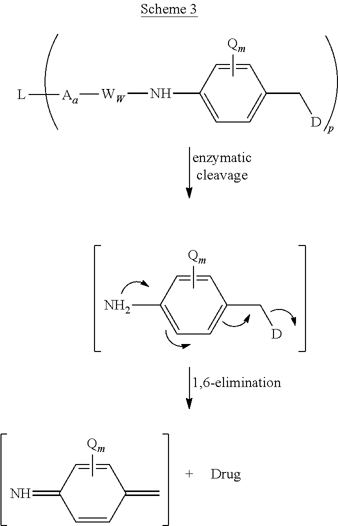

wherein Q is —(C₁-C₈)alkyl, —O—(C₁-C₈)alkyl, -halogen, -nitro or -cyano; m is an integer ranging from 0 to 4, preferably m is 0, 1 or 2, more preferably m is 0 or 1, still more preferably m is 0; and p ranges from 1 to 20.

Other examples of self-immolative spacers include, but are not limited to, aromatic compounds that are electronically similar to the PAB group such as 2-aminoimidazol-5-methanol derivatives (Hay et al. (1999) Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223), appropriately substituted bicyclo[2.2.1] and bicyclo[2.2.2] ring systems (Storm, et al., J. Amer. Chem. Soc., 1972, 94, 5815) and 2-aminophenylpropionic acid amides (Amsberry, et al., J. Org. Chem., 1990, 55, 5867). Elimination of amine-containing drugs that are substituted at the alpha-position of glycine (Kingsbury, et al., J. Med. Chem., 1984, 27, 1447) are also examples of self-immolative spacer useful in exemplary compounds.

In one embodiment, the first spacer unit is a branched bis(hydroxymethyl)styrene (BHMS) unit as depicted in Scheme 4, which can be used to incorporate and release multiple drugs (D). Herein, the drug moiety D is also denoted as cytotoxic moiety CM.

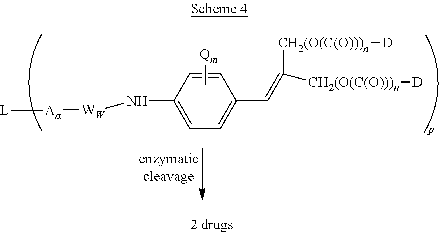

wherein Q is —(C₁-C₈)alkyl, —O—(C₁-C₈)alkyl, -halogen, -nitro or -cyano; m is an integer ranging from 0 to 4; preferably m is 0, 1 or 2; more preferably m is 0 or 1; still more preferably m is 0; and p ranges from 1 to 10; n is 0 or 1; and p ranges from 1 to 20.

In preferred embodiments, the first spacer unit is represented by formula (X):

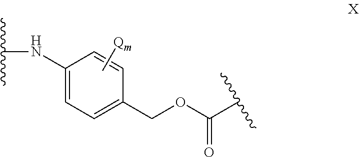

wherein Q is —(C₁-C₈)alkyl, —O—(C₁-C₈)alkyl, -halogen, -nitro or -cyano; and m is an integer ranging from 0 to 4; preferably m is 0, 1 or 2; more preferably m is 0 or 1; in very preferred embodiments m is 0. Preferably, when an amino acid unit is present, in formula (X), the NH group is bound to a C-terminus of the amino acid unit. Preferably, in formula (X), the C(O) group is bound to the cytotoxic moiety (CM), such as, for example, a camptothecin moiety.

In very preferred embodiments, the first spacer unit is a PAB group having the following structure:

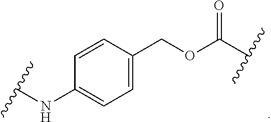 [SMILES: *Nc1ccc(COC(*)=O)cc1]

Preferably, when an amino acid unit is present, the NH group is bound to an amino acid unit (—W_{w}—), more preferably to a C-terminus of the amino acid unit. Preferably, the C(O) group is bound to the cytotoxic moiety (CM), such as, for example, a camptothecin moiety.

In some embodiments, the first spacer group (—B—) is a heterocyclic “self-immolating moiety” of Formulas I, II or III bound to the cytotoxic moiety and incorporates an amide group that upon hydrolysis by an intracellular protease initiates a reaction that ultimately cleaves the first spacer unit (—B—) from the cytotoxic moiety such that the cytotoxic moiety is released from the conjugate in an active form. The connector unit further comprises an amino acid unit (—W_{w}—) adjacent to the first spacer group (—B—) that is a substrate for an intracellular enzyme, for example an intracellular protease such as a cathepsin (e.g., cathepsin B), that cleaves the peptide at the amide bond shared with the first spacer group (—B—). Heterocyclic self-immolating moieties are described, e.g., in WO 2019/236954.

In some embodiments, the first spacer unit (—B—) is a heterocyclic self-immolating group selected from Formulas I, II and III:

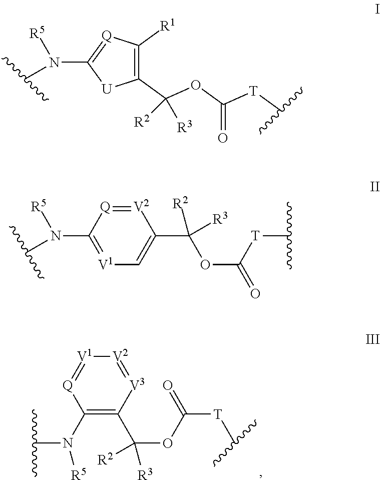

wherein the wavy lines indicate the covalent attachment sites to the amino acid unit —W_{w}— and the cytotoxic moiety CM, and wherein U is O, S or NR⁶; Q is CR⁴ or N; V¹, V² and V³ are independently CR⁴ or N provided that for formula II and III at least one of Q, V¹ and V² is N; T may be O pending from a cytotoxic moiety (—CM); R¹, R², R³ and R⁴ are independently selected from the group consisting of H, F, Cl, Br, I, OH, —N(R⁵)₂, —N(R⁵)₃⁺, —(C₁-C₈)alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, —(C₁-C₈)alkoxy, —(C₁-C₈)halosubstituted alkyl, polyethyleneoxy, phosphonate, phosphate, —(C₁-C₈)alkyl, —(C₁-C₈)substituted alkyl, —(C₂-C₈)alkenyl, —(C₂-C₈)substituted alkenyl, —(C₂-C₈)alkynyl, —(C₂-C₈)substituted alkynyl, —(C₆-C₂₀)aryl, —(C₆-C₂₀)substituted aryl, —(C₃-C₂₀)heterocycle, and —(C₃-C₂₀)substituted heterocycle; or when taken together, R² and R³ form a carbonyl (═O), or spiro carbocyclic ring of 3 to 7 carbon atoms; and R⁵ and R⁶ are independently selected from H, —(C₁-C₈)alkyl, —(C₁-C₈)substituted alkyl, —(C₂-C₃)alkenyl, —(C₂-C₈)substituted alkenyl, —(C₂-C₈)alkynyl, —(C₂-C₈)substituted alkynyl, —(C₆-C₂₀)aryl, —(C₆-C₂₀)substituted aryl, —(C₃-C₂₀)heterocycle, and —(C₃-C₂₀)substituted heterocycle; wherein —(C₁-C₈)substituted alkyl, —(C₂-C₈) substituted alkenyl, —(C₂-C₈)substituted alkynyl, —(C₆-C₂₀)substituted aryl, and —(C₃-C₂₀)substituted heterocycle are independently substituted with one or more substituents selected from the group consisting of F, Cl, Br, I, OH, —N(R⁵)₂, —N(R⁵)₃⁺, —(C₁-C₈)alkylhalide, carboxylate, sulfate, sulfamate, sulfonate, —(C₁-C₈)alkylsulfonate, —(C₁-C₃)alkylamino, 4-dialkylaminopyridinium, —(C₁-C₈)alkylhydroxyl, —(C₁-C₈)alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, —(C₁-C₈)alkoxy, —(C₁-C₈)trifluoroalkyl, —(C₁-C₈)alkyl, —(C₃-C₁₂)carbocycle, —(C₆-C₂₀)aryl, —(C₃-C₂₀)heterocycle, polyethyleneoxy, phosphonate, and phosphate.

The antibody drug conjugate (ADC) comprising a heterocyclic self-immolative moiety is stable extracellularly, or in the absence of an enzyme capable of cleaving the amide bond of the self-immolative moiety. However, upon entry into a cell, or exposure to a suitable enzyme, an amide bond is cleaved initiating a spontaneous self-immolative reaction resulting in the cleavage of the bond covalently linking the self-immolative moiety to the camptothecin moiety, to thereby effect release of the drug in its underivatized or pharmacologically active form.

The self-immolative moiety in conjugates either incorporates one or more heteroatoms and thereby may provide improved solubility, may improve the rate of cleavage and/or may decrease propensity for aggregation of the antibody drug conjugate (ADC). Thus, the heterocyclic self-immolative connector unit constructs in some instances may result in increased efficacy, decreased toxicity, and/or desirable pharmacokinetic and/or pharmacodynamic properties.

When the cytotoxic moiety CM is a camptothecin moiety, it is understood that T in formulae I-III may be for example O, as it can be derived from the tertiary hydroxyl (—OH) on the lactone ring portion of a camptothecin moiety. When the cytotoxic moiety CM is a camptothecin moiety, it is also possible that T in formulae I-III may be for example NH, as it can be derived from an amino group (—NH₂) of a camptothecin moiety, e.g. of exatecan.

Not to be limited by theory or any particular mechanism, the presence of electron-withdrawing groups on the heterocyclic ring of formula I, II or III may moderate the rate of cleavage.

In one embodiment, the self-immolative moiety is the group of formula I in which Q is N, and U is O or S. Such a group has a non-linearity structural feature which improves solubility of the conjugates. In this context R is sometimes H, methyl, nitro, or CF₃. In one embodiment, Q is N and U is O thereby forming an oxazole ring and R is H. In another embodiment, Q is N and U is S thereby forming a thiazole ring optionally substituted at R with an Me or CF₃ group.

In another exemplary embodiment, the self-immolative moiety is the group of formula II in which Q is N and V¹ and V² are independently N or CH. In another embodiment, Q, V¹ and V² are each N. In another embodiment, Q and V¹ are N while V² is CH. In another embodiment, Q and V² are N while V¹ is CH. In another embodiment, Q and V¹ are both CH and V² is N. In another embodiment, Q is N while V¹ and V² are both CH.

In another embodiment, the self-immolative moiety is the group of formula III in which Q, V¹, V² and V³ are each independently N or CH. In another embodiment Q is N while V¹, V² and V³ are each N. In another embodiment, Q, V¹, and V² are each CH while V³ is N. In another embodiment Q, V² and V³ are each CH while V¹ is N. In another embodiment, Q, V¹ and V³ are each CH while V² is N. In another embodiment, Q and V² are both N while V¹ and V³ are both CH. In another embodiment Q and V² are both CH while V¹ and V³ are both N. In another embodiment, Q and V³ are both N while V¹ and V² are both CH.

Preferably, the connector unit (CU) has the formula: *-Aₐ-Wᵥ—B_{b}—##, wherein the integer a is 1, the integer b is 1, and the integer w is 2, 3 or 4, more preferably the integer w is 2 or 3; in very preferred embodiments the integer w is 2; and -A-, each —W— and —B— are as defined herein; * denotes the attachment point to the Y; and ## denotes the attachment point to the cytotoxic moiety (CM).

Preferably, the connector unit (CU) has the following structure:

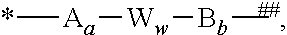

wherein -A- is a second spacer unit as described herein; a is an integer as described herein;
preferably a is 1;
—B— is a first spacer unit as described herein; b is an integer as described herein; preferably b is 1;
* denotes the attachment point to the Y; and ## denotes the attachment point to the cytotoxic moiety (—CM);
—W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). Preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Connector units (CU) according to these embodiments can be illustrative examples for a connector unit which is cleavable, in particular by a protease, such as e.g. a cathepsin (e.g., cathepsin B).

Preferably, the connector unit CU has the following structure:

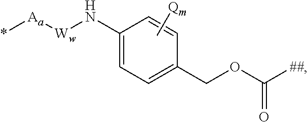

wherein -A- is a second spacer unit as described herein; a is an integer as described herein;
preferably a is 1;
—W_{w}— is an amino acid unit as described herein; w is an integer as described herein; preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), e.g. w may be 1 or 2; in very preferred embodiments w is 2 (i.e. still more preferably —W_{w}— is a dipeptide);
Q is as defined herein;
m is an integer as defined herein, preferably m is 0;
* denotes the attachment point to the Y; and ## denotes the attachment point to the cytotoxic moiety (—CM). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Connector units (CU) according to these embodiments can be illustrative examples for a connector unit (CU) which is cleavable, in particular by a protease, such as e.g. a cathepsin (e.g., cathepsin B).

More preferably, the connector unit CU has the following structure:

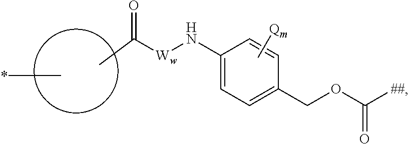

wherein:

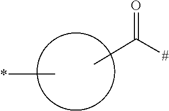

is as defined herein; * denotes the attachment point to the Y; and # denotes the attachment point to the amino acid unit —W_{w}—, when present, or to the NH group;
—W_{w}— is an amino acid unit as described herein; w is an integer as described herein, preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide);
Q is as defined herein;
m is an integer as defined herein, preferably m is 0;
* denotes the attachment point to the Y; and ## denotes the attachment point to the cytotoxic moiety (—CM). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Connector units (CU) according to these embodiments can be illustrative examples for a connector unit which is cleavable, in particular by a protease, such as e.g. a cathepsin (e.g., cathepsin B).

Still more preferably, the connector unit CU has the following structure:

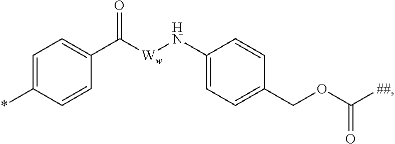

wherein:
—W_{w}— is an amino acid unit as described herein; w is an integer as described herein, preferably w is 2, 3 or 4 (i.e. preferably —W_{w}— is a dipeptide, a tripeptide or a tetrapeptide), more preferably w is 2 or 3 (i.e. more preferably —W_{w}— is a dipeptide or a tripeptide), in very preferred embodiments w is 2 (i.e. still more preferably —W_{w}— is a dipeptide);
* denotes the attachment point to the Y; and ## denotes the attachment point to the cytotoxic moiety (—CM). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ). Connector units (CU) according to these embodiments can be illustrative examples for a connector unit which is cleavable, in particular by a protease, such as e.g. a cathepsin (e.g., cathepsin B).

In a preferred embodiment, the connector unit CU has the following structure:

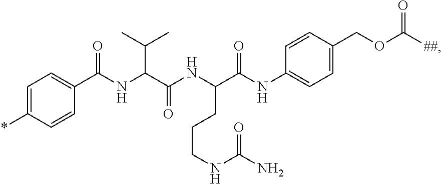

which comprises the dipeptide valine-citrullin as the amino acid unit —W_{w}—; and
wherein * denotes the attachment point to the Y; and ## denotes the attachment point to the cytotoxic moiety (—CM). Such connector unit is an illustrative example for a connector unit which is cleavable, in particular by a protease, such as e.g. a cathepsin (e.g., cathepsin B).

In another preferred embodiment, the connector unit CU has the following structure:

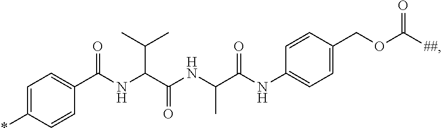

which comprises the dipeptide valine-alanine as the amino acid unit —W_{w}—; and
wherein * denotes the attachment point to the Y; and ## denotes the attachment point to the cytotoxic moiety (—CM). Such connector unit (CU) is an illustrative example for a connector unit which is cleavable, in particular by a protease, such as e.g. a cathepsin (e.g., cathepsin B).

The connector unit CU may have the following structure:

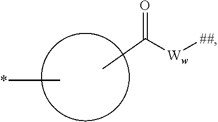

wherein:

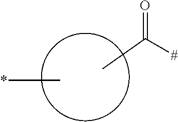

is as defined herein; * denotes the attachment point to the Y; and # denotes the attachment point to the amino acid unit —W_{w}—;
W_{w}— is an amino acid unit as described herein; w is an integer as described herein, preferably w is 2, 3 or 4 (i.e. preferably —W_{w}— is a dipeptide, a tripeptide or a tetrapeptide), more preferably the integer w is 2 or 3 (i.e. more preferably —W_{w}— is a dipeptide or a tripeptide), still more preferably w is 2 (i.e. still more preferably —W_{w}— is a dipeptide);
* denotes the attachment point to the Y; and ## denotes the attachment point to the cytotoxic moiety (—CM). Preferably, in these embodiments, the amino acid unit —W_{w}— is a dipeptide selected from the group consisting of valine-citrulline (i.e. Val-Cit or VC), valine-alanine (i.e. Val-Ala or VA), alanine-alanine (i.e. Ala-Ala or AA) and phenylalanine-lysine (i.e. Phe-Lys or FK). More preferably, in these embodiments the amino acid unit is a dipeptide selected from the group consisting of valine-citrulline (i.e. Val-Cit or VC), valine-alanine (i.e. Val-Ala or VA), and phenylalanine-lysine (i.e. Phe-Lys or FK). Still more preferably, in these embodiments the amino acid unit is valine-citrulline (i.e. Val-Cit or VC) or valine-alanine (i.e. Val-Ala or VA). Even more preferably, in these embodiments the amino acid unit is valine-citrulline (i.e. Val-Cit or VC). Alternatively, in these embodiments, the amino acid unit —W_{w}— may be a dipeptide selected from the group consisting of valine-glutamine (i.e. Val-Gln or VQ), leucine-glutamine (i.e. Leu-Gln or LQ), phenylalanine-glutamin (i.e. Phe-Gln or FQ) and threonine-threonine (i.e. Thr-Thr or TT). In these embodiments, the amino acid unit may be a dipeptide selected from the group consisting of valine-glutamine (i.e. Val-Gln or VQ), leucine-glutamine (i.e. Leu-Gln or LQ), and phenylalanine-glutamin (i.e. Phe-Gln or FQ). In these embodiments, the amino acid unit may be valine-glutamine (i.e. Val-Gln or VQ) or leucine-glutamine (i.e. Leu-Gln or LQ).

In some embodiments, the connector unit CU may have the following structure:

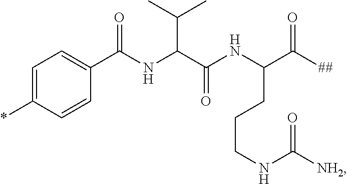

which comprises the dipeptide valine-citrulline as the amino acid unit —W_{w}—; and
wherein * denotes the attachment point to the Y; and ## denotes the attachment point to the cytotoxic moiety (—CM).

In some embodiments, the connector unit CU may have the following structure:

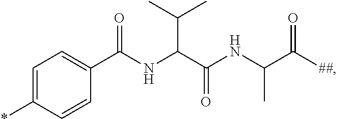

which comprises the dipeptide valine-alanine as the amino acid unit —W_{w}—; and
wherein * denotes the attachment point to the Y; and ## denotes the attachment point to the cytotoxic moiety (—CM).

The connector unit (—CU—) may have the following structure:

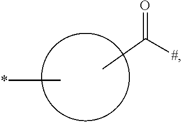

wherein:

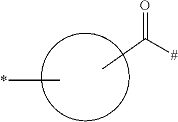

is as defined herein; * denotes the attachment point to the Y; and # denotes the attachment point to the cytotoxic moiety (—CM).

In some embodiments, the connector unit CU may have the following structure:

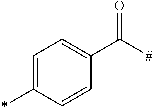

wherein * denotes the attachment point to the Y; and # denotes the attachment point to the cytotoxic moiety (—CM).

### Cytotoxic Moiety (—CM)

The present disclosure provides antibody drug conjugates (ADCs) comprising a cytotoxic moiety. The term “cytotoxic moiety”, “drug”, “drug moiety”, “payload” or “cytotoxic payload”, both of which can be used interchangeably, as used herein refers to a chemical or biochemical moiety that is conjugated to an anti-TPBG antibody (Ab), or antigen binding fragment thereof. In this regard, it is again referred to the antibody drug conjugate (ADC) of formula (I) described herein. The antibody (Ab) can be conjugated to several identical or different cytotoxic moieties using any methods described herein or known in the art. In some embodiments, the cytotoxic moiety may be a molecule which has a cytotoxic effect on mammalian cells, may lead to apoptosis, and/or may have a modulating effect on malignant cells. The cyototoxic moiety may be hydrophobic.

In some preferred embodiments the cytotoxic moiety is an anti-cancer agent. Accordingly, the cyototoxic moiety may be selected from the group consisting of camptothecins, maytansinoids, calicheamycins, tubulysins, amatoxins, dolastatins and auristatins such as monomethyl auristatin E (MMAE) or monomethyl auristatin F (MMAF), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof.

### Camptothecin Moiety

Preferably, the cytotoxic moiety CM is a camptothecin moiety. The term “camptothecin moiety” includes camptothecin itself and analogues of camptothecin. Camptothecin is a topoisomerase poison, which was discovered in 1966 by M. E. Wall and M. C. Wani in systematic screening of natural products for anticancer drugs. Camptothecin was isolated from the bark and stem of Camptotheca acuminata (Camptotheca, Happy tree), a tree native to China used as a cancer treatment in Traditional Chinese Medicine. Camptothecin has the following structure:

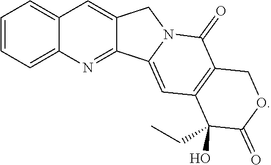 [SMILES: CC[C@@]1(O)C(=O)OCc2c1cc1n(c2=O)Cc2cc3ccccc3nc2-1]

The term “campthothecin moiety” also comprises camptothecin analogues. In this regard, the term “camptothecin moiety” denotes any moiety which comprises the structure of camptothecin:

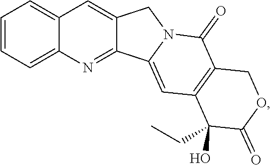 [SMILES: CC[C@@]1(O)C(=O)OCc2c1cc1n(c2=O)Cc2cc3ccccc3nc2-1]

and which may be optionally substituted. The optional substituents may include, as illustrative non-limiting examples, (C₁-C₁₀)alkyl, (C₃-C₈)carbocyclo, (C₃-C₈)heterocyclo, aryl, an amino group, a hydroxy group, a carbonyl group, an amide group, an ester group, a carbamate group, a carbonate group and/or a silyl group. The camptothecin moiety may have one or more functional group(s) which are capable to form a bond to the linker L. A person skilled in the will readily select a suitable camptothecin moiety having a desired biological activity. Camptothecin analogues have been approved and are used in cancer chemotherapy today, such as e.g. topotecan, irinotecan, or belotecan.

The following camptothecin analogues are also envisioned by the term camptothecin moiety:

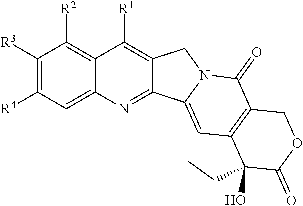

| Analogue | R¹ | R² | R³ | R⁴ |
|---|---|---|---|---|
| Topotecan | —H | 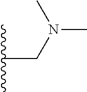 [SMILES: *CN(C)C] | —OH | —H |
| Irinotecan (CPT-11) | 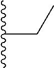 [SMILES: *CC] | —H | 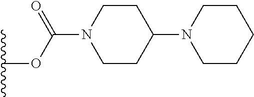 [SMILES: *OC(=O)N1CCC(N2CCCCC2)CC1] | —H |
| Silatecan (DB-67, AR-67) | 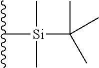 [SMILES: *[Si](C)(C)C(C)(C)C] | —H | —OH | H |
| Cositecan (BNP-1350) | 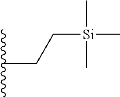 [SMILES: *CC[Si](C)(C)C] | —H | —H | H |
| Exatecan | 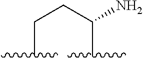 [SMILES: *CC[C@@H](*)N] | | —CH₃ | —F |
| Lurtotecan | 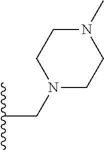 [SMILES: *CN1CCN(C)CC1] | —H | 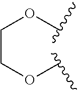 [SMILES: *OCCO*] | |
| Gimatecan (ST1481) | 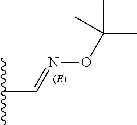 [SMILES: */C=N/OC(C)(C)C] | —H | —H | —H |
| Belotecan (CKD-602) | 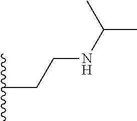 [SMILES: *CCNC(C)C] | —H | —H | —H |
| Rubitecan | —H | 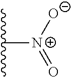 [SMILES: *[N+](=O)[O-]] | —H | —H |

Further camptothecin analogues, which may be used as camptothecin moiety, are described in WO 2019/236954 and EP 0 495 432.

In some embodiments, the camptothecin moiety (CM) is selected from the group consisting of exatecan, DXD, SN38, camptothecin, topotecan, irinotecan, belotecan, lurtotecan, rubitecan, silatecan, cositecan and gimatecan. Preferably, the camptothecin moiety is selected from the group consisting of exatecan, DXD, SN38, camptothecin, topotecan, irinotecan and belotecan. SN38 has the following structure:

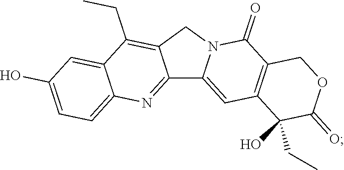 [SMILES: CCc1c2c(nc3ccc(O)cc13)-c1cc3c(c(=O)n1C2)COC(=O)[C@]3(O)CC]

and the structures of exatecan, camptothecin, topotecan, irinotecan and belotecan are as described herein.

More preferably, in any one of the embodiments described herein, the camptothecin moiety CM is exatecan having the following structure:

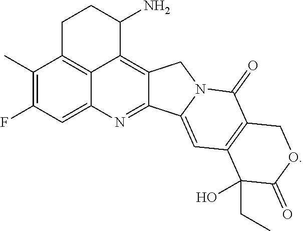 [SMILES: CCC1(O)C(=O)OCc2c1cc1n(c2=O)Cc2c-1nc1cc(F)c(C)c3c1c2C(N)CC3]

Still more preferably, the camptothecin moiety (CM) is exatecan having the following structure:

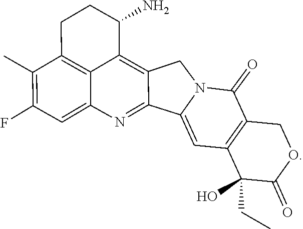 [SMILES: CC[C@@]1(O)C(=O)OCc2c1cc1n(c2=O)Cc2c-1nc1cc(F)c(C)c3c1c2[C@@H](N)CC3]

Preferably, in any one of these embodiments the exatecan is bound to the connector unit CU via the amino group (i.e., via the NH₂ group of exatecan). When exatecan is bound to the connector unit CU via the amino group, one hydrogen atom of the amino group of exatecan is replaced by the connector unit CU. Accordingly, exatecan bound to the connector unit CU via the amino group can be depicted, e.g., as follows:

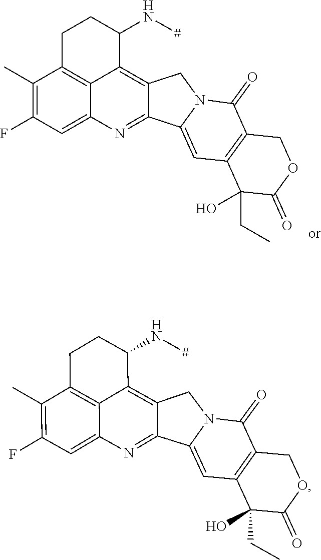

wherein # indicates the attachment point to the connector unit CU.

In some aspects/embodiments the present invention also relates to a conjugate having the formula (I):

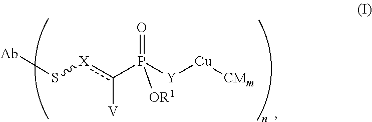

or a pharmaceutically acceptable salt or solvate thereof;
wherein:
Ab is an anti-TPBG antibody as described herein;
[symbol] is a double bond; or
[symbol] is a bond;
V is absent when [symbol] is a double bond; or
V is H when [symbol] is a bond;
X is R₃—C when [symbol] is a double bond; or
X is

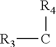

when [symbol] is a bond;
Y is NH;
R¹ is a polyethylene glycol unit having the structure:

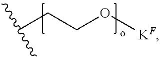

wherein:
[symbol] indicates the position of the O;
K^{F} is as defined herein; preferably K^{F} is H; and
o is an integer as defined herein; preferably o is an integer ranging from 8 to 30; more preferably from 16 to 30; still more preferably from 20 to 28; still more preferably, o is 22, 23, 24, 25 or 26; still more preferably, o is 23, 24 or 25; even more preferably o is 24;
R³ is H;
R⁴ is H;
CU is a connector unit having the following structure:

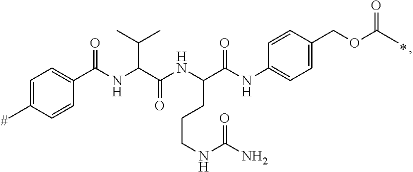

wherein # indicates the attachment point to the Y and * indicates the attachment point to the camptothecin moiety (CM);
CM is a camptothecin moiety;
m is 1; and
n is an integer as defined herein;
   preferably n is an integer ranging from 1 to 10; more preferably from 2 to 10; still more preferably from 4 to 10; still more preferably from 6 to 10, still more preferably from 7 to 10, even more preferably n is 8; or
   preferably n is an integer ranging from 1 to 10, more preferably from 2 to 8, still more preferably from 3 to 6, still more preferably n is 4 or 5, even more preferably n is 4.

Preferably, [symbol] is a double bond; V is absent; X is R₃—C; and R³ is H.

In some embodiments, [symbol] may be a bond; V may be H; X may be

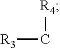

R³ may be H; and R⁴ may be H.

Preferably, the camptothecin moiety CM is exatecan having the following structure:

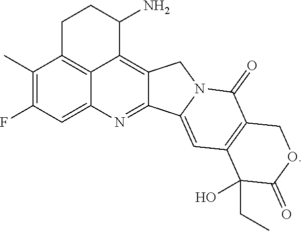 [SMILES: CCC1(O)C(=O)OCc2c1cc1n(c2=O)Cc2c-1nc1cc(F)c(C)c3c1c2C(N)CC3]

More preferably, the camptothecin moiety is exatecan having the following structure:

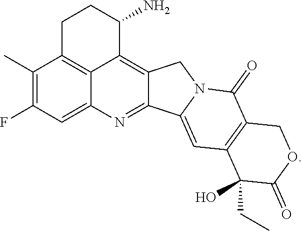 [SMILES: CC[C@@]1(O)C(=O)OCc2c1cc1n(c2=O)Cc2c-1nc1cc(F)c(C)c3c1c2[C@@H](N)CC3]

Preferably, in any one of these embodiments the exatecan is bound to the connector unit CU via the amino group.

In some aspects/embodiments the present invention also relates to an antibody drug conjugate (ADC) having the following formula (Ia):

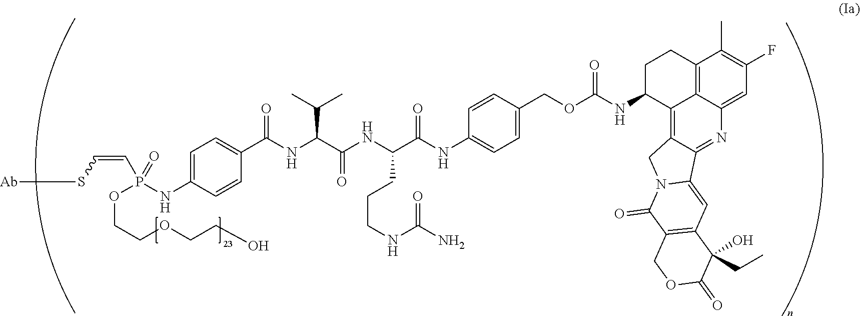

wherein: Ab is an anti-TPBG antibody as described herein; and
n is an integer a defined herein;
preferably n is an integer ranging from 1 to 10; more preferably from 2 to 10; still more preferably from 4 to 10; still more preferably from 6 to 10, still more preferably from 7 to 10, even more preferably n is 8; or
preferably n is an integer ranging from 1 to 10, more preferably from 2 to 8, still more preferably from 3 to 6, still more preferably n is 4 or 5, even more preferably n is 4. Auristatins

In some embodiments the cytotoxic moiety CM is an auristatin. Preferably, the auristatin is monomethyl auristatin F (MMAF) or monomethyl auristatin E (MMAE). More preferably, the auristatin is monomethyl auristatin E (MMAE).

In some embodiments the cytotoxic moiety CM is monomethyl auristatin F (also known as MMAF). MMAF is represented by the following structural formula:

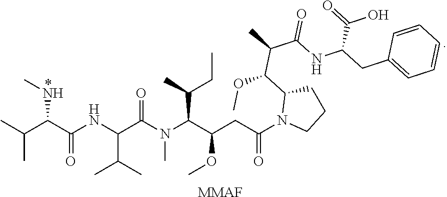

Preferably, MMAF is bound to the connector unit CU via the N terminus indicated with an asterisk (“*”). Accordingly, when MMAF is bound to the connector unit CU via the N terminus, the hydrogen atom of the N terminus of MMAF is replaced by the connector unit CU.

In some embodiments the auristatin drug moiety is monomethyl auristatin E (also known as MMAE). MMAE is represented by the following structural formula:

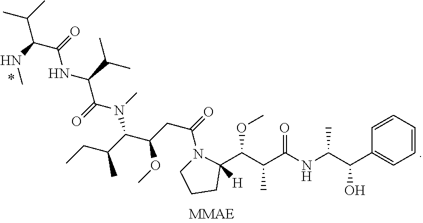 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

Preferably, MMAE is bound to the connector unit CU via the N terminus indicated with an asterisk (“*”). Accordingly, when MMAE is bound to the connector unit CU via the N terminus, the hydrogen atom of the N terminus of MMAE is replaced by the connector unit CU.

These molecules noncompetitively inhibit binding of vincristine to tubulin (at a location known as the vinca/peptide region) but have been shown to bind to the RZX/MAY region. Compounds of Formula (II)

In some aspects/embodiments the present invention also relates to a compound having the formula (II):

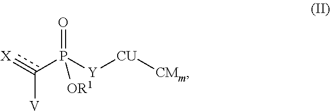

or a pharmaceutically acceptable salt or solvate thereof,
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

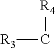

when [symbol] is a double bond;
Y is NR⁵, S, O, or CR⁶R⁷;
R¹ is an optionally substituted aliphatic residue or an optionally substituted aromatic residue;
R³ is H; or an optionally substituted aliphatic or optionally substituted aromatic residue;
R⁴ is H; or an optionally substituted aliphatic or optionally substituted aromatic residue;
R⁵ is H; or an optionally substituted aliphatic or optionally substituted aromatic residue;
R⁶ is H; or an optionally substituted aliphatic or optionally substituted aromatic residue;
R⁷ is H; or an optionally substituted aliphatic or optionally substituted aromatic residue CU is a connector unit;
CM is a cytotoxic moiety; and
m is an integer ranging from 1 to 10.

Preferably R³ is H or (C₁-C₈)alkyl; more preferably R³ is H. Preferably R⁴, when present, is H or (C₁-C₈)alkyl; more preferably R⁴, when present, is H. Preferably R⁵, when present is H or (C₁-C₈)alkyl; more preferably R⁵, when present, is H. Preferably R⁶, when present is H or (C₁-C₈)alkyl; more preferably R⁶, when present, is H. Preferably R⁷, when present is H or (C₁-C₈)alkyl; more preferably R⁷, when present, is H.

Preferably, [symbol] is a triple bond; V is absent; X is R₃—C; and R³ is H or an optionally substituted aliphatic residue or an optionally substituted aromatic residue; preferably R³ is H or (C₁-C₈)alkyl; more preferably R³ is H.

More preferably, [symbol] represents a triple bond; V is absent; X represents R₃—C and R₃ represents H or (C₁-C₈)alkyl. Preferably, R₃ represents H or (C₁-C₆)alkyl, more preferably H or (C₁-C₄)alkyl, still more preferably H or (C₁-C₂)alkyl. Even more preferably, R₃ is H.

In some embodiments, [symbol] may be a double bond; V is H or (C₁-C₈)alkyl, preferably V is H; X is

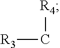

R₃ is H or an optionally substituted aliphatic residue or an optionally substituted aromatic residue; more preferably R³ is H or (C₁-C₈)alkyl, more preferably R³ is H; R⁴ is H or an optionally substituted aliphatic residue or an optionally substituted aromatic residue; preferably, R⁴ is H or (C₁-C₈)alkyl, preferably R⁴ is H.

In some embodiments, [symbol] may represent a double bond; V may be H or (C₁-C₈)alkyl; X may represent

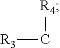

and R₃ and R₄ may independently represent H or (C₁-C₈)alkyl. Preferably, R₃ and R₄ independently represent H or (C₁-C₆)alkyl, more preferably H or (C₁-C₄)alkyl, still more preferably H or (C₁-C₂)alkyl. Preferably, R₃ and R₄ are the same; even more preferably, R₃, R₄ and V are the same. More preferably, R₃ and R₄ are both H. Preferably, V is H or (C₁-C₆)alkyl, more preferably H or (C₁-C₄)alkyl, still more preferably H or (C₁-C₂)alkyl. Even more preferably, V is H. In preferred embodiments, R₃, R₄ and V are each H.

In any one of the compounds of formula (II), any variable may be defined as described herein, in particular as with regard to the antibody drug conjugates (ADCs) of formula (I) and/or the thiol-containing molecule of formula (III). Accordingly, Ab, [symbol] , V, X, Y, R¹, R³, R⁴, R⁵, R⁶, R⁷, CU, CM, m and n may be as defined herein. Preferably, Y is NH.

### Method of Preparing an Antibody Drug Conjugate (ADC)

In some aspects/embodiments the present invention relates to a method of synthesis of the antibody drug conjugates (ADCs) of the present invention.

In some aspects/embodiments the present invention also relates to a method of preparing an antibody conjugate (ADC) of formula (I), said method comprising:
reacting a compound of formula (II)

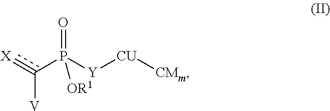

or a pharmaceutically acceptable salt or solvate thereof,
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

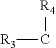

when [symbol] is a double bond;
Y is NR⁵, S, O, or CR⁶R⁷;
R¹ is an optionally substituted aliphatic residue or an optionally substituted aromatic residue;
R³ is H; or an optionally substituted aliphatic or optionally substituted aromatic residue;
R⁴ is H; or an optionally substituted aliphatic or optionally substituted aromatic residue;
R⁵ is H; or an optionally substituted aliphatic or optionally substituted aromatic residue;
R⁶ is H; or an optionally substituted aliphatic or optionally substituted aromatic residue;
R⁷ is H; or an optionally substituted aliphatic or optionally substituted aromatic residue;
CU is a connector unit;
CM is a cytotoxic moiety; and
m is an integer ranging from 1 to 10;
with a thiol-containing molecule of formula (III)

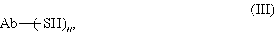

wherein Ab is an anti-TPBG antibody as described herein; and
n is an integer ranging from 1 to 20;
resulting in an antibody drug conjugate (ADC) of formula (I)

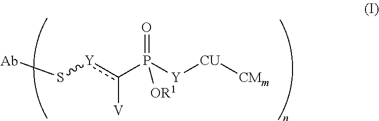

or a pharmaceutically acceptable salt or solvate therein;
wherein:
Ab is an anti-TPBG antibody as described herein;
[symbol] is a double bond when [symbol] in a compound of formula (II) is a triple bond; or
[symbol] is a bond when [symbol] in a compound of formula (II) is a double bond;
V is absent when [symbol] is a double bond; or
V is H or (C₁-C₈)alkyl when [symbol] is a bond;
X is R₃—C when [symbol] is a double bond; or
X is

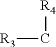

when [symbol] is a bond;
Y is NH, S, O, or CH₂;
R¹ is an optionally substituted aliphatic residue or an optionally substituted aromatic residue;
R³ is H; or an optionally substituted aliphatic or optionally substituted aromatic residue;
R⁴ is H; or an optionally substituted aliphatic or optionally substituted aromatic residue;
R⁵ is H; or an optionally substituted aliphatic or optionally substituted aromatic residue;
R⁶ is H; or an optionally substituted aliphatic or optionally substituted aromatic residue;
R⁷ is H; or an optionally substituted aliphatic or optionally substituted aromatic residue;
CU is a connector unit;
CM is a cytotoxic moiety;
m is an integer ranging from 1 to 10; and
n is an integer ranging from 1 to 20.

Preferably R³ is H or (C₁-C₈)alkyl; more preferably R³ is H. Preferably R⁴, when present, is H or (C₁-C₈)alkyl; more preferably R⁴, when present, is H. Preferably R⁵, when present is H or (C₁-C₈)alkyl; more preferably R⁵, when present, is H. Preferably R⁶, when present is H or (C₁-C₈)alkyl; more preferably R⁶, when present, is H. Preferably R⁷, when present is H or (C₁-C₈)alkyl; more preferably R⁷, when present, is H.

Preferably, [symbol] is a triple bond; V is absent; X is R₃—C; and R³ is H or an optionally substituted aliphatic residue or an optionally substituted aromatic residue; preferably R³ is H or (C₁-C₈)alkyl; more preferably R³ is H; and [symbol] represents a double bond.

More preferably, [symbol] represents a triple bond; V is absent; X represents R₃—C, R₃ represents H or (C₁-C₈)alkyl; and [symbol] represents a double bond. Preferably, R₃ represents H or (C₁-C₆)alkyl, more preferably H or (C₁-C₄)alkyl, still more preferably H or (C₁-C₂)alkyl. Even more preferably, R₃ is H.

In some embodiments, [symbol] may be a double bond; V is H or (C₁-C₈)alkyl, preferably V is H; X is

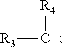

R₃ is H or an optionally substituted aliphatic residue or an optionally substituted aromatic residue; and [symbol] may represent a bond; more preferably R³ is H or (C₁-C₈)alkyl, more preferably R³ is H; R⁴ is H or an optionally substituted aliphatic residue or an optionally substituted aromatic residue; preferably, R⁴ is H or (C₁-C₈)alkyl, preferably R⁴ is H.

In some embodiments, may represent a double bond; V may be H or (C₁-C₈)alkyl; X may represent

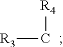

R₃ and R₄ may independently represent H or (C₁-C₈)alkyl; and [symbol] may represents a bond. Preferably, R₃ and R₄ independently represent H or C₁-C₆-alkyl, more preferably H or C₁-C₄-alkyl, still more preferably H or C₁-C₂-alkyl. Preferably, R₃ and R₄ are the same; even more preferably, R₃, R₄ and V are the same. More preferably, R₃ and R₄ are both H. Preferably, V is H or C₁-C₆-alkyl, more preferably H or C₁-C₄-alkyl, still more preferably H or C₁-C₂-alkyl. Even more preferably, V is H. In preferred embodiments, R₃, R₄ and V are each H.

With regard to the representations [symbol] and [symbol] used herein, it is noted that, as commonly known to a person skilled in the art, each carbon atom is tetravalent. Accordingly, a structure

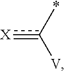

wherein X and V are as defined herein and the asterisk (*) indicates attachment to the phosphorus, includes the structures

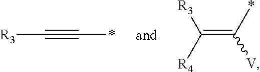

wherein R₃, R₄ and V are as defined herein. A structure

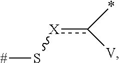

wherein X and V are as defined herein, the asterisk (*) indicates attachment to the phosphorus and # indicates attachment to the receptor binding molecule (RBM), includes the structures

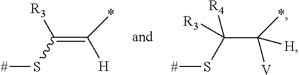

wherein R₃, R₄ and V are as defined herein, and H is hydrogen. A wavy bond indicates that the configuration of the double bond may be E or Z. It is also possible that the compound is present as a mixture of the E and Z isomers.

When the anti-TPBG antibody (Ab) comprises one or more disulfide bridges, the method may further comprise reducing at least one disulfide bridge of the antibody in the presence of a reducing agent to form a thiol group (SH). The resulting compound of formula (III) may then be reacted with a compound of formula (II) to yield an antibody drug conjugate (ADC) of formula (I). The reducing agent may be selected from the group consisting of tris(2-carboxyethyl)phosphine (TCEP), dithiothreitol (DTT), sodium dithionite, sodium thiosulfate, and sodium sulfite. Accordingly, the reducing agent may be dithiothreitol (DTT). The reducing agent may be sodium dithionite. The reducing agent may be sodium sulfite. Preferably, the reducing agent is tris(2-carboxyethyl)phosphine (TCEP).

Preferably, the reducing of at least one disulfide bridge comprises using about 1 to about 3 equivalents, preferably about 1 to about 2 equivalents, more preferably about 1 equivalent of the reducing agent per 1 disulfide bridge to be reduced. In this context, it is noted that in theory 1 eq. of the reducing agent, in particular of a reducing agent as described herein, is necessary to reduce 1 disulfide bridge to give 2 thiol groups (SH).

Preferably, the thiol-containing molecule of formula (III) is reacted with about 1 to about 4 equivalents, preferably about 1 to about 3 equivalents, more preferably about 1 to about 2 equivalents, still more preferably about 1 to 1.5 equivalents of the compound of formula (II) per thiol group (SH).

Preferably, the reaction of a compound of formula (II) with a thiol-containing molecule of formula (III) is carried out in an aqueous medium.

Preferably, the reaction of the compound of formula (II) with the thiol-containing molecule of formula (III) is performed under neutral pH or slightly basic conditions. Still more preferably the reaction is performed at a pH of from 6 to 10. Even more preferably, the reaction is performed at a pH of from 7 to 9.

In any one of the methods, any variable may be defined as described herein, in particular as with regard to the antibody drug conjugates (ADCs) of formula (I) and/or the compound of formula (II). Accordingly, Ab, [symbol] , [symbol] , V, X, Y, R¹, R³, R⁴, R⁶, R⁶, R⁷, CU, CM, m and n may be as defined herein. Preferably, Y is NH.

Methods of preparing compounds of formula (II) are known in the art. As illustrative examples, compounds of formula (II), wherein the group Y is NH, may be prepared by using techniques and conditions, e.g. a Staudinger phosphonite reaction, as e.g. described in WO 2018/041985 A1, which is hereby incorporated by reference. Compounds of formula (II), wherein Y is S or O, may be prepared by using techniques and conditions as e.g. described in WO 2019/170710, which is hereby incorporated by reference. In an analogous manner to the compounds of formula (II), wherein Y is S or O, as described in WO 2019/170710, compounds of formula (II), wherein Y is CR⁶R⁷, may be prepared, as illustrative examples, by substitution at the phosphorus atom using, e.g., a suitable organometallic compound, such as e.g. a Grignard compound or an organolithium compound. A person skilled in the art readily selects suitable methods and conditions to prepare compounds of formula (II). The Examples section of the present disclosure also comprises guidance on how to prepare or obtain compounds of formula (II) and/or antibody drug conjugates (ADCs) of formula (I).

The present invention also relates to an antibody drug conjugate of formula (I) obtainable or being obtained by any method of preparing an antibody drug conjugate of formula (I) as described herein.

In some aspects/embodiments the present invention relates to cancer. Cancer can be any cancer. Preferably a cancer is selected from the group consisting of: Breast cancer, Head and neck cancer, Ovary cancer, Endometrium cancer, Uterine cervix cancer, Rectum cancer, Colon cancer, Esophagus cancer, Stomach cancer, Lung cancer, Kidney cancer, Adrenal gland cancer, Bladder cancer, Liver cancer, Sarcoma, Brain cancer, Nevi and Melanomas, Urogenital cancer, Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers, Retinoblastom, Thyroid cancer, Fallopian tube cancer; further preferably said cancer is a solid cancer; most preferably said cancer is selected from the group consisting of: Breast cancer, Head and neck cancer, Ovarian cancer, Endometrial cancer, Uterus cancer (e.g., cancers of the muscle sheets), Cervical cancer, Rectum cancer, Colon cancer, Anal cancer, Esophagus cancer, Stomach cancer, Lung cancer, Kidney cancer, Adrenal gland cancer, Bladder cancer, Liver cancer, Sarcoma (e.g., osteosarcoma and Kaposi sarcoma), Brain cancer (e.g., pituitary tumor/s), Nevi and Melanoma cancers, Skin cancers (e.g., squamous cell carcinoma and melanoma), Urogenital cancer (e.g., ureter and bladder cancer, testicular cancer, prostate cancer, penile cancer), Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers (e.g., lymphoma, leukemia, myeloma, Myelodysplastic syndromes or myelofibrosis), Eye cancers (e.g., Retinoblastoma), Neuroendocrine tumors, Cancer of unknown primary (CUP).

In some aspects/embodiments the present invention relates to a composition or kit comprising the anti-TPBG, antibody drug conjugate (ADC), hybridoma, nucleic acid, expression vector and/or host cell of the present invention.

In some aspects/embodiments the present invention relates to methods of treatment (e.g., of a patient) and uses of the antibody, antibody drug conjugate (ADC), nucleic acid, expression vector, host cell, composition and/or kit of the present invention.

In some aspects of the present invention, the antibody of the present invention is expressed as an Fc-silenced (LALA mutation) IgG1 in CHO cells, purified via Protein A chromatography.

In some aspects of the present invention, the mAb of the present invention binds to human 5T4, e.g., dependent on 5T4/antigen-glycosylation, with apparent dissociation constants (K_{D}) of 0.11 nM in an ELISA setting.

In some aspects of the present invention, the mAb of the present invention binds to recombinant 5T4-ECD (extracellular domain) and with K_{D} of 0.25 nM to MDA-MB-468 cells endogenously expressing 5T4.

In some aspects of the present invention, the mAb of the present invention having cross-reactive with non-human primate (NHP) i.e., marmoset 5T4.

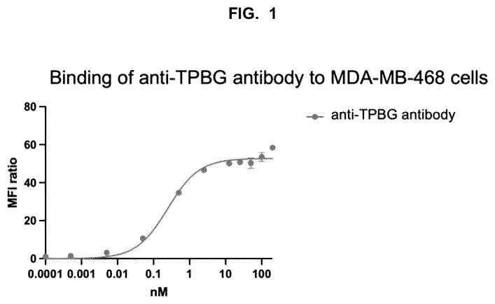

The present invention further relates to the following items:
1. An anti-TPBG (also can be referred to as anti-5T4) antibody (e.g., an antigen binding portion thereof, preferably antibody against TPBG_HUMAN Trophoblast glycoprotein, e.g., having UniProt Accession Number: Q13641 or SEQ ID NO: 1), wherein said anti-TPBG antibody comprising Fc silencing mutations such as leucine (L) to alanine (A) substitution at the position 234 and 235 (LALA mutations) (e.g., in Fc region of said antibody) and is capable of the following:
   a) binding to human Trophoblast glycoprotein (TPBG) (e.g., having UniProt Accession Number: Q13641 or SEQ ID NO: 1);
   b) having cross-reactivity with white-tufted-ear marmoset (e.g., Callithrix jacchus) TPBG (e.g., having UniProtKB Accession Number: F710T3 or SEQ ID NO: 2); and
   c) internalization, preferably by the means of the antigen-mediated antibody internalization.
2. The anti-TPBG antibody according to any one of the preceding items, wherein said antibody comprising a mutated (e.g., according to any one of the preceding items) Fc region (or fragment crystallizable region), e.g., the tail region of said antibody that when unmutated interacts with cell surface Fc receptors (e.g., wherein said Fc region of an immunoglobulin molecule composed of the constant regions of the heavy chains, e.g., and when unmutated is capable of binding to antibody receptors (Fc receptor) on cells and the C1q component of complement).
3. The anti-TPBG antibody according to any one of the preceding items, wherein said antibody comprising heavy chain constant region.
4. The anti-TPBG antibody according to any one of the preceding items, wherein said antibody is capable of binding (e.g., specifically binding) to an extracellular domain of said TPBG (e.g., amino acids 32-355 of SEQ ID NO: 1).
5. The anti-TPBG antibody according to any one of the preceding items, wherein said antibody is capable of binding (e.g., specifically binding) to extracellular epitope of said TPBG (e.g., amino acids 32-355 of SEQ ID NO: 1).
6. The anti-TPBG antibody according to any one of the preceding items, wherein said anti-TPBG antibody is:
   a) having K_{D} to an endogenously expressed human TPBG (e.g., expressed on a cell surface) in the range from about 0.01 to about 10 nmol/L, preferably in the range from about 0.15 to about 0.35 nmol/L (e.g., in MDA-MB-468 cells (e.g., having DSMZ No.: ACC 738) endogenously expressing TPBG), further preferably said K_{D} is measured by the means of a FACS assay, most preferably having said K_{D} in the range from about 0.20 to about 0.30 nmol/L (e.g., 0.25 nmol/L); and/or
   b) optionally, having K_{D} in the range from about 0.01 to about 10 nmol/L to an immobilized exogenous full-length TPBG and/or one or more fragments thereof, preferably said one or more fragments comprising at least an extracellular domain (ECD) of said TPBG (e.g., said ECD comprising at least amino acids 32-355 of the human TPBG having UniProt Accession Number: Q13641 or SEQ ID NO: 1) and/or one or more fragments of said ECD (e.g., having length from about 15 to about 30 amino acids), wherein said full-length TPBG and/or one or more fragments thereof are fused or not fused to one or more protein tags (e.g., 6× His-tags, FLAG, HA, V⁵, Fc-fusion, MBP, SUMO, TEV, GFP, TST), further preferably said K_{D} is in the range from about 0.05 to about 0.30 nmol/L, most preferably said K_{D} is measured by the means of an ELISA assay, further most preferably said K_{D} is in the range from about 0.05 to about 0.2 nmol/L (e.g., from about 0.10 (e.g., glycosylated) to about 0.16 (deglycosylated) nmol/L).
7. The anti-TPBG antibody according to any one of the preceding items, wherein said anti-TPBG antibody is an antibody comprising a heavy chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 3 and a light chain variable region having an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 4; preferably said anti-TPBG antibody comprising:
   a) a light chain comprising an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 5 and
   b) a heavy chain comprising an amino acid sequence with at least 80% (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity to SEQ ID NO: 6.
8. The anti-TPBG antibody according to any one of the preceding items, wherein said anti-TPBG antibody is an antibody comprising:
   a) a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9 and
   b) a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12.
9. The anti-TPBG antibody according to any one of the preceding items, wherein said anti-TPBG antibody has one or more of the following characteristics:
   a) a monoclonal antibody;
   b) a chimeric antibody and/or humanized antibody;
   c) specifically recognizes TPBG (e.g., human) overexpressed on cancer cell/s;
   d) human IgG antibody, preferably a human IgG1 antibody;
   e) comprising kappa (κ) light chain;
   f) comprising lambda (A) light chain;
   g) capable of being internalized by target cells (e.g., cancer cells) expressing TPBG; preferably said internalized antibody is directed to lysosomes;
   h) a tumor-selective antibody, preferably said tumor is a liquid and/or solid tumor, preferably solid tumor;
   i) a malignant-cell selective antibody;
   j) binding to said human TPBG in a glycosylation-dependent manner, preferably wherein said antibody binds to the glycosylated form of said TPBG;
   k) comprising Fc silencing mutations such as leucine (L) to alanine (A) substitution at the position 234 and 235 (LALA mutations, i.e., Leu234Ala and Leu235Ala), wherein said LALA mutations are capable of reducing effector function/s of immune cell/s.
10. The anti-TPBG antibody according to any one of the preceding items, wherein said anti-TPBG antibody is a monoclonal antibody.
11. The anti-TPBG antibody according to any one of the preceding items, wherein said anti-TPBG antibody is a chimeric antibody.
12. The anti-TPBG antibody according to any one of the preceding items, wherein said anti-TPBG antibody is a humanized antibody.
13. The anti-TPBG antibody according to any one of the preceding items, wherein said anti-TPBG antibody specifically recognizes TPBG (e.g., human) overexpressed on cancer cell/s.
14. The anti-TPBG antibody according to any one of the preceding items, wherein said anti-TPBG antibody is human IgG antibody, preferably a human IgG1 antibody.
15. The anti-TPBG antibody according to any one of the preceding items, wherein said anti-TPBG antibody is a human IgG1 antibody.
16. The anti-TPBG antibody according to any one of the preceding items, wherein said anti-TPBG antibody comprising kappa (κ) light chain.
17. The anti-TPBG antibody according to any one of the preceding items, wherein said anti-TPBG antibody comprising lambda (A) light chain.
18. The anti-TPBG antibody according to any one of the preceding items, wherein said anti-TPBG antibody capable of being internalized by target cells (e.g., cancer cells) expressing TPBG; preferably said internalized antibody is directed to lysosomes.
19. The anti-TPBG antibody according to any one of the preceding items, wherein said anti-TPBG antibody is a tumor-selective antibody, preferably said tumor is a liquid and/or solid tumor, preferably a solid tumor.
20. The anti-TPBG antibody according to any one of the preceding items, wherein said anti-TPBG antibody is a malignant-cell selective antibody.
21. The anti-TPBG antibody according to any one of the preceding items, wherein said anti-TPBG antibody is capable of binding to human TPBG in a glycosylation-dependent manner, preferably wherein said antibody binds to the glycosylated form of said TPBG.
22. The anti-TPBG antibody according to any one of the preceding items, wherein said anti-TPBG antibody comprising Fc silencing mutations such as leucine (L) to alanine (A) substitution at the position 234 and 235 (LALA mutations), wherein said LALA mutations are capable of reducing effector function/s of immune cells.
23. The anti-TPBG antibody according to any one of the preceding items, wherein said antibody is coupled to a labelling group.
24. The anti-TPBG antibody according to any one of the preceding items, wherein said antibody is obtainable by a hybridoma (e.g., said antibody is a recombinant antibody).
25. The anti-TPBG antibody according to any one of the preceding items, wherein said antibody comprises one or more signal sequences according to SEQ ID NOs: 13 and/or 14.
26. The anti-TPBG antibody according to any one of the preceding items, wherein the antibody comprises one or more CDRs (e.g., 2, 3, 4, 5 or 6), heavy chain variable regions (e.g., 2), light chain variable regions (e.g., 2), heavy chains (e.g., 2), light chains (e.g., 2) and/or signal sequences according to any one of the preceding items, preferably selected from the group consisting of: SEQ ID NOs: 3-14.
27. The anti-TPBG antibody according to any one of the preceding items, obtained according to Example 1, 2 or 3 herein and/or having characteristics as described in Example 1, 2 or 3 herein (e.g., FIG. 1-21, particularly FIGS. 1, 2, 3, 4, 5 and/or 21).
28. The anti-TPBG antibody according to any one of the preceding items, comprising one or more (e.g., 2, 3, 4, 5, 6, 7, 8, 9 or 10) amino acid substitution/s (or mutations), preferably located in one or more regions selected from the group consisting of: CDRs (e.g., CDR1, CDR2, CDR3, e.g., CDR-H1, CDR-H2, CDR-H3, CDR-H1, CDR-L1, CDR-L2 and/or CDR-L3, e.g., according to any one of the preceding items, e.g., sequence listing as disclosed herein), V_{H} (variable region heavy chain), V_{L} (variable region light chain), C_{H} (constant region heavy chain) or C_{L} (constant region light chain), signal sequences, F(ab) and/or Fc region (e.g., as defined in FIG. 1 herein)
29. The anti-TPBG antibody according to any one of the preceding items, comprising one or more (e.g., 2, 3, 4, 5, 6, 7, 8, 9 or 10) highly conservative, conservative or equivalent amino acid substitution/s (or mutations), e.g., “Conservative or equivalent substitution/s” meaning substitutions as listed as “Exemplary Substitutions” in Table I below, “Highly conservative” substitutions as used herein meaning substitutions as shown under the heading “Preferred Substitutions” in Table I below:

TABLE I
Amino Acid Substitutions

| Original | Exemplary Substitutions | Preferred Substitutions |
|---|---|---|
| Ala (A) | val; leu; ile | Val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| Leu (L) | norleucine; ile; val; met; ala; | ile |
| Lys (K) | arg; gin; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | Phe |
| Val (V) | ile; leu; met; phe; ala; | leu |

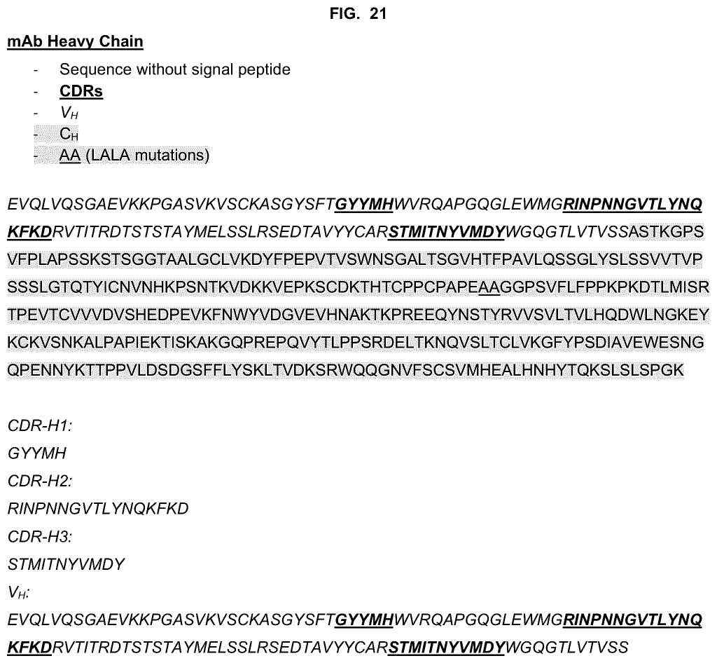

preferably said one or more (e.g., 2, 3, 4, 5, 6, 7, 8, 9 or 10) highly conservative, conservative or equivalent amino acid substitution/s (or mutations) are located in one or more regions selected from the group consisting of: CDRs (e.g., CDR1, CDR2, CDR3, e.g., CDR-H1, CDR-H2, CDR-H3, CDR-H1, CDR-L1, CDR-L2 and/or CDR-L3, e.g., according to any one of the preceding items, e.g., sequence listing as disclosed herein), V_{H} (variable region heavy chain), V_{L} (variable region light chain), C_{H} (constant region heavy chain) or C_{L} (constant region light chain), F(ab) and/or Fc region (e.g., as defined in FIG. 21 herein).
30. A hybridoma, wherein said hybridoma produces the monoclonal antibody according to any one of the preceding items.
31. A nucleic acid encoding the antibody according to any one of the preceding items.
32. An expression vector comprising at least one of the nucleic acid molecules according to any one of the preceding items.
33. An isolated host cell (e.g., an isolated recombinant host cell) comprising the vector and/or nucleic acid according to any one of the preceding items.
34. An antibody drug conjugate (ADC) comprising the anti-TPBG antibody according to any one of the preceding items (e.g., obtained according to Example 1, 2 or 3 herein and/or having characteristics as described in Example 1 and/or Example 2 and/or Example 3 herein).
35. The antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties (e.g., cytotoxic payloads, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan (e.g., CAS Nr: 171335-80-1), preferably via one or more linkers, further preferably via one or more phosphonamidate linkers.
36. The antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan) cytotoxic moieties, preferably via one or more linkers, further preferably via one or more phosphonamidate linkers.
37. The antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) Exatecan cytotoxic moieties via one or more linkers, preferably via one or more phosphonamidate linkers.
38. The antibody drug conjugate (ADC) according to any one of the preceding items, wherein said ADC comprising a humanized monoclonal TPBG-specific IgG1 antibody conjugated to a cytotoxic payload:
   a) wherein the cytotoxic payload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or
   b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or
   c) wherein the cytotoxic payload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or
   d) wherein the linker L comprises a valine-citrulline-PAB moiety or a valine-alanine-PAB moiety; and/or
   e) wherein cytotoxic payload is Exatecan, conjugated via a chemical valine-citrulline-PAB or a valine-alanine-PAB release unit, wherein said release unit is cleavable by a protease.
39. The antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan) cytotoxic moieties via an ethynylphosphonamidate-linker/s conjugation (e.g., to all eight interchain-cysteine residues), preferably each phosphonamidate linker carries at least one PEG24 moiety (e.g., to prevent aggregation of the ADC), further preferably said ADC carries up to said eight linker payload moieties and eight PEG24 moieties.
40. An antibody drug conjugate (ADC), optionally according to any one of items 34 to 39, having the formula (I):

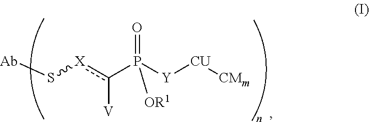

or a pharmaceutically acceptable salt or solvate thereof;
wherein:
Ab is an anti-TPBG antibody according to any one of the preceding items;
[symbol] is a double bond; or
[symbol] is a bond;
V is absent when [symbol] is a double bond; or
V is H or (C₁-C₈)alkyl when [symbol] is a bond;
X is R₃—C when [symbol] is a double bond; or
X is

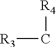

when [symbol] is a bond;
Y is NR⁵, S, O, or CR⁶R⁷;
R¹ is an optionally substituted aliphatic residue or an optionally substituted aromatic residue;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
CU is a connector unit;
CM is a cytotoxic moiety;
m is an integer ranging from 1 to 10; and
n is an integer ranging from 1 to 20.
41. The antibody drug conjugate (ADC) according to item 40, wherein:
   R³ is H or (C₁-C₈)alkyl; preferably R³ is H;
   R⁴ when present is H or (C₁-C₈)alkyl; preferably R⁴, when present, is H;
   R⁵ when present is H or (C₁-C₈)alkyl; preferably R⁵, when present, is H;
   R⁶ when present is H or (C₁-C₈)alkyl; preferably R⁶, when present, is H; and
   R⁷ when present is H or (C₁-C₈)alkyl; preferably R⁷, when present, is H.
42. The antibody drug conjugate (ADC) according to item 40 or 41, wherein [symbol] is a double bond; V is absent; X is R₃—C; and R³ is H or an optionally substituted aliphatic residue or an optionally substituted aromatic residue; preferably R³ is H or (C₁-C₈)alkyl; more preferably R³ is H.
43. The antibody drug conjugate (ADC) according to item 40 or 41, wherein [symbol] is a bond; V is H or (C₁-C₈)alkyl, preferably V is H; X is

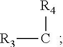

R₃ is H or an optionally substituted aliphatic residue or an optionally substituted aromatic residue; more preferably R³ is H or (C₁-C₈)alkyl, more preferably R³ is H; R⁴ is H or an optionally substituted aliphatic residue or an optionally substituted aromatic residue; preferably, R⁴ is H or (C₁-C₈)alkyl, preferably R⁴ is H.
44. The antibody drug conjugate (ADC) according to any one of items 40 to 43, wherein Y is NH.
45. The antibody drug conjugate (ADC) according to any one of items 40 to 44, wherein the connector unit CU is cleavable.
46. The antibody drug conjugate (ADC) according to item 45, wherein the connector unit CU is cleavable by a protease, a glucuronidase, a sulfatase, a phosphatase, an esterase, or by disulfide reduction.
47. The antibody drug conjugate (ADC) according to item 46, wherein the connector unit CU is cleavable by a protease, preferably by a cathepsin such as cathepsin B.
48. The antibody drug conjugate (ADC) according to any one of items 40 to 47, wherein the connector unit (CU) comprises a valine-citrulline moiety.
49. The antibody drug conjugate (ADC) according to item 48, wherein the connector unit CU is:

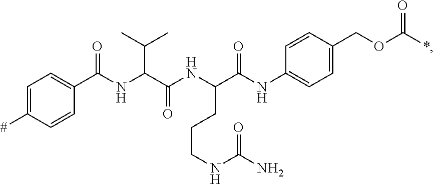

wherein # indicates the attachment point to the Y and * indicates the attachment point to the cytotoxic moiety CM.
50. The antibody drug conjugate (ADC) according to any one of items 40 to 47, wherein the connector unit CU comprises a valine-alanine moiety.
51. The antibody drug conjugate (ADC) according to item 50, wherein the connector unit CU is:

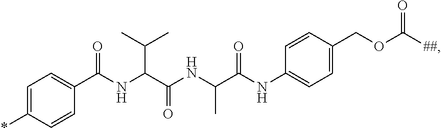

wherein * indicates the attachment point to the Y and ## indicates the attachment point to the cytotoxic moiety.
52. The antibody drug conjugate (ADC) according to any one of items 40 to 44, wherein the linker is non-cleavable.
53. The antibody drug conjugate (ADC) according to any one of items 40 to 52, wherein R¹ is a polyethylene glycol unit.
53a. The antibody drug conjugate (ADC) according to item 53, wherein the polyethylene glycol unit comprises 1 to 100 subunits having the structure:

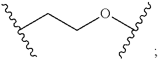 [SMILES: *CCO*]

54. The antibody drug conjugate (ADC) according to any one of items 40 to 53a, wherein R¹ is:

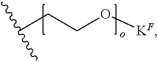

wherein
[symbol] indicates the position of the 0;
K^{F} is selected from the group consisting of —H, —PO₃H, —(C₁-C₁₀)alkyl, —(C₁-C₁₀)alkyl-SO₃H, —(C₂-C₁₀)alkyl-CO₂H, —(C₂-C₁₀)alkyl-OH, —(C₂-C₁₀)alkyl-NH₂, —(C₂-C₁₀)alkyl-NH(C₁-C₈)alkyl and —(C₂-C₁₀)alkyl-N((C₁-C₈)alkyl)₂; and
o is an integer ranging from 1 to 100.
55. The antibody drug conjugate (ADC) according to item 54, wherein K^{F} is H.
56. The antibody drug conjugate (ADC) according item 54 or 55, wherein o ranges from 8 to 30.
57. The antibody drug conjugate (ADC) according to item 56, wherein o ranges from 20 to 28.
58. The antibody drug conjugate (ADC) according to item 57, wherein o is 22, 23, 24, 25 or 26.
59. The antibody drug conjugate (ADC) according to item 56, wherein o ranges from 8 to 16.
60. The antibody drug conjugate (ADC) according to item 59, wherein o is 10, 11, 12, 13 or 14.
61. The antibody drug conjugate (ADC) according to any one of items 40 to 60, wherein the cytotoxic moiety CM is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof.
62. The antibody drug conjugate (ADC) according to item 61, wherein the cytotoxic moiety CM is a camptothecin moiety.
63. The antibody drug conjugate (ADC) according to item 62, wherein the camptothecin moiety is selected from the group consisting of exatecan, DXD, SN38, camptothecin, topotecan, irinotecan, belotecan, lurtotecan, rubitecan, silatecan, cositecan, and gimatecan.
64. The antibody drug conjugate (ADC) according to item 63, wherein the cytotoxic moiety CM is exatecan having the formula:

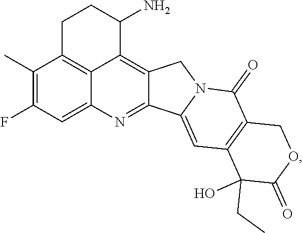 [SMILES: CCC1(O)C(=O)OCc2c1cc1n(c2=O)Cc2c-1nc1cc(F)c(C)c3c1c2C(N)CC3]

65. The antibody drug conjugate (ADC) according to item 64, wherein the cytotoxic moiety CM is exatecan having the formula:

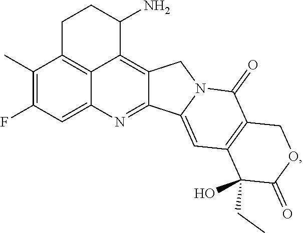 [SMILES: CC[C@@]1(O)C(=O)OCc2c1cc1n(c2=O)Cc2c-1nc1cc(F)c(C)c3c1c2C(N)CC3]

66. The antibody drug conjugate (ADC) according to item 64 or 65, wherein the exatecan is bound to the connector unit CU via the amino group.
67. The antibody drug conjugate (ADC) according to any one of items 40 to 66, wherein the number of cytotoxic moieties CM, in particular the number of camptothecin moieties, per receptor binding molecule is from 1 to 14, preferably from 2 to 14, more preferably from 4 to 14, still more preferably from 5 to 12, still more preferably from 6 to 12, still more preferably from 7 to 10, even more preferably 8.
68. The antibody drug conjugate (ADC) according to any one of items 40 to 66, wherein the number of cytotoxic moieties CM, in particular the number of camptothecin moieties, per receptor binding molecule is from 1 to 14, preferably from 1 to 12, more preferably from 2 to 10, still more preferably from 2 to 8, still more preferably from 2 to 6, still more preferably from 3 to 5, even more preferably 4.
69. The antibody drug conjugate (ADC) according to any one of items 40 to 66, wherein m is an integer ranging from 1 to 4, preferably 1 or 2, more preferably 1; and
n is an integer ranging from 1 to 20, preferably from 1 to 10, more preferably from 2 to 10, still more preferably from 4 to 10, still more preferably from 6 to 10, still more preferably from 7 to 10, even more preferably 8.
70. The antibody drug conjugate according to any one of items 40 to 66, wherein m is an integer ranging from 1 to 4, preferably 1 or 2, more preferably 1; and
   n is an integer ranging from 1 to 20, preferably from 1 to 10, more preferably from 2 to 8, still more preferably from 3 to 6, still more preferably 4 or 5, even more preferably 4.
71. An antibody drug conjugate (ADC), optionally according to any one of items 40 to 70, having the formula (I):

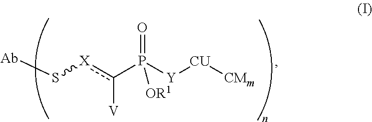

or a pharmaceutically acceptable salt or solvate thereof;
wherein:
Ab is an anti-TPBG antibody according to any one of the preceding items;
[symbol] is a double bond; or
[symbol] is a bond;
V is absent when [symbol] is a double bond; or
V is H when [symbol] is a bond;
X is R₃—C when [symbol] is a double bond; or
X is

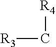

when [symbol] is a bond;
Y is NH;
R¹ is a polyethylene glycol unit having the structure:

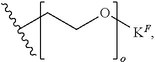

wherein:
[symbol] indicates the position of the 0;
K^{F} is H; and
o is an integer ranging from 8 to 30;
R³ is H;
R⁴ is H;
CU is a connector unit having the following structure:

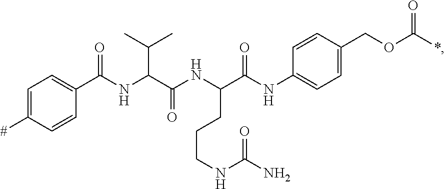

wherein # indicates the attachment point to the Y and * indicates the attachment point to the camptothecin moiety (CM);
CM is a camptothecin moiety;
m is 1; and
n is an integer ranging from 1 to 10.
72. The antibody drug conjugate (ADC) according to item 71, wherein [symbol] is a double bond; V is absent; X is R₃—C; and R³ is H.
72a. The antibody drug conjugate (ADC) according to item 71, wherein [symbol] is a bond; V is H; X is

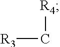

R³ is H; and R⁴ is H.
73. The antibody drug conjugate (ADC) according to any one of items 71 to 72a, wherein the camptothecin moiety CM is exatecan having the formula:

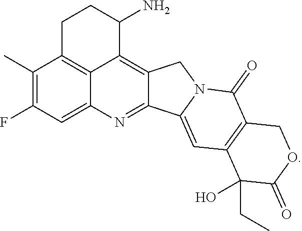 [SMILES: CCC1(O)C(=O)OCc2c1cc1n(c2=O)Cc2c-1nc1cc(F)c(C)c3c1c2C(N)CC3]

74. The antibody drug conjugate (ADC) according to item 73, wherein the exatecan is bound to the connector unit CU via the amino group.
75. The antibody drug conjugate (ADC) according to any one of items 71 to 74, wherein o ranges from 20 to 28.
76. The antibody drug conjugate (ADC) according to item 75, wherein o is 22, 23, 24, 25 or 26.
77. The antibody drug conjugate (ADC) according to any one of items 71 to 76, wherein n ranges from 2 to 10,
78. The antibody drug conjugate (ADC) according to item 77, wherein n is 8.
79. The antibody drug conjugate (ADC) according to item 77, wherein n is 4.
80. An antibody drug conjugate (ADC), optionally according to any one of items 71 to 78, having the following formula (Ia):

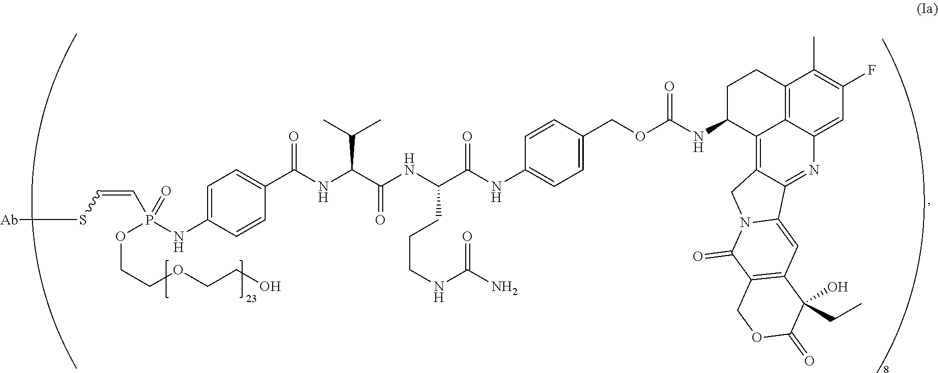

wherein Ab is an anti-TPBG antibody according to any one of the preceding items.
81. The antibody drug conjugate (ADC) according to item 61, wherein the cytotoxic moiety CM is an auristatin.
82. The antibody drug conjugate (ADC) according to item 81, wherein the cytotoxic moiety is monomethyl auristatin E (MMAE) or monomethyl auristatin F (MMAF).
83. The antibody drug conjugate (ADC) according to item 82, wherein the cytotoxic moiety is monomethyl auristatin E (MMAE).
84. The antibody drug conjugate (ADC) according to any one of the preceding items, wherein said ADC having a formula selected from the group consisting of:

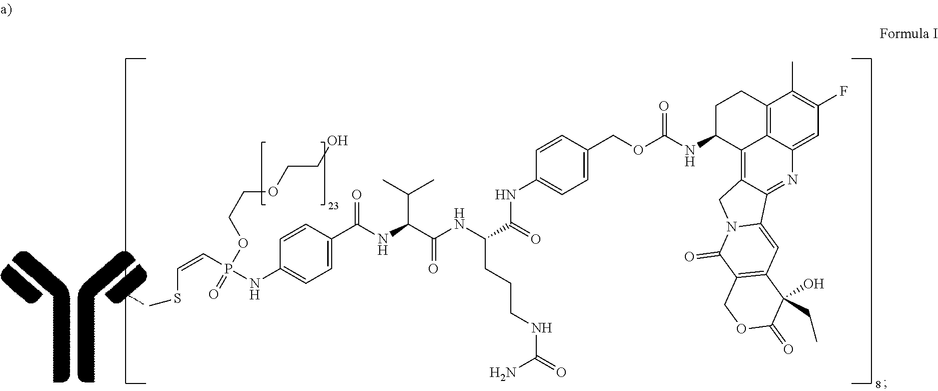

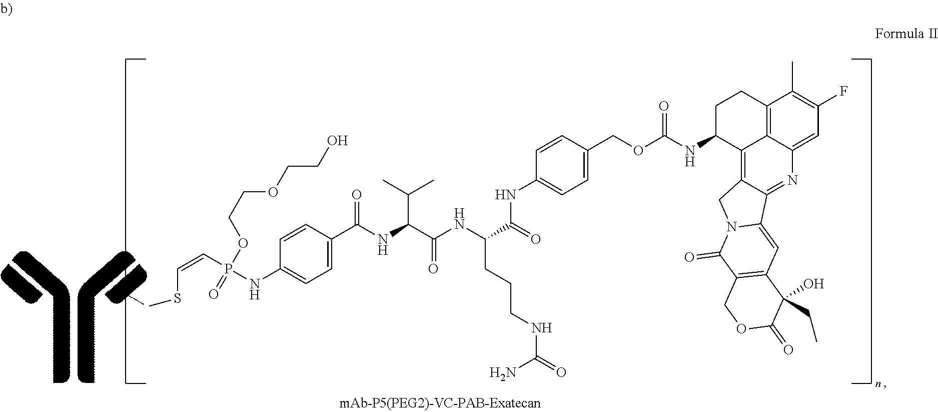

wherein n is in the range from 0 to 20 (e.g., 1, 2, 3, 4, 5, 6, 7 or 8);

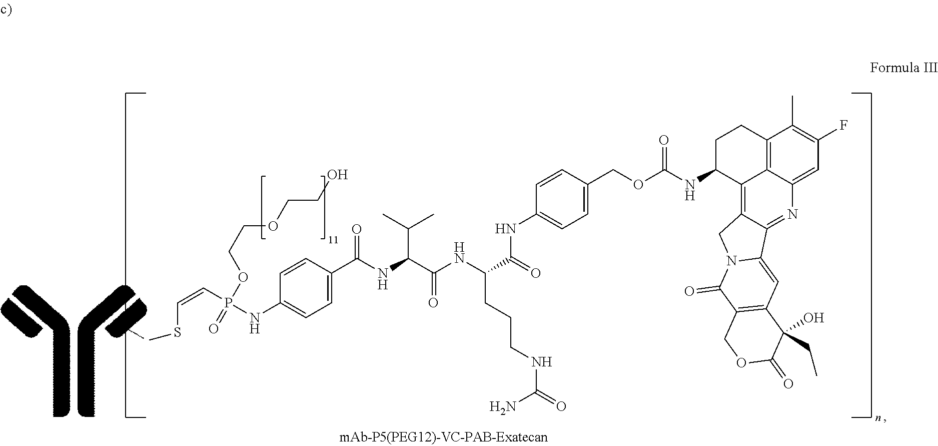

wherein n is in the range from 0 to 20 (e.g., 1, 2, 3, 4, 5, 6, 7 or 8);

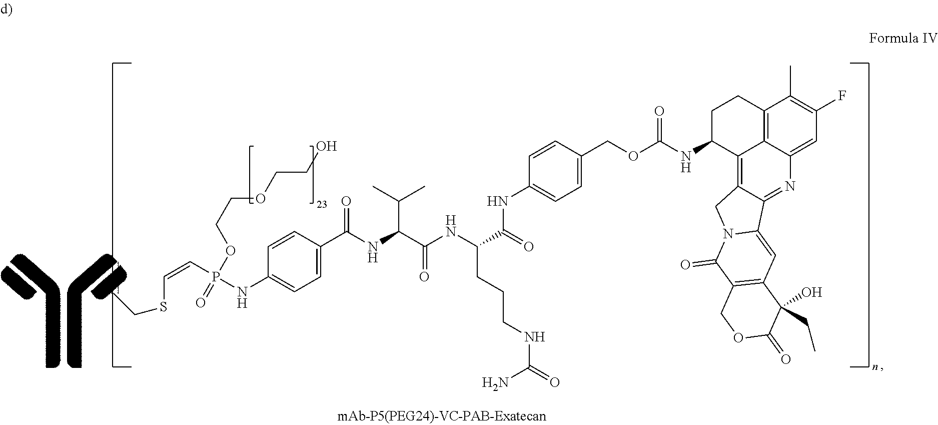

wherein n is in the range from 0 to 20 (e.g., 1, 2, 3, 4, 5, 6, 7 or 8);

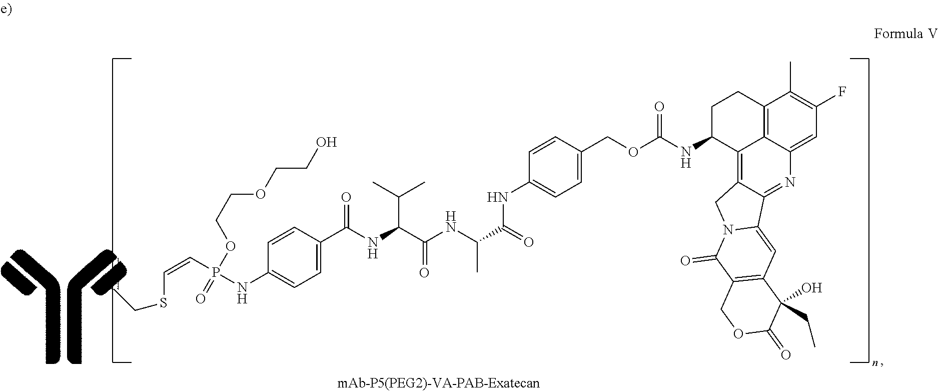

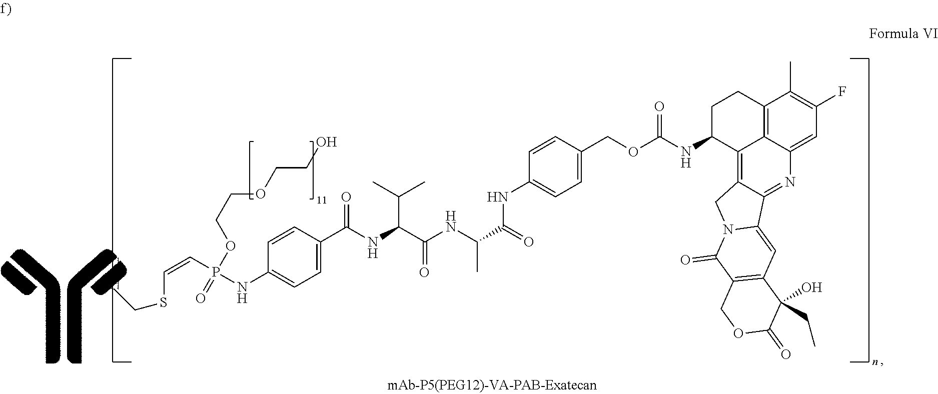

wherein n is in the range from 0 to 20 (e.g., 1, 2, 3, 4, 5, 6, 7 or 8);
wherein n is in the range from 0 to 20 (e.g., 1, 2, 3, 4, 5, 6, 7 or 8);

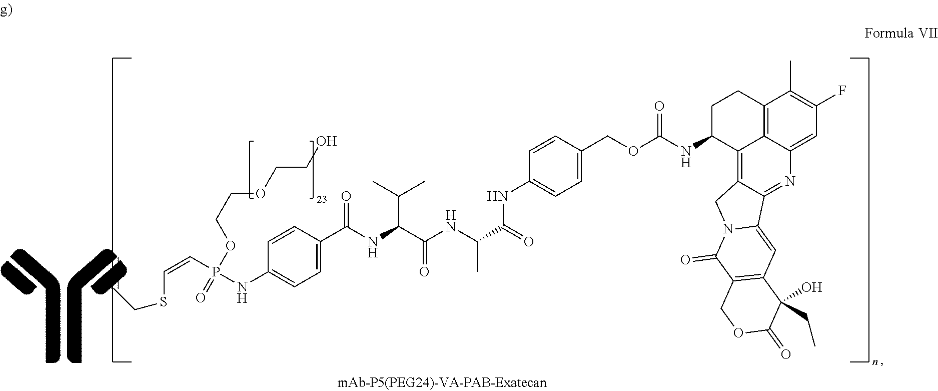

wherein n is in the range from 0 to 20 (e.g., 1, 2, 3, 4, 5, 6, 7 or 8);

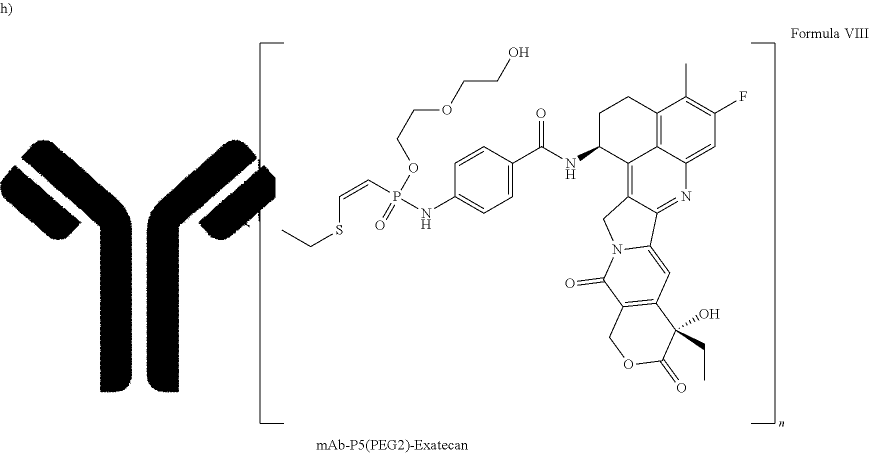

wherein n is in the range from 0 to 20 (e.g., 1, 2, 3, 4, 5, 6, 7 or 8);

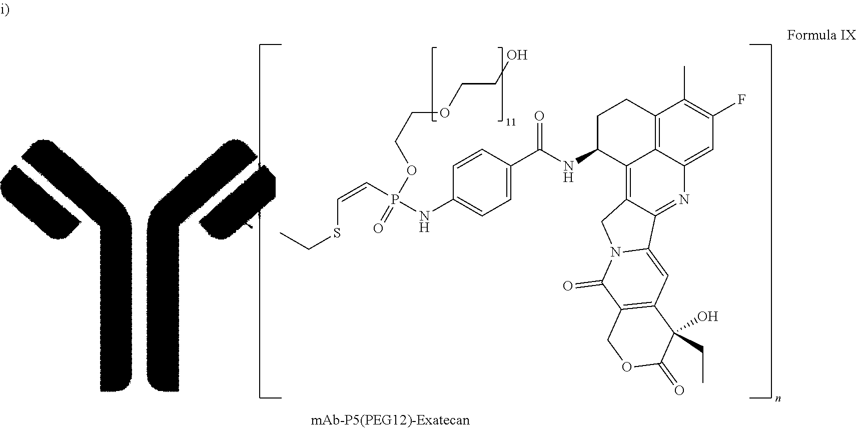

wherein n is in the range from 0 to 20 (e.g., 1, 2, 3, 4, 5, 6, 7 or 8);

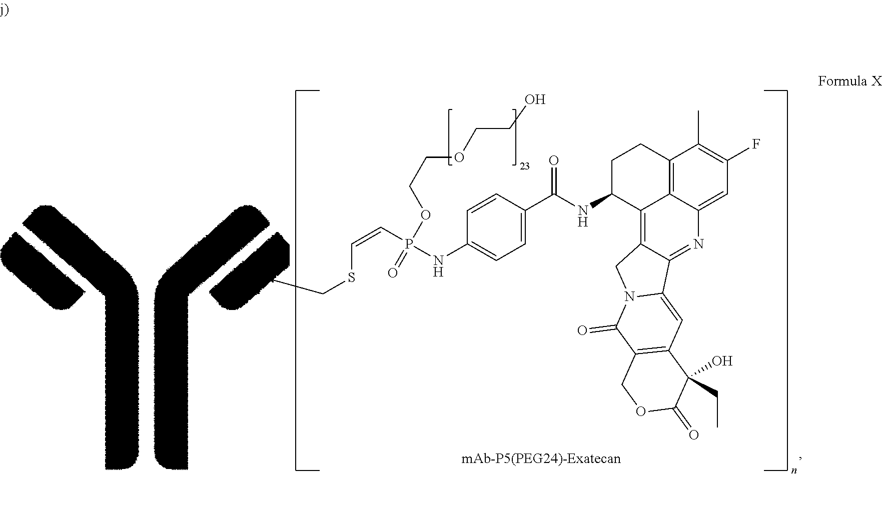

wherein n is in the range from 0 to 20 (e.g., 1, 2, 3, 4, 5, 6, 7 or 8);

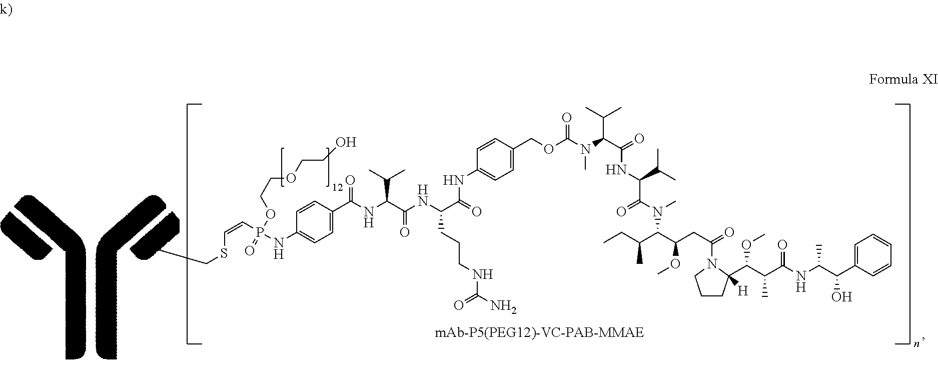

wherein n is in the range from 0 to 20 (e.g., 1, 2, 3, 4, 5, 6, 7 or 8);

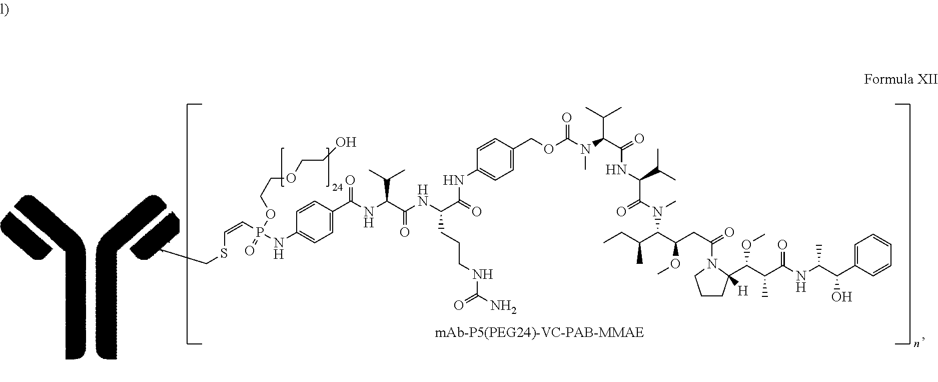

wherein n is in the range from 0 to 20 (e.g., 1, 2, 3, 4, 5, 6, 7 or 8);

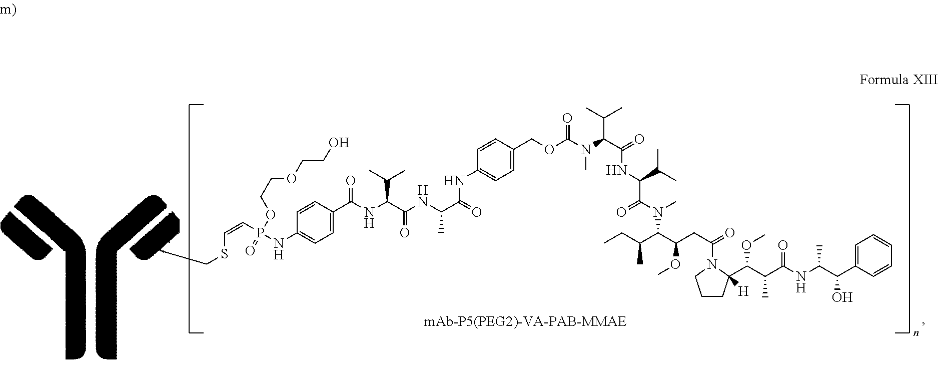

wherein n is in the range from 0 to 20 (e.g., 1, 2, 3, 4, 5, 6, 7 or 8);

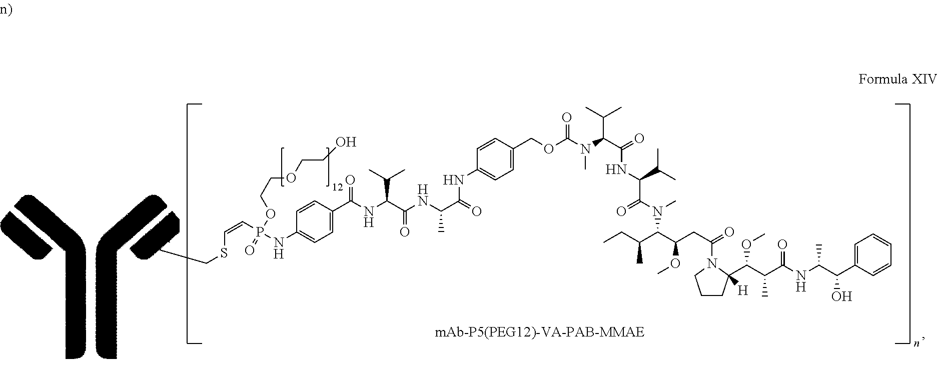

wherein n is in the range from 0 to 20 (e.g., 1, 2, 3, 4, 5, 6, 7 or 8);

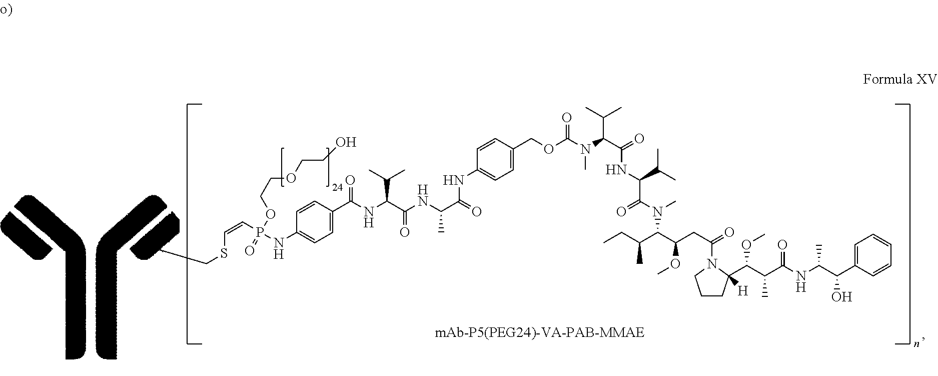

wherein n is in the range from 0 to 20 (e.g., 1, 2, 3, 4, 5, 6, 7 or 8);

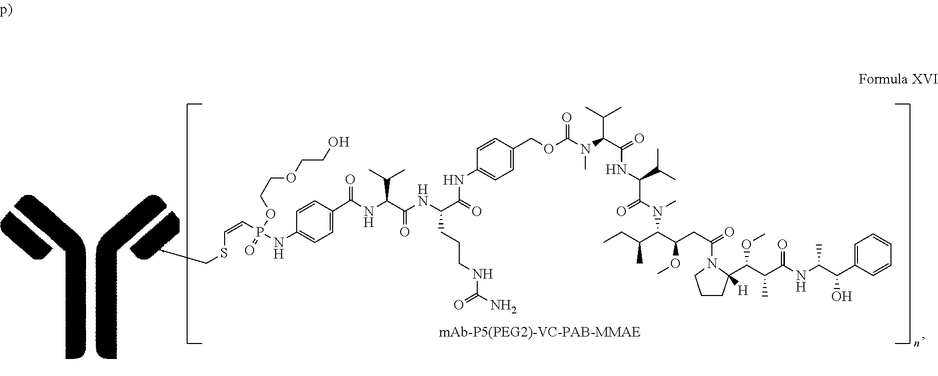

wherein n is in the range from 0 to 20 (e.g., 1, 2, 3, 4, 5, 6, 7 or 8);
   wherein [symbol] is an anti-TPBG antibody according to any one of the preceding items.
85. The antibody drug conjugate (ADC) according to any one of the preceding items, wherein:
   a) said anti-TPBG monoclonal antibody is capable of specifically recognizing TPBG (e.g., human) overexpressed on cancer cell/s; and
   b) upon binding of said ADC to said TPBG overexpressed on cancer cells said ADC is capable of being internalized by the cells and trafficked into the lysosomal compartment, in which preferably a lysosomal protease (e.g., Cathepsin B) is capable of releasing said cytotoxic payload from the said ADC.
86. A compound having the formula (II):

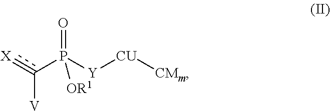

or a pharmaceutically acceptable salt or solvate thereof;
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

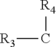

when [symbol] is a double bond;
Y is NR⁵, S, O, or CR⁶R⁷;
R¹ is an optionally substituted aliphatic residue or an optionally substituted aromatic residue;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
CU is a connector unit;
CM is a cytotoxic moiety; and
m is an integer ranging from 1 to 10.
87. The compound according to item 86, wherein:
   R³ is H or (C₁-C₈)alkyl; preferably R³ is H;
   R⁴ when present is H or (C₁-C₈)alkyl; preferably R⁴, when present, is H;
   R⁵ when present is H or (C₁-C₈)alkyl; preferably R⁵, when present, is H;
   R⁶ when present is H or (C₁-C₈)alkyl; preferably R⁶, when present, is H; and
   R⁷ when present is H or (C₁-C₈)alkyl; preferably R⁷, when present, is H.
88. The compound according to item 86 or 87, wherein [symbol] is a triple bond; V is absent; X is R₃—C; and R³ is H or an optionally substituted aliphatic residue or an optionally substituted aromatic residue; preferably R³ is H or (C₁-C₈)alkyl, more preferably R³ is H.
89. The compound according to item 86 or 87, wherein [symbol] is a double bond; V is H or (C₁-C₈)alkyl, preferably V is H; X is

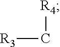

R³ is H or an optionally substituted aliphatic residue or an optionally substituted aromatic residue; preferably R³ is H or (C₁-C₈)alkyl, more preferably R³ is H; R⁴ is H or an optionally substituted aliphatic residue or an optionally substituted aromatic residue; preferably R⁴ is H or (C₁-C₈)alkyl, more preferably R⁴ is H.
90. The compound according to any one of items 86 to 89, wherein Ab, V, X, Y, R¹, R³, R⁴, R⁵, R⁶, R⁷, CU, CM, m and n are as defined in any one of items 40 to 85; preferably, Y is NH.
91. A method of producing an antibody drug conjugate (ADC), said method comprising:
   (a) conjugating the antibody according to any one of the preceding items to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) cytotoxic moieties (e.g., cytotoxic payloads, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan), preferably via one or more linkers, further preferably via one or more phosphonamidate linkers.
92. The method of producing an antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan) cytotoxic moieties, preferably via one or more linkers, further preferably via one or more phosphonamidate linkers.
93. The method of producing an antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) Exatecan cytotoxic moieties via one or more linkers, preferably via one or more phosphonamidate linkers.
94. The method of producing an antibody drug conjugate (ADC) according to any one of the preceding items, wherein said anti-TPBG antibody is conjugated to one or more (e.g., 1, 2, 3, 4, 5, 6, 7, 8, 9, 10, 11, 12, 13, 14, 15, 16, 17, 18, 19 or 20, preferably from 1 to 10, further preferably from 2 to 10, most preferably from 4 to 10, further most preferably from 6 to 10, further most preferably from 7 to 10, further most preferably 4 or 8, most preferably to 8) camptothecin (e.g., Exatecan) cytotoxic moieties via an ethynylphosphonamidate-linker/s conjugation (e.g., to all eight interchain-cysteine residues), preferably each phosphonamidate linker carries at least one PEG moiety, with up to 24 PEG units (e.g., to prevent aggregation of the ADC), further preferably said ADC carries up to said eight linker payload moieties and eight PEG24 moieties.
95. The method of producing an antibody drug conjugate (ADC) according to any one of the preceding items, wherein said ADC comprising a humanized monoclonal TPBG-specific IgG1 antibody conjugated to a cytotoxic payload:
   a) wherein the cytotoxic payload is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof; and/or
   b) wherein cytotoxic payload is a camptothecin moiety C selected from the group consisting of exatecan, DXD, SN38, camptothecin, topotecan, irinotecan, belotecan, lurtotecan, rubitecan, silatecan, cositecan, and gimatecan; and/or
   c) wherein the cytotoxic payload is conjugated via a cleavable linker (L), preferably wherein the linker L is cleavable by a protease, a glucuronidase, a sulfatase, a phosphatase, an esterase, or by disulfide reduction, more preferably wherein the linker is cleavable by a protease, preferably by a cathepsin such as cathepsin B; and/or
   d) wherein the linker L comprises a valine-citrulline-PAB moiety or a valine-alanine-PAB moiety; and/or
   e) wherein cytotoxic payload is Exatecan, conjugated via a chemical valine-citrulline-PAB or a valine-alanine-PAB release unit, wherein said release unit is cleavable by a protease.
96. A method of preparing an antibody drug conjugate (ADC), optionally according to any one of items 91 to 95, said method comprising:
   reacting a compound of formula (II)

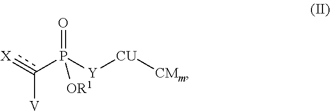

or a pharmaceutically acceptable salt or solvate thereof;
wherein:
[symbol] is a triple bond; or
[symbol] is a double bond;
V is absent when [symbol] is a triple bond; or
V is H or (C₁-C₈)alkyl when [symbol] is a double bond;
X is R₃—C when [symbol] is a triple bond; or
X is

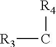

when [symbol] is a double bond;
Y is NR⁵, S, O, or CR⁶R⁷;
R¹ is an optionally substituted aliphatic residue or an optionally substituted aromatic residue;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
CU is a connector unit;
CM is a cytotoxic moiety; and
m is an integer ranging from 1 to 10;
with a thiol-containing molecule of formula (III)

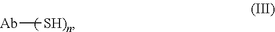

wherein Ab is an anti-TPBG antibody according to any one of the preceding items; and
n is an integer ranging from 1 to 20;
resulting in an antibody drug conjugate (ADC) of formula (I)

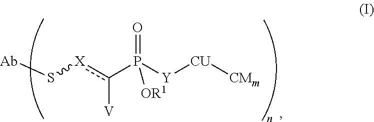

or a pharmaceutically acceptable salt or solvate thereof;
wherein:
Ab is an anti-TPBG antibody according to any one of the preceding items;
[symbol] is a double bond when [symbol] in a compound of formula (II) is a triple bond; or
[symbol] is a bond when [symbol] in a compound of formula (II) is a double bond;
V is absent when [symbol] is a double bond; or
V is H or (C₁-C₈)alkyl when [symbol] is a bond;
X is R₃—C when [symbol] is a double bond; or
X is

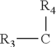

when [symbol] is a bond;
Y is NR⁵, S, O, or CR⁶R⁷;
R¹ is an optionally substituted aliphatic residue or an optionally substituted aromatic residue;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
CU is a connector unit;
CM is a cytotoxic moiety;
m is an integer ranging from 1 to 10; and
n is an integer ranging from 1 to 20.
97. The method according to item 96, wherein:
   R³ is H or (C₁-C₈)alkyl; preferably R³ is H;
   R⁴ when present is H or (C₁-C₈)alkyl; preferably R⁴, when present, is H;
   R⁵ when present is H or (C₁-C₈)alkyl; preferably R⁵, when present, is H;
   R⁶ when present is H or (C₁-C₈)alkyl; preferably R⁶, when present, is H; and
   R⁷ when present is H or (C₁-C₈)alkyl; preferably R⁷, when present, is H.
98. The method according to item 96 or 97, wherein [symbol] is a triple bond; V is absent; X is R₃—C; R³ is H or an optionally substituted aliphatic residue or an optionally substituted aromatic residue, preferably R³ is H or (C₁-C₈)alkyl, more preferably R³ is H, and [symbol] is a double bond.
99. The method according to item 96 or 97, wherein [symbol] is a double bond; V is H or (C₁-C₈)alkyl, preferably V is H; X is

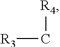

R³ is H or an optionally substituted aliphatic residue or an optionally substituted aromatic residue, preferably R³ is H or (C₁-C₈)alkyl, more preferably R³ is H; R⁴ is H or (C₁-C₈)alkyl, preferably R⁴ is H; and [symbol] is a bond.
100. The method according to any one of items 96 to 99, wherein the reaction is performed under neutral pH or slightly basic conditions, preferably at a pH of from 6 to 10.
101. The method according to any one of items 96 to 100, further comprising reducing at least one disulfide bridge of the antibody in the presence of a reducing agent to form a thiol group (SH).
102. The method according to item 101, wherein the reducing agent is selected from the group consisting of tris(2-carboxyethyl)phosphine (TCEP), dithiothreitol (DTT), sodium dithionite, sodium thiosulfate, and sodium sulfite;
   preferably wherein the reducing agent is tris(2-carboxyethyl)phosphine (TCEP).
103. The method according to item 101 or 102, wherein the reducing of at least one disulfide bridge comprises using about 1 to about 3 equivalents, preferably about 1 to about 2 equivalents, more preferably about 1 equivalent of the reducing agent per disulfide bridge to be reduced.
104. The method according to any one of items 96 to 103, wherein the thiol-containing molecule of formula (III) is reacted with about 1 to about 4 equivalents, preferably about 1 to about 3 equivalents, more preferably about 1 to about 2 equivalents, still more preferably about 1.5 equivalents of the compound of formula (II) per thiol group (SH).
105. The method according to any one of items 96 to 104, wherein the reacting a compound of formula (II) with a thiol-containing molecule of formula (III) is carried out in an aqueous medium.
106. The method according to any one of items 96 to 105, wherein Ab, V, X, Y, R¹, R³, R⁴, R⁵, R⁶, R⁷, CU, CM, m and n are as defined in any one of items 40 to 85; preferably Y is NH₂.
107. An antibody drug conjugate (ADC) produced by the method according to any one of the preceding items.
108. The antibody drug conjugate (ADC) according to any one of the preceding items, wherein the drug to antibody ratio (DAR) is in the range between 0 and 20, preferably is in the range between 1 and 20, further preferably is in the range between 2 and 12, most preferably is in the range between 4 and 10, further most preferably is in the range between 4 and 8.
109. The antibody drug conjugate (ADC) according to any one of the preceding items, wherein DAR of said ADC is 4 or 8, preferably 8.
110. A composition or kit comprising said anti-TPBG, antibody drug conjugate (ADC), hybridoma, nucleic acid, expression vector and/or host cell according to any one of the preceding items.
111. The composition according to any one of the preceding items, wherein said composition is a pharmaceutical and/or diagnostic composition.
112. The composition or kit according to any one of preceding items, wherein a drug (e.g., cytotoxic moieties (e.g., cytotoxic payloads, e.g. Tubulin disrupting agents, e.g., Topoisomerase-I inhibitor/s, e.g., Auristatins or camptothecin, e.g., MMAE (monomethyl auristatin E) or MMAF (monomethyl auristatin F), e.g., Exatecan) to antibody ratio (DAR) is in the range between 0 and 20, preferably is in the range between 1 and 20, further preferably is in the range between 2 and 12, most preferably is in the range between 4 and 10, further most preferably is in the range between 4 and 8 (e.g., 4 or 8).
113. A method for treatment, amelioration, prophylaxis and/or diagnostics of cancer, preferably said cancer is selected from the group consisting of: Breast cancer, Head and neck cancer, Ovary cancer, Endometrium cancer, Uterine cervix cancer, Rectum cancer, Colon cancer, Esophagus cancer, Stomach cancer, Lung cancer, Kidney cancer, Adrenal gland cancer, Bladder cancer, Liver cancer, Sarcoma, Brain cancer, Nevi and Melanomas, Urogenital cancer, Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers, Retinoblastom, Thyroid cancer, Fallopian tube cancer; further preferably said cancer is a solid cancer, most preferably most preferably said cancer is selected from the group consisting of: Breast cancer, Head and neck cancer, Ovarian cancer, Endometrial cancer, Uterus cancer (e.g., cancers of the muscle sheets), Cervical cancer, Rectum cancer, Colon cancer, Anal cancer, Esophagus cancer, Stomach cancer, Lung cancer, Kidney cancer, Adrenal gland cancer, Bladder cancer, Liver cancer, Sarcoma (e.g., osteosarcoma and Kaposi sarcoma), Brain cancer (e.g., pituitary tumor/s), Nevi and Melanoma cancers, Skin cancers (e.g., squamous cell carcinoma and melanoma), Urogenital cancer (e.g., ureter and bladder cancer, testicular cancer, prostate cancer, penile cancer), Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers (e.g., lymphoma, leukemia, myeloma, Myelodysplastic syndromes or myelofibrosis), Eye cancers (e.g., Retinoblastoma), Neuroendocrine tumors, Cancer of unknown primary (CUP), breast cancer, colon cancer, endometrial cancer, sarcoma, gastric cancer, head and neck cancer, pancreatic cancer, lung cancer, mesothelioma, ovarian cancer (e.g., as in Example 2 or 3 herein), said method comprising: administering a therapeutically or prophylactically effective amount of the antibody, antibody drug conjugate (ADC), nucleic acid, expression vector, host cell, composition or kit according to any one of the preceding items.
114. The antibody, antibody drug conjugate (ADC), nucleic acid, expression vector, host cell, composition or kit according to any one of the preceding items, for use as a medicament and/or in therapy.
115. The antibody, antibody drug conjugate (ADC), nucleic acid, expression vector, host cell, composition or kit according to any one of the preceding items, for use in one or more of the following methods:
   (a) method for treatment, amelioration, prophylaxis and/or diagnostics of cancer, preferably said cancer is selected from the group consisting of: Breast cancer, Head and neck cancer, Ovary cancer, Endometrium cancer, Uterine cervix cancer, Rectum cancer, Colon cancer, Esophagus cancer, Stomach cancer, Lung cancer, Kidney cancer, Adrenal gland cancer, Bladder cancer, Liver cancer, Sarcoma, Brain cancer, Nevi and Melanomas, Urogenital cancer, Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers, Retinoblastom, Thyroid cancer, Fallopian tube cancer; further preferably said cancer is a solid cancer; most preferably said cancer is selected from the group consisting of: Breast cancer, Head and neck cancer, Ovarian cancer, Endometrial cancer, Uterus cancer (e.g., cancers of the muscle sheets), Cervical cancer, Rectum cancer, Colon cancer, Anal cancer, Esophagus cancer, Stomach cancer, Lung cancer, Kidney cancer, Adrenal gland cancer, Bladder cancer, Liver cancer, Sarcoma (e.g., osteosarcoma and Kaposi sarcoma), Brain cancer (e.g., pituitary tumor/s), Nevi and Melanoma cancers, Skin cancers (e.g., squamous cell carcinoma and melanoma), Urogenital cancer (e.g., ureter and bladder cancer, testicular cancer, prostate cancer, penile cancer), Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers (e.g., lymphoma, leukemia, myeloma, Myelodysplastic syndromes or myelofibrosis), Eye cancers (e.g., Retinoblastoma), Neuroendocrine tumors, Cancer of unknown primary (CUP), breast cancer, colon cancer, endometrial cancer, sarcoma, gastric cancer, head and neck cancer, pancreatic cancer, lung cancer, mesothelioma, ovarian cancer (e.g., as in Example 2 or 3 herein);
   (b) method for monitoring development of cancer and/or for assessing the efficacy and/or toxicity of cancer therapy or anti-cancer compound/agent;
   (c) method for screening a candidate compound for anti-cancer activity;
   (d) method for altering resistance of cancer cells to chemotherapy;
   (e) method for sensitizing cancer cells to chemotherapy;
   (f) method for inhibiting the growth of cancer cell expressing TPBG;
   (g) method for production or preparation of an antibody;
   (h) method for immunizing a non-human animal;
   (i) method for preparation of a hybridoma;
   (j) method according to any one of the preceding items;
   (k) method according to any one of (a)-(j), wherein said method is an in vivo, in vitro, or ex vivo method.
116. Use of the antibody, antibody drug conjugate (ADC), nucleic acid, expression vector, host cell, composition or kit according to any one of the preceding items, for one or more of the following:
   (a) for treatment, amelioration, prophylaxis and/or diagnostics of cancer, preferably said cancer is selected from the group consisting of: Breast cancer, Head and neck cancer, Ovary cancer, Endometrium cancer, Uterine cervix cancer, Rectum cancer, Colon cancer, Esophagus cancer, Stomach cancer, Lung cancer, Kidney cancer, Adrenal gland cancer, Bladder cancer, Liver cancer, Sarcoma, Brain cancer, Nevi and Melanomas, Urogenital cancer, Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers, Retinoblastom, Thyroid cancer, Fallopian tube cancer; further preferably said cancer is a solid cancer; most preferably said cancer is selected from the group consisting of: Breast cancer, Head and neck cancer, Ovarian cancer, Endometrial cancer, Uterus cancer (e.g., cancers of the muscle sheets), Cervical cancer, Rectum cancer, Colon cancer, Anal cancer, Esophagus cancer, Stomach cancer, Lung cancer, Kidney cancer, Adrenal gland cancer, Bladder cancer, Liver cancer, Sarcoma (e.g., osteosarcoma and Kaposi sarcoma), Brain cancer (e.g., pituitary tumor/s), Nevi and Melanoma cancers, Skin cancers (e.g., squamous cell carcinoma and melanoma), Urogenital cancer (e.g., ureter and bladder cancer, testicular cancer, prostate cancer, penile cancer), Prostate cancer, Vulva Squamous cell carcinoma, Oropharyngeal cancer, Endocrine gland cancer, Thoracic Cancer, Mesothelioma, Pancreas cancer, Cholangiocarcinoma, Blood cancers (e.g., lymphoma, leukemia, myeloma, Myelodysplastic syndromes or myelofibrosis), Eye cancers (e.g., Retinoblastoma), Neuroendocrine tumors, Cancer of unknown primary (CUP), breast cancer, colon cancer, endometrial cancer, sarcoma, gastric cancer, head and neck cancer, pancreatic cancer, lung cancer, mesothelioma, ovarian cancer (e.g., as in Example 2 or 3 herein);
   (b) for monitoring development of cancer and/or for assessing the efficacy and/or toxicity of cancer therapy or anti-cancer compound/agent;
   (c) for screening a candidate compound for anti-cancer activity;
   (d) for altering resistance of cancer cells to chemotherapy;
   (e) for sensitizing cancer cells to chemotherapy;
   (f) for inhibiting the growth of cancer cell expressing TPBG;
   (g) for production or preparation of an antibody;
   (h) for immunizing a non-human animal;
   (i) for preparation of a hybridoma;
   (j) in a method according to any one of the preceding items;
   (k) use according to any one of (a)-(j), wherein said use is an in vivo, in vitro, or ex vivo use.
117. The antibody drug conjugate (ADC), composition, kit, method and/or use according to any one of the preceding items, wherein a dose (e.g., therapeutic dose, e.g., effective therapeutic dose, and/or daily dose, e.g., single or multiple daily dose, and/or total dose) comprises or consists of the ADC according to any one of the preceding items in the amount of at least about 10 mg/kg body weight of the treatment subject (e.g., animal or human patient), preferably in the amount from about 10 to about 15 mg/kg, e.g., 10, 11, 12, 13, 14, 15 or more mg/kg), or in the total amount of at least about 700-2000 mg of the ADC according to any one of the preceding items (e.g., as in Example 2 or 3 herein).
118. The antibody drug conjugate (ADC), composition, kit, method and/or use according to any one of the preceding items, wherein a dose (e.g., therapeutic dose, e.g., effective therapeutic dose, and/or daily dose, e.g., single or multiple daily dose, and/or total dose) of the ADC according to any one of the preceding items is administered (e.g., orally or intravenously) one or more times (e.g., 2, 3, 4, 5, 6, 7, 8, 9 or 10, e.g., every second or third day or once or twice a week) during a period of at least about 1 week (e.g., 1-4 weeks, e.g., 3 weeks, one or two months) with or without dose escalation (e.g., as in Example 2 or 3 herein).
119. The antibody drug conjugate (ADC), composition, kit, method and/or use according to any one of the preceding items, wherein a treatment regiment comprises or consists of administration (e.g., orally or intravenously) of a dose (e.g., therapeutic dose, e.g., effective therapeutic dose, and/or daily dose, e.g., single or multiple daily dose, and/or total dose) of the ADC according to any one of the preceding items one or more times (e.g., 2, 3, 4, 5, 6, 7, 8, 9 or 10, e.g., every second or third day or once or twice a week) during a period of at least about 1 week (e.g., 1-4 weeks, e.g., 3 weeks, one or two months) with or without dose escalation (e.g., as in Example 2 or 3 herein).
120. The antibody drug conjugate (ADC), composition, kit, method and/or use according to any one of the preceding items, further comprising one or more therapeutic agents (e.g., anti-cancer agent or medications).
121. The antibody drug conjugate (ADC), composition, kit, method and/or use according to any one of the preceding items, further comprising administration (before, simultaneously or consequently) of one or more therapeutic agents (e.g., anti-cancer agent or medication).

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

The term “less than” or in turn “more than” does not include the concrete number.

For example, less than 20 means less than the number indicated. Similarly, more than or greater than means more than or greater than the indicated number, e.g., more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

### EXAMPLES OF THE INVENTION

An even better understanding of the present invention and of its advantages will be evident from the following examples, offered for illustrative purposes only. The examples are not intended to limit the scope of the present invention in any way.

### Example 1

### General Information

### Chemicals, Solvents and Antibodies

Chemicals and solvents were purchased from Merck (Merck group, Germany), TCI (Tokyo chemical industry CO., LTD., Japan), Iris Biotech (Iris Biotech GmbH, Germany), MCE (MedChemExpress, USA) and Carl Roth (Carl Roth GmbH+Co. KG, Germany) and used without further purification. Dry solvents were purchased from Merck (Merck group, Germany). PEG24 was purchased from BiochemPEG (Pure Chemistry Scientific Inc., United States).

### Preparative HPLC

Preperative HPLC was performed on a BÜCHI Pure C-850 Flash-Prep system (BÜCHI Labortechnik AG, Switzerland) using a VP 250/10 Macherey-Nagel Nucleodur C18 HTec Spum column (Macherey-Nagel GmbH & Co. Kg, Germany) for smaller scales. The following gradients were used: Method C: (A=H2O+0.1% TFA (trifluoroacetic acid), B=MeCN (acetonitrile)+0.1% TFA, flow rate 6 ml/min, 30% B 0-5 min, 30-70% B 5-35 min, 99% B 35-45 min. For bigger scales, a VP 250/21 Macherey-Nagel Nucleodur C18 HTec Spum column (Macherey-Nagel GmbH & Co. Kg, Germany) was used with the following gradients were used: Method D: (A=H2O+0.1% TFA (trifluoroacetic acid), B=MeCN (acetonitrile)+0.1% TFA, flow rate 14 ml/min, 30% B 0-5 min, 30-70% B 5-35 min, 99% B 35-45 min.

### LC/MS

Small molecules, linker-payloads, antibodies and ADCs were analyzed using a Waters H-class instrument equipped with a quaternary solvent manager, a Waters sample manager-FTN, a Waters PDA detector and a Waters column manager with an Acquity UPLC protein BEH C4 column (300 Å, 1.7 μm, 2.1 mm×50 mm) for antibodies and ADCs. Here, samples were eluted at a column temperature of 80° C. The following gradient was used: A: 0.1% formic acid in H₂O; B: 0.1% formic acid in MeCN. 25% B 0-1 min, 0.4 mL/min, 25-95% B 1-3.5 min 0.2 mL/min, 95% B 3.5-4.5 min 0.2 mL/min, 95-25% B 4.5-5 min 0.4 mL/min, 25-95% B 5-5.5 min 0.4 mL/min, 95-25% B 5.5-7.5 min 0.4 mL/min. Mass analysis was conducted with a Waters XEVO G2-XS QT of analyzer. Proteins were ionized in positive ion mode applying a cone voltage of 40 kV. Raw data was analyzed with MaxEnt 1. Small molecules and linker-payloads were analyzed with an Acquity UPLC-BEH C18 column (300 Å, 1.7 μm, 2.1 mm×50 mm). Here, samples were eluted at a column temperature of 45° C. with a flow rate of 0.4 mL/min. The following gradient was used: A: 0.1% formic acid in H₂O; B: 0.1% formic acid in MeCN. 2% B 0-1 min, 2-98% B 1-5 min, 98% B 5-5.5 min, 98-2% B 5.5-6 min, 2% B 6-7 min.

### Antibody Synthesis and Expression

DNA coding for the light and heavy chain of the anti-TPBG antibody was synthesized (Geneart, Thermo Fisher), with the heavy chain constant region containing the silencing mutations L234A and L235A (LALA mutations, see FIG. 21). Signal sequences for heavy chain (SEQ ID NO: 13, MDWTWRILFLVAAATGAHS) and light chain (SEQ ID NO: 14, MLPSQLIGFLLLWVPASRG) were added. Light chain and heavy chain sequences were cloned into pcDNA3.4-TOPO (Thermo Fisher) expression plasmid. Antibodies were then transiently expressed in Expi-CHO-S cells (Thermo Fisher) by co-transfecting cells with pcDNA3.4 expression plasmids (Thermo Fisher), coding for the heavy and light chain of the respective sequences in a 1:1 ratio, using the Expi-CHO transfection system (Thermo Fisher). Cells were harvested by centrifugation at 300 g for 5 minutes at 4° C. To clear micro particles from supernatant, supernatants were centrifuged at 4000-5000 g for 30 min at 4° C. For further clarification supernatants were passed through a 0.22 μm filter. Antibodies were purified from cleared and filtered supernatants via Protein A chromatography and analyzed by HPLC-SEC, HPLC-HIC, LC-MS and SDS-PAGE.

### Preparative Size-Exclusion-Chromatography

Protein purification by size-exclusion chromatography was conducted with an AKTA Pure FPLC system (GE Healthcare, United States) equipped with a F9-C-fraction collector.

### ADC Concentration Determination

The ADC concentrations were determined in a 96-well plate with a Pierce™ Rapid Gold BCA Protein Assay Kit (Thermo Fisher Scientific, USA) and a Bradford reagent B6916 (Merck, Germany) with pre-diluted protein assay standards of bovine gamma globulin (Thermo Fisher Scientific, USA). Results of both Assays were arithmetically averaged.

### Sample Preparation of ADCs and Antibodies for MS

0.5 μl PNGase-F solution (Pomega, Germany, Recombinant, cloned from Elizabethkingia miricola 10 u/μl) and 5 μL of a 100 mM solution of DTT in water were added to 50 μl of 0.2 mg/mL antibody or ADC in PBS and the solution was incubated at 37° C. for at least 2 hours. Samples were subjected to LC/MS, injecting 2 μl for each sample.

### Analytical Size-Exclusion Chromatography

Analytical size-exclusion chromatography (A-SEC) of the ADCs was conducted on a Vanquish Flex UHPLC System with a DAD detector, Split Sampler FT (4° C.), Column Compartment H (25° C.) and binary pump F (Thermo Fisher Scientific, USA) using a MAbPac SEC-1 300 Å, 4×300 mm column (Thermo Fisher Scientific, USA) with a flow rate of 0.15 mL/min. Separation of different ADC/mAb populations have been achieved during a 30 minute isocratic gradient using a phosphate buffer at pH 7 (20 mM Na2HPO4/NaH2PO4, 300 mM NaCl, 5% v/v isopropyl alcohol as a mobile phase. 8 μg ADC/mAb where loaded onto the column for A-SEC analysis. UV chromatograms were recorded at 220 and 280 nm.

### Analytical Hydrophobic Interaction Chromatography

The measurements were conducted on a Vanquish Flex UHPLC System (2.9) with a MabPac HIC Butyl 4.6×100 mm column (Thermo Fischer Scientific, USA). Separation of different ADCs/antibodies have been achieved with the following gradient: A: 1 M (NH4)2SO4, 500 mM NaCl, 100 mM NaH2PO4 pH 7.4 B: 20 mM NaH2PO4, 20% (v/v) Isopropyl alcohol, pH 7.4. 0% B: 0-1 min, 0-95% B: 1-15 min, 95% B: 15-20 min, 95-0% B: 20-23 min, 0% B: 23-25 min, with a flow of 700 uL/min. 15 μg sample where loaded onto the column for each analysis. UV chromatograms were recorded at 220 and 280 nm.

### SDS-PAGE

Samples were prepared for SDS-PAGE by incubation in SDS sample buffer (BioRad) supplemented with 25 mM DTT (95° C., 5 minutes) and separated on a 4-20% Polyacrylamide gel (BioRad 4-20% Mini Protean TGX), followed by Coomassie staining (Thermo Fisher Scientific Imperial Protein Stain).

### ADC Synthesis: General Method for the Conjugation of P5-Based Linker-Payload Constructs to Achieve DAR8.

50 μl of the anti-TPBG antibody comprising Fc silencing mutations such as leucine (L) to alanine (A) substitution at the position 234 and 235 (LALA mutations) in a solution of 10.0 mg/ml in P5-conjugation buffer (50 mM Tris, 1 mM EDTA, 100 mM NaCl, pH 8.3 at RT) were mixed with 3.33 μl of a 10 mM TCEP solution in P5-conjugation buffer. Directly afterwards, 1.67 μl of a 40 mM solution of the P5-Exatecan construct dissolved in DMSO were added. The mixture was shaken at 350 rpm and 25° C. for 16 hours. The reaction mixtures were purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra-2 mL MWCO: 30 kDa, Merck, Germany).

### Binding to Human 5T4, Evaluated by Flow Cytometry

To determine equilibrium binding constants (K_{D}) to endogenously expressed human 5T4, 5T4-expressing MDA-MB-468 cells were incubated with antibodies in concentrations ranging from 0.002 to 200 nM and stained with an Alexa-dye-labeled anti-human IgG H+L secondary antibody (Thermo Fisher Scientific) and analyzed by flow cytometry. Mean fluorescence intensity (MFI) ratios were normalized to the non-specific binding control. The assay was performed in duplicates and data points were analyzed by a non-linear regression using a one-site specific binding model to derive K_{D} values using Prism 9 software. A K_{D} of 0.2473 nM was determined for the anti-TBPG antibody on MDA-MB-468 cells. Graph shows means of n=2±SEM (FIG. 1).

### Binding to Human 5T4, Evaluated by ELISA

Binding of increasing concentrations of the anti-TPBG antibody to a purified recombinant immobilized human 5T4 antigen was tested in an ELISA setting.

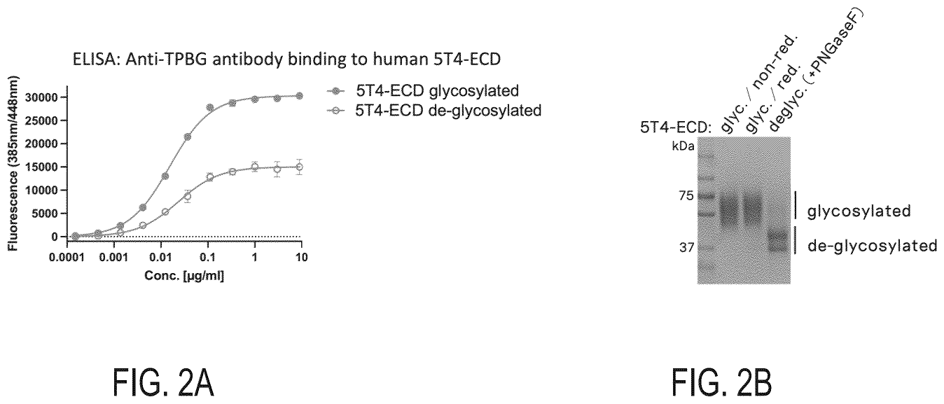

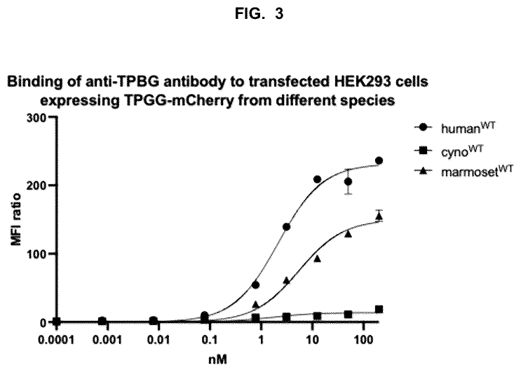

To determine binding of the anti-TPBG antibody to 5T4 antigen, 96-well-, F-Bottom black, MaxiSorp, Nunc-Immuno plates (Thermo Fisher) were coated with 1 μg/mL purified recombinant human 5T4 antigen, consisting of the extracellular domain (ACD) of 5T4 expressed as a 6×-Histidine fusion protein in Expi-HEK293 cells. For de-glycosylation, the antigen was incubated with PNGase F (Promega) over night at 37° C. De-glyosylation was confirmed by Coomassie-stained SDS-PAGE (FIG. 2B). After blocking with 2% bovine serum albumin (Carl Roth) in 1×PBS-Tween 20 (0.05%), increasing antibody concentrations (0.00015, 0.00046, 0.00137, 0.00412, 0.01235, 0.03704, 0.11111, 0.33333, 1.00000, 3.00000, 9.00000 μg/ml) were allowed to bind for 2 hr at room temperature. Bound antibodies were detected by incubation with Alkaline Phosphatase AffiniPure Rabbit Anti-Human IgG (Fcγ fragment specific) (Jackson Immunoresearch, 309-055-008) (dilution 1:5000 in blocking solution) for 1 hr at RT. 4-Methylumbelliferyl phosphate (4-MUP) (Merck) substrate was added and incubated at room temperature for 15 min, after which 25 μl/well of 3 M NaOH was added. The hydrolysis of 4-MUP to the soluble fluorescent substance methylumbelliferone is quantified by the fluorescence signal of methylumbelliferone (385 nm excitation wavelength, 448 nm emission wavelength) using a microplate reader Infinite M1000 Pro (Tecan). Apparent dissociation constants (K_{D}) were derived by non-linear regression using a one-site specific binding model using Prism 9 software. K_{D} values (nM), 0.10 for glycosylated 5T4, 0.16 nM for de-glycosylated 5T4. Graph shows means of n=2±SEM (FIG. 2A).

### Binding to White-Tufted-Ear Marmoset 5T4, Evaluated by Flow Cytometry

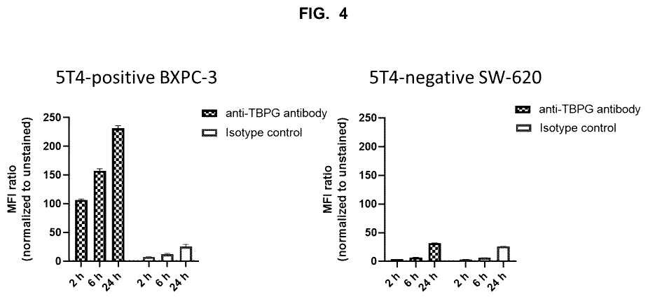

HEK293 cells were transiently transfected with expression plasmids coding for human, cyno or marmoset full-length 5T4-(GGGGS)-mCherry To determine equilibrium binding constants (K_{D}), HEK293 cells transiently expressing human, cyno or marmoset full-length 5T4-mCherry were incubated with antibodies in concentrations ranging from 0.002 to 200 nM and stained with an Alexa-dye-labeled anti-human IgG H+L secondary antibody (Thermo Fisher Scientific) and analyzed by flow cytometry. Binding of antibodies was investigated on mCherry-positive cells. Mean fluorescence intensity (MFI) ratios were normalized to the secondary antibody control. The assay was performed in duplicates and data points were analyzed by a non-linear regression using a one-site specific binding model to derive K_{D} values using Prism 9 software. K_{D} (nM), human 2.155, cyno not applicable, marmoset 5.455. Graph shows means of n=2±SD (FIG. 3).

### Internalization Evaluated Via Flow Cytometry

For pHrodo-based investigation of internalization, antibodies were labeled with pHrodo™ Deep Red Antibody Labeling Kit (Thermo Fisher Scientific) according to manufacturer's instructions. 5T4-positive (BXPC-3) and negative (SW-620) cells were incubated with 5 μg/ml of pHrodo-labeled antibodies for 1 h, 5 h and 24 h at 37° C. An increase in MFI indicates the presence of antibodies in late endosomal and lysosomal compartments. The MFI ratio was determined by dividing the MFI of pHrodo-incubated cells by the MFI of unstained cells (FIG. 4).

### In Vitro Cytotoxicity Evaluated Via Resazurin Assay

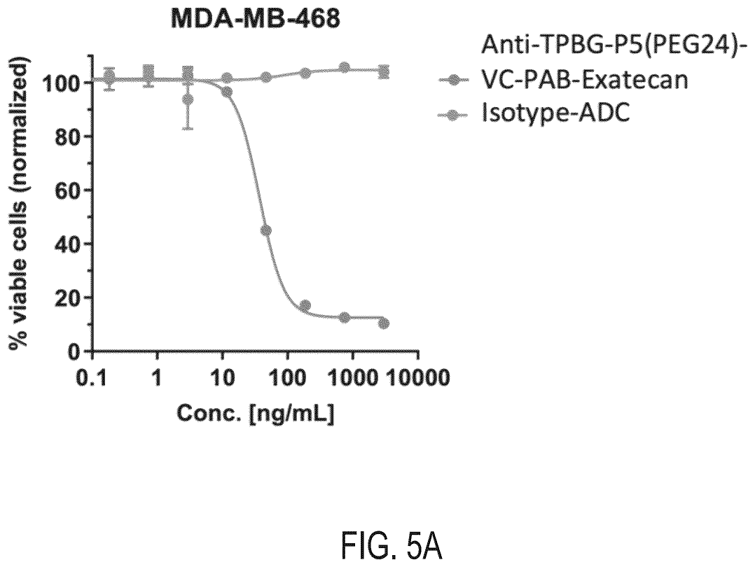

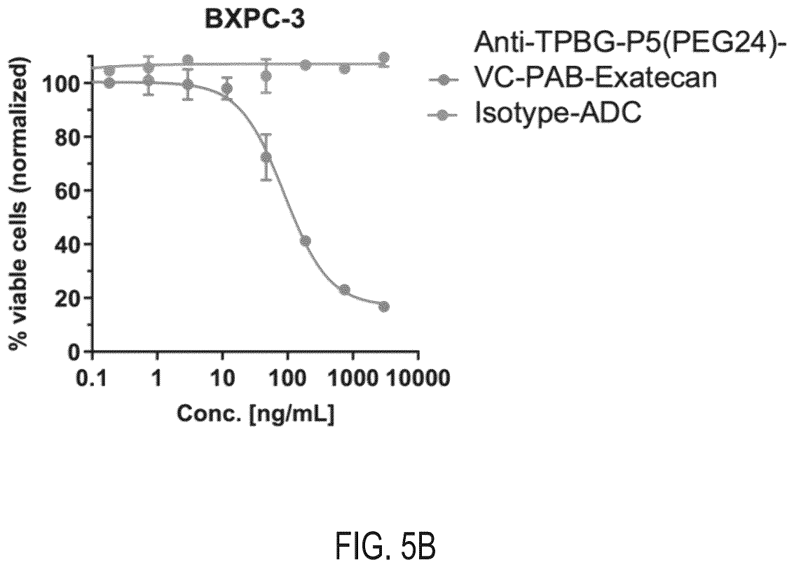

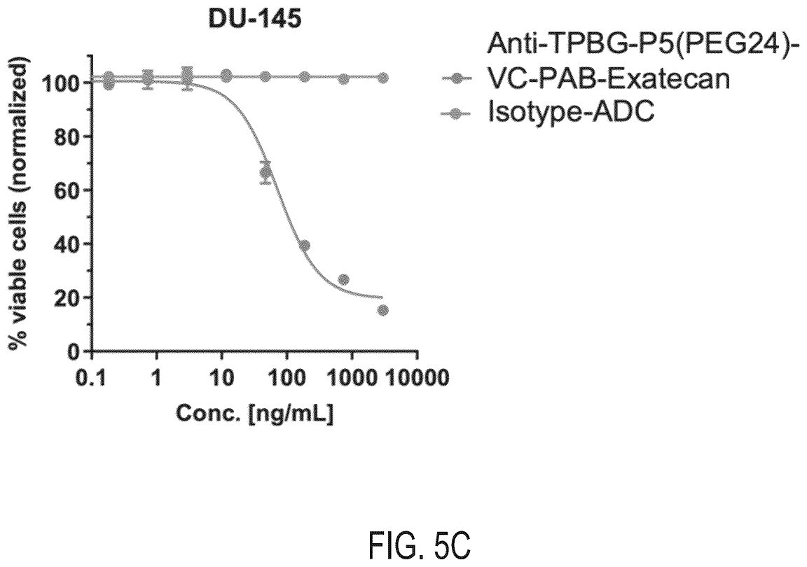

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-3 μg/ml) to generate a dose-response curve. Before viability analysis, the supernatant over the adherent cells was removed and replaced by fresh medium. Killing was analyzed afterwards, using resazurin (Sigma-Aldrich) as the cell viability dye at a final concentration of 55 μM. Fluorescence emission at 590 nM was measured on a Microplate reader Infinite M1000 Pro (Tecan). Cell viability was measured by dividing the fluorescence of ADC-treated cells by the fluorescence from control cells, treated in the same way with medium only. Data points were analyzed by a non-linear regression using a one-site specific binding model to derive IC₅₀ values using Prism 9 software. TUB-030 ADC IC₅₀ (ng/mL), 34.3 ng/mL MDA-MB-468, 89.0 ng/mL BXPC-3, 72.3 ng/mL DU-145. Minimal viability (E_{Max} in %), 10.41% MDA-MB-468, 16.8% BXPC-3, 15.3% DU-145. Graphs show means of n=2±SEM (FIGS. 5A to 5C).

### Bystander Activity of the ADC

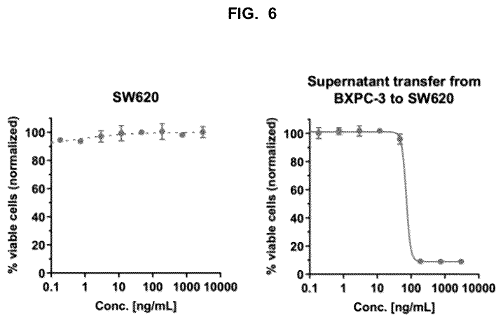

To analyze bystander activity of ADCs on target-negative cells, 20.000 5T4-positive cells (BXPC-3) were incubated with increasing concentrations of ADCs (0-3 μg/ml). After 5 days, half of the cell culture supernatant volumes was transferred to 5.000 5T4-negative cells (SW-620) and incubated for another 5 days. Killing was analyzed by a resazurin-based viability measurement as described above (FIG. 6).

### In Vitro Inhibition of Topoisomerase-1, by the Delivery of Exatecan Via the ADC

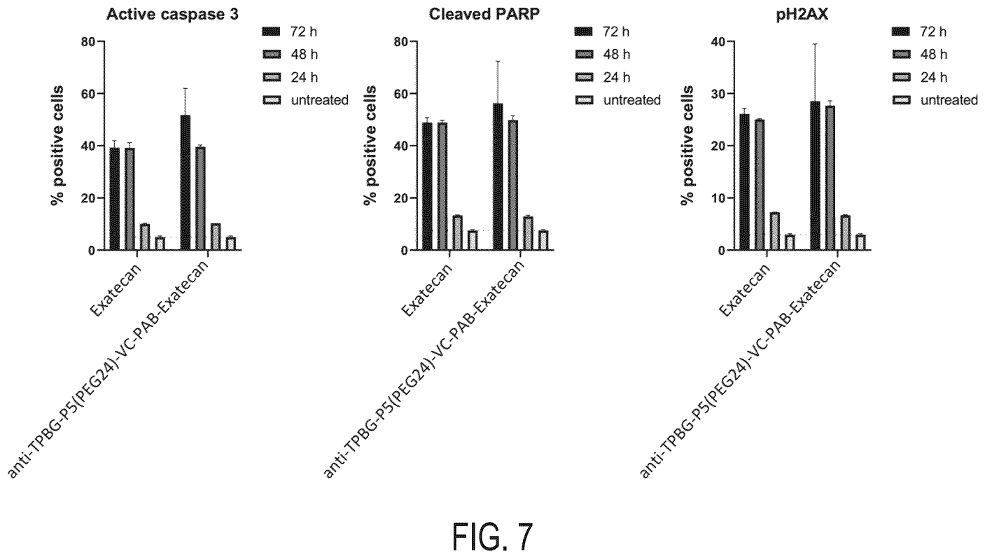

TOPOisomerase-I inhibition by delivery of Exatecan via the TUB-030 ADC induces DNA-damage markers. MDA-MB-468 cells were treated with 5 μg/mL TUB-030 or 5 nM free Exatecan for 24, 48 and 72 h. Cells were stained for the DNA-damage markers active caspase-3, cleaved PARP and phosphorylated H2A.X (Ser-139) and analyzed by flow cytometry. Graphs show means of n=2±SEM (FIG. 7).

### In Vivo Efficacy of the ADC in a Mice Xenograft Model

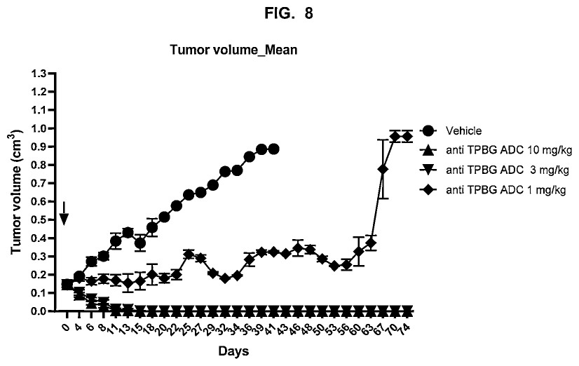

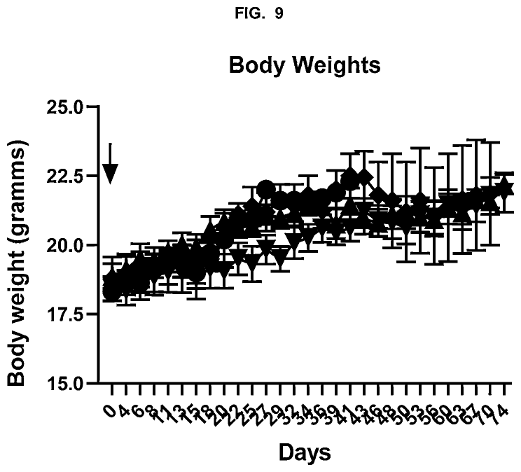

All animal experiments were conducted in accordance with German animal welfare law and approved by local authorities. In brief, 1×10⁷ MDA-MB-468 cells were subcutaneously injected to CB17-Scid mice. Treatment was initiated when tumours reached a mean tumour volume of 0.188 cm³ 28 days after implantation. 5 animals per group were treated once with either 1 mg/kg, 3 mg/kg or 10 mg/kg of anti-TPBG-P5(PEG24)-VC-PAB-Exatecan DAR 8 or vehicle, as intravenous injection after randomisation into treatment and control groups. Tumour volumes, body weights and general health conditions were recorded throughout the whole study (FIGS. 8-9).

### In Vivo PK Evaluation of the ADC

In vivo PK-experiments have been performed with anti-TPBG-P5(PEG24)-VC-PAB-Exatecan. Female Sprague Dawley rats have been treated with 10 mg/kg of the unconjugated TPBG antibody or the ADC. Blood sampling has been performed after different time points and the ADC amount was quantified in a total antibody and an intact ADC ELISA-assay. To evaluate the PK of the ADCs in vivo, the total antibody concentration was measured at different time points in serum of ADC-treated SD rats. Total humanized anti-5T4 antibody was analyzed in rat serum over the range 2000-15.6 ng/ml. Nunc 96-well plate with (100 μl/well) were coated with 5T4 diluted in PBS (required concentration: 0.25 μg/ml) and sealed with PCR Foil. Plates were incubated in a fridge to maintain a temperature between 2-8° C. overnight. The coated plates were washed 3× with 300 μl PBST. 200 μl/well of blocking solution (2% Albumin in PBST) was added, the plate was sealed and an incubated at room temperature for 1 hour. The coated plates were washed 3× with 300 μl PBST. 100 μl/well of prepared standards (2000-15.6 ng/ml of the respective ADCs, QCs and test samples were added, the plates were sealed and incubated at room temperature for 1 hour. The plates were washed 3× with 300 μl PBST. 100 μl/well Anti-Human IgG (γ-chain specific)-Peroxidase antibody (dilution 1:60000 in PBS) was added and incubated for 1 h at rt. The plates were washed 3× with 300 μl PBST. 50 μl/well TMB was added, the plates were sealed and incubated at room temperature for 15 min. 50 μl/well of 1 M Sulfuric Acid was added. Using a Tecan Plate Reader, the absorbance at a wavelength of 450 nm was measured.

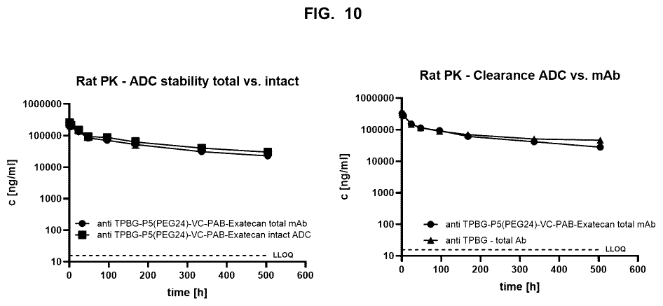

To evaluate the stability of the ADCs in vivo, the intact ADC concentration was measured at different time points in serum of ADC-treated SD rats. Intact ADC was analyzed in rat serum over the range 2000-15.6 ng/ml. Nunc 96-well plate with (100 μl/well) were coated with rabbit anti-Exatecan mAb diluted in PBS (required concentration: 1 μg/ml) and sealed with PCR Foil. Plates were incubated in a fridge to maintain a temperature between 2-8° C. overnight. The coated plates were washed 3× with 300 μl PBST. 200 μl/well of blocking solution (2% Albumin in PBST) was added, the plate was sealed and an incubated at room temperature for 1 hour. The coated plates were washed 3× with 300 μl PBST. 100 μl/well of prepared standards (2000-15.6 ng/ml of the respective ADCs, QCs and test samples were added, the plates were sealed and incubated at room temperature for 1 hour. The plates were washed 3× with 300 μl PBST. 100 μl/well Goat Anti-Human IgG (H+L) Preabsorbed (dilution 1:25000 in PBS was added and incubated for 1 h at rt. The plates were washed 3× with 300 μl PBST. 100 μl/well TMB was added, the plates were sealed and incubated at room temperature for 10 min. 100 μl/well of 1 M Sulfuric Acid was added. Using a Tecan Plate Reader, the absorbance at a wavelength of 450 nm was measured (FIG. 10).

### Linker-Payload Synthesis

### General Method 2 for the Synthesis of PEGylated P5 Building Blocks Via the Staudinger Phosphonite Reaction

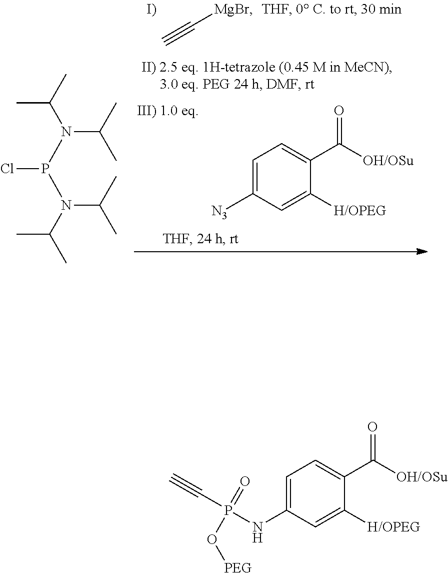

A 25-ml Schlenk flask was charged with 267 mg bis(diisopropylamino)chlorophosphine (1.00 mmol, 1.00 eq.) under an argon atmosphere, cooled to 0° C. and 2.20 mL ethynylmagnesium bromide solution (0.5 M in THF, 1.10 mmol, 1.10 eq.) was added drop wise. The yellowish solution was allowed to warm to room temperature and stirred for further 30 minutes. 3.00 mmol (3.0 eq.) of the desired PEG-alcohol, dissolved in 5.56 mL 1H tetrazole solution (0.45 M in MeCN, 2.50 mmol, 2.50 eq.) were added and the white suspension was stirred overnight at room temperature. The formation of the desired phosphonite was monitored by ³¹P-NMR. 1.0 mmol (1.0 eq.) of the desired azide dissolved in 2 mL of DMF, THF or MeCN was added and the suspension further stirred for 24 h at room temperature. The crude reaction mixture was purified using preparative HPLC.

### P5(PEG12)-OSu

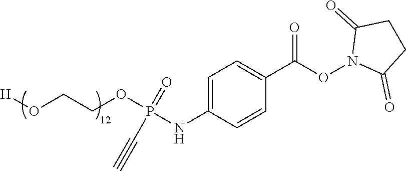

The title compound was synthesized in accordance to general Method 2 from 19.5 mg bis(diisopropylamino)chlorophosphine (73 μmol, 1.00 eq.), 146 μL ethynylmagnesium bromide solution (0.5 M in THF, 73 μmol, 1.00 eq.), 100 mg of dodecaethylene glycol (183 μmol, 2.50 eq), 400 μL 1H-tetrazole solution (0.45 M in MeCN, 183 μmol) and 19 mg 4-azidobenzoic-acid-N-hydroxysuccinimide ester (73 μmol, 1.00 eq.). The product was obtained as colourless oil after preparative HPLC (Method D) and lyophilization. (42.5 mg, 50 μmol, 68%). ¹H NMR (300 MHz, Acetonitrile-d3) δ 8.06 (d, J=8.7 Hz, 2H), 7.32 (d, J=8.8 Hz, 2H), 4.40-4.14 (m, 2H), 3.79-3.69 (m, 2H), 3.66-3.47 (m, 40H), 3.21 (d, J=13.1 Hz, 1H), 2.86 (s, 4H), 1.30 (m, 2H), 1.13-0.79 (m, 2H). ¹³C NMR (151 MHz, CDCl₃) δ 169.77, 169.46, 161.66, 161.47, 152.75, 146.09, 132.90, 132.24, 117.82, 113.97, 113.29, 89.25, 88.92, 77.27, 77.06, 76.85, 74.69, 72.57, 71.19, 70.62, 70.54, 70.51, 70.47, 70.44, 70.36, 70.27, 70.20, 69.74, 69.70, 68.14, 65.77, 65.73, 61.63, 61.60, 40.72, 30.34, 25.68. ³¹P NMR (122 MHz, Acetonitrile-d₃) 5-10.87. HRMS C₃₇H₆₀N₂O₁₉P⁺ calc.: 851.3573 [M+H]⁺, 851.3571.

### P5(PEG12)-COOH

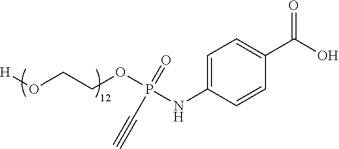

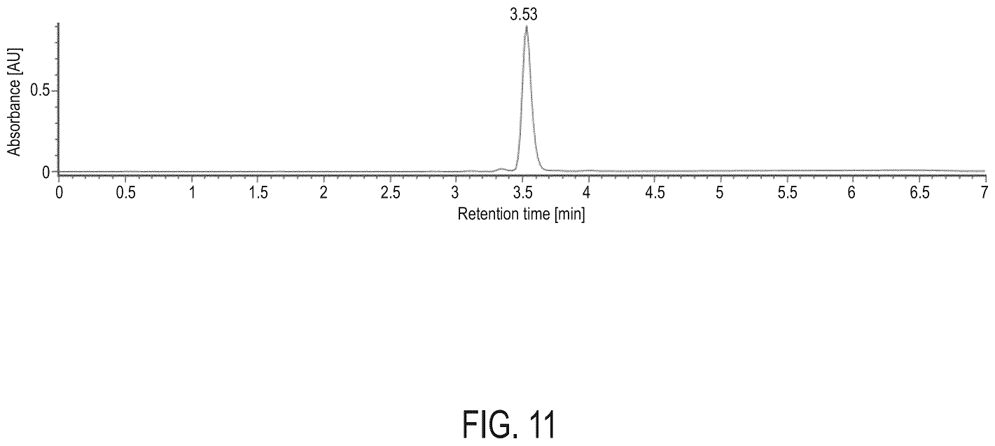

The title compound was synthesized in accordance to general Method 2 from 40 mg bis(diisopropylamino)chlorophosphine (150 μmol, 1.00 eq.), 360 μL ethynylmagnesium bromide solution (0.5 M in THF, 180 μmol, 1.2 eq.), 245 mg of PEG12 (450 μmol, 3.0 eq), 0.83 mL 1H-tetrazole solution (0.45 M in MeCN, 450 μmol, 2.5 eq.) and 39 mg 4-azidobenzoic-acid (150 μmol, 1.00 eq.). The product was obtained as colourless oil after preparative HPLC (Method D) and lyophilization. (25 mg, 34 μmol, 23%). HR-MS for C₃₃H₅₇NO₁₆P⁺ [M+H]⁺ calcd.: 754.3410, found 754.3398 (FIG. 11).

### P5(PEG24)-OSu

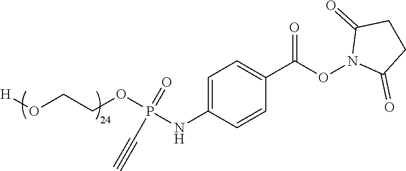

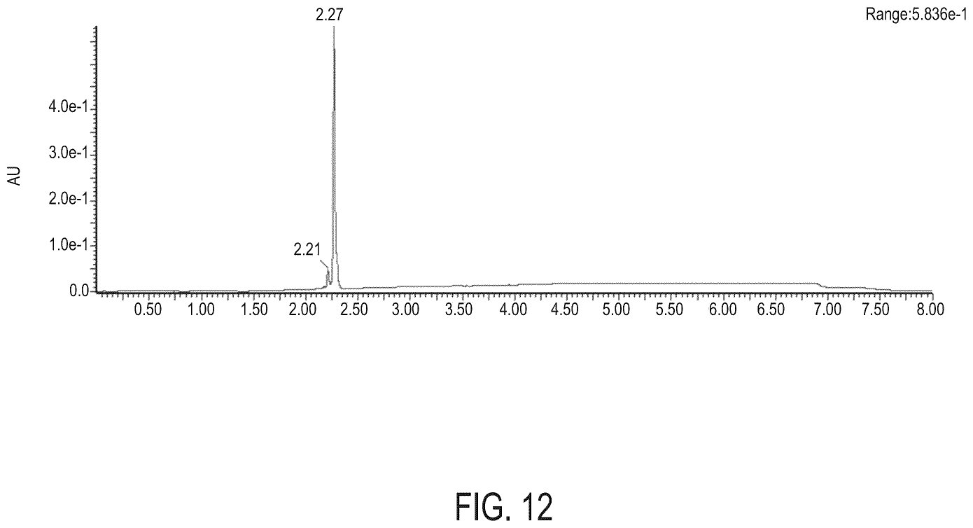

The title compound was synthesized in accordance to general Method 2 from 41 mg bis(diisopropylamino)chlorophosphine (159 μmol, 1.00 eq.), 370 μL ethynylmagnesium bromide solution (0.5 M in THF, 185 μmol, 1.2 eq.), 450 mg of PEG24 (388 μmol, 2.50 eq), 1.02 mL 1H-tetrazole solution (0.45 M in MeCN, 466 μmol, 3.0 eq.) and 40 mg 4-azidobenzoic-acid-N-hydroxysuccinimide ester (155 μmol, 1.00 eq.). The product was obtained as colourless oil after preparative HPLC (Method D) and lyophilization. (79 mg, 57 μmol, 37%). MS for C₆₁H₁₀₉N₂O₃₀P²⁺ [M+2H]²⁺ calcd.: 690.3396, found 690.81. (FIG. 12).

### NH2-VC-PAB-Exatecan TFA Salt

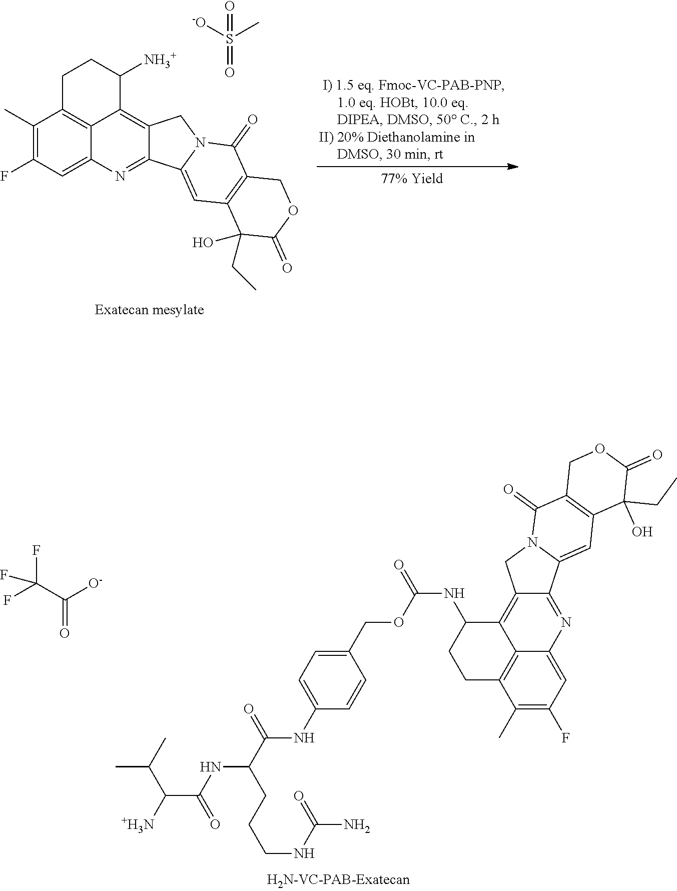

A screw-cap-vial was charged with 34.3 mg of Exatecan Mesylate (0.0645 mmol, 1.0 eq.) and suspended in 645 μL of dry DMSO. 241 μL of a solution of a 0.4 mol/L solution of Fmoc-VC-PAB-PNP in dry DMSO (0.0967 mmol, 1.5 eq.), 64.5 μL of a 1 mol/L solution of HOBt hydrate in dry DMSO (0.0645 mmol, 1.0 eq.) and 113 μL of DIPEA were added (0.645 mmol, 10.0 eq.). The yellow solution was stirred for 2 h at 50° C. Afterwards, 425 μL of a solution of 50% Diethanolamine in dry DMSO (w/w) was added and the reaction mixture was allowed to stir at room temperature for another 30 minutes. 1.5 ml MeCN and 2.5 mL H₂O added and the yellow solution was directly purified by preparative HPLC, using Method D. After lyophilization, 47.3 mg (76.7%, 0.0495 mmol) of a yellowish solid were obtained as TFA-salt.

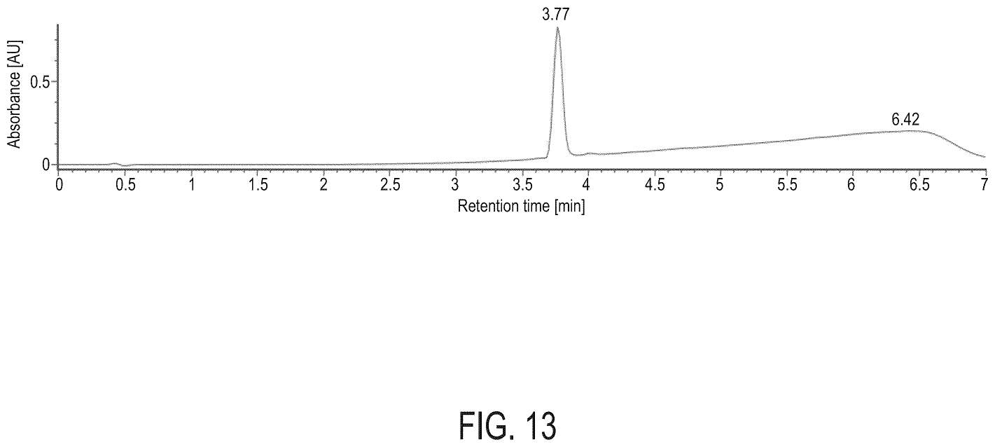

HR-MS for C₄₃H₅₀FN₈O₉⁺ [M+H]⁺ calcd.: 841.3680, found 841.3696 (FIG. 13).

### NH2-VA-PAB-Exatecan TFA Salt

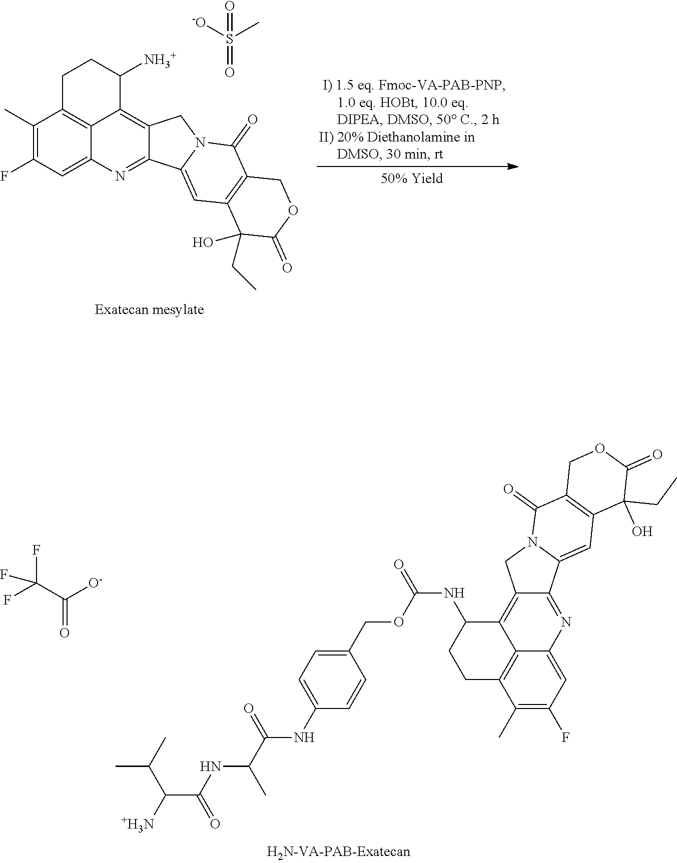

A screw-cap-vial was charged with 1.23 mg of Exatecan Mesylate (0.00232 mmol, 1.0 eq.) and suspended in 23 μL of dry DMSO. 8.7 μL of a solution of a 0.4 mol/L solution of Fmoc-VA-PAB-PNP in dry DMSO (0.00348 mmol, 1.5 eq.), 2.3 μL of a 1 mol/L solution of HOBt hydrate in dry DMSO (0.00232 mmol, 1.0 eq.) and 4 μL of DIPEA were added (0.0232 mmol, 10.0 eq.). The yellow solution was stirred over night at room temperature. Afterwards, 15 μL of a solution of 50% Diethanolamine in dry DMSO (w/w) was added and the reaction mixture was allowed to stir at room temperature for another 30 minutes. 1.5 ml MeCN and 2.5 mL H₂O added and the yellow solution was directly purified by preparative HPLC, using Method C. After lyophilization, 1.01 mg (50.0%, 0.00116 mmol) of a yellowish solid were obtained as TFA-salt.

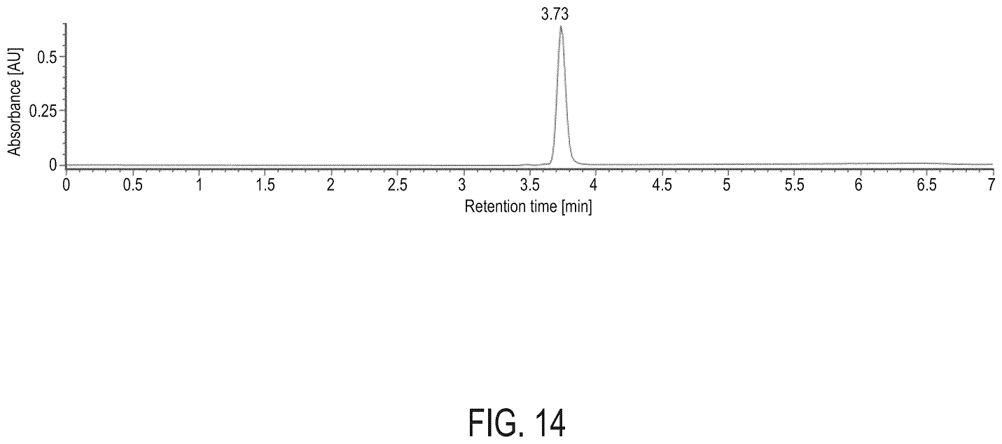

HR-MS for C₄₀H₄₄FN₆O₈⁺ [M+H]⁺ calcd.: 755.3200, found 755.3201. (FIG. 14).

### P5(PEG2)-VC-PAB-Exatecan

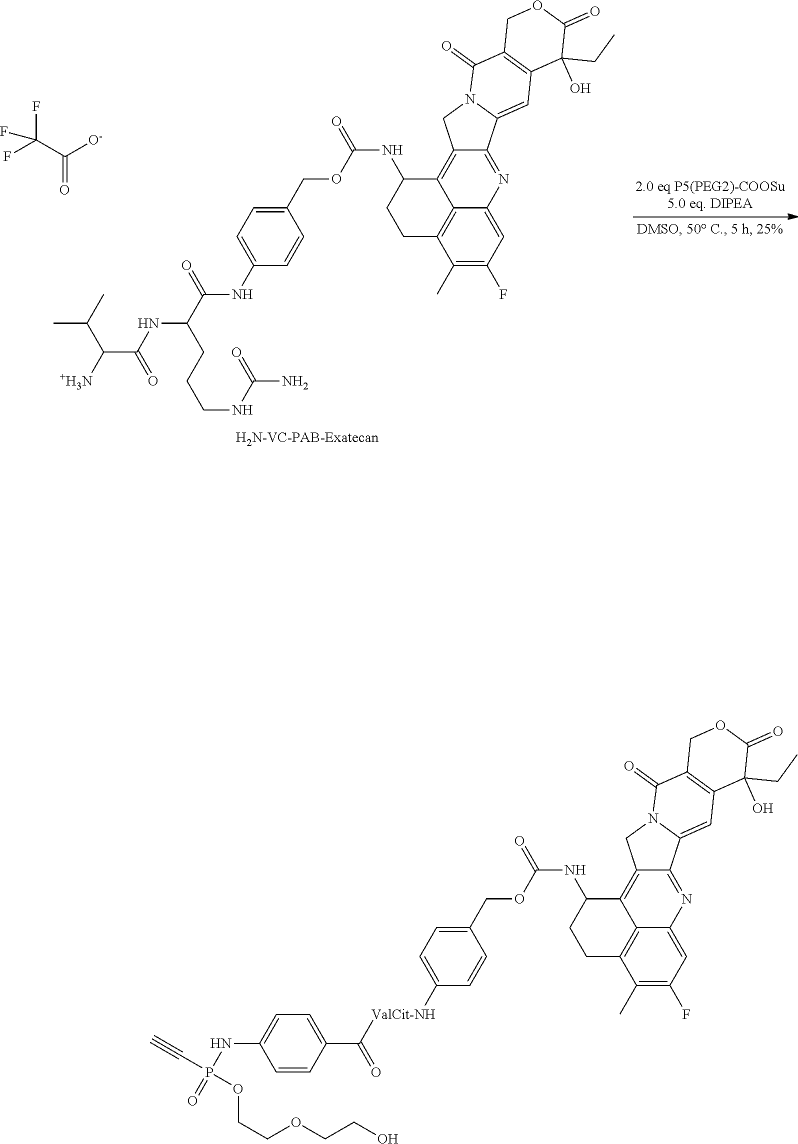

A screw-cap-vial was charged with 23.4 μL of a 200 mM solution of NH2-VC-PAB-Exatecan TFA salt in dry DMSO (0.00468 mmol, 1.0 eq.), 46.8 μL of a 200 mM solution of 2-(2-Hydroxyethoxy)ethyl-N-(4-benzoic-acid-N-hydroxysuccinimideester)-P-ethynyl phosphonamidate (P5(PEG2)-COOSu, 0.00936 mmol, 2.0 eq.) and 4.08 μL DIPEA (0.0234 mmol, 5.0 eq.). The solution was shaken for 5 hours at 50° C., cooled to room temperature, 1.5 ml MeCN and 2.5 mL H₂O were added and the solution was directly purified by preparative HPLC, using Method C. After lyophilization, 1.33 mg (25.0%, 0.00117 mmol) of a yellowish solid were obtained.

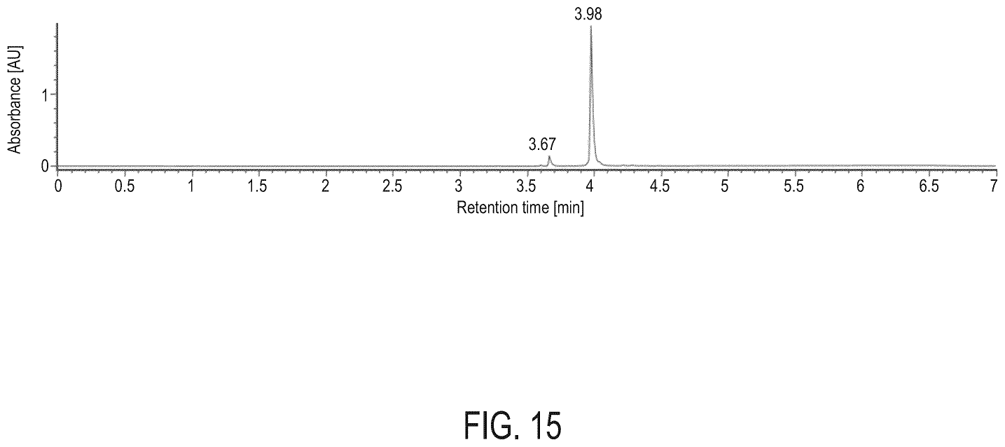

HR-MS for C₅₆H₆₄FN₉O₁₄P⁺ [M+H]⁺ calcd.: 1136.4289, found 1136.4306. (FIG. 15).

### P5(PEG12)-VC-PAB-Exatecan

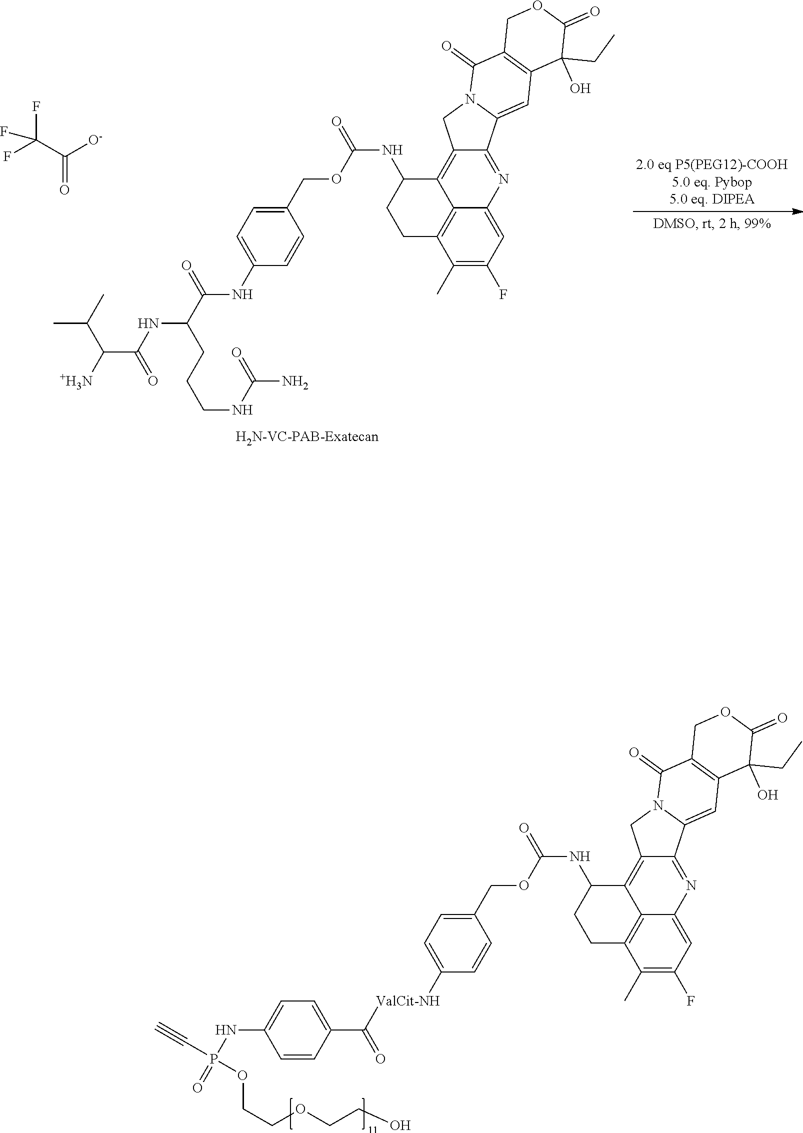

A screw-cap-vial was charged with 51 μL of a 200 mM solution of NH2-VC-PAB-Exatecan TFA salt in dry DMSO (0.0102 mmol, 1.0 eq.), 102 μL of a 200 mM solution of PEG12-N-(4-benzoic-acid)-P-ethynyl phosphonamidate (P5(PEG12)-COOH, 0.0204 mmol, 2.0 eq.) in dry DMSO, 102 μL of a 250 mM solution of Pybop (0.0255 mmol, 2.5 eq.) in dry DMSO and 8.89 μL DIPEA (0.051 mmol, 5.0 eq.). The solution was shaken for 2 hours at room temperature, 1.5 ml MeCN and 2.5 mL H₂O were added and the solution was directly purified by preparative HPLC, using Method D. After lyophilization, 15.91 mg (99.0%, 0.0101 mmol) of a yellowish solid were obtained.

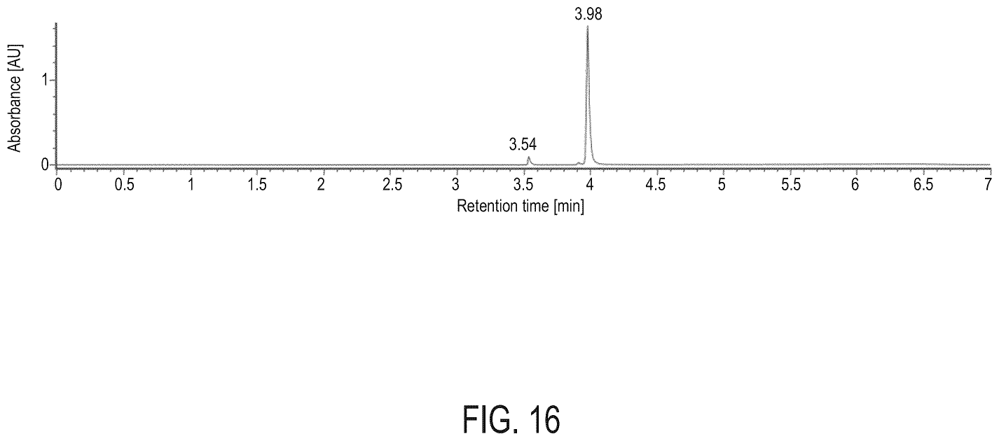

HR-MS for C₇₆H₁₀₅FN₉O₂₄P²⁺ [M+H]²⁺ calcd.: 788.8492, found 788.8485. (FIG. 16).

### P5(PEG24)-VC-PAB-Exatecan

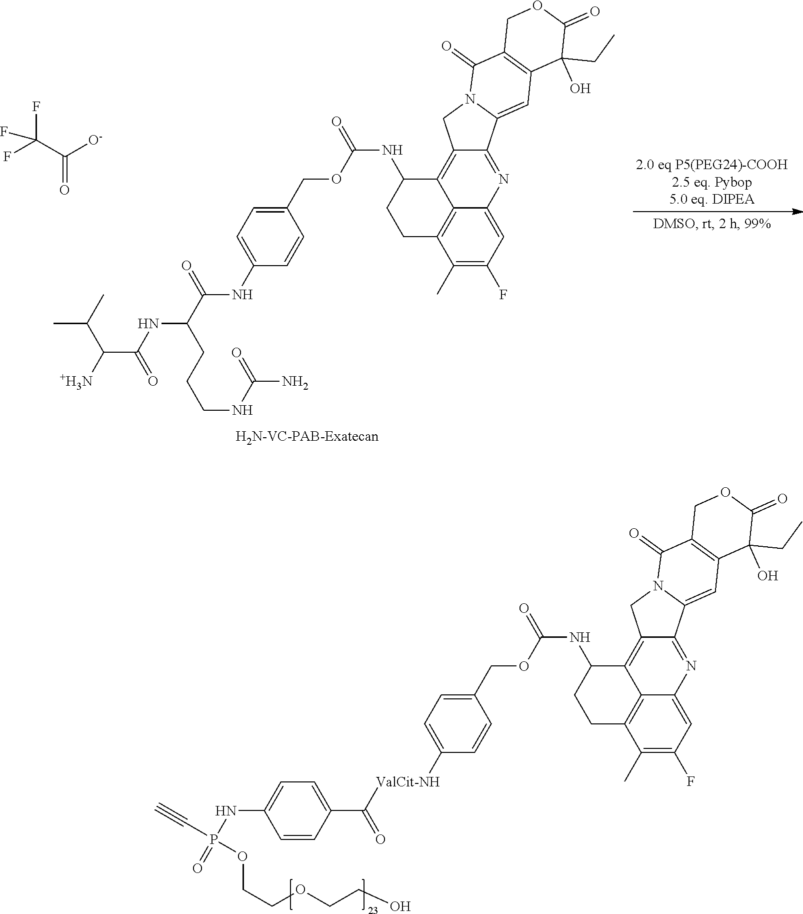

A screw-cap-vial was charged with 102 μL of a 200 mM solution of NH2-VC-PAB-Exatecan TFA salt in dry DMSO (0.0204 mmol, 1.0 eq.), 204 μL of a 200 mM solution of (P5(PEG24)-OSu, 0.0408 mmol, 2.0 eq.) in dry DMSO, and 17.78 μL DIPEA (0.102 mmol, 5.0 eq.). The solution was shaken over night at room temperature, 1.5 ml MeCN and 2.5 mL H₂O were added and the solution was directly purified by preparative HPLC, using Method D. After lyophilization, 25.76 mg (60.0%, 0.01224 mmol) of a yellowish solid were obtained.

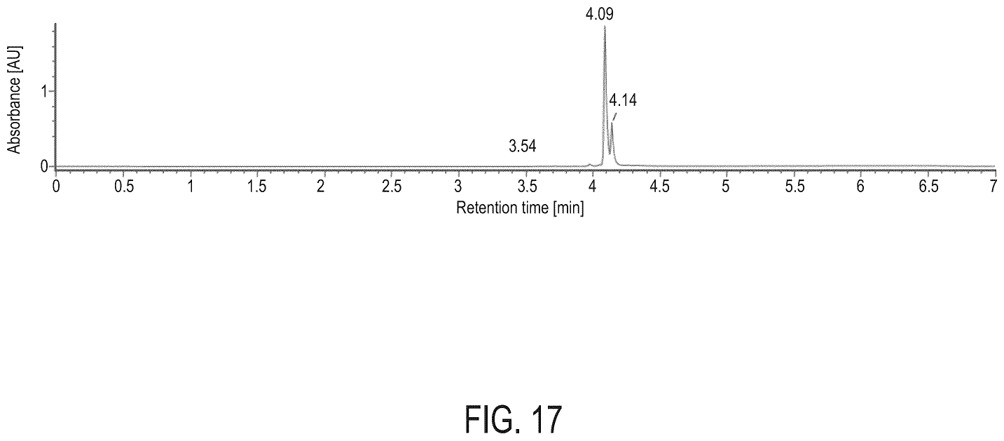

HR-MS for C₁₀₀H₁₅₃FN₉O₃₆P²⁺ [M+H]²⁺ calcd.: 1053.5081, found 1053.50833. (FIG. 17).

### P5(PEG12)-VA-PAB-Exatecan

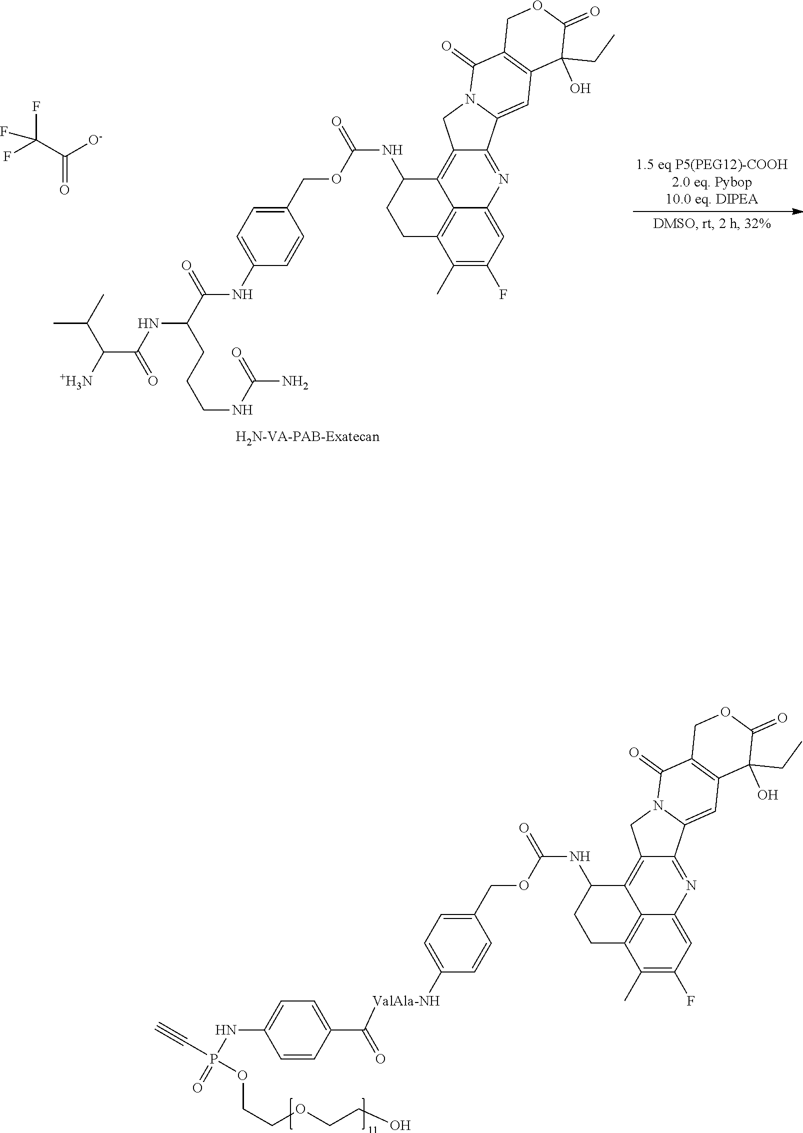

A screw-cap-vial was charged with 11.6 μL of a 100 mM solution of NH2-VA-PAB-Exatecan TFA salt in dry DMSO (0.00116 mmol, 1.0 eq.), 8.7 μL of a 200 mM solution of PEG12-N-(4-benzoic-acid)-P-ethynyl phosphonamidate (P5(PEG12)-COOH, 0.00174 mmol, 1.5 eq.) in dry DMSO, 11.6 μL of a 200 mM solution of Pybop (0.00232 mmol, 2.0 eq.) in dry DMSO and 2.02 μL DIPEA (0.0116 mmol, 10.0 eq.). The solution was shaken for 2 hours at room temperature, 1.5 ml MeCN and 2.5 mL H₂O were added and the solution was directly purified by preparative HPLC, using Method C. After lyophilization, 0.56 mg (32.2%, 0.000375 mmol) of a yellowish solid were obtained.

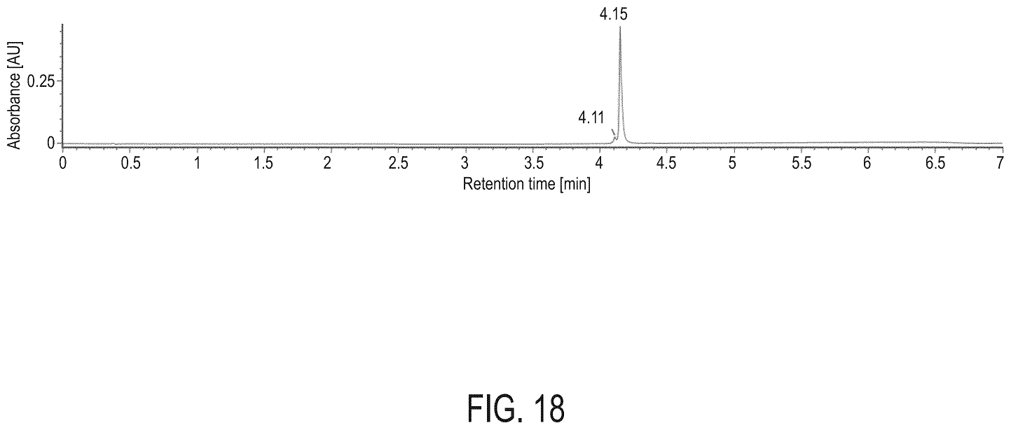

HR-MS for C₇₃H₉₉FN₇O₂₃P²⁺ [M+H]²⁺ calcd.: 745.8252, found 745.8255. (FIG. 18).

### P5(PEG12)-Exatecan

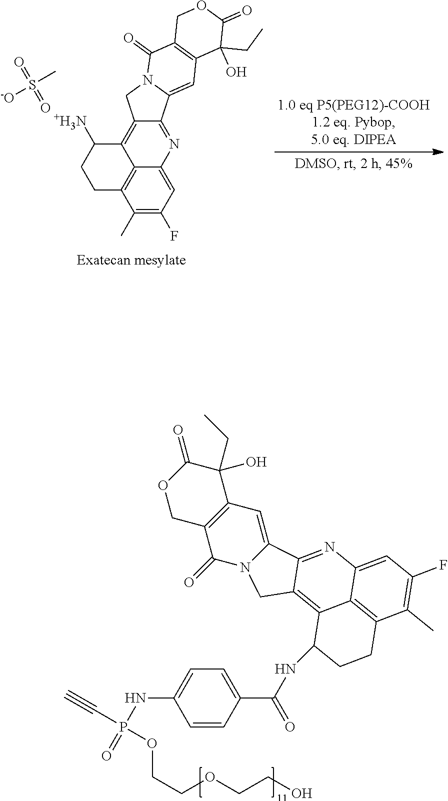

A screw-cap-vial was charged with 50 μL of a 100 mM suspension of Exatecan Mesylate in dry DMSO (0.005 mmol, 1.0 eq.), 20 μL of a 250 mM solution of PEG12-N-(4-benzoic-acid)-P-ethynyl phosphonamidate (P5(PEG12)-COOH, 0.005 mmol, 1.0 eq.) in dry DMSO, 20 μL of a 300 mM solution of Pybop (0.006 mmol, 1.2 eq.) in dry DMSO and 4.33 μL DIPEA (0.025 mmol, 5.0 eq.). The solution was shaken for 2 hours at room temperature, 1.5 ml MeCN and 2.5 mL H₂O were added and the solution was directly purified by preparative HPLC, using Method C. After lyophilization, 2.63 mg (45.0%, 0.0023 mmol) of a yellowish solid were obtained.

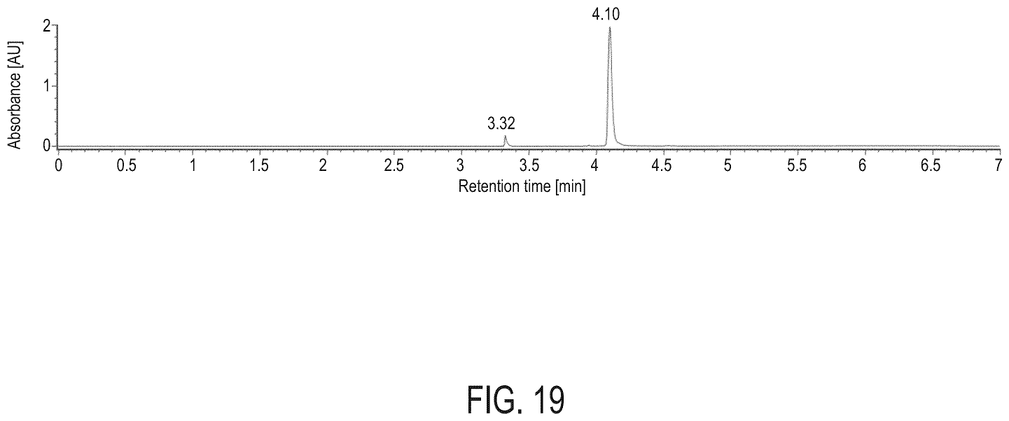

HR-MS for C₅₇H₇₇FN₄O₁₉P⁺ [M+H]⁺ calcd.: 1171.4899, found 1171.4852. (FIG. 19).

### Results

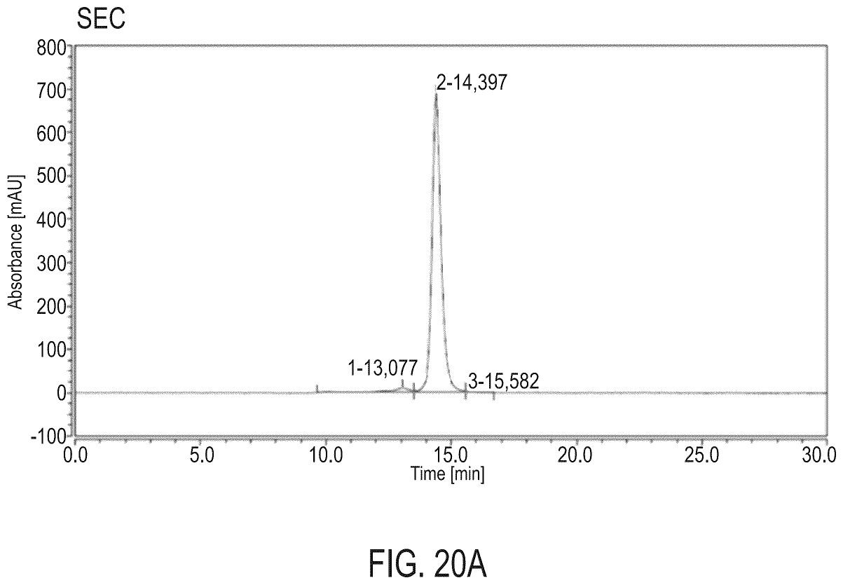

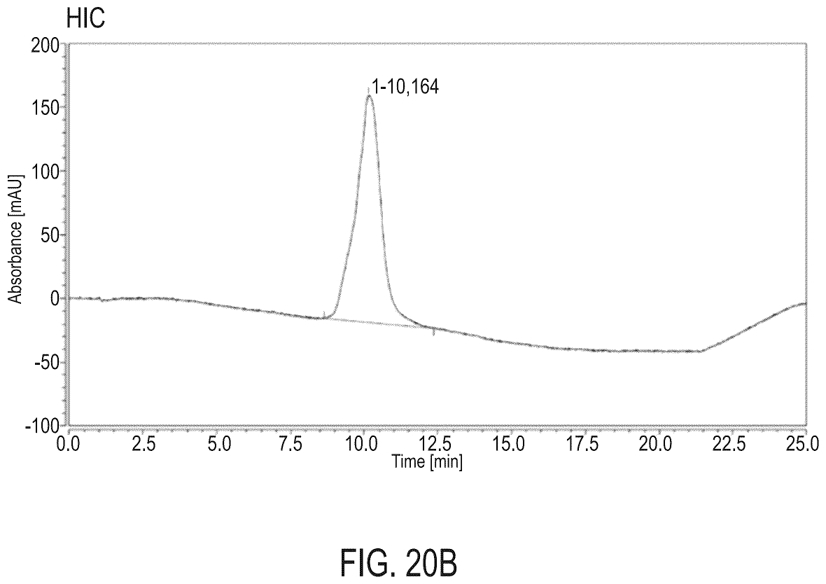

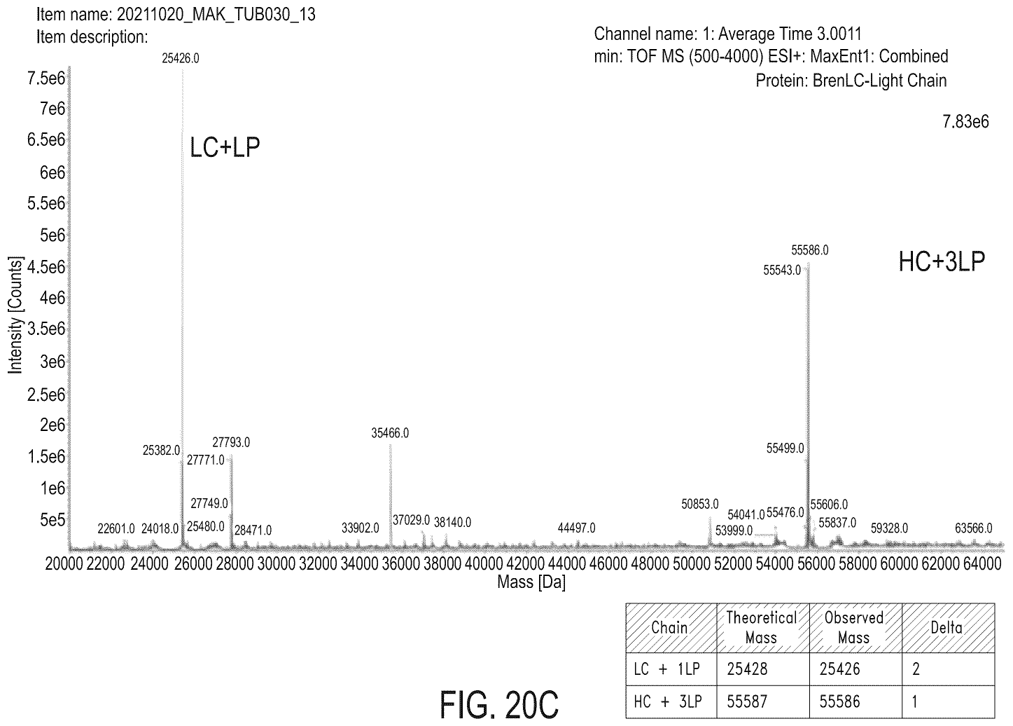

Analytics of the Synthesized ADC from P5(PEG24)-VC-PAB-Exatecan (FIGS. 20A to 20C).

Analytical characterization of the ADC, synthesized and purified via CEX from the anti-TPBG antibody, conjugated to P5(PEG24)-VC-PAB-Exatecan. FIG. 20A) Analytical SEC, FIG. 20B) Analytical HIC, FIG. 20C) Analytical MS spectrum after conjugation. The Data clearly shows that the ADC is fully conjugated to DAR8 with only low amounts of aggregates being present after purification (FIGS. 20A to 20C).

### Example 2: Ex Vivo Serum Stability, In Vivo Efficacy in Patient Derived Xenograft Models (PDX) and In Vivo Toxicity of an Exemplary of the ADC of the Present Invention

### Ex Vivo Serum Stability:

Serum samples of the respective species were spiked with anti-TPBG-P5(PEG24)-VC-PAB-Exatecan at a concentration of 0.2 mg/ml in at least 80% serum. Samples were sterile filtered with UFC30GVOS centrifugal filter units (Merck, Germany) and incubated at 37° C. for 1, 2, 3, 5 and 7 days. Samples for day 0 were directly processed further.

Recombinant 5T4-antigen was coupled to Thermo NHS Magnetic beads according to the manufacturer's instructions. The bead storage solution was removed from 40 μl of the 5T4-coupled bead suspension. The beads were incubated with 100 μl of the serum-ADC mix, premixed with 200 μl PBS, for 2 h at room temperature. Afterwards, the supernatant was removed and the resin washed 2 times with 1 mL PBS-T. Following by incubation for 15 minutes with 10 μl 100 mM Glycin buffer pH 2.5 at room temperature. This solution was rebuffered to PBS by using 75 μL Zeba™ Spin Desalting Columns with 7K MWCO (Thermo Fisher Scientific, USA). The samples were processed further for MS-measurements, as described above. The Drug-to-Antibody ratio (DAR) was calculated from the MS intensities of the light chain adducts conjugated to 0 or 1 and heavy chain adducts conjugated to 0-3 molecules of P5(PEG24)-VC-PAB-Exatecan.

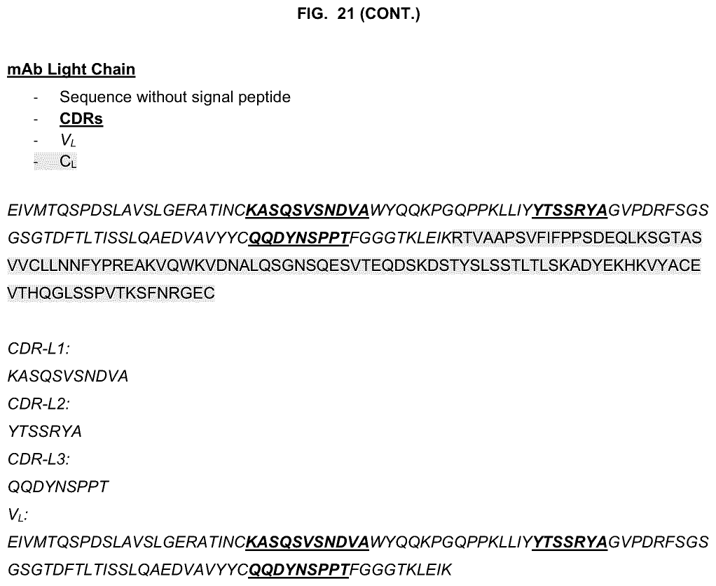

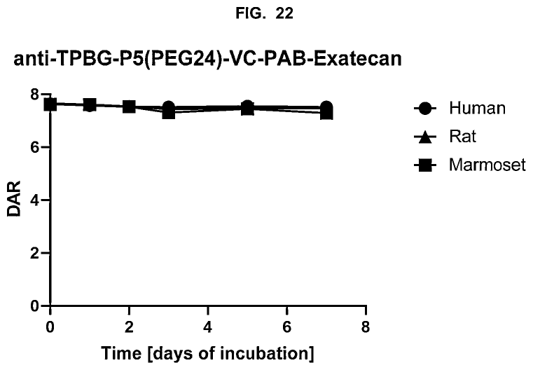

The results clearly show that the linker between the anti-TPBG-antibody and the Exatecan drug molecules is highly stable in sera of different species, without a significant amount of payload loss after several days of incubation of the ADC (No change in the Drug-to-Antibody Ratio (DAR) (FIG. 22).

### In Vivo Efficacy of the ADC in Patient Derived Xenograft Models (PDX):

All animal experiments were conducted in accordance with German animal welfare law and approved by local authorities. In brief, a 3×3 mm sample of a patient derived tumor sample was subcutaneously implanted into flanks of female immunodeficient NMRI nu/nu mice. Treatment was initiated when tumours reached a mean tumour volume of 0.1-0.15 cm³. Animals per group were treated once at day 0 with 10 mg/kg of anti-TPBG-P5(PEG24)-VC-PAB-Exatecan DAR8, Isotype-P5(PEG24)-VC-PAB-Exatecan DAR8 or vehicle, as intravenous injection. Tumour volumes, body weights and general health conditions were recorded throughout the whole study.

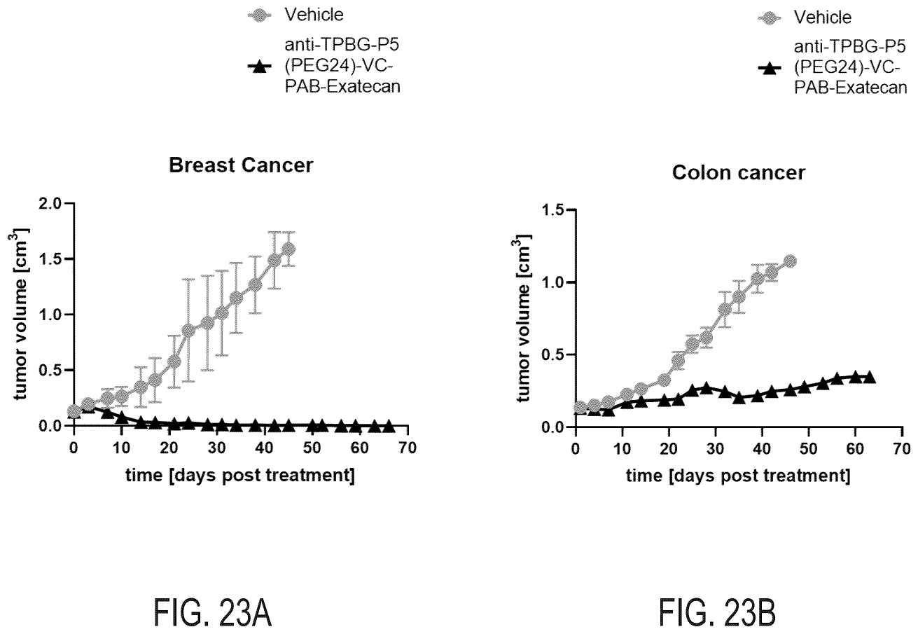

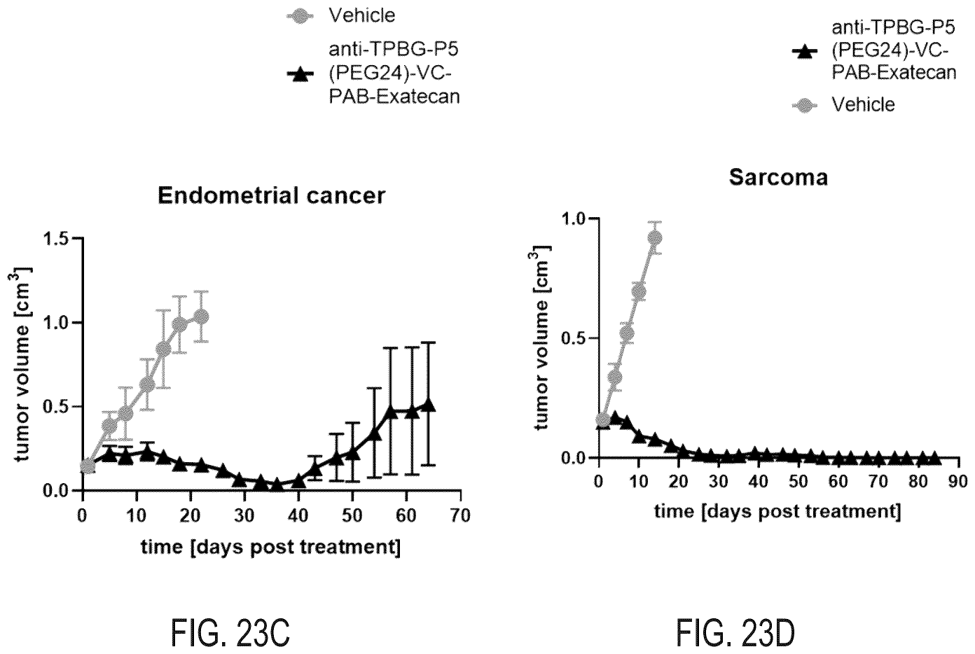

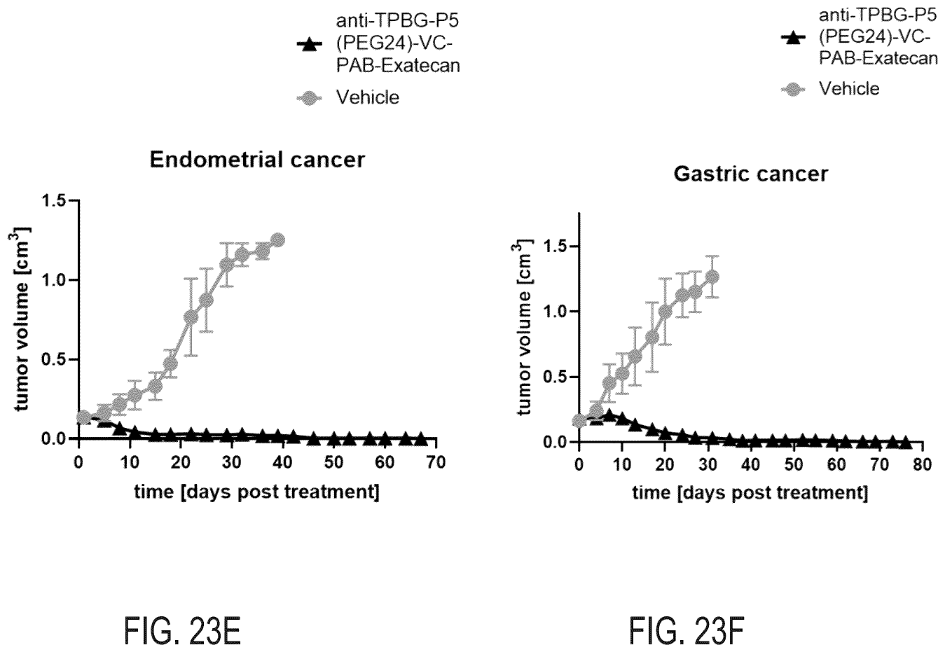

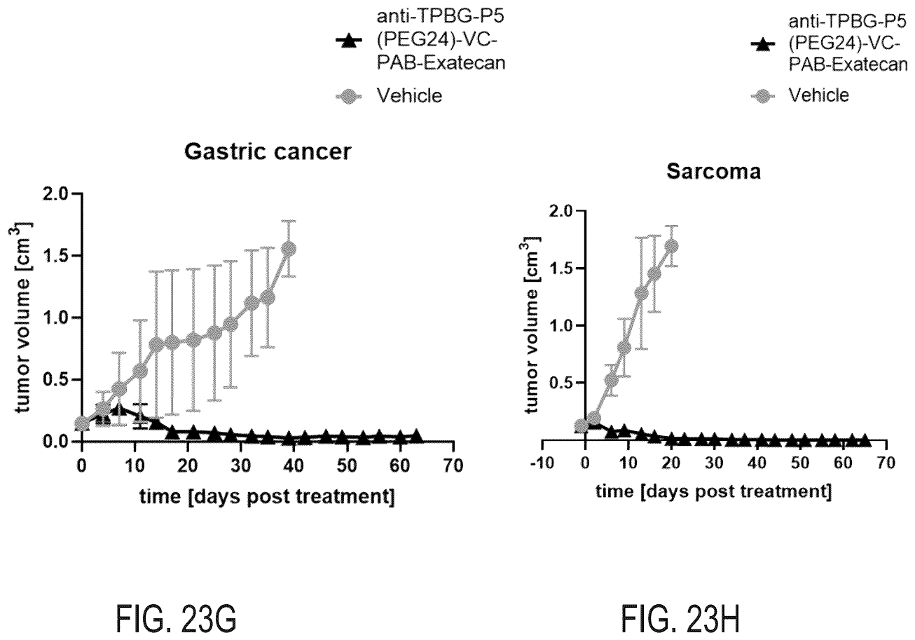

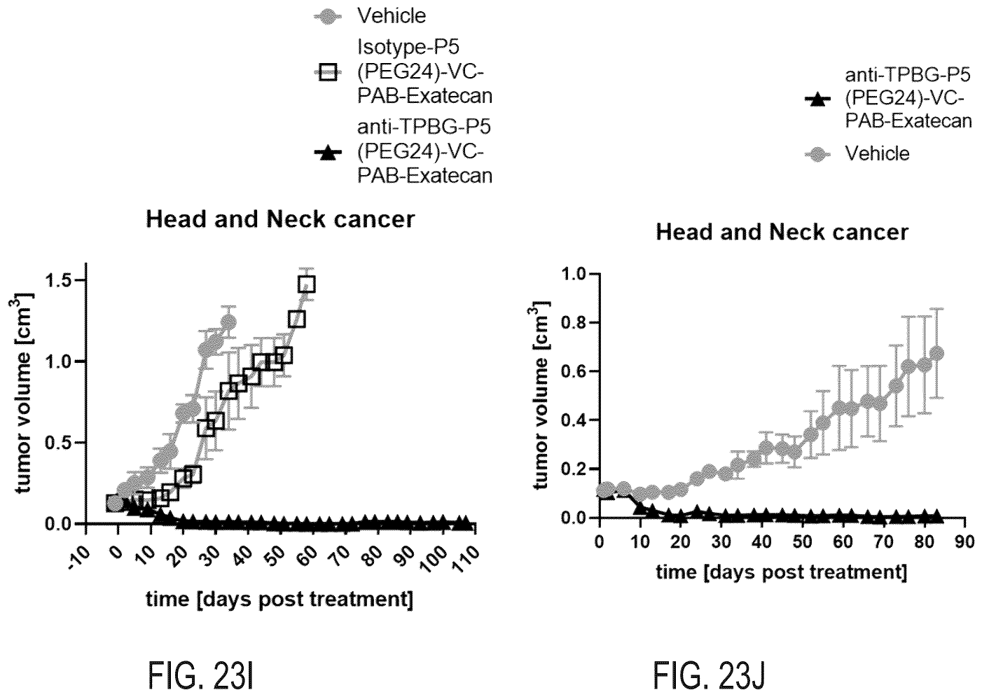

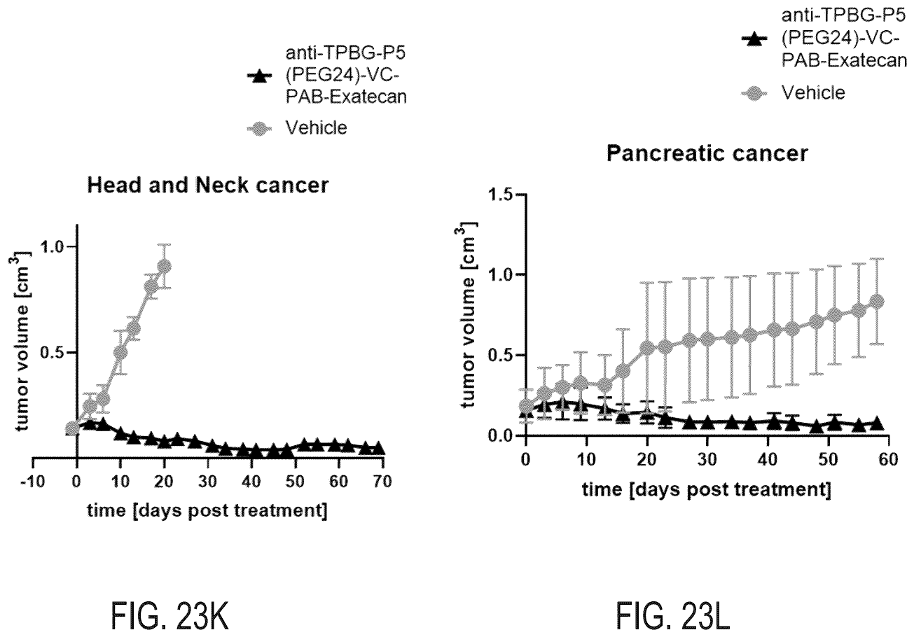

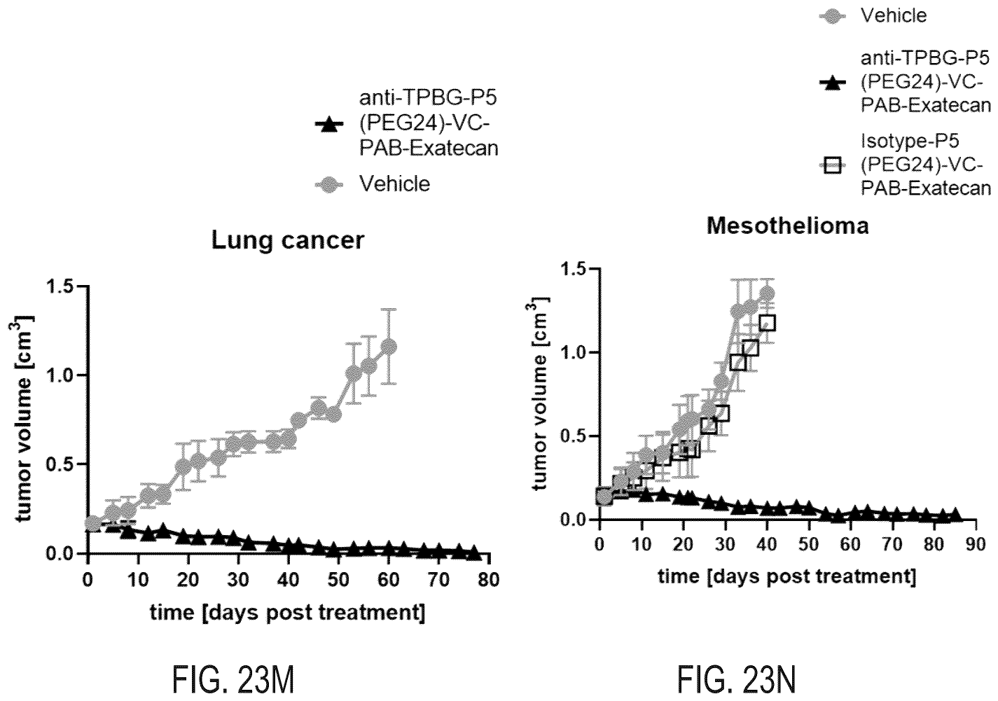

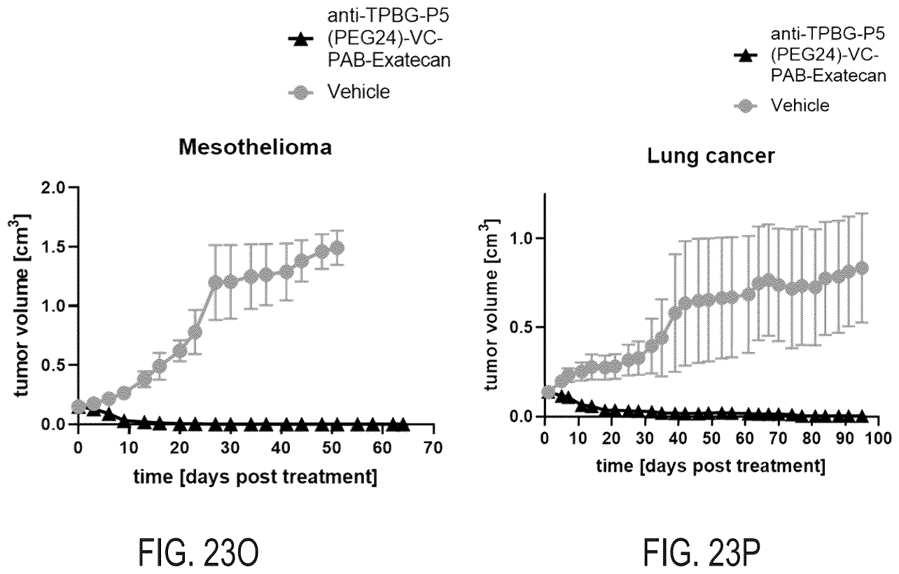

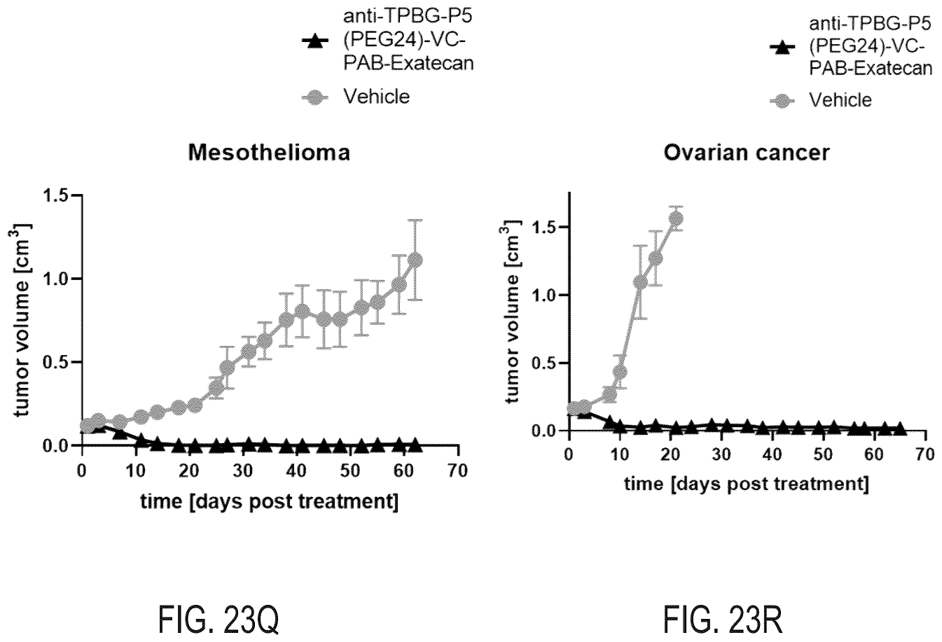

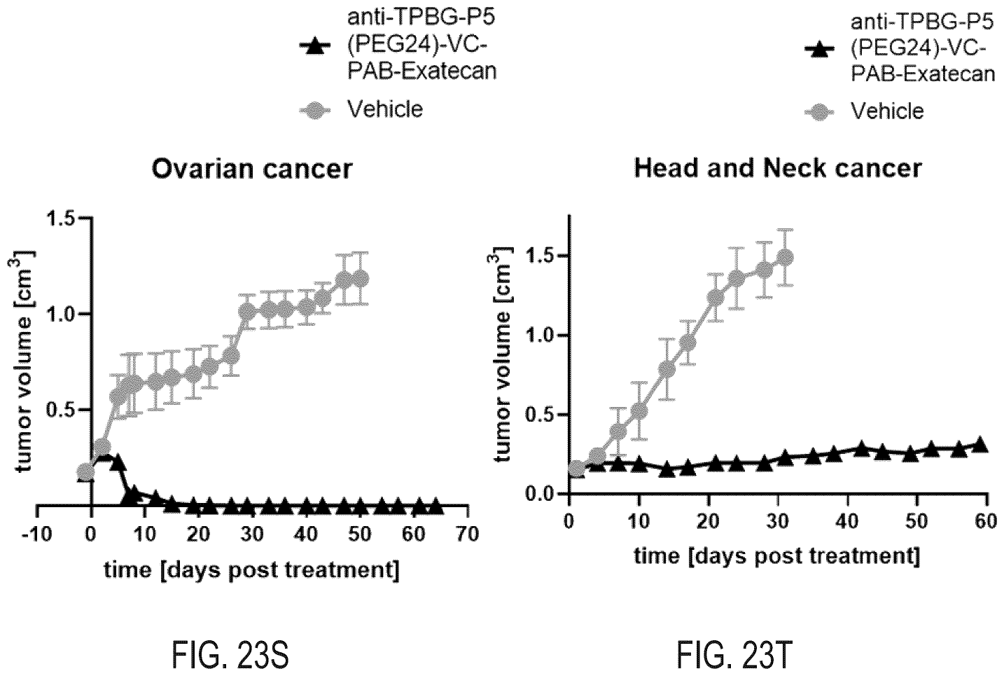

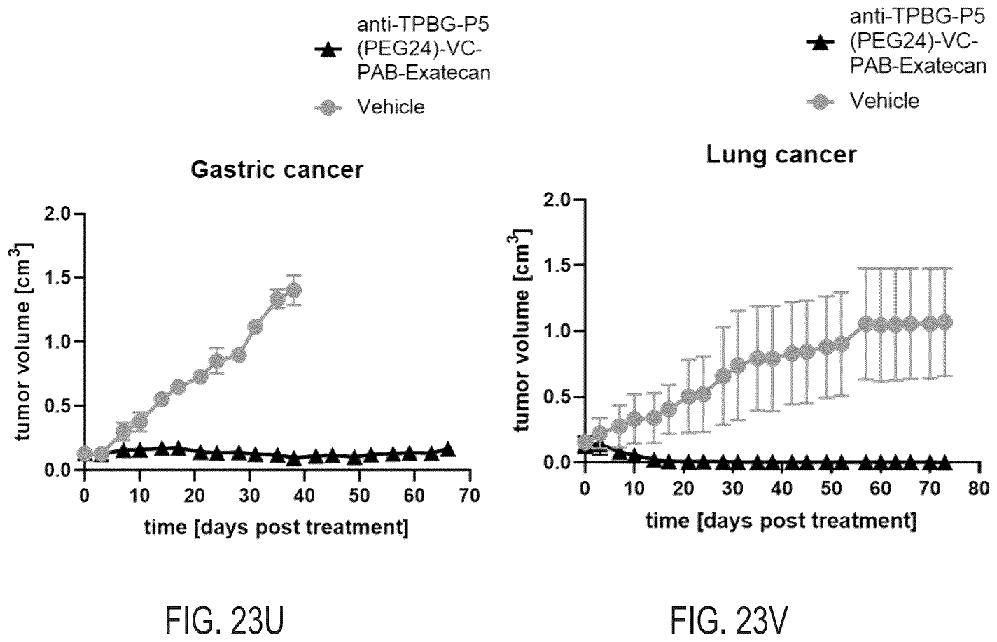

The data clearly demonstrates very high efficacy in patient derived tumor models in vivo compared to the vehicle control in different tumor types such as lung, endometrial, breast, colon, gastric, Head and Neck, pancreatic and ovarian cancer and Sarcoma and Mesothelioma. Highest selectivity has been demonstrated in the models in which the Isotype control ADC exhibits almost no anti-tumor activity, when administered at the same dose (FIGS. 23A to 23V).

### In Vivo Toxicity in Marmoset Monkeys:

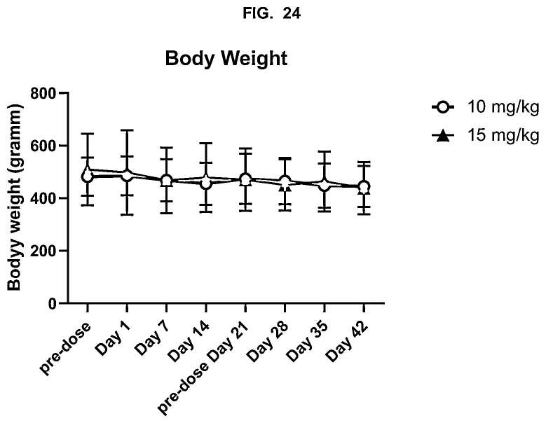

Toxicology studies were performed in marmoset monkeys. At least two animals for each group were dosed intravenously with 10 and 15 mg/kg anti-TPBG-P5(PEG24)-VC-PAB-Exatecan on days 1 and 22, with terminal necropsy at day 43. Total ADC and intact ADC were analyzed in the animal plasma samples through ELISA. No severe changes in body weight, clinical chemistry and hematology were observed at those dose levels (FIG. 24).

The applied doses are around 10-times higher compared to doses that were explored in toxicology studies with other 5T4-targeting ADCs.

This significant improvement in increasing tolerability of the ADC could be attributed to the reduced aggregation tendency of the ADC described herein. Antibody and ADC aggregation has been shown to be one of the major off-target toxicity drivers of ADCs. Moreover, Fc silencing as introduced in the anti-TPBG antibody described herein could contribute to reduced toxicity and therefore higher tolerability in the toxicology experiment. This important preclinical toxicity experiment to assess the safety of the molecule could be performed due to the above-described cross reactivity of the anti-TPBG antibody with marmoset 5T4. Without Marmoset cross reactivity, no meaningful preclinical data could be generated before entering clinical studies in human.

### Example 3: Further Characterization of the Anti-TPBG and ADCs Based Thereon

For all data shown, anti-TPBG-P5(PEG24)-VC-PAB-Exatecan refers to the LALA-mutated version of the anti-TPBG mAb.

### Comparison Antibody Synthesis and Expression

Anti-TPBG comparison antibody 1:
DNA coding for the light
(SEQ ID NO: 15
MLPSQLIGFLLLWVPASRGDIQMTQSPSSLSASVGDRVTITCKASQSVSNDVAWYQQKPGKAP
KLLIYFATNRYTGVPSRFSGSGYGTDFTLTISSLQPEDFATYYCQQDYSSPWTFGQGTKVEIKR
TVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC*; LC is shown in italic)
and
heavy chain
(SEQ ID NO: 16
MDWTWRILFLVAAATGAHSEVQLVESGGGLVQPGGSLRLSCAASGYTFTNFGMNWVRQAPG
KGLEWVAWINTNTGEPRYAEEFKGRFTISRDNAKNSLYLQMNSLRAEDTAVYYCARDWDGAY
FFDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQ
GNVFSCSVMHEALHNHYTQKSLSLSPGK*; HC is shown in italic)

of the anti-TPBG comparison antibody 1 was synthesized (Geneart, Thermo Fisher). Signal sequences for heavy chain (SEQ ID NO: 17, MDWTWRILFLVAAATGAHS) and light chain (SEQ ID NO: 18, MLPSQLIGFLLLWVPASRG) were added. Light chain and heavy chain sequences were cloned into pcDNA3.4-TOPO (Thermo Fisher) expression plasmid. Antibodies were then transiently expressed in Expi-CHO-S cells (Thermo Fisher) by co-transfecting cells with pcDNA3.4 expression plasmids (Thermo Fisher), coding for the heavy and light chain of the respective sequences in a 1:1 ratio, using the Expi-CHO transfection system (Thermo Fisher). Cells were harvested by centrifugation at 300 g for 5 minutes at 4° C. To clear micro particles from supernatant, supernatants were centrifuged at 4000-5000 g for 30 min at 4° C. For further clarification supernatants were passed through a 0.22 μm filter. Antibodies were purified from cleared and filtered supernatants via Protein A chromatography.

Anti-TPBG comparison antibody 2:
DNA coding for the light
(SEQ ID NO: 19
MLPSQLIGFLLLWVPASRGDIQMTQSPSTLSASVGDRVTITCQASQSIGSELAWYQQKPGKAPK
LLIYRASTLESGVPSRFSGSGSGTEFTLTISSLQPDDFATYYCQQGYTYSEIDNAFGQGTKVEIK
RTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC*; LC is shown in italic)
and
heavy chain
(SEQ ID NO: 20
MDWTWRILFLVAAATGAHSEVQLEESGGGLVKPGGSLRLSCAASGIDLSHYVVGWVRQAPGK
GLEWVSIIYGSGRTYYANWAKGRFTISRDNSKNTLYLQMNSLRAEDTAVYYCARDASVSVYYW
GYFDLWGRGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCDKTHTCPP
CPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQ
QGNVFSCSVMHEALHNHYTQKSLSLSPGK*; HC is shown in italic)

of the anti-TPBG comparison antibody 2 was synthesized (Geneart, Thermo Fisher). Signal sequences for heavy chain (SEQ ID NO: 21, MDWTWRILFLVAAATGAHS) and light chain (SEQ ID NO: 22, MLPSQLIGFLLLWVPASRG) were added. Light chain and heavy chain sequences were cloned into pcDNA3.4-TOPO (Thermo Fisher) expression plasmid. Antibodies were then transiently expressed in Expi-CHO-S cells (Thermo Fisher) by co-transfecting cells with pcDNA3.4 expression plasmids (Thermo Fisher), coding for the heavy and light chain of the respective sequences in a 1:1 ratio, using the Expi-CHO transfection system (Thermo Fisher). Cells were harvested by centrifugation at 300 g for 5 minutes at 4° C. To clear micro particles from supernatant, supernatants were centrifuged at 4000-5000 g for 30 min at 4° C. For further clarification supernatants were passed through a 0.22 μm filter. Antibodies were purified from cleared and filtered supernatants via Protein A chromatography.

### Comparison ADC Synthesis

### Anti-TPBG Comparison ADC1:

50 μl of a solution of a 10 mg/mL Anti-TPBG comparison antibody1 (66.67 μM) in Dulbecco's-PBS (Merck KGaA) were mixed with 1.33 μl of a TCEP solution (0.5 mM in buffered solution, Merck KGaA diluted to 10 mM with PBS, 4 eq. TCEP with respect to the antibody). After 30 Min incubation at RT, 0.83 μl of a 40 mM solution of Maleimidocaproyl monomethylauristatin F (MC-MMAF) in DMSO (10.0 eq. with respect to the antibody) were added. The mixture was shaken (350 RPM) at room temperature (25° C.) for 1 hour. The reaction mixture was purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra—2 mL MWCO: 30 kDa, Merck, Germany) and analyzed by MS, as described in example 1.

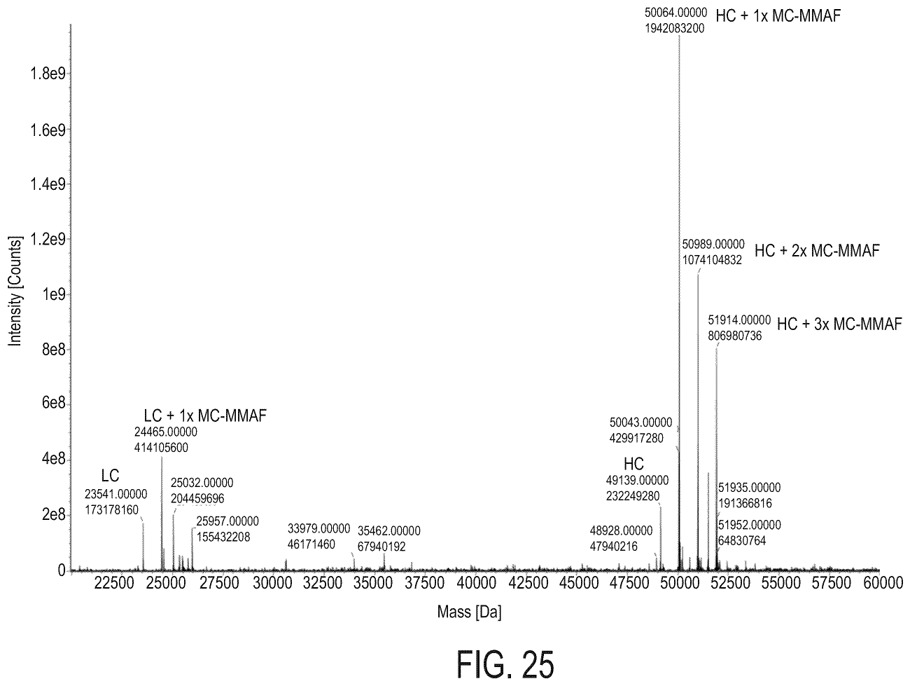

FIG. 25 shows the MS analysis of the Anti-TPBG comparison ADC1, synthesized by the method described above. Signals are annotated with the measured mass in Dalton and the absolute intensities. The Drug-to-Antibody ration has been estimated from the MS signals to 4.2. LC: light chain of Anti-TPBG comparison antibody1, HC: heavy chain of Anti-TPBG comparison antibody 1.

### Anti-TPBG Comparison ADC 2:

50 μl of a solution of a 10 mg/mL Anti-TPBG comparison antibody2 (66.67 μM) in Dulbecco's-PBS (Merck KGaA) were mixed with 13.3 μl of a TCEP solution (0.5 mM in buffered solution, Merck KGaA diluted to 10 mM with PBS, 40 eq. TCEP with respect to the antibody). After 1 hour incubation at RT, the solution was rebuffered to PBS using 0.5 mL Zeba™ Spin Desalting Columns with 7K MWCO (Thermo Fisher Scientific, USA) in accordance with the manufacturer's instructions. 13.33 μl of a 10 mM of Dehydroascorbic acid (Merck KGaA, 20 mM in PBS, 40 eq. with respect to the antibody) were added and the mixture incubated for 3 h at RT. Afterwards, 6.66 μl of a 10 mM solution of MC-VC-SECO-DUBA (MedChemExpress, 20 eq. with respect to the antibody) dissolved in DMSO were added. The mixture was shaken (350 RPM) at room temperature (25° C.) for 1 hour. The reaction mixture was purified by preparative size-exclusion chromatography with a 25 ml Superdex™ 200 Increase 10/300GL (Cytiva, Sweden) and a flow of 0.8 ml/min eluting with sterile PBS (Merck, Germany). The antibody containing fractions were pooled and concentrated by spin-filtration (Amicon® Ultra—2 mL MWCO: 30 kDa, Merck, Germany) and analyzed by MS, as described in example 1.

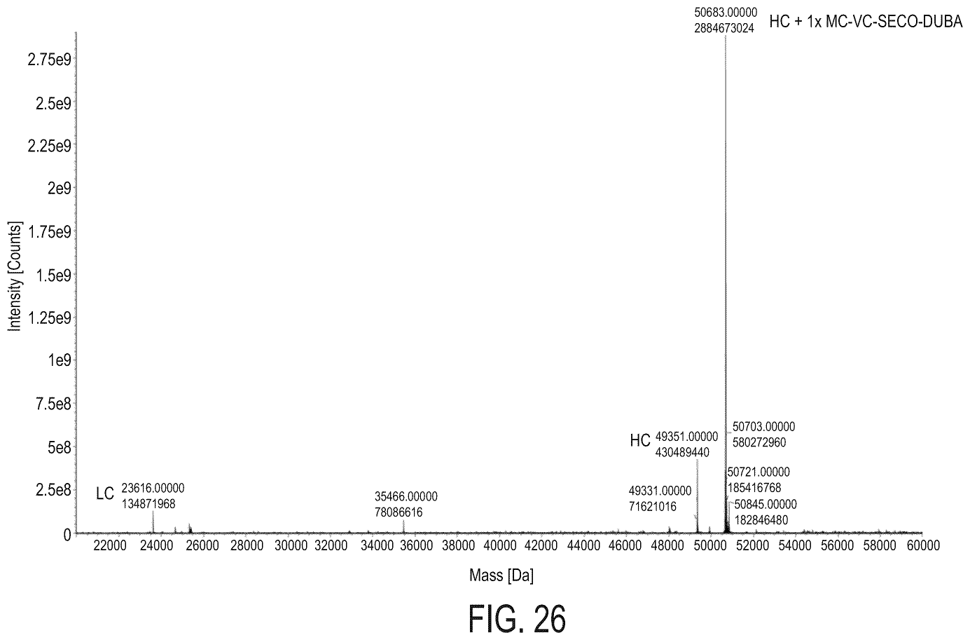

FIG. 26 shows the MS analysis of the Anti-TPBG comparison ADC 2, synthesized by the method described above. Signals are annotated with the measured mass in Dalton and the absolute intensities. The Drug-to-Antibody ratio has been estimated from the MS signals to 1.7. LC: light chain of Anti-TPBG comparison antibody2, HC: heavy chain of Anti-TPBG comparison antibody 2.

### In Vitro and In Vivo Experiments:

### In Vitro Cytotoxicity Evaluated Via Resazurin Assay—Anti-TPBG Comparison ADC1 vs Anti-TPBG-P5(PEG24)-VC-PAB-Exatecan DAR8:

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-12 μg/ml) to generate a dose-response curve. Before viability analysis, the supernatant over the adherent cells was removed and replaced by fresh medium. Killing was analyzed afterwards, using resazurin (Sigma-Aldrich) as the cell viability dye at a final concentration of 55 μM. Fluorescence emission at 590 nM was measured on a Microplate reader Infinite M200 Pro (Tecan). Cell viability was measured by dividing the fluorescence of ADC-treated cells by the fluorescence from control cells, treated in the same way with medium only. Graph shows means of n=2±SEM.

In this experiment, the cytotoxicity of anti-TPBG-P5(PEG24)-VC-PAB-Exatecan DAR 8 compared to Anti-TPBG comparison ADC1, targeting 5T4 was investigated. On all tested cell lines with various TPBG expression levels anti-TPBG-P5(PEG24)-VC-PAB-Exatecan shows 18-66-fold improved killing IC50 values compared to Anti-TPBG comparison ADC1.

Killing of the antigen-positive tumor cells of anti-TPBG-P5(PEG24)-VC-PAB-Exatecan is 18-66-fold improved, when IC50 values are compared to Anti-TPBG comparison ADC1. Specificity has been demonstrated with an isotype control ADC, which showed no effect. The results clearly demonstrate superiority of the conjugates disclosed herein over previously developed ADCs directed against the same target.

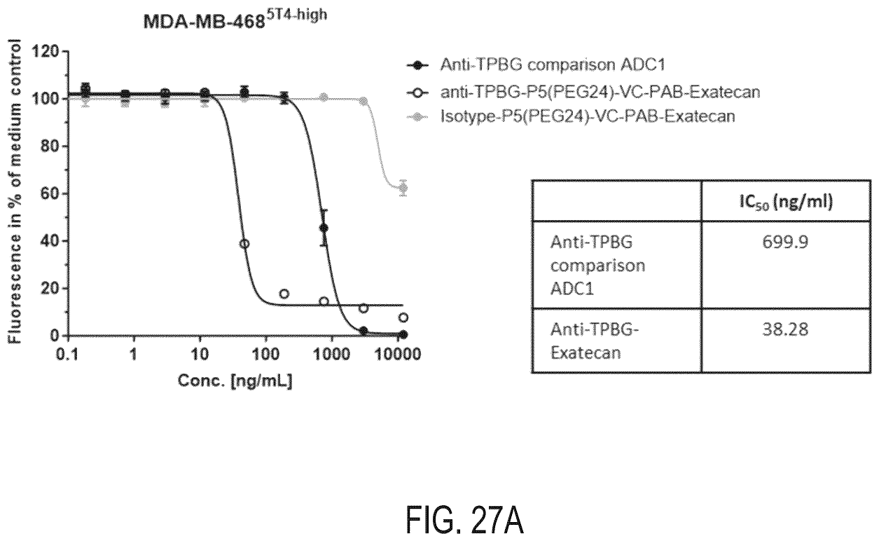

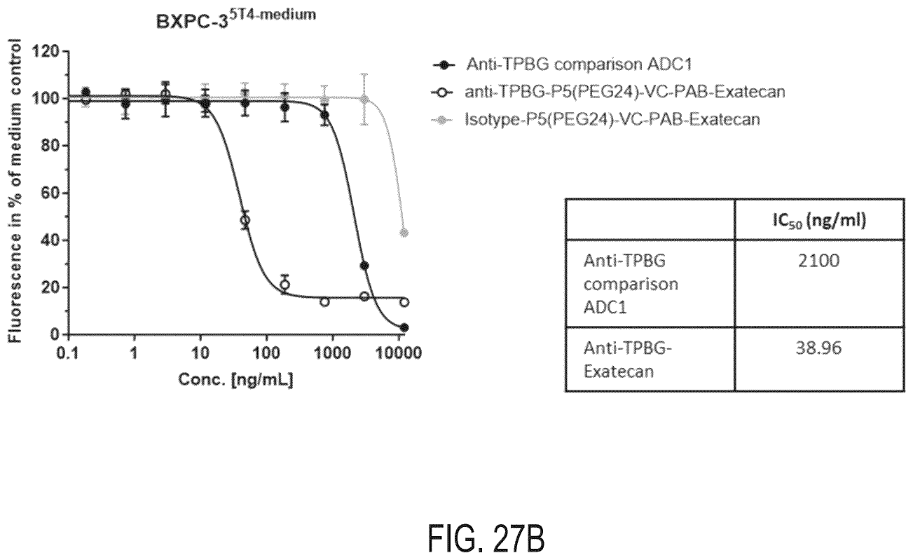

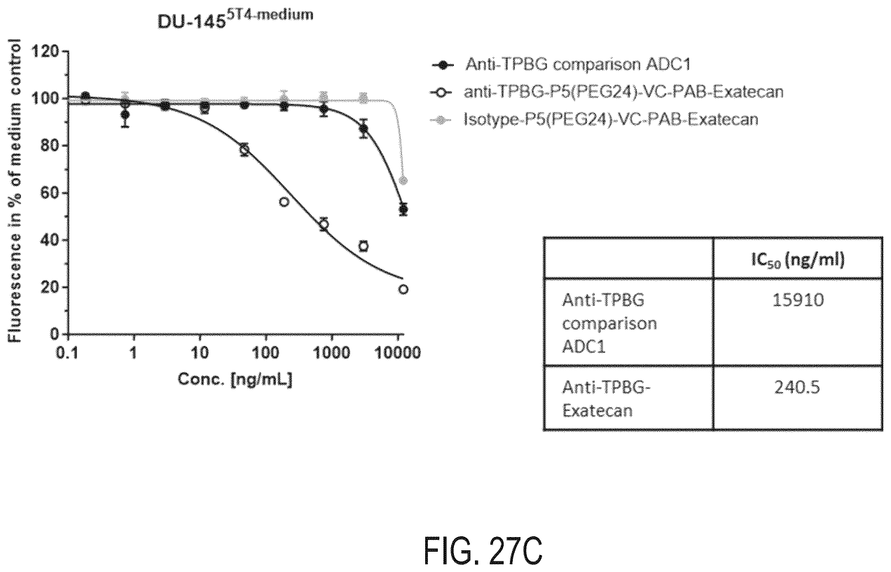

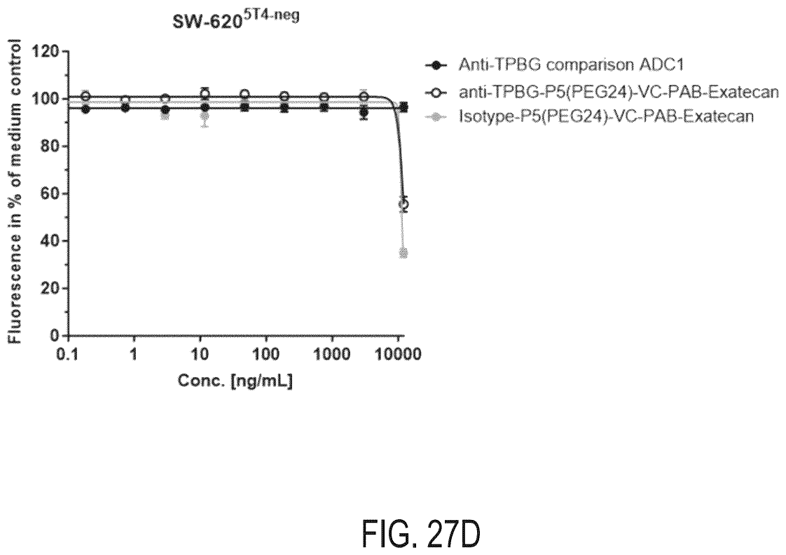

FIGS. 27A to 27D show the cytotoxicity dose-response of anti-TPBG-P5(PEG24)-VC-PAB-Exatecan DAR 8 and a corresponding non-targeting isotype control conjugate compared to anti-TPBG comparison ADC1 on 4 different cell lines. Shown are mean and SD of two measurements, as well as a dose-response-fit. Fluorescence in % of medium control is equal to % of viable cells.

### In Vitro Cytotoxicity Evaluated Via Resazurin Assay—Anti-TPBG Comparison ADC2 vs Anti-TPBG-P5(PEG24)-VC-PAB-Exatecan DAR8

To investigate direct cytotoxicity of ADCs, respective cells were seeded in a 96-well plate (flat bottom, 5000 cells/well, suspended in 100 μl medium) and incubated for 7 days with increasing concentrations of the ADCs in medium (0-12 μg/ml) to generate a dose-response curve. Before viability analysis, the supernatant over the adherent cells was removed and replaced by fresh medium. Killing was analyzed afterwards, using resazurin (Sigma-Aldrich) as the cell viability dye at a final concentration of 55 μM. Fluorescence emission at 590 nM was measured on a microplate reader Infinite M200 Pro (Tecan). Cell viability was measured by dividing the fluorescence of ADC-treated cells by the fluorescence from control cells, treated in the same way with medium only. Graph shows means of n=2±SEM.

In this experiment, the cytotoxicity of anti-TPBG-P5(PEG24)-VC-PAB-Exatecan DAR8 compared Anti-TPBG comparison ADC2, targeting 5T4 was investigated. On MDA-MB-468 cells with high 5T4 level, anti-TPBG-P5(PEG24)-VC-PAB-Exatecan shows 1.9-fold improved killing IC50 values compared to Anti-TPBG comparison ADC2. On BXPC-3 cells with lower 5T4 expression, Anti-TPBG comparison ADC2 was not active at all while anti-TPBG-P5(PEG24)-VC-PAB-Exatecan DAR8 showed high cytotoxic activity.

Killing of the antigen-positive tumor cells highly improved killing compared to Anti-TPBG comparison ADC2. Specificity has been demonstrated with an isotype control ADC, which showed no effect. The results clearly demonstrate superiority of the conjugates disclosed herein over previously developed ADCs directed against the same target.

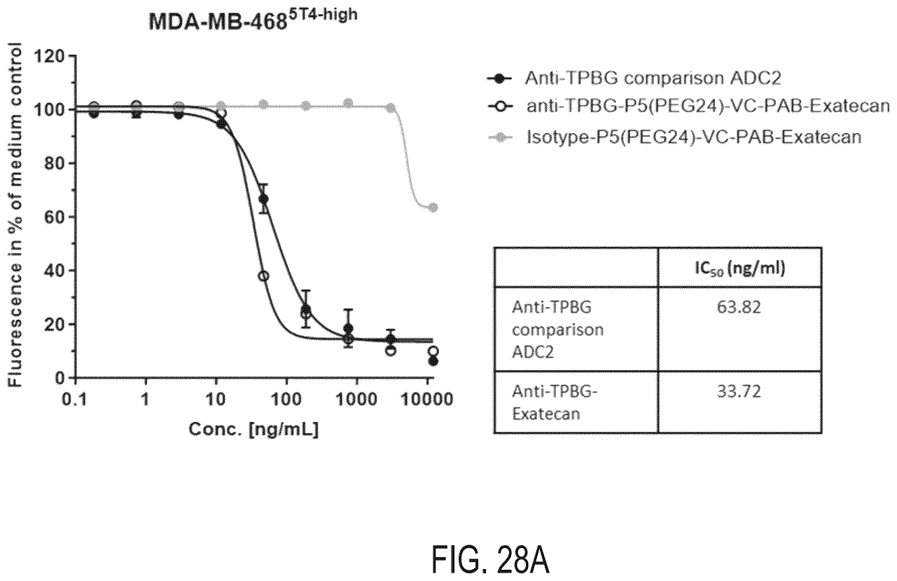

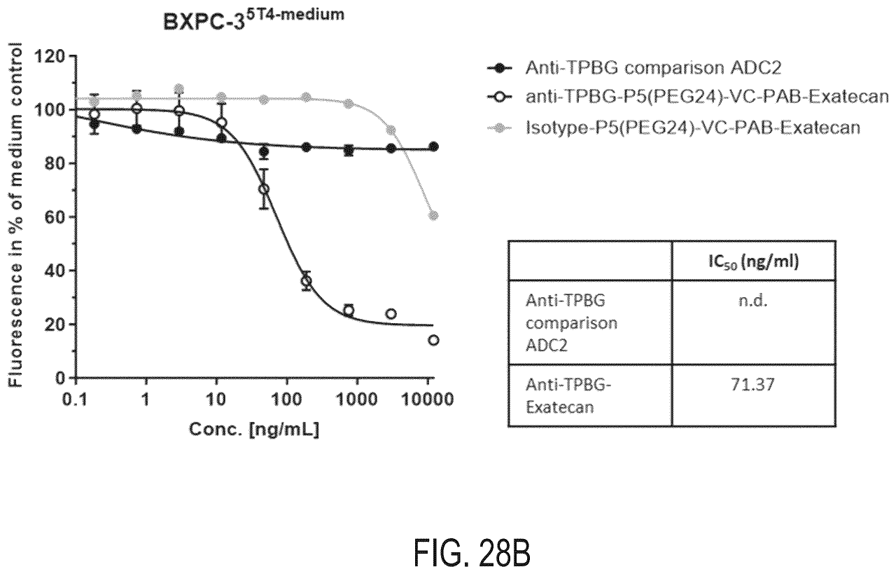

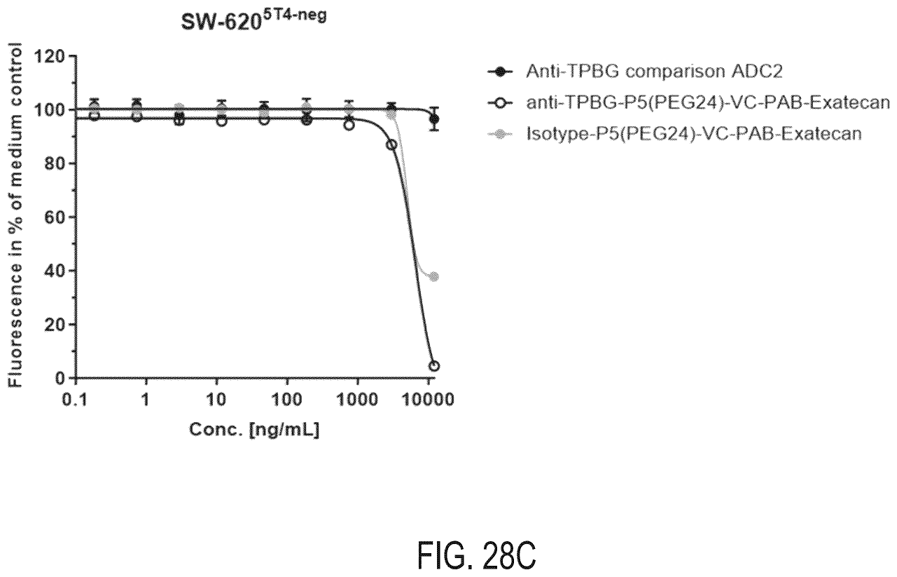

FIGS. 28A to 28C show the cytotoxicity dose-response of anti-TPBG-P5(PEG24)-VC-PAB-Exatecan DAR 8 and a corresponding non-targeting isotype control conjugate compared to Anti-TPBG comparison ADC2 on 3 different cell lines. Shown are mean and SD of two measurements, as well as a dose-response-fit. Fluorescence in % of medium control is equal to % of viable cells.

### Bystander Killing—Supernatant Transfer

To analyze bystander activity of ADCs on target-negative cells, 20.000 5T4-positive cells (MDA-MB-468 or BXPC-3) were incubated with increasing concentrations of ADCs (0-3 μg/ml). After 5 days, half of the cell culture supernatant volumes was transferred to 5.000 5T4-negative cells (SW-620) and incubated for another 5 days. Killing was analyzed by a resazurin-based viability measurement as described above.

In this experiment, the bystander activity of anti-TPBG-P5(PEG24)-VC-PAB-Exatecan DAR 8 compared to Anti-TPBG comparison ADC2, targeting 5T4 was investigated in a supernatant transfer-based bystander experiment. While anti-TPBG-P5(PEG24)-VC-PAB-Exatecan showed high bystander activity on target-negative cells after supernatant transfer from target-positive cells, Anti-TPBG comparison ADC2 did not exhibit bystander effects at all. It should be noted that both ADCs did not have an effect on the antigen negative cell line SW-620, when not preincubated with the antigen positive cell line MDA-MB-468, as shown in the previous example.

Bystander killing is a key feature of ADCs to target heterogeneous tumors. The results clearly demonstrate superiority of the conjugates disclosed herein over previously developed ADCs directed against the same target.

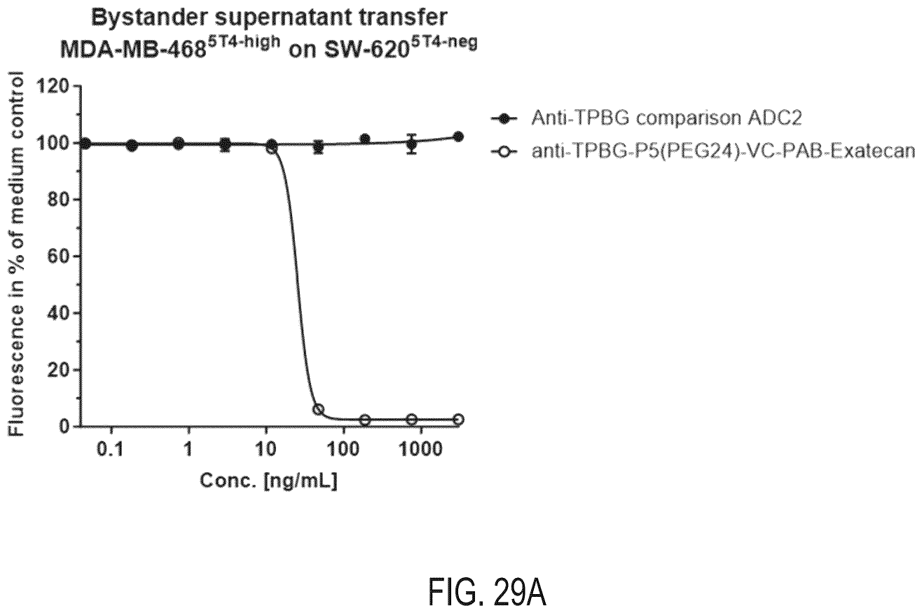

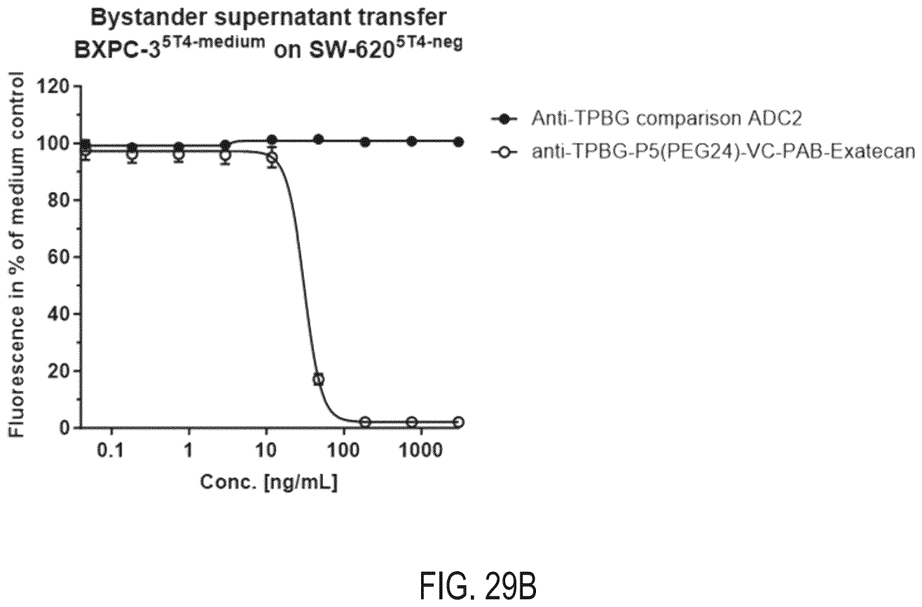

FIGS. 29A and 29B show the cytotoxicity dose-response of target negative cells (SW-620) after the transfer of cell culture supernatant of two different 5T4-positive cell lines (MDA-MB-468 and BXPC-3) that were pre-treated with serial dilutions of anti-TPBG-P5(PEG24)-VC-PAB-Exatecan DAR 8 or Anti-TPBG comparison ADC2 Shown are mean and SD of two measurements, as well as a dose-response-fit. Fluorescence in % of medium control is equal to % of viable cells.

### Bystander Killing—Co-Culture

For bystander assay performed as co-culture, 10.000 5T4-positive BXPC-3 cells and 2.000 5T4-negative SW-620 cells were incubated with increasing concentrations of ADC (0-3 μg/ml). After 5 days, cells were harvested and stained with a live/dead stain (Thermo Fisher Scientific) and fluorescently-labeled αTROP-2 (BioLegend) to distinguish between BXPC-3 (TROP-2 positive) and SW-620 (TROP-2 negative) cells. The percentage of viable BXPC-3 and SW-620 cells was analyzed by flow cytometry.

(Similarly to the supernatant transfer assay, described in example 1) The anti-TPBG-P5(PEG24)-VC-PAB-Exatecan DAR 8 ADC shows strong bystander activity in the co-culture assay.

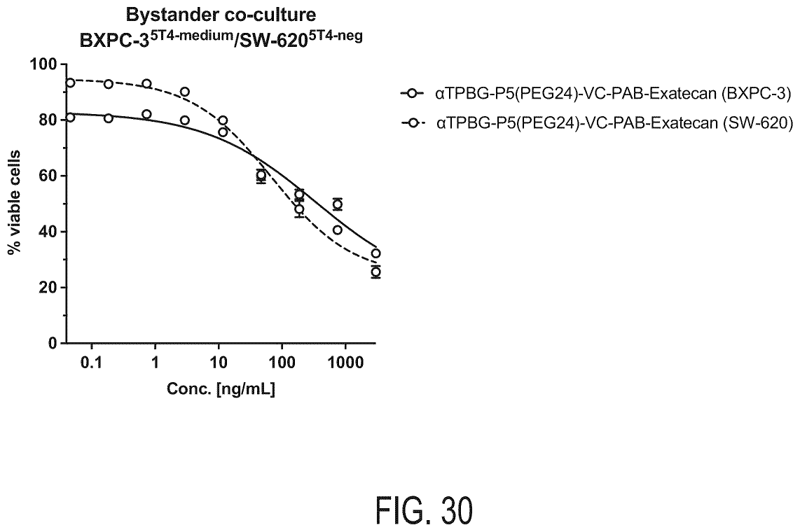

FIG. 30 shows the cytotoxicity dose-response of serial dilutions of the anti-TPBG-P5(PEG24)-VC-PAB-Exatecan DAR 8 ADC in co-cultures of a target-positive (BX-PC-3) and target-negative cell line (SW-620). Cell killing curves are shown separately for each cell-line. Shown are mean and SD of two measurements, as well as a dose-response-fit.

### In Vitro Inhibition of Topoisomerase-I by the Delivery of Exatecan Via the ADC

Topoisomerase-I inhibition by delivery of exatecan via anti-TPBG-P5(PEG24)-VC-PAB-Exatecan DAR8 induces DNA damage as detected by the accumulation of cleaved PARP, active caspase 3 and phosphorylated histon 2AX (pH2AX). MDA-MB-468 cells (5T4^{high}) were treated with increasing concentrations (0.05-12 μg/ml) of anti-TPBG-P5(PEG24)-VC-PAB-Exatecan DAR8 or an Isotype ADC (Isotype-P5(PEG24)-VC-PAB-Exatecan DAR8) for 72 h. Cells were stained with live/dead stain and after fixation and permeabilization using the Fixation/Permeabilization kit (BD Biosciences) for the DNA damage markers active caspase 3, cleaved PARP and pH2AX (Ser-139) and analyzed by flow cytometry. Graphs show means of n=2±SEM.

anti-TPBG-P5(PEG24)-VC-PAB-Exatecan DAR8 induces a concentration-dependent accumulation of DNA damage as shown by the increase of cleaved PARP-, active caspase 3- and pH2AX-positive MDA-MB-468 cells, whereas the isotype control remains inactive.

The experiment clearly shows that the ADCs are delivering the exatecan selectively into the targeted cell and induce cell killing by topoisomerase-I-inhibition.

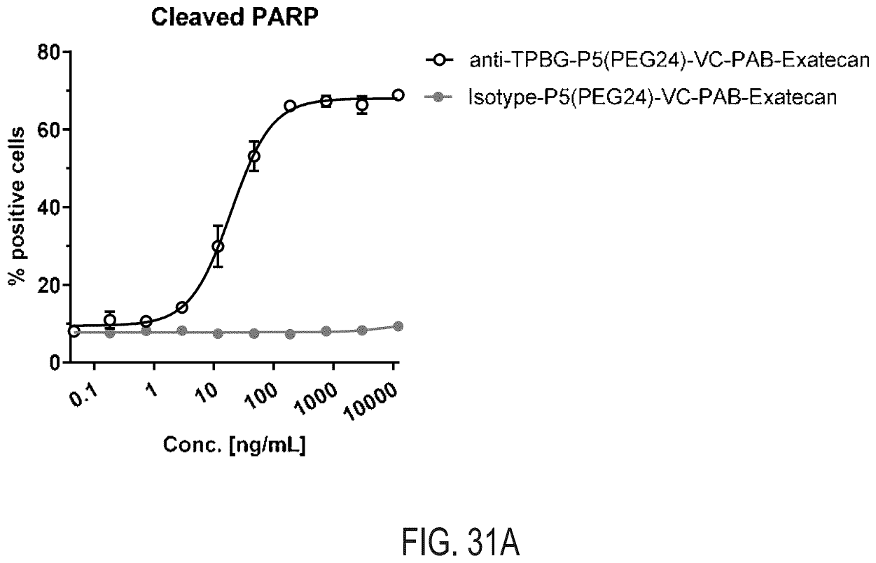

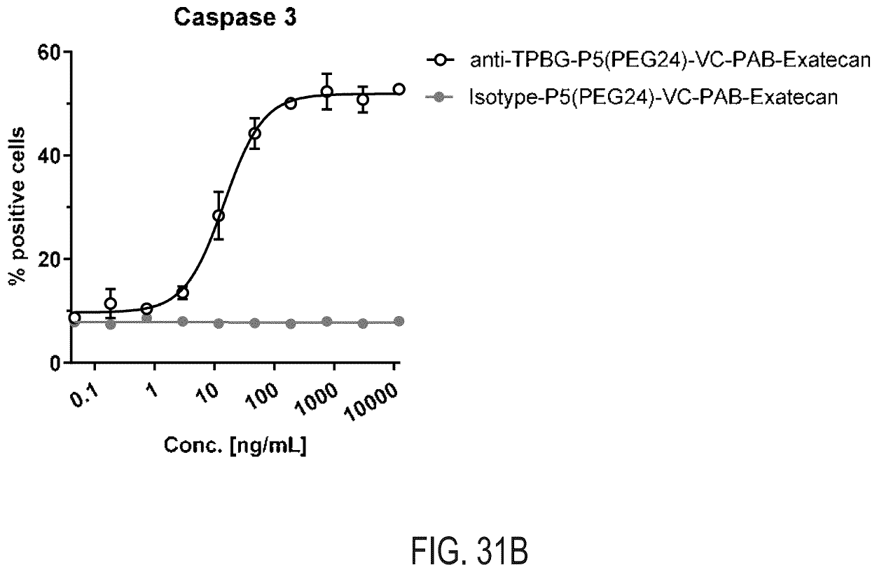

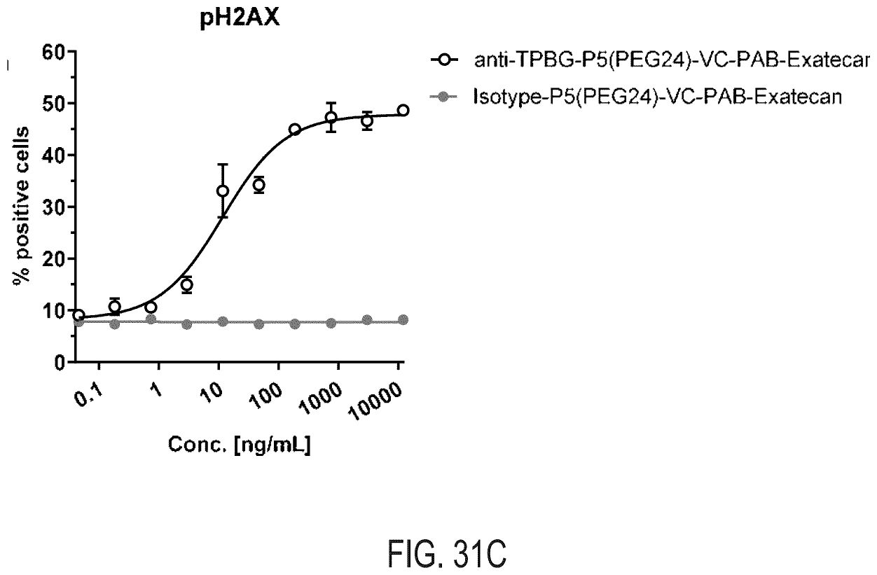

FIGS. 31A to 31C show the dose-dependent induction of DNA-damage and apoptosis markers in response to treatment with increasing concentrations of anti-TPBG-P5(PEG24)-VC-PAB-Exatecan DAR8. A corresponding non-targeting isotype control conjugate was included as negative control. MDA-MB-468 cells (5T4^{high}) were treated with increasing concentrations (0.05-12 μg/ml) of anti-TPBG-P5(PEG24)-VC-PAB-Exatecan DAR8 or the Isotype ADC (Isotype-P5(PEG24)-VC-PAB-Exatecan DAR8) for 72 h, Cells were stained for cleaved PARP (left), Caspase 3 (middle) and pH2AX (right) and analyzed via flow cytometry. Graphs show means of n=2±SEM.

Interaction with Complement Factor C1q

To reduce or even prevent interaction of the IgG1 backbone anti-TPBG antibody with complement factors or Fc receptors (FcRs) that might trigger unwanted immune activation and/or FcR-mediated cellular uptake, the Fc-part of the anti-TPBG antibody and the derived ADC anti-TPBG-P5(PEG24)-VC-PAB-Exatecan has been silenced using two point mutations L234A and L235A (LALA).

In this experiment, interaction of the LALA-silenced anti-TPBG antibody (SEQ ID NO: 23,

MDWTWRILFLVAAATGAHSEVQLVQSGAEVKKPGASVKVSCKASGYSFTGYYMHWVRQAPG
QGLEWMGRINPNNGVTLYNQKFKDRVTITRDTSTSTAYMELSSLRSEDTAVYYCARSTMITNYV
MDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQ
GNVFSCSVMHEALHNHYTQKSLSLSPGK*)
vs Fc-wildtype anti-TPBG antibody
(HC-wt: SEQ ID NO: 24,
MDWTWRILFLVAAATGAHSEVQLVQSGAEVKKPGASVKVSCKASGYSFTGYYMHWVRQAPG
QGLEWMGRINPNNGVTLYNQKFKDRVTITRDTSTSTAYMELSSLRSEDTAVYYCARSTMITNYV
MDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQ
GNVFSCSVMHEALHNHYTQKSLSLSPGK*)

with complement factor C1q was analyzed. C1q interaction studies were performed using the HTRF Human C1q Binding Kit (Cisbio) according to the manufacturer's instructions. In brief, anti-TPBG HC-wt and HC-LALA, a standard and an anti-MHC-I positive control (Invivogen) known to interact with C1q are captured and aggregated by an anti-human IgG Fab-biotin complexed to streptavidin, which binds streptavidin-labelled d2 (fluorescence acceptor). If the antibodies bind to human C1q, an anti-C1q antibody labelled with Europium cryptate (fluorescence donor) can come in close proximity to the fluorescence acceptor and fluorescence resonance energy transfer (FRET) is triggered. Fluorescence emission at 665 nm is measured on a microplate reader Infinite M200 Pro (Tecan). Graphs show means of n=2±SD.

While the standard and the anti-MHC-I positive control show high interaction capacity with C1q, anti-TPBG HC-wt only exhibits low binding to C1q. This is completely abolished for the HC-LALA-mutated antibody.

The reduced interaction with C1q is expected to decrease undesired activation of the innate immune system.

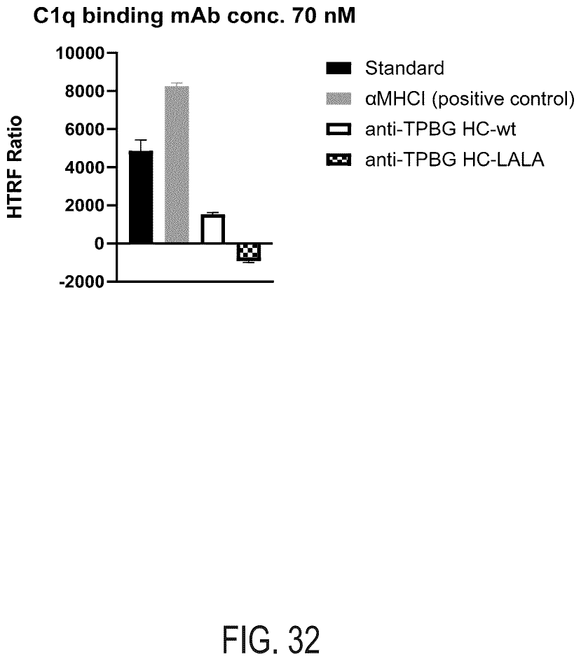

FIG. 32 shows binding of the Fc region of the anti-TPBG-HC-LALA antibody versus the anti-TPBG-HC-wt antibody to recombinant hexameric C1q complement protein was measured in a HTRF (Homogenous Time-Resolved Fluorescence) based human C1q binding assay (HTRF Human C1q Binding Kit, Cisbio) according to manufacturer's instructions. In brief, serial dilutions 280 nM-11.6 nM of all tested antibodies (anti-TPBG mAb HC-wt, anti-TPBG mAb HC-LALA, α-MHC-I positive, IgG1 kit standard) were measured. HTRF ratio was calculated by dividing the acceptor emission signal at 665 nm by the donor emission signal at 620 nm and multiplying by 10000. Graph shows HTRF ratio at 70 nM concentration with the background (diluent only) HTRF ratio subtracted. Graph shows means of n=2±SD.

Interaction with FcRs

To reduce or even prevent interaction of the IgG1 backbone anti-TPBG antibody with complement factors or Fc receptors (FcRs) that might trigger unwanted immune activation and/or FcR-mediated cellular uptake, the Fc-part of the anti-TPBG antibody and the derived ADC anti-TPBG-P5(PEG24)-VC-PAB-Exatecan DAR8 has been silenced using two point mutations L234A and L235A (LALA).

In this experiment, interaction of the LALA-silenced anti-TPBG antibody

(HC-LALA: SEQ ID NO: 25
MDWTWRILFLVAAATGAHSEVQLVQSGAEVKKPGASVKVSCKASGYSFTGYY
MHWVRQAPGQGLEWMGRINPNNGVTLYNQKFKDRVTITRDTSTSTAYMELSSLRSEDTAVYY
CARSTMITNYVMDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPK
SCDKTHTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK*; HC is shown in italic)
and
anti-TPBG-P5(PEG24)-VC-PAB-Exatecan DAR8 vs Fc-wildtype anti-TPBG
antibody
(HC-wt: SEQ ID NO: 26
MDWTWRILFLVAAATGAHSEVQLVQSGAEVKKPGASVKVSCKASGYSFTGYY
MHWVRQAPGQGLEWMGRINPNNGVTLYNQKFKDRVTITRDTSTSTAYMELSSLRSEDTAVYY
CARSTMITNYVMDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK *; HC is shown in italic)

with Fc gamma receptors (FcγRs) was analyzed. FcγR interaction studies were performed using the Lumit™ FcγR Binding Immunoassays (FcγRn, FcγRI, FcγRIIa/CD32 R131/H131 polymorphism, FcγRIIIa/CD16 V158/F158 polymorphism; Promega) based on a competition principle and luciferase detection according to the manufacturer's instructions. In brief, anti-TPBG HC-wt, HC-LALA and anti-TPBG-P5(PEG24)-VC-PAB-Exatecan DAR8, a standard and trastuzumab as positive control were incubated with a Tracer-LgBiT and a FcγR-SmBiT. In the absence of an antibody analyte or if no interaction of the tested antibody with FcγRs takes place, Tracer-LgBiT binds to the FcγR-SmBiT target, resulting in maximum luminescence signal. In the case of successful interaction with FcγRs, the tested antibody/ADC will compete with Tracer-LgBiT for binding to the FcγR target, resulting in a concentration-dependent decrease in the luminescent signal. Luminescence is measured on a microplate reader Infinite M200 Pro (Tecan). Graphs show n=1.

While anti-TPBG HC-wt binds similarly well to all FcγR as the IgG1 positive control, interaction with FcγR is either highly reduced or in most cases completely abolished for anti-TPBG HC-LALA and anti-TPBG-P5(PEG24)-VC-PAB-Exatecan. Interestingly, the positive control trastuzumab even shows reduced interaction with all FcγR compared to the positive control. In the FcγRn interaction experiment, the murine anti-TPBG served as another negative control.

The experiment clearly showed reduction of undesired interaction with FcγRI (CD64), CD16 and CD32, mediated by the incorporation of the LALA mutation into the anti-TPBG antibody. The reduced interaction with those receptors is expected to decrease undesired uptake of the anti-TGPB-related ADC conjugates into non-targeted cells and thereby reduce undesired toxicities and unwanted immune cell activation. Interaction with FcRn was only mildly reduced.

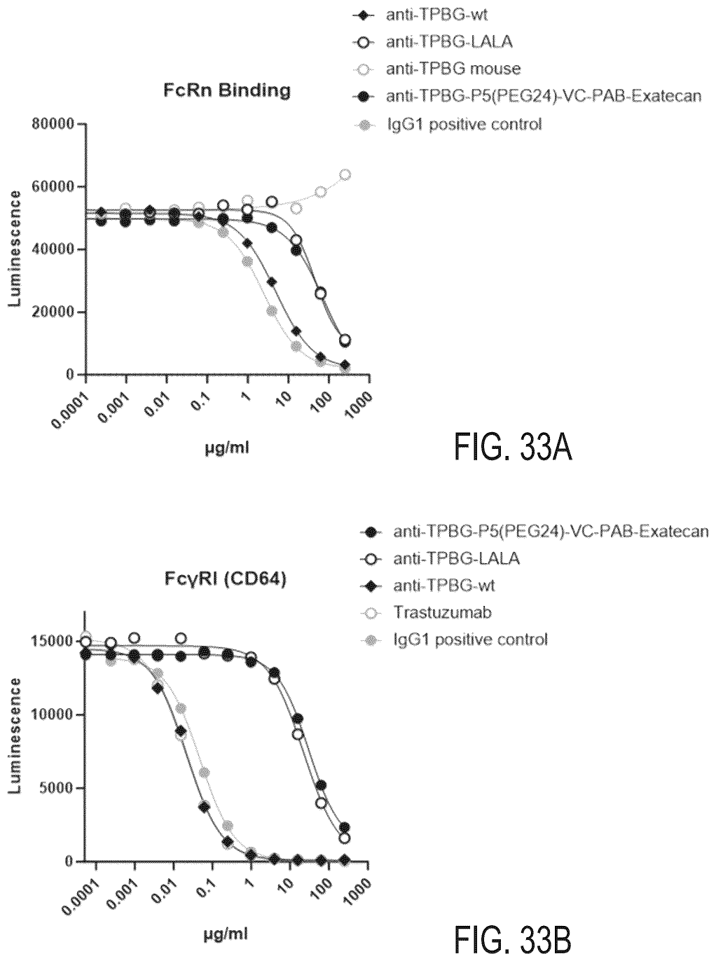

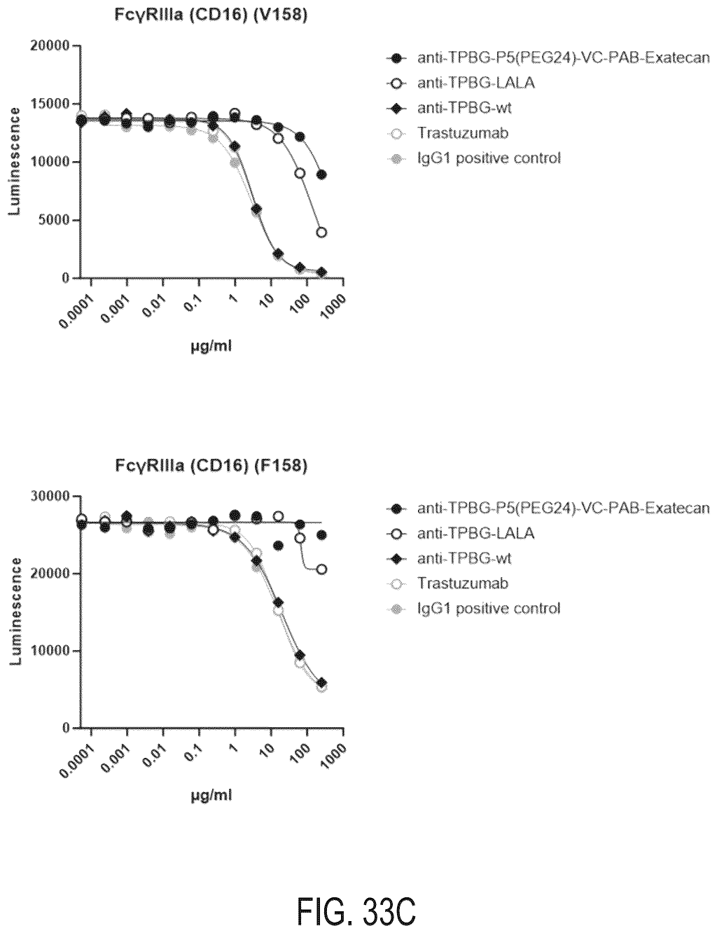

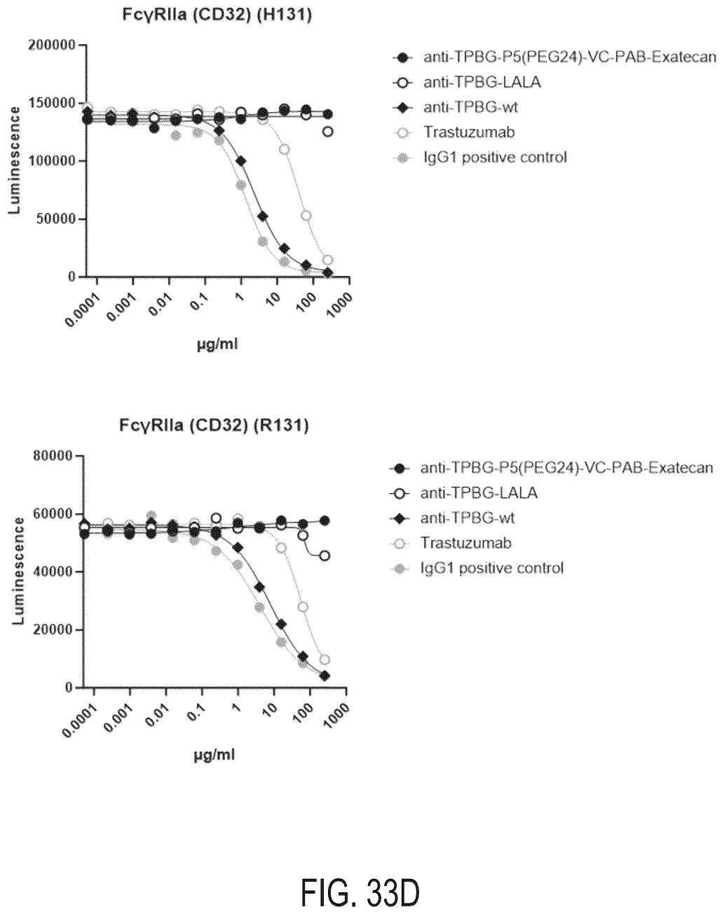

FIGS. 33A to 33D show dose-dependent binding of the Fc region of the anti-TPBG-HC-LALA antibody and the anti-TPBG-P5(PEG24)-VC-PAB-Exatecan DAR8 ADC versus the anti-TPBG-HC-wt antibody to recombinant human FcRn and FcγR was measured using Lumit™ FcγR Binding Immunoassays (FcγRn, FcγRI, FcγRIIa/CD32 R131/H131 polymorphism, FcγRIIIa/CD16 V158/F158 polymorphism; Promega) according to the manufacturer's instructions. Serial dilutions of anti-TPBG HC-wt, anti-TPBG HC-LALA and anti-TPBG-P5(PEG24)-VC-PAB-Exatecan DAR8, a standard and trastuzumab as positive control were incubated with the Tracer-LgBiT and a FcγR-SmBiT. In the absence of an antibody analyte or if no interaction of the tested antibody with FcγRs takes place, Tracer-LgBiT binds to the FcγR-SmBiT target, resulting in maximum luminescence signal. In the case of successful interaction with FcγRs, the tested antibody/ADC will compete with Tracer-LgBiT for binding to the FcγR target, resulting in a concentration-dependent decrease in the luminescent signal. Luminescence was measured on a microplate reader Infinite M200 Pro (Tecan). Graphs show n=1.

### ADCC

To reduce or even prevent interaction of the IgG1 backbone anti-TPBG antibody with complement factors or Fc receptors (FcRs) that might trigger unwanted immune activation and/or FcR-mediated cellular uptake, the Fc-part of the anti-TPBG antibody and the derived ADC anti-TPBG-P5(PEG24)-VC-PAB-Exatecan DAR8 has been silenced using two point mutations L234A and L235A (LALA).

In this experiment, the antibody-dependent cellular cytotoxicity (ADCC) of the Fc-wildtype (HC-wt: SEQ ID NO: 27:

MDWTWRILFLVAAATGAHSEVQLVQSGAEVKKPGASVKVSCKASGYSFTGYY
MHWVRQAPGQGLEWMGRINPNNGVTLYNQKFKDRVTITRDTSTSTAYMELSSLRSEDTAVYY
CARSTMITNYVMDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK*; HC is shown in italic)
and
LALA-silenced anti-TPBG antibody
(HC-LALA: SEQ ID NO. 28:
MDWTWRILFLVAAATGAHSEVQLVQSGAEVKKPGASVKVSCKASGYSFTGYYMHWVRQAPG
QGLEWMGRINPNNGVTLYNQKFKDRVTITRDTSTSTAYMELSSLRSEDTAVYYCARSTMITNYV
MDYWGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQ
GNVFSCSVMHEALHNHYTQKSLSLSPGK*; HC is shown in italic)

and anti-TPBG-P5(PEG24)-VC-PAB-Exatecan DAR8 vs the isotypes and an anti-MHC-I positive control (Invivogen) was analyzed, which is mediated after interaction with FcγRIIIa (CD16). For the Calcein release-based antibody-dependent cellular cytotoxicity (ADCC) assay, peripheral blood mononuclear cells (PBMCs) were isolated from healthy donor buffy coats (purchased from DONAS GmbH) using LeucoSep tubes (Greiner Bio-One) and Histopaque®-1077 (density 1.077 g/ml, Merck) density gradient according to standard protocols. Afterwards, natural killer (NK) cells were MACS (magnetic cell separation)-sorted using human NK cell isolation kit (Miltenyi Biotec) by negative selection according to the manufacturer's instructions yielding untouched human primary NK cells. 5T4-positive target cells MDA-MB-468 (5T4^{high}) and DU-145 (5T4^{med}) were stained with 16 μM Calcein AM (Thermo Fisher Scientific). 40.000 NK cells and 10.000 Calcein-stained target cells were incubated at a 4:1 ratio in the presence of 15 μg/ml antibodies or ADCs. Cells permeabilized with 2.5% Triton X 100 (Merck) served as a positive control for maximum Calcein release. After 4 h, supernatants were transferred to a flat black non-binding 96-well plate (Greiner Bio-One) and fluorescence was measured at 485/535 nM via the Infinite M200 Pro reader (Tecan). The percent specific killing was calculated by dividing the Calcein released by antibody-mediated killing minus background Calcein release (NKs+targets) from Calcein released by Triton X-permeabilized cells (maximum killing) minus background Calcein release (targets only). Graphs show means of n=2±SEM.

The anti-MHC-1 positive control and anti-TPBG HC-wt triggered high ADCC activity in human NK cells, whereas all LALA-silenced antibodies and ADCs (anti-TPBG HC-LALA, anti-TPBG-P5(PEG24)-VC-PAB-Exatecan and isotype controls) did not induce ADCC. Reduced ADCC is expected to decrease undesired toxicities of anti-TPBG related antibodies.

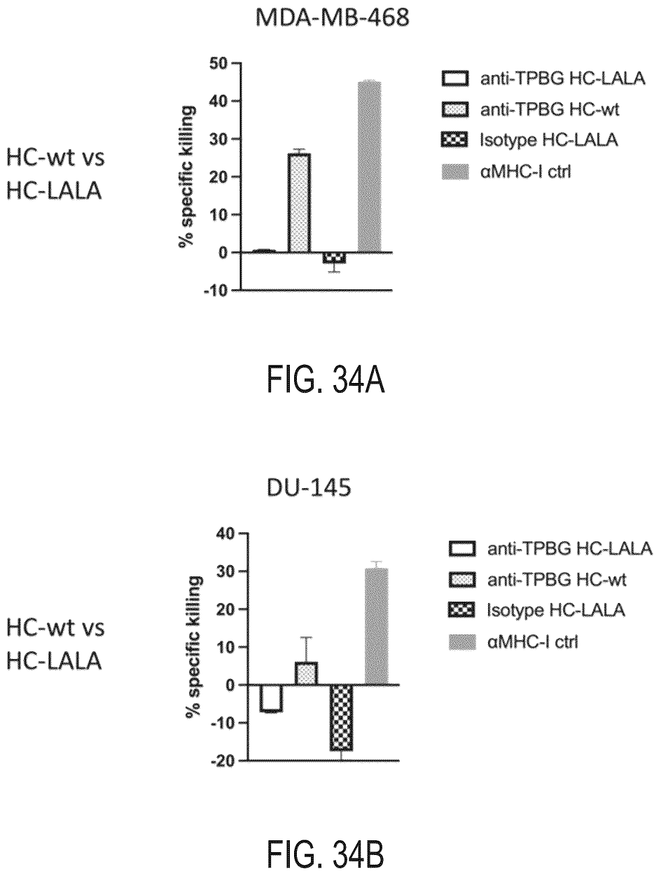

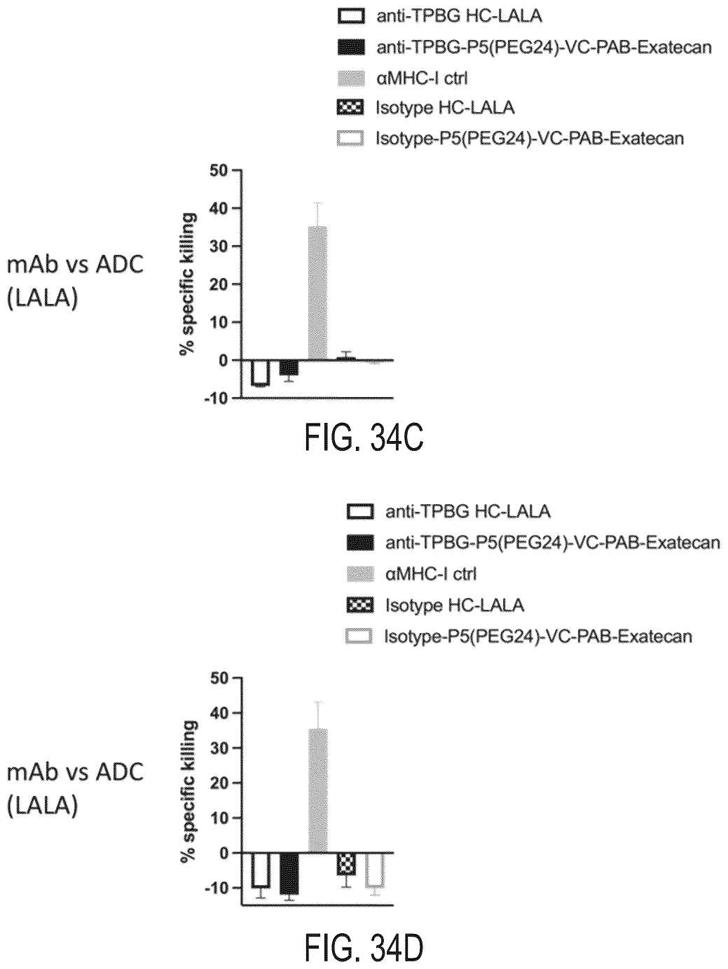

FIGS. 34A to 34D show results of a calcein release-based antibody-dependent cellular cytotoxicity (ADCC) assay. Co-cultures of healthy donor (HD)-derived NK cells and calcein-stained target-positive tumor cells (MDA-MB-468 or DU-145), in a 4:1 ratio, were incubated with 15 μg/ml of indicated antibodies or ADCs (anti-MHC-1 antibody served as positive control). The percentage of specific killing was calculated by dividing the calcein released by antibody-mediated cell killing from calcein released by Triton X-permeabilized cells (maximum killing). Graphs show means of n=2±SEM.

### Anti-TPBG-P5(PEG24)-VC-PAB-Exatecan Dose Response and Exposure PDX Study (HN11218)

All animal experiments were conducted in accordance with German animal welfare law and approved by local authorities. In brief, a 3×3 mm sample of a patient derived tumor sample (HN11218 Head and neck cancer (H&N) model, EPO Experimentelle Pharmakologie & Onkologie Berlin-Buch GmbH) was subcutaneously implanted into flanks of female immunodeficient NMRI nu/nu mice. Treatment was initiated when tumours reached a mean tumour volume of 0.1-0.15 cm³. Animals per group were treated once at day 0 with 5, 3 or 1 mg/kg of anti-TPBG-P5(PEG24)-VC-PAB-Exatecan DAR 8, 5 mg/kg of isotype-P5(PEG24)-VC-PAB-Exatecan DAR 8 or vehicle, as intravenous injection. Tumour volumes, body weights and general health conditions were recorded throughout the whole study.

Complete tumor remission with the 5 mg/kg dose level in a head and neck cancer PDX at a single injection was observed for the targeted anti-TPBG-P5(PEG24)-VC-PAB-Exatecan DAR8 ADC. The 3 mg/kg dosing in the head and neck cancer model also showed strong tumor growth inhibition, while the tumors of the 1 mg/kg dosing started re-growing. No impact in bodyweight gain is highlighting the good tolerability in mice treated with anti-TPBG-P5(PEG24)-VC-PAB-Exatecan DAR8 over all dose-levels.

The PK analysis shows dose proportional exposure profiles and high stability of the anti-TPBG-P5(PEG24)-VC-PAB-Exatecan DAR 8 ADC at the tested dose levels 5, 3 and 1 mg/kg.

The data demonstrate high and long-lasting efficacy for all tested ADC dose levels of anti-TPBG-P5(PEG24)-VC-PAB-Exatecan DAR 8 in the PDX model in vivo compared to the vehicle. The effect is highly specific for the targeted anti-TPBG-antibody, exemplified by the non-targeted isotype control ADC group at the highest dose. No reduction in bodyweight suggests highest tolerability. The PK analysis shows dose proportional exposure profiles and high stability of the anti-TPBG-P5(PEG24)-VC-PAB-Exatecan DAR 8 ADC at the tested dose levels 5, 3 and 1 mg/kg.

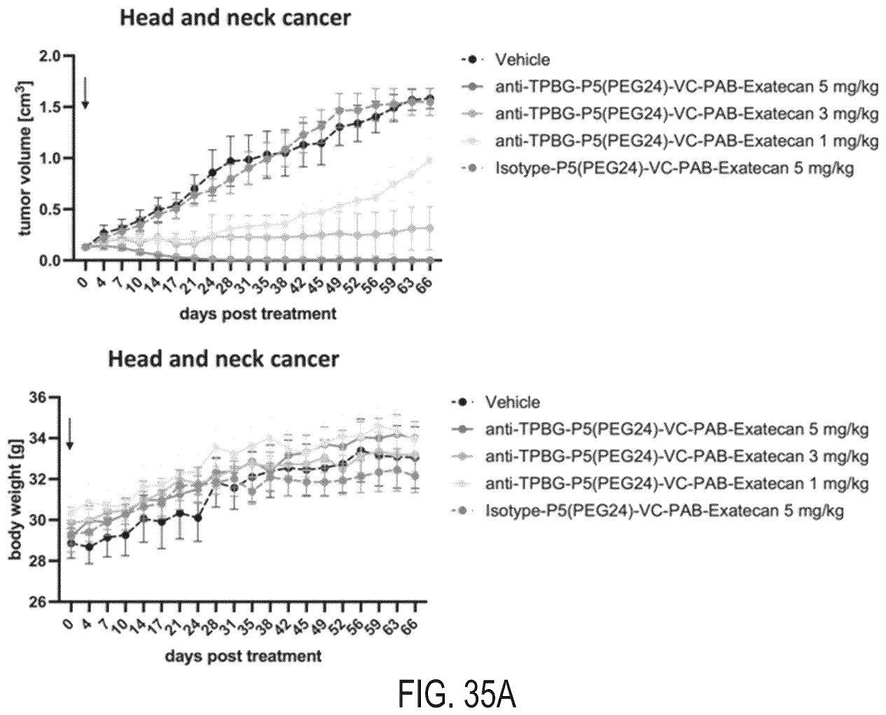

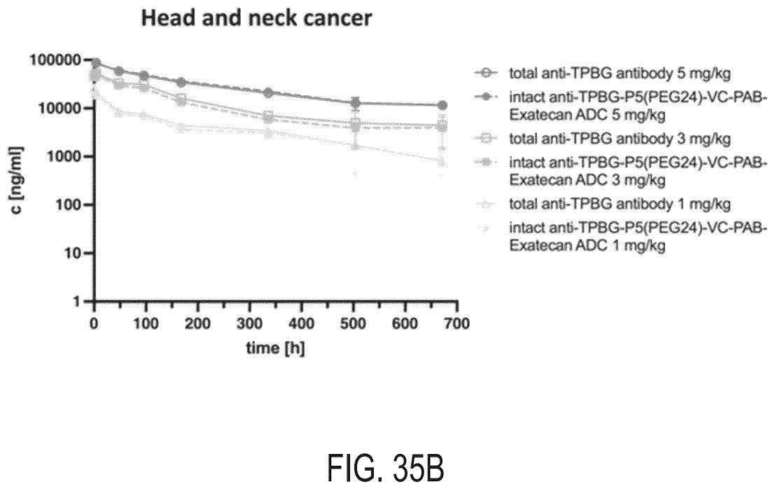

FIG. 35A shows the results of an in vivo efficacy analysis of the anti-TPBG-P5(PEG24)-VC-PAB-Exatecan (DAR 8), which is a representative ADC of the present invention, in a patient-derived head and neck cancer xenograft model (PDX, HN11218, EPO Experimentelle Pharmakologie & Onkologie Berlin-Buch GmbH). Shown on the top is tumor volume over time after treatment once at day 0 with different dose levels of the anti-TPBG-P5(PEG24)-VC-PAB-Exatecan (DAR 8) (1, 3 and 5 mg ADC/kg bodyweight), an isotype control carrying the same amount of linker-payload (5 mg/kg) versus untreated (vehicle). Shown on the bottom are bodyweights of the animals treated with different dose levels of the anti-TPBG-P5(PEG24)-VC-PAB-Exatecan (DAR 8), an isotype control carrying the same amount of linker-payload versus untreated (vehicle). All results are shown as Mean and SEM of 8 animals per group. FIG. 35B shows the in vivo PK evaluation for total and intact ADC for the three dose levels of the dose-response efficacy study as Mean and SD from three measurements per time point from mice treated once at day 0 with different dose levels of the anti-TPBG-P5(PEG24)-VC-PAB-Exatecan (1, 3 and 5 mg ADC/kg bodyweight). The in vivo PK evaluation has been performed as described in example 1.

### Anti-TPBG-P5(PEG24)-VC-PAB-Exatecan Dose Response and Exposure PDX Study (Lu9744)

All animal experiments were conducted in accordance with German animal welfare law and approved by local authorities. In brief, a 3×3 mm sample of a patient derived tumor sample (Lu9744 squamous non-small cell lung cancer (NSCLC) model, EPO Experimentelle Pharmakologie & Onkologie Berlin-Buch GmbH) was subcutaneously implanted into flanks of female immunodeficient NMRI nu/nu mice. Treatment was initiated when tumours reached a mean tumour volume of 0.1-0.15 cm³. Animals per group were treated once at day 0 with 5, 3 or 1 mg/kg of anti-TPBG-P5(PEG24)-VC-PAB-Exatecan DAR 8 or vehicle, as intravenous injection. Tumour volumes, body weights and general health conditions were recorded throughout the whole study.

The data demonstrate high and long-lasting efficacy for all tested ADC dose levels of anti-TPBG-P5(PEG24)-VC-PAB-Exatecan DAR 8 in the PDX model in vivo compared to the vehicle.

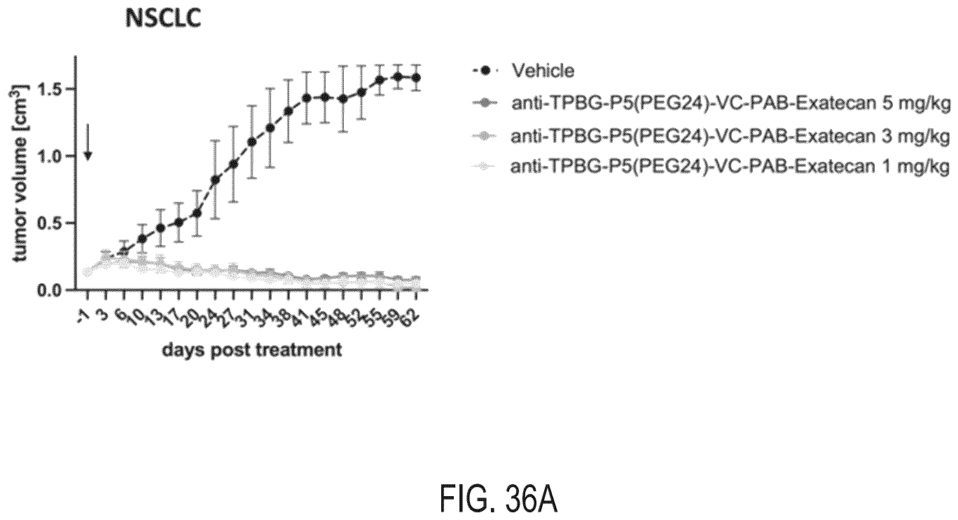

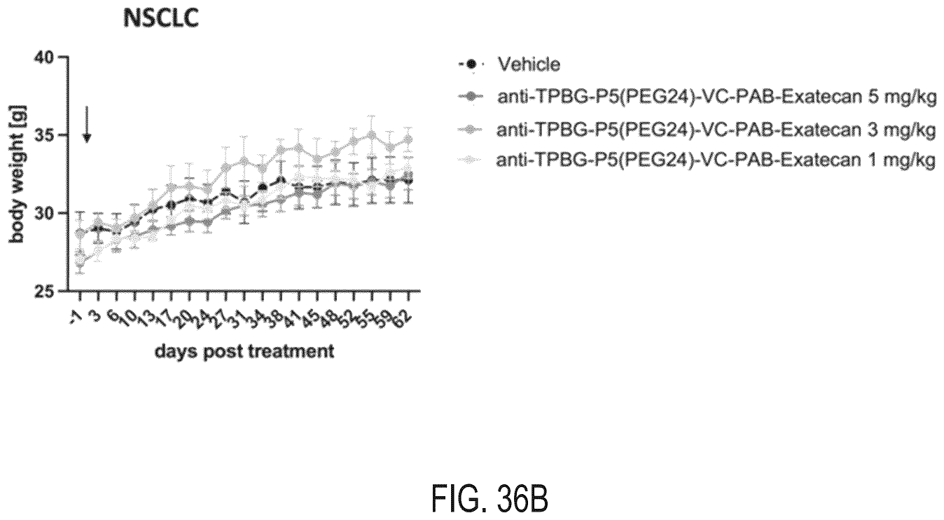

FIGS. 36A and 36B show the results of an in vivo efficacy analysis of the anti-TPBG-P5(PEG24)-VC-PAB-Exatecan (DAR 8), which is a representative ADC of the present invention, in a patient-derived NSCLC xenograft model (PDX, Lu9744). Shown on the left is tumor volume over time after treatment once at day 0 with different dose levels of the anti-TPBG-P5(PEG24)-VC-PAB-Exatecan (DAR 8) (1, 3 and 5 mg ADC/kg bodyweight) versus untreated (vehicle). Shown on the right are bodyweights of the animals treated with different dose levels of the anti-TPBG-P5(PEG24)-VC-PAB-Exatecan (DAR 8) versus untreated (vehicle). All results are shown as Mean and SEM of 4 animals per group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of certain embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied herein may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. All documents, including patent applications and scientific publications, referred to herein are incorporated herein by reference for all purposes.

Other embodiments are within the following claims. In addition, where features or aspects of the invention are described in terms of Markush groups, those skilled in the art will recognize that the invention is also thereby described in terms of any individual member or subgroup of members of the Markush group.

1. An anti-TPBG antibody, wherein said anti-TPBG antibody comprising Fc silencing mutations such as leucine (L) to alanine (A) substitution at the position 234 and 235 (LALA mutations) and is capable of the following:
a) binding to human Trophoblast glycoprotein (TPBG);
b) having cross-reactivity with white-tufted-ear marmoset TPBG; and
c) internalization, optionally by the means of the antigen-mediated antibody internalization.

2. The anti-TPBG antibody according to claim 1, wherein:
(i) said anti-TPBG antibody is an antibody comprising a heavy chain variable region having an amino acid sequence with at least 80% identity to SEQ ID NO: 3 and a light chain variable region having an amino acid sequence with at least 80% identity to SEQ ID NO: 4; optionally said anti-TPBG antibody comprising:
a) a light chain comprising SEQ ID NO: 5 and
b) a heavy chain comprising SEQ ID NO: 6;
and/or
(ii) said anti-TPBG antibody is an antibody comprising:
a′) a heavy chain variable region comprising heavy chain CDR1 having the amino acid sequence as set forth in SEQ ID NO: 7, heavy chain CDR2 having the amino acid sequence as set forth in SEQ ID NO: 8, and heavy chain CDR3 having the amino acid sequence as set forth in SEQ ID NO: 9 and
b′) a light chain variable region comprising light chain CDR1 having the amino acid sequence as set forth in SEQ ID NO: 10, light chain CDR2 having the amino acid sequence as set forth in SEQ ID NO: 11, and light chain CDR3 having the amino acid sequence as set forth in SEQ ID NO: 12.

3. An antibody drug conjugate (ADC) comprising the anti-TPBG antibody according to claim 1.

4. The antibody drug conjugate (ADC) according to claim 3 having the formula (I): 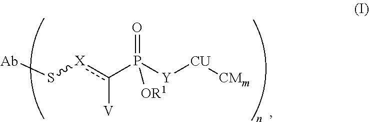
or a pharmaceutically acceptable salt or solvate thereof,
wherein:
Ab is an anti-TPBG antibody according to claim 1; [symbol]
is a double bond; or [symbol]
is a bond;
V is absent when [symbol] is a double bond; or
V is H or (C₁-C₈)alkyl when [symbol] is a bond;
X is R₃—C when [symbol] is a double bond; or
X is 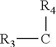
when [symbol] is a bond;
Y is NR⁵, S, O, or CR⁶R⁷;
R¹ is an optionally substituted aliphatic residue or an optionally substituted aromatic residue;
R³ is H; or an optionally substituted aliphatic residue or an optionally substituted aromatic residue;
R⁴ is H; or an optionally substituted aliphatic residue or an optionally substituted aromatic residue;
R⁵ is H; or an optionally substituted aliphatic residue or an optionally substituted aromatic residue;
R⁶ is H; or an optionally substituted aliphatic residue or an optionally substituted aromatic residue;
R⁷ is H; or an optionally substituted aliphatic residue or an optionally substituted aromatic residue;
CU is a connector unit;
CM is a cytotoxic moiety;
m is an integer ranging from 1 to 10; and
n is an integer ranging from 1 to 20.

5. The antibody drug conjugate (ADC) according to claim 4, wherein the cytotoxic moiety CM is selected from the group consisting of camptothecins, maytansinoids, calicheamycins, duocarmycins, tubulysins, amatoxins, dolastatins and auristatins such as monomethyl auristatin E (MMAE), pyrrolobenzodiazepine dimers, indolino-benzodiazepine dimers, radioisotopes, therapeutic proteins and peptides (or fragments thereof), nucleic acids, PROTACs, kinase inhibitors, MEK inhibitors, KSP inhibitors, and analogues or prodrugs thereof.

6. The antibody drug conjugate (ADC) according to claim 5, wherein the cytotoxic moiety CM is a camptothecin moiety.

7. The antibody drug conjugate (ADC) according to claim 6, wherein the camptothecin moiety is selected from the group consisting of exatecan, DXD, SN38, camptothecin, topotecan, irinotecan, belotecan, lurtotecan, rubitecan, silatecan, cositecan, and gimatecan.

8. The antibody drug conjugate (ADC) according to claim 7, wherein the cytotoxic moiety CM is exatecan having the formula:

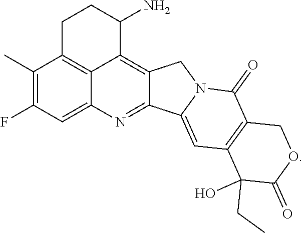 [SMILES: CCC1(O)C(=O)OCc2c1cc1n(c2=O)Cc2c-1nc1cc(F)c(C)c3c1c2C(N)CC3]

9. The antibody drug conjugate (ADC) according to claim 8, wherein the cytotoxic moiety CM is exatecan having the formula:

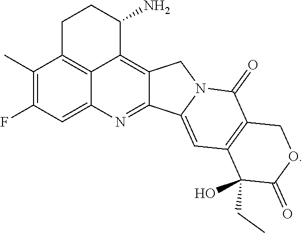 [SMILES: CC[C@@]1(O)C(=O)OCc2c1cc1n(c2=O)Cc2c-1nc1cc(F)c(C)c3c1c2[C@@H](N)CC3]

10. The antibody drug conjugate (ADC) according to claim 8, wherein the exatecan is bound to the connector unit CU via the amino group.

11. The antibody drug conjugate (ADC) according to claim 5,
wherein:
Ab is an anti-TPBG antibody according to claim 1; [symbol]
is a double bond; or [symbol]
is a bond;
V is absent when [symbol] is a double bond; or
V is H when [symbol] is a bond;
X is R₃—C when [symbol] is a double bond; or
X is 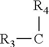
when [symbol] is a bond;
Y is NH;
R¹ is a polyethylene glycol unit having the structure: 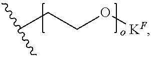
wherein [symbol]
indicates the position of the O;
K^{F} is H; and
o is an integer ranging from 8 to 30;
R³ is H;
R⁴ is H;
CU is a connector unit having the following structure: 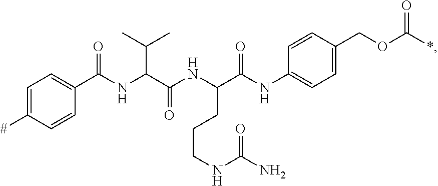
wherein # indicates the attachment point to the Y and * indicates the attachment point to the camptothecin moiety (CM);
CM is a camptothecin moiety;
m is 1; and
n is an integer ranging from 1 to 10.

12. The antibody drug conjugate (ADC) according to claim 11, wherein [symbol] is a double bond; V is absent; X is R₃—C; and R³ is H.

13. The antibody drug conjugate (ADC) according to claim 11, wherein the camptothecin moiety CM is exatecan having the formula:

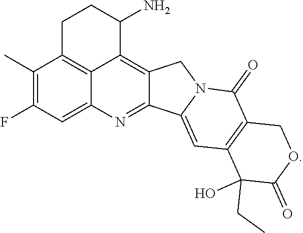 [SMILES: CCC1(O)C(=O)OCc2c1cc1n(c2=O)Cc2c-1nc1cc(F)c(C)c3c1c2C(N)CC3]

14. The antibody drug conjugate (ADC) according to claim 13, wherein the exatecan is bound to the connector unit CU via the amino group.

15. The antibody drug conjugate (ADC) according to claim 14, wherein o ranges from 20 to 28.

16. The antibody drug conjugate (ADC) according to claim 15, wherein o is 22, 23, 24, 25 or 26.

17. The antibody drug conjugate (ADC) according to any one of claim 11, wherein n ranges from 2 to 10, optionally wherein n is 4 or 8.

18. The antibody drug conjugate (ADC) according to claim 17 having the following formula (Ia): 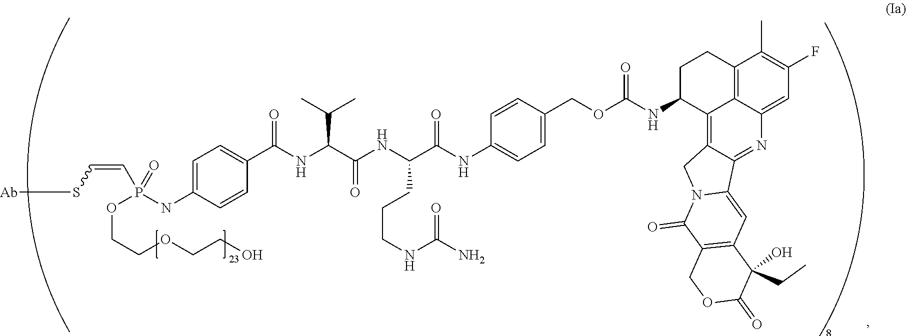
wherein Ab is an anti-TPBG antibody according to claim 1.

19. The antibody drug conjugate (ADC) according to claim 5, wherein the cytotoxic moiety CM is an auristatin.

20. A method of producing an antibody drug conjugate (ADC), said method comprising:
conjugating the antibody according to claim 1 to one or more cytotoxic moieties.

21. A method for treatment, amelioration, prophylaxis and/or diagnostics of cancer, said method comprising: administering a therapeutically or prophylactically effective amount of the antibody drug conjugate (ADC) according to claim 3.
